(19) United States
(12) Patent Application Publication
(10) Pub. No.: US 2020/0278355 A1
(43) Pub. Date: Sep. 3, 2020

(54) CONJUGATED PROTEINS AND USES THEREOF

(71) Applicant: THE SCRIPPS RESEARCH INSTITUTE, La Jolla, CA (US)

(72) Inventors: Benjamin F. CRAVATT, La Jolla, CA (US); Liron BAR-PELED, San Diego, CA (US); Esther KEMPER, San Diego, CA (US)

(21) Appl. No.: 16/650,810

(22) PCT Filed: Sep. 27, 2018
(86) PCT No.: PCT/US2018/053157

§ 371 Date: Mar. 25, 2020

Related U.S. Application Data
(60) Provisional application No. 62/564,223, filed on Sep. 27, 2017.

(51) Int. Cl. G01N 33/68; C07C 233/05

(52) U.S. Cl. CPC G01N 33/6872; G01N 33/6845; C07C 233/05

(57) ABSTRACT

Disclosed herein, in certain embodiments, are protein-probe adducts and synthetic ligands that inhibit protein-probe adduct formation, in which the proteins are regulated by NRF2. In some instances, also described herein are protein-binding domains that interact with a probe and/or a ligand described herein, in which the proteins are regulated by NRF2.

### CROSS-REFERENCE

This application claims the benefit of U.S. Provisional Application No. 62/564,223, filed Sep. 27, 2017, which application is incorporated herein by reference in its entirety.

### STATEMENT AS TO FEDERALLY SPONSORED RESEARCH

The invention disclosed herein was made, at least in part, with the support of the United States government under Grant No. CA132630, by the National Institutes of Health. Accordingly, the U.S. Government has certain rights in this invention.

### BACKGROUND OF THE DISCLOSURE

Protein function assignment has been benefited from genetic methods, such as target gene disruption, RNA interference, and genome editing technologies, which selectively disrupt the expression of proteins in native biological systems. Chemical probes offer a complementary way to perturb proteins that have the advantages of producing graded (dose-dependent) gain- (agonism) or loss- (antagonism) of-function effects that are introduced acutely and reversibly in cells and organisms. Small molecules present an alternative method to selectively modulate proteins and to serve as leads for the development of novel therapeutics.

### SUMMARY OF THE DISCLOSURE

In certain embodiments, described herein are compositions that comprise cysteine-containing proteins that are regulated by NRF2. In some embodiments, disclosed herein is a protein-probe adduct wherein the probe binds to a cysteine residue illustrated in Tables 1A, 2, 3A, and 4; wherein the probe has a structure represented by Formula (I):

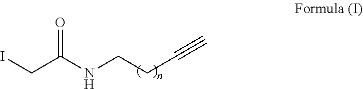

wherein,
n is 0-8.

In some embodiments, disclosed herein is a synthetic ligand that inhibits a covalent interaction between a protein and a probe, wherein in the absence of the synthetic ligand, the probe binds to a cysteine residue illustrated in Tables 1A, 2, 3A, and 4; and wherein the probe has a structure represented by Formula (I):

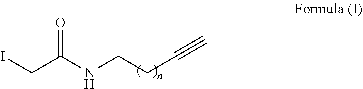

wherein,
n is 0-8

In some embodiments, disclosed herein is a protein binding domain wherein said protein binding domain comprises a cysteine residue illustrated in Tables 1A, 2, 3A, and 4, wherein said cysteine forms an adduct with a compound of Formula I,

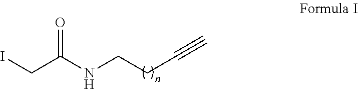

and wherein a compound of Formula IIA or Formula IIB interferes with the formation of the cysteine adduct by the compound of Formula I, wherein Formula (IIA) or Formula (IIB) have the structure:

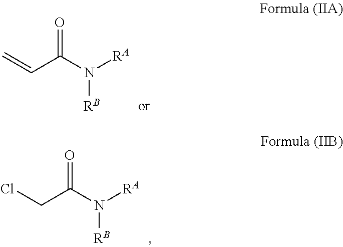

wherein,
each R^{A} and R^{B} is independently selected from the group consisting of H, D, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl; or
R^{A} and R^{B} together with the nitrogen to which they are attached form a 5, 6, 7 or 8-membered heterocyclic ring A, optionally having one additional heteroatom moiety independently selected from NR¹, O, or S; wherein A is optionally substituted; and
   R¹ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1I illustrate chemical proteomic map of NRF2-regulated cysteines in NSCLC cells. FIG. 1A shows proliferation of KEAP1-mutant (H2122) and KEAP1-WT (H1975) cells expressing shRNAs targeting NRF2 (shNRF2) or a control (shGFP), as determined by measuring intracellular ATP concentrations. Data represent mean values+SD (n=6/group). FIG. 1B shows immunoblot of NRF2 in shNRF2- or shGFP-H2122 cells. FIG. 1C shows isoTOP-ABPP (R) ratios for cysteines in shNRF2- or shGFP-H2122 of -H1975 cells. Red data points mark R values≥2.5, which was used as a cutoff for NRF2-dependent changes in cysteine reactivity. Average R values from n=4-5 biological replicates per group are shown. FIG. 1D shows distribution of proteins harboring NRF2-regulated cysteines by functional class. FIG. 1E shows distribution of NRF2-regulated cysteines reflecting changes in reactivity versus protein expression. FIG. 1F shows representative proteins with NRF2-regulated changes in cysteine reactivity. Representative parent mass (MS1) profiles for tryptic peptides with IA-alkyne-reactive cysteines in shNRF2- (red) and shGFP- (blue) H2122 cells. Two cysteines are shown per protein, one with altered and the other with unaltered reactivity between shNRF2- and shGFP-H2122 cells. FIG. 1G shows representative MS1 profiles for cysteine-containing tryptic peptides in SQSTM1 in shNRF2- (red) and shGFP- (blue) H2122 cells (F). FIG. 1H shows immunoblot of GAPDH and PDIA3 expression in shNRF2- and shGFP-H1975 and H2122 cells. FIG. 1I shows GAPDH activity in shNRF2- and shGFP-H2122 and -H1975 cells. Data represent mean values+SD (n=16/group). ****p<0.0001 for shNRF2 versus shGFP groups. FIG. 1J glycolytic flux is impaired in shNRF2-H2122 cells. ECAR=extracellular acidification rate. Data represent mean values+SD (n=20-26/group) from three biological replicates. ***p<0.001, *p<0.05 for shNRF2 versus shGFP groups.

FIG. 2A-FIG. 2E illustrate cysteine ligandability mapping of KEAP1-mutant and KEAP1-WT NSCLC cells. FIG. 2A shows isoTOP-ABPP ratios (R values; DMSO/compound) for cysteines in H2122 cell (KEAP1-mutant) and H358 cell (KEAP1-WT) proteomes treated with DMSO or ‘scout’ fragments 2 or 3 (500 μM, 1 h). Red data points mark R values≥5, which was used as a cutoff for defining liganded cysteines. Average R values from n=3 biological replicates per group are shown. FIG. 2B shows a pie chart of NRF2-regulated genes/proteins in NSCLC cell lines denoting the subset that contain liganded cysteines (red). FIG. 2C shows cysteine ligandability map for representative NRF2 pathways. Blue marks proteins with liganded cysteines in NSCLC cells. ND, not detected. FIG. 2D shows Circos plot showing the overlap in liganded cysteines between KEAP1-mutant (red) and KEAP1-WT (black) NSCLC cells. Gray and blue chords represent liganded cysteines found in both KEAP1-WT and KEAP1-mutant cell lines and selectively in KEAP1-mutant cell lines, respectively. Numbers in parenthesis indicate total liganded cysteines per cell line. FIG. 2E shows immunoblot of AKR1B10, CYP4F11 and NR0B1 in shNRF2- and shGFP-H2122 cells.

FIG. 3A-FIG. 3B illustrate Characterization of liganded proteins selectively expressed in KEAP1-mutant NSCLC cells. FIG. 3A shows Heat map depicting RNAseq data in KEAP1-WT and KEAP1-mutant NSCLC cell lines for genes encoding NRF2-regulated proteins with liganded cysteines. RNAseq data obtained from (Klijn et al., Nat Biotechnol 33, 306-312, 2015) (also see FIG. 9A). FIG. 3B shows NR0B1, AKR1B10, and CYP4F11 expression in lung adenocarcinoma (LUAD) tumors grouped by NRF2/KEAP1 mutational status. Data obtained from TCGA.

FIG. 4A-FIG. 4E illustrate NR0B1 nucleates a transcriptional complex that supports the NRF2 gene-expression program. FIG. 4A shows intersection between NR0B1-regulated genes and transcriptional start sites (TSSs) bound by NR0B1. Outer circle: Chromosomes with cytogenetic bands. Middle circle: Whole genome plot of mapped NR0B1 reads (black) determined by ChIP-Seq corresponding to the transcriptional start sites (TSSs) of genes differentially expressed (up- (blue) or down- (red) regulated >1.5-fold) in shNR0B1-H460 cells compared to shGFP-H460 cells (inner circle). FIG. 4B shows overlap (left) and correlation (right) between genes up- (red) or down- (blue) regulated (>1.5-fold) in shNR0B1- and shNRF2-H460 cells compared to shGFP-H460 control cells. r and p values were determined by Pearson correlation analysis. FIG. 4C shows Heat map depicting RNAseq data for the indicated genes in shNR0B1-, shNRF2-, or shGFP-H460 cells. Expression was normalized by row. FIG. 4D shows Heat map representing NR0B1-interacting proteins in NSCLC cells. FIG. 4E shows endogenous NR0B1 co-immunoprecipitates with FLAG-RBM45 and FLAG-SNW1, but not control protein FLAG-RAP2A, in H460 cells, as determined by immunoblotting (left); right: schematic of NR0B1 protein interactions.

FIG. 5A-FIG. 5G show covalent ligand targeting C274 disrupts NR0B1 protein complexes. FIG. 5A shows co-crystal structure of mouse NR0B1 (white) and LRH1 (burnt orange) from (Sablin et al., 2008) highlighting the location of C274 (orange) at the protein interaction interface that is also flanked by AHC mutations: R267, V269 and L278 (red). FIG. 5B shows a schematic for an NR0B1-SNW1 in vitro-binding assay (Left) and an immunoblot showing that NR0B1 interacts with SNW1, but not a control (METAP2) protein (Right). FIG. 5C shows small molecule screen of electrophilic compounds (50 μM) for disruption of binding of FLAG-SNW1 to NR0B1 as shown in (B). Percentage of NR0B1 bound to SNW1 was normalized to vehicle (DMSO). A hit compound BPK-26 is marked in red. FIG. 5D shows structures of NR0B1 ligands (BPK-26 and BPK-29), clickable probe (BPK-29yne), and inactive control compounds (BPK-9 and BPK-27). FIG. 5E shows BPK-26 and BPK-29, but not BPK-9 and BPK-27, disrupt the in vitro interaction of FLAG-SWN1 with NR0B1. FIG. 5F shows BPK-29yne labels WT-NR0B1, but not an NR0B1-C274V mutant. HEK293T cells expressing the indicated proteins were treated with BPK-29 or vehicle (3 h) prior to treatment with BPK-29yne (30 min). Immunoprecipiated proteins were analyzed by in-gel fluorescence-scanning and immunoblotting. FIG. 5G shows BPK-29 disrupts protein interactions for NR0B1-WT, but not a NR0B1-C274V mutant. HEK293T cells expressing HA-NR0B1-WT or HA-NR0B1-C274V proteins were treated with DMSO or BPK-29, after which lysates were generated and evaluated for binding to FLAG-SNW1, as shown in (B).

FIG. 6A-FIG. 6F show characterization of NR0B1 ligands in KEAP1-mutant NSCLC cells. FIG. 6A shows isoTOP-ABPP of H460 cells treated with NR0B1 ligands and control compounds (40 μM, 3 h). Dashed lines designate R values≥3 (DMSO/compound), which was used as a cutoff to define cysteines liganded by the indicated compounds. Insets show MS1 profiles for C274 in NR0B1 for DMSO (blue) versus compound (red) treatment. Data are from individual experiments representative of at least three biological replicates. FIG. 6B shows a Venn diagram comparing the proteome-wide selectivity of NR0B1 ligands BPK-29 and BPK-26 and control compounds BPK-9 and BPK-27 in H460 cells as determined in (A). (See also Table 5). FIG. 6C shows BPK-29 and BPK-26 block the RBM45-NR0B1 interaction in H460 cells. H460 cells stably expressing FLAG-RBM45 were incubated with indicated compounds for 3 h, whereupon FLAG immunoprecipitates were performed and analyzed by immunoblotting. FIG. 6D shows concentration-dependent blockade of NR0B1 binding to FLAG-RBM45 by BPK-29 (left) and BPK-26 (right) in H460 cells. Experiments performed as described in (C). FIG. 6E shows SILAC ratio plots for light amino acid-labeled cells (pulse phase) switched into media containing heavy amino acids for 3 h (chase phase) followed by proteomic analysis. Dashed line designates R values (light/heavy) of <8, which was used as a cutoff for fast-turnover proteins. Inset shows MS1 peak ratio for NR0B1, which is among the top 5% of fast-turnover proteins. FIG. 6F shows proteins regulated by NRF2 in NSCLC cells are enriched in fast-turnover proteins. Charts comparing fraction of NRF2-regulated genes (as determined by RNAseq) for which the corresponding proteins are designated as fast or slow turnover (as determined in G) further divided into groups showing reduced expression (left) or not (right) on day 1 following NRF2 knockdown (as determined by isoTOP-ABPP).

FIG. 7A-FIG. 7L illustrate chemical proteomic map of NRF2-regulated cysteines in NSCLC cells. FIG. 7A shows immunoblot of NRF2 in H1975 (KEAP1-WT) and H2122 (KEAP1-mutant) cells. FIG. 7B shows immunoblot of NRF2 in H460 and A549 cells expressing shRNAs targeting NRF2 or GFP (control). FIG. 7C shows proliferation rates of KEAP1-mutant NSCLC cells expressing shRNAs targeting NRF2 (shNRF2) or a GFP control (shGFP), as determined by measuring intracellular ATP concentrations. Data represent mean values+SD (n=6/group). FIG. 7D shows proliferation rate of KEAP1-WT NSCLC H2009 cells expressing shRNAs targeting NRF2 (shNRF2) or a GFP control (shGFP), as determined by measuring intracellular ATP concentrations. Data represent mean values+SD (n=6/group). FIG. 7E shows intracellular GSH content in shNRF2- or shGFP-H2122 or -H1975 cells. Data represent mean values+SD (n=11/group), ****p<0.0001 for shNRF2 vs shGFP. FIG. 7F shows cytosolic H₂O₂ content is increased in shNRF2-H2122, but not shGFP-H2122 cells or shNRF2- or shGFP-H1975 cells. FACS analysis of cells treated with a PF6-AM probe that measures cytosolic H₂O₂. Data are representative plots from two biological replicates. FIG. 7G shows a schematic for the identification of NRF2-regulated cysteines by isoTOP-ABPP. Proteomes from cells expressing shRNAs as described in FIG. 7A are labeled with an alkynylated iodoacetamide probe (IA-alkyne, compound 1). Cysteines that are oxidized or modified with an electrophile (denoted as X) following NRF2 knockdown cannot further react with IA-alkyne. IA-alkyne-modified cysteines are conjugated by copper-catalyzed azide-alkyne cycloaddition (CuAAC or click) chemistry to isotopically differentiated azide-biotin tags, each containing a TEV cleavage sequence. The light (shNRF2) and heavy (shGFP) samples are mixed, and the IA-alkyne modified peptides are enriched and identified by liquid chromatography tandem mass-spectrometry (LC-MS/MS). The relative reactivity of cysteine residues in shGFP and shNRF2 samples is measured by quantifying the MS1 chromatographic peak ratios (heavy/light). In the theoretical example on the right, two cysteines are identified, with the one residue showing a five-fold quantified decrease in reactivity following NRF2 knockdown. FIG. 7H shows a timeline for measuring changes in cysteine reactivity by isoTOP-ABPP following NRF2 knockdown. FIG. 7I shows changes in cysteine reactivity following NRF2 knockdown at the indicated time points. FIG. 7J shows comparison of cysteine reactivity changes in H2122 or H1975 cells following NRF2 knockdown or treatment with staurosporine or AZD9291. H2122 and H1975 cells were treated with staurosporine (1 μM, 4 h). H1975 cells were treated with AZD9291 (1 μM, 24 h). Changes in cysteine reactivity were determined by isoTOP-ABPP as described in FIG. 7G. FIG. 7K shows analysis of apoptosis induction in NSCLC cells treated with staurosporine and EGFR blockade in H1975 cells treated with AZD9291. H2122 and H1975 cells were treated with staurosporine (1 μM, 4 h). H1975 cells were treated with AZD9291 (1 μM, 24 h). Apoptosis induction was assessed by measuring PARP1 cleavage; EGFR blockade was assessed by measuring autophosphorylation of residue Y1068. Proteins were analyzed by immunoblotting. FIG. 7L shows representative MS1 chromatograms of tryptic peptides containing IA-alkyne-reactive cysteines identified in isoTOP-ABPP experiments comparing shNRF2- (red) and shGFP- (blue) H1975 cells.

FIG. 8A-FIG. 8F illustrate cysteine ligandability landscape of KEAP1-mutant and KEAP1-WT NSCLC cells. FIG. 8A shows identification of liganded cysteines in NSCLC cell lines. isoTOP-ABPP ratios (R values; DMSO/compound) for cysteines in KEAP1-mutant (H460, A549) proteomes treated with DMSO or ‘scout’ fragments 2 or 3 (500 μM, 1 h). Red data points mark R values≥5, which was used as a cutoff for defining 2- or 3-liganded cysteines. Aggregate R values from n=3 biological replicates per group are shown. For cysteines quantified in more than one biological replicate, average ratios are reported. FIG. 8B shows identification of liganded cysteines in NSCLC cell lines. isoTOP-ABPP ratios (R values; DMSO/compound) for cysteines in KEAP1-WT (H1975, H2009 (expressing the luciferase protein)) proteomes treated with DMSO or ‘scout’ fragments 2 or 3 (500 μM, 1 h). Red data points mark R values≥5, which was used as a cutoff for defining 2- or 3-liganded cysteines. Aggregate R values from n=3 biological replicates per group are shown. For cysteines quantified in more than one biological replicate, average ratios are reported. FIG. 8C shows NRF2-regulated proteins and genes, defined as proteins showing reductions in cysteine reactivity (R values≥2.5) in isoTOP-ABPP experiments and genes showing reduction (≥2) in mRNA expression in RNA-seq experiments (see FIG. 1F). Gene expression changes were compiled from shNRF2-H2122 and shNRF2-H460 cells and siNRF2-A549 cells. Genes were defined as NRF2-regulated if they showed a two-fold or greater reduction in expression in two or more data sets. Proteins found to be regulated by NRF2 by both isoTOP-ABPP and RNA-seq are designated as “cysteine reactivity” in the graph. FIG. 8D shows Heat map summarizing liganded cysteines found in NRF2-regulated proteins across KEAP1-mutant and KEAP1-WT NSCLC cell lines. Cysteines were required to be liganded (R values≥5) by fragments 2 and/or 3 in two or more KEAP1-mutant or KEAP1-WT NSCLC lines for inclusion in the heat map. FIG. 8E shows immunoblot of AKR1B10, CYP4F11 and NR0B1 proteins in shNRF2- and shGFP-H460 cells. FIG. 8F shows NRF2 regulates the transcription of NR0B1, AKR1B10, and CYP4F11 genes as determined by RNAseq of H2122 or H460 cells expressing the indicated shRNAs. Data were normalized to shGFP and represent mean values+SD (n=3/group).

FIG. 9A-FIG. 9C illustrate characterization of liganded proteins selectively expressed in KEAP1-mutant NSCLC cells. FIG. 9A shows AKR1B10, CYP4F11 and NR0B1 expression is restricted to KEAP1-mutant cells. RNAseq analysis of genes encoding proteins with cysteine reactivity changes in NSCLC cell lines (see FIG. 8D) was determined across a panel of KEAP1-WT and KEAP1-mutant NSCLC cell lines. The graph displays the ratio of the average expression of the indicated genes (KEAP1-mutant/KEAP1-WT), with genes having a three-fold or greater difference marked in red. Also see FIG. 3A. FIG. 9B shows immunoblot of NR0B1, ARK1B10, and CYP4F11 expression across a representative panel of KEAP1-WT and KEAP1-mutant NSCLC cell lines. FIG. 9C shows expression of NRF2-regulated proteins/genes across normal tissues as measured by RNAseq. Expression was assessed for 53 human tissues from the GTEx portal (gtexportal.org). Genes were considered expressed in a given tissue if they had RPKM values>1. Liganded NRF2-regulated proteins were defined as those showing R values≥2.5 in isoTOP-ABPP experiments of shNRF2-NSCLC cells or reduced by gene expression (e.g., see FIG. 1E and FIG. 2D) and supplemented by NRF2-regulated genes as determined in (Goldstein et al., 2016). The subset of NRF2-regulated proteins/genes that were found to be liganded by scout fragments 2 and/or 3, including AKR1B10, CYP4F11, and NR0B1, are designated.

FIG. 10A-FIG. 10G illustrate NR0B1 nucleates a transcriptional complex that supports the NRF2 gene-expression program. FIG. 10A shows representative top-scoring functional terms enriched in genes down-regulated in shNR0B1-H460 cells compared to shGFP-H460 cells. Scores are calculated based on Benjamini-Hochberg corrected p-values. FIG. 10B shows Myc and E2F gene signatures are enriched in NR0B1-regulated genes. Gene set enrichment analysis (GSEA) was applied to all genes that were differentially expressed between shNR0B1-H460 cells and shGFP-H460 cells. Genes were ranked based on their FDR value. The FDR q-value was computed by GSEA. FIG. 10C shows identification of NR0B1-interacting proteins. FLAG immunoprecipitates were prepared from A549 cells expressing FLAG-NR0B1 or FLAG-METAP2 (control), and the proteins found in these immunoprecipitates were identified by LC-MS/MS. Enrichment of FLAG-NR0B1-interacting proteins was determined by taking the ratio between protein interactions with FLAG-NR0B1 and the control protein FLAG-METAP2. The dashed line marks proteins with a ratio above 20 (red) designated as FLAG-NR0B1 binding partners. FIG. 10D shows endogenous NR0B1 co-immunoprecipitates with FLAG-RBM45 or FLAG-SNW1 in A549 and H2122 cells. FLAG immunoprecipitates were prepared from A549 and H2122 cells stably expressing FLAG-SNW1 (left) or FLAG-RBM45 (right), or FLAG-RAP2A as a control. Cell lysates and immunoprecipitates were analyzed by immunoblotting for the indicated proteins. FIG. 10E shows NR0B1 nucleates a complex with SNW1 and RBM45. Recombinant HA-SNW1 co-immunoprecipitates FLAG-RBM45 in the presence, but not absence, of FLAG-NR0B1. HA immunoprecipitates were prepared from the indicated transfected HEK293T cells. HA immunoprecipitates were analyzed as above (D). FIG. 10F shows NR0B1 and NR0B1-interacting proteins (SNW1 and RBM45) colocalize to the nucleus. Images of A549 cells stably expressing FLAG-SNW1 or FLAG-RBM45 were co-immunostained for NR0B1, FLAG, HOECHST, and NQO1. Insets show selected fields that were magnified five times and their overlays. Scale bar=10 μm. FIG. 10G shows NR0B1 and SNW1-regulated genes in H460 cells are positively correlated as determined by Pearson correlation analysis. Genes in red are co-downregulated (≤1.5 fold) and genes in blue are co-upregulated (≥1.5 fold).

FIG. 11A-FIG. 11F illustrate a covalent ligand targeting Cys274 disrupts NR0B1 protein complexes. FIG. 11A shows structures and activities of BPK-26 and related compounds. See also FIG. 5C. FIG. 11B shows generating an advanced NR0B1 ligand. Top: Structures of screening hit BPK-28 and synthesized derivatives. Middle: Relative inhibition of FLAG-SNW1 binding to NR0B1 by BPK-28 and derivatives identifies BPK-29 as the most potent analogue (red). The In vitro-binding assay was performed as described in FIG. 5B using compounds at a concentration of 50 μM. Bottom: Data represent mean values±SD normalized to DMSO control. n=4/group. FIG. 11C shows concentration-dependent inhibition of the NR0B1-SNW1 interaction by NR0B1 ligands BPK-26 and BPK-29 and control compounds BPK-27 and BPK-9. Top: Compounds were tested as described in FIG. 5B. Bottom: Graph of concentration-dependent inhibition of NR0B1-SNW1 interactions by the indicated compounds. Percent binding was normalized to vehicle (DMSO). Data represent mean values±SD (n=2-5/group). FIG. 11D and FIG. 11E show NR0B1 ligands BPK-26 (D) and BPK-29 (E) covalently modify C274 in NR0B1. Lysate generate from HEK293T cell expressing FLAG-NR0B1 was treated with DMSO or BPK-26 (100 μM, 3 h, D). Alternatively, HEK293T cell expressing FLAG-NR0B1 were treated with DMSO or BPK-29 (50 μM, 3 h) in serum/dye-free RPMI (E) and lysates were generated. FLAG-immunoprecipitates were prepared from each lysate and subjected to proteolytic digestion, whereupon tryptic peptides harboring C274 were analyzed by LC-MS/MS. Extracted ion chromatogram for m/z value of the NR0B1 BPK-26- or BPK-29-modified tryptic peptide (m/z=1228.5992 and 1289.126, respectively) showing signals in BPK-26 or BPK-29-treated (blue), but not DMSO-treated (red) HEK293T cell samples. FIG. 11F shows BPK-29 competition of BPK-29yne labeling of NR0B1. HEK293T cells transiently expressing FLAG-NR0B1 were treated with BPK-29, control compound BPK-27, or vehicle for 3 h prior to treatment with BPK-29yne (30 min). Following cell lysis, FLAG-tagged proteins were immunoprecipiated and conjugated to an azide-TAMRA tag by CuAAC conjugation. Immunoprecipitates were analyzed by in-gel fluorescence-scanning to assess BPK-29yne labeling or by immunoblot for FLAG-NR0B1. C274 is required for BPK-26 inhibition of NR0B1. In a modified in vitro binding assay shown in FIG. 5B, HEK293T cells expressing HA-NR0B1-WT or an HA-NR0B1-C274V mutant were treated with DMSO or BPK-26 (20 μM, 3 h), after which lysates were and interaction with FLAG-SNW1 assessed.

FIG. 12A-FIG. 12G show characterization of NR0B1 ligands in Keap1-mutant NSCLC cells. FIG. 12A shows representative MS1 profiles showing concentration-dependent blockade of IA-alkyne labeling of C274 of NR0B1 (left) or C29 of TXN2 (middle) by BPK-29 and/or BPK-26 (right). Data obtained from isoTOP-ABPP experiments of H460 cells treated with compound (red traces) or DMSO (blue traces) for 3 h. FIG. 12B shows BPK-29 and BPK-26 selectively block IA-alkyne labeling of C274 among several other cysteine residues in NR0B1 quantified by isoTOP-ABPP. Shown are MS1 profiles for quantified cysteines in NR0B1 following treatment with BPK-29 (40 μM, red; top) BPK-26 (40 μM, red; bottom) or DMSO (blue) for 3 h. FIG. 12C shows schematic for BPK-29 competition experiments using the BPK-29yne probe in NSCLC cell lines. FIG. 12D shows CRISPR-generated KEAP1-null and NRF2-null HEK293T cells were analyzed for the expression of the indicated proteins by immunoblotting. FIG. 12 E shows BPK-29 and BPK-26 inhibit NR0B1 interaction with FLAG-RBM45 or FLAG-SNW1 in KEAP1-null HEK293T cells. KEAP1-null HEK293T cells stably expressing FLAG-RBM45 or FLAG-SNW1 were incubated with the indicated compounds for 3 h, after which FLAG immunoprecipitates were prepared from cell lysates. Immunoprecipitates and lysates were analyzed by immunoblotting for the indicated proteins. Dashed lines represent a lane that was cropped from this immunoblot. FIG. 12F shows BPK-29 and BPK-26 block NR0B1 binding to FLAG-RBM45 in H2122 and A549 cells. H2122 or A549 cells stably expressing FLAG-RBM45 were incubated with the indicated compounds for 3 h, after which FLAG immunoprecipitates were prepared. Immunoprecipitates and lysates were analyzed as described in (E). FIG. 12G shows concentration-dependent blockade of NR0B1 binding to its interacting proteins by BPK-29 and BPK-26 in H2122 and A549 cells. H2122 cells stably expressing FLAG-RBM45 or A549 cells stably expressing FLAG-SNW1 were incubated with indicated compounds for 3 h and FLAG immunoprecipitates were prepared and analyzed as described in (E).

FIG. 13A-FIG. 13E illustrate characterization of NR0B1 ligands in Keap1-mutant NSCLC cells. FIG. 13A shows representative genes co-downregulated in BPK-29-treated, shNR0B1, and shNRF2 H460 cells. Top: Heat map depicting changes in gene expression between H460 cells expressing shNRF2, shNR0B1 or a control (shGFP) and those treated with vehicle (DMSO), BPK-29 or BPK-9 (30 μM, 12 h). Expression for each condition was first normalized to appropriate controls (shGFP or DMSO) and then normalized by row. Bottom: Overlap between gene sets regulated in BPK-29-treated vs shNR0B1 H460 cells. Gene set enrichment analysis (GSEA) was applied to all genes that were differentially expressed between shNR0B1-H460 cells and shGFP-H460 cells or between H460 cells treated with BPK-29 or DMSO. Genes were ranked based on their FDR. The FDR q-value was computed by GSEA on the C2.all collection and a cut off of FDR<0.05 was required for a gene set to be considered enriched. FIG. 13B shows BPK-29 alters the expression of representative genes in KEAP1-mutant H460 cells, but not KEAP1-WT H2009 cells. H460 (left) or H2009 (right) cells were treated with vehicle, BPK-29, or BPK-9 (25 μM, 12 h). Gene expression changes for CRY1, DEPDC1, and CPLX2 were determined by qPCR and data represents mean values+SD (n=4-10). FIG. 13C shows BPK-29 alters the expression of representative genes in KEAP1-mutant H2122 cells. Cells were treated with the vehicle, BPK-29, or BPK-9 (30 μM, 12 h). Gene expression changes for Cry1, DEPDC1, and CPLX2 were determined by qPCR and data represents mean values+SD (n=4-6). FIG. 13D shows BPK-29 reduces CRY1 protein content in H460 cells. H460 cells were treated with vehicle or BPK-29 or BPK-9 at the indicated concentrations for 9 h. Protein expression was analyzed by immunoblotting. FIG. 13E shows NR0B1 is a rapidly degraded protein. Top: H460 cells were treated with cycloheximide (100 μg/mL) for the indicated time points and NR0B1 protein content assessed by immunoblotting. Bottom: NR0B1 half-life analysis. NR0B1 protein content was determined following cycloheximide treatment and data were fit into a one-phase exponential decay model. Data represent mean values+SD (n=4-10).

FIG. 14A-FIG. 14D illustrate an exemplary compound library described herein.

### DETAILED DESCRIPTION OF THE DISCLOSURE

Cancer cells rewire central metabolic networks to provide a steady source of energy and building blocks needed for cell division and rapid growth. This demand for energy produces toxic metabolic byproducts, including reactive oxygen species (ROS), that, if left unchecked in some cases, promotes oxidative stress and impair cancer cell viability. Many cancers counter a rise in oxidative stress by activating the NRF2 pathway, a master regulator of the cellular antioxidant response. Under basal conditions, the bZip transcription factor NRF2 binds to the negative regulator KEAP1, which directs rapid and constitutive ubiquitination and proteasomal degradation of NRF2. Under conditions of oxidative stress, one or more cysteines in KEAP1 are oxidatively modified to block interaction with NRF2, stabilizing the transcription factor to allow for nuclear translocation and coordination of a gene expression program that induces detoxification and metabolic enzymes to restore redox homeostasis. Cancers stimulate NRF2 function in multiple ways, including genetic mutations in NRF2 and KEAP1 that disrupt their interaction and are found in >20% of non-small cell lung cancers (NSCLCs). Despite maturation in understanding how NRF2 becomes activated and promotes a transcriptional program that responds to oxidative stress, the underlying molecular mechanisms by which stimulation of this pathway imparts a survival and growth advantage to cancer cells remain poorly defined. Moreover, to date, only a handful of early-stage small molecules have been identified that inhibit NRF2 function, and as a consequence, oncogenic mutations in the KEAP1-NRF2 complex remain unactionable from a therapeutic perspective.

In some instances, cysteine plays several roles in protein regulations, including as nucleophiles in catalysis, as metal-binding residues, and as sites for post-translational modification. While low levels of ROS can stimulate cell growth, excessive ROS has damaging effects on many fundamental biochemical processes in cells, including, for instance, metabolic and protein homeostasis pathways. In some cases, activation of NRF2 in cancer cells serves to protect biochemical pathways from ROS-induced functional impairments.

Cysteine residues not only constitute sites for redox regulation of protein function, but also for covalent drug development. Both catalytic and non-catalytic cysteines in a wide range of proteins have been targeted with electrophilic small molecules to create covalent inhibitors for use as chemical probes and therapeutic agents. Some include, for example, ibrutinib, which targets Bruton's tyrosine kinase BTK for treatment of B-cell cancers and afatinib and AZD9291, which target mutant forms of EGFR for treatment of lung cancer.

Described herein, in certain embodiments, are protein-probe adducts and synthetic ligands that inhibit protein-probe adduct formation, in which the proteins are regulated by NRF2. In some instances, also described herein are protein-binding domains that interact with a probe and/or a ligand described herein, in which the proteins are regulated by NRF2.

In some embodiments, further described herein is a method of modulating or altering recruitment of neosubstrates to the ubiquitin proteasome pathway. In some instances, the method comprises covalent binding of a reactive residue on one or more proteins described below for modulation of substrate interaction. In some cases, the method comprises covalent binding of a reactive cysteine residue on one or more proteins described below for substrate modulation.

### Small Molecule Compounds

In some embodiments, described herein is a probe with a structure represented by Formula (I):

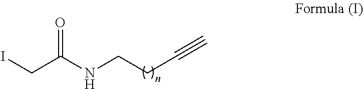

in which n is 0-8. In some instances, n is 1, 2, 3, 4, 5, 6, 7, or 8. In some instances, n is 1. In some instances, n is 2. In some instances, n is 3. In some instances, n is 4. In some instances, n is 5. In some instances, n is 6. In some instances, n is 7. In some instances, n is 8.

In some embodiments, described herein is a synthetic ligand having a structure represented by Formula II:

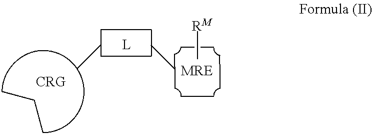

wherein,
CRG-L is optional, and when present is a covalent reactive group comprising a Michael acceptor moiety, a leaving group moiety, or a moiety capable of forming a covalent bond to the thiol group of a cysteine residue, and L is a linker;
MRE is a molecular recognition element that is capable of interacting with the protein; and
R^{M} is optional, and when present comprises a binding element that binds to a second protein or another compound.

In some embodiments, the Michael acceptor moiety comprises an alkene or an alkyne moiety. In some embodiments, the Michael acceptor moiety comprises an alkene moiety. In some embodiments, the Michael acceptor moiety comprises an alkyne moiety.

In some embodiments, L is a cleavable linker.

In some embodiments, L is a non-cleavable linker.

In some embodiments, MRE comprises a small molecule compound, a polynucleotide, a polypeptide or fragments thereof, or a peptidomimetic. In some embodiments, MRE comprises a small molecule compound. In some embodiments, MRE comprises a polynucleotide. In some embodiments, MRE comprises a polypeptide or fragments thereof. In some embodiments, MRE comprises a peptidomimetic.

In some embodiments, the synthetic ligand has a structure represented by Formula (IIA) or Formula (IIB):

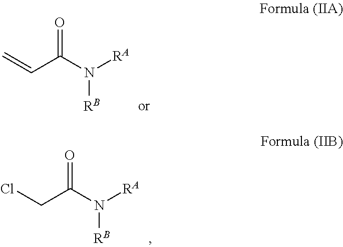

wherein,
each R^{A} and R^{B} is independently selected from the group consisting of H, D, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl; or
R^{A} and R^{B} together with the nitrogen to which they are attached form a substituted or unsubstituted 5, 6, 7 or 8-membered heterocyclic ring A, optionally having one additional heteroatom moiety independently selected from NR¹, O, or S; and
R¹ is H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl.

In some embodiments, R^{A} is substituted or unsubstituted aryl, substituted or unsubstituted C₁-C₃alkylene-aryl, substituted or unsubstituted heteroaryl, or substituted or unsubstituted C₁-C₃alkylene-heteroaryl. In some embodiments, R^{A} is substituted or unsubstituted aryl. In some embodiments, R^{A} is substituted or unsubstituted C₁-C₃alkylene-aryl. In some embodiments, R^{A} is substituted or unsubstituted heteroaryl. In some embodiments, R^{A} is substituted or unsubstituted C₁-C₃alkylene-heteroaryl.

In some embodiments, R^{B} is substituted or unsubstituted C₂-C₇heterocycloalkyl, substituted or unsubstituted aryl, or substituted or unsubstituted heteroaryl. In some embodiments, R^{B} is substituted or unsubstituted C₂-C₇heterocycloalkyl. In some embodiments, R^{B} is substituted or unsubstituted aryl. In some embodiments, R^{B} is substituted or unsubstituted heteroaryl.

In some embodiments, R^{B} is substituted C₅-C₇heterocycloalkyl, substituted with —C(═O)R², wherein R² is substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl. In some embodiments, R² is substituted or unsubstituted C₁-C₆alkyl. In some embodiments, R² is substituted or unsubstituted C₁-C₆fluoroalkyl. In some embodiments, R² is substituted or unsubstituted C₁-C₆heteroalkyl. In some embodiments, R² is substituted or unsubstituted aryl. In some embodiments, R² is substituted or unsubstituted heteroaryl.

In some embodiments, R^{B} is substituted aryl. In some embodiments, R^{B} is substituted or unsubstituted C₁-C₃alkylene-aryl.

In some embodiments, R^{A} is H or D.

In some embodiments, R^{A} and R^{B} together with the nitrogen to which they are attached form a substituted 6 or 7-membered heterocyclic ring A.

In some embodiments, the heterocyclic ring A is substituted with —Y¹—R¹, wherein,
—Y¹— is selected from the group consisting of —O—, —S—, —S(═O)—, —S(═O)₂—, —S(═O)(═NR¹)—, —CH₂—, and —C(═O)—, and
R¹ is H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl.

Exemplary compounds include the compounds described in the following Tables:

TABLE 6

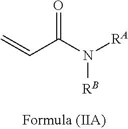

| 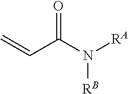 | Name |
|---|---|
| 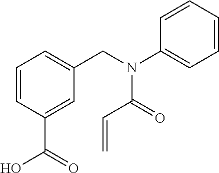 [SMILES: C=CC(=O)N(Cc1cccc(C(=O)O)c1)c1ccccc1] | 3-((N-phenylacrylamido)methyl) benzoic acid |
| 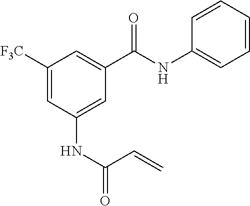 [SMILES: C=CC(=O)Nc1cc(C(=O)Nc2ccccc2)cc(C(F)(F)F)c1] | 3-acrylamido-N-phenyl-5- (trifluoromethyl)benzamide |
| 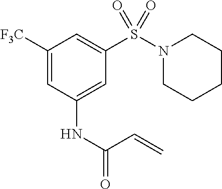 [SMILES: C=CC(=O)Nc1cc(C(F)(F)F)cc(S(=O)(=O)N2CCCCC2)c1] | N-(3-(piperidin-1-ylsulfonyl)-5- (trifluoromethyl)phenyl) acrylamide |
| 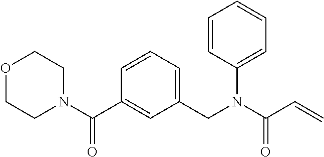 [SMILES: C=CC(=O)N(Cc1cccc(C(=O)N2CCOCC2)c1)c1ccccc1] | N-(3-(morpholine-4-carbonyl)benzyl)- N-phenylacrylamide |
| 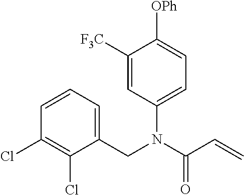 [SMILES: C=CC(=O)N(Cc1cccc(Cl)c1Cl)c1ccc(Oc2ccccc2)c(C(F)(F)F)c1] | N-(2,3-dichlorobenzyl)-N- (4-phenoxy-3- (trifluoromethyl)phenyl) acrylamide |
| 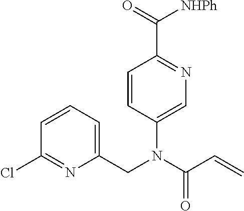 | 5-(N-((6-chloropyridin-2-yl)methyl) acrylamido)-N- phenylpicolinamide |

In one aspect, provided herein is an acceptable salt or solvate of a compound described in Table 6.

TABLE 7

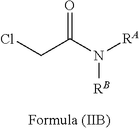

| 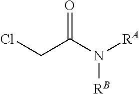 | Name |
|---|---|
| 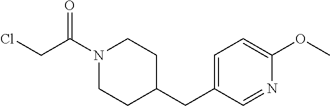 [SMILES: COc1ccc(CC2CCN(C(=O)CCl)CC2)cn1] | 2-chloro-1-(4- ((6-methoxypyridin-3-yl) methyl)piperidin-1- yl)ethan-1-one |
| 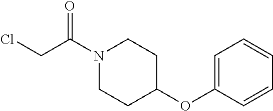 [SMILES: O=C(CCl)N1CCC(Oc2ccccc2)CC1] | 2-chloro-1-(4-phenoxypiperidin- 1-yl)ethan-1-one |
| 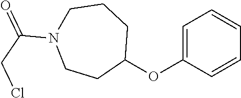 [SMILES: O=C(CCl)N1CCCC(Oc2ccccc2)CC1] | 2-chloro-1-(4-phenoxyazepan- 1-yl)ethan-1-one |
| 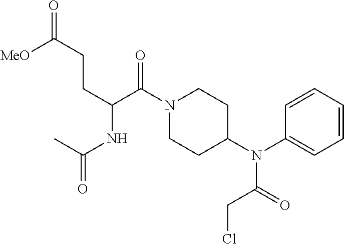 [SMILES: COC(=O)CCC(NC(C)=O)C(=O)N1CCC(N(C(=O)CCl)c2ccccc2)CC1] | methyl 4-acetamido-5- (4-(2-chloro-N- phenylacetamido)piperidin- 1-yl)-5-oxopentanoate |
| 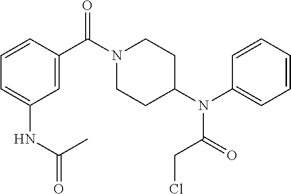 [SMILES: CC(=O)Nc1cccc(C(=O)N2CCC(N(C(=O)CCl)c3ccccc3)CC2)c1] | N-(1-(3-acetamidobenzoyl) piperidin-4-yl)-2-chloro-N- phenylacetamide |
| 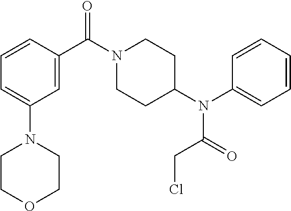 [SMILES: O=C(c1cccc(N2CCOCC2)c1)N1CCC(N(C(=O)CCl)c2ccccc2)CC1] | 2-chloro-N-(1-(3- morpholinobenzoyl) piperidin-4-yl)- N-phenylacetamide |
| 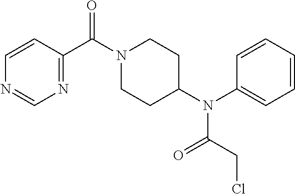 [SMILES: O=C(c1ccncn1)N1CCC(N(C(=O)CCl)c2ccccc2)CC1] | 2-chloro-N-phenyl-N- (1-(pyrimidine- 4-carbonyl)piperidin- 4-yl)acetamide |
| 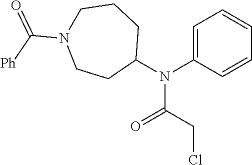 [SMILES: O=C(c1ccccc1)N1CCCC(N(C(=O)CCl)c2ccccc2)CC1] | N-(1-benzoylazepan-4-yl)-2- chloro- N-phenylacetamide |
| 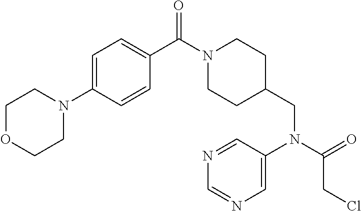 [SMILES: O=C(c1ccc(N2CCOCC2)cc1)N1CCC(CN(C(=O)CCl)c2cncnc2)CC1] | 2-chloro-N-((1-(4- morpholinobenzoyl) piperidin-4- yl)methyl)-N-(pyrimidin-5-yl) acetamide |
| 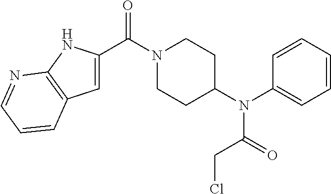 [SMILES: O=C(c1cc2cccnc2[nH]1)N1CCC(N(C(=O)CCl)c2ccccc2)CC1] | N-(1-(1H-pyrrolo[2,3-b]pyridine- 2-carbonyl)piperidin-4- yl)-2-chloro-N-phenylacetamide |
| 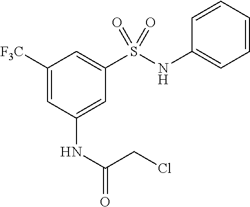 [SMILES: O=C(CCl)Nc1cc(C(F)(F)F)cc(S(=O)(=O)Nc2ccccc2)c1] | 2-chloro-N-(3-(N- phenylsulfamoyl)-5- (trifluoromethyl)phenyl) acetamide |
| 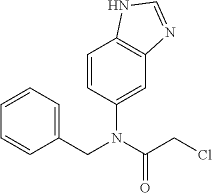 [SMILES: O=C(CCl)N(Cc1ccccc1)c1ccc2[nH]cnc2c1] | N-(1H-benzo[d]imidazol-5-yl)- N-benzyl-2- chloroacetamide |
| 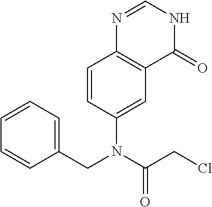 [SMILES: O=C(CCl)N(Cc1ccccc1)c1ccc2nc[nH]c(=O)c2c1] | N-benzyl-2-chloro-N-(4-oxo-3,4- dihydroquinazolin-6- yl)acetamide |
| 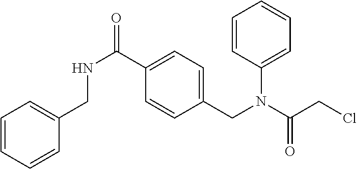 [SMILES: O=C(NCc1ccccc1)c1ccc(CN(C(=O)CCl)c2ccccc2)cc1] | N-benzyl-4-((2-chloro-N- phenylacetamido)methyl)benzamide |
| 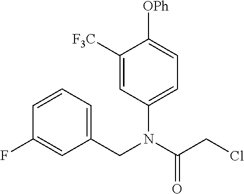 [SMILES: O=C(CCl)N(Cc1cccc(F)c1)c1ccc(Oc2ccccc2)c(C(F)(F)F)c1] | 2-chloro-N-(3-fluorobenzyl)- N-(4-phenoxy-3- (trifluoromethyl)phenyl) acetamide |
| 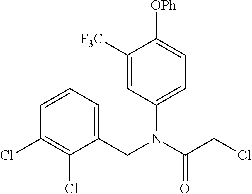 [SMILES: O=C(CCl)N(Cc1cccc(Cl)c1Cl)c1ccc(Oc2ccccc2)c(C(F)(F)F)c1] | 2-chloro-N-(2,3-dichlorobenzyl)- N-(4-phenoxy-3- (trifluoromethyl)phenyl) acetamide |
| 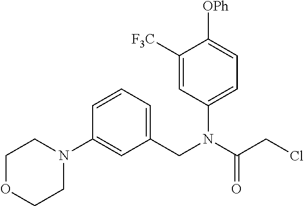 [SMILES: O=C(CCl)N(Cc1cccc(N2CCOCC2)c1)c1ccc(Oc2ccccc2)c(C(F)(F)F)c1] | 2-chloro-N-(3-morpholinobenzyl)- N-(4-phenoxy-3- (trifluoromethyl)phenyl) acetamide |
| 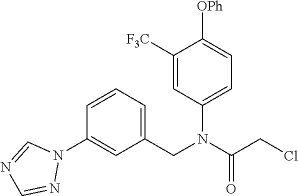 [SMILES: O=C(CCl)N(Cc1cccc(-n2cncn2)c1)c1ccc(Oc2ccccc2)c(C(F)(F)F)c1] | N-(3-(1H-1,2,4-triazol-1-yl)benzyl)- 2-chloro-N-(4- phenoxy-3-(trifluoromethyl) phenyl)acetamide |
| 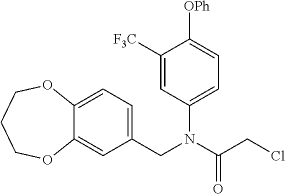 [SMILES: O=C(CCl)N(Cc1ccc2c(c1)OCCCO2)c1ccc(Oc2ccccc2)c(C(F)(F)F)c1] | 2-chloro-N-((3,4-dihydro-2H-benzo[b] [1,4]dioxepin-7- yl)methyl)-N-(4-phenoxy-3- (trifluoromethyl)phenyl)acetamide |
| 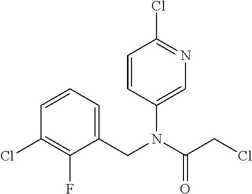 [SMILES: O=C(CCl)N(Cc1cccc(Cl)c1F)c1ccc(Cl)nc1] | 2-chloro-N-(3-chloro-2-fluorobenzyl)- N-(6-chloropyridin- 3-yl)acetamide |
| 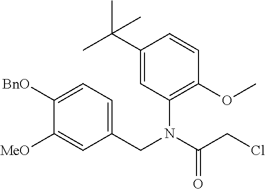 [SMILES: COc1cc(CN(C(=O)CCl)c2cc(C(C)(C)C)ccc2OC)ccc1OCc1ccccc1] | N-(4-(benzyloxy)-3-methoxybenzyl)- N-(5-(tert-butyl)-2- methoxyphenyl)-2- chloroacetamide |
| 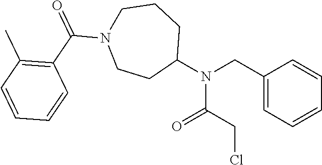 [SMILES: Cc1ccccc1C(=O)N1CCCC(N(Cc2ccccc2)C(=O)CCl)CC1] | N-benzyl-2-chloro-N-(1-(2- methylbenzoyl)azepan-4- yl)acetamide |
| 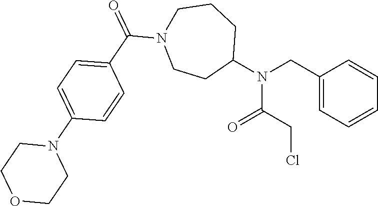 [SMILES: O=C(c1ccc(N2CCOCC2)cc1)N1CCCC(N(Cc2ccccc2)C(=O)CCl)CC1] | N-benzyl-2-chloro-N-(1- (4-morpholinobenzoyl) azepan-4- yl)acetamide |
| 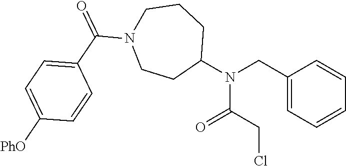 [SMILES: O=C(c1ccc(Oc2ccccc2)cc1)N1CCCC(N(Cc2ccccc2)C(=O)CCl)CC1] | N-benzyl-2-chloro-N-(1- (4-phenoxybenzoyl) azepan-4- yl)acetamide |
| 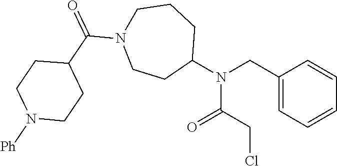 [SMILES: O=C(C1CCN(c2ccccc2)CC1)N1CCCC(N(Cc2ccccc2)C(=O)CCl)CC1] | N-benzyl-2-chloro-N-(1- (1-phenylpiperidine-4- carbonyl)azepan-4-yl) acetamide |
| 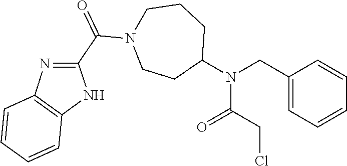 [SMILES: O=C(c1nc2ccccc2[nH]1)N1CCCC(N(Cc2ccccc2)C(=O)CCl)CC1] | N-(1-(1H-benzo[d]imidazole- 2-carbonyl)azepan-4-yl)-N- benzyl-2-chloroacetamide |
| 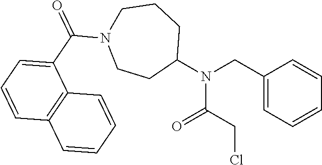 [SMILES: O=C(c1cccc2ccccc12)N1CCCC(N(Cc2ccccc2)C(=O)CCl)CC1] | N-(1-(1-naphthoyl)azepan- 4-yl)-N-benzyl-2- chloroacetamide |
| 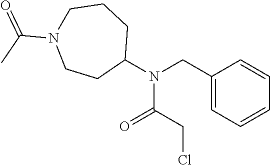 [SMILES: CC(=O)N1CCCC(N(Cc2ccccc2)C(=O)CCl)CC1] | N-(1-acetylazepan-4-yl)- N-benzyl-2-chloroacetamide |
| 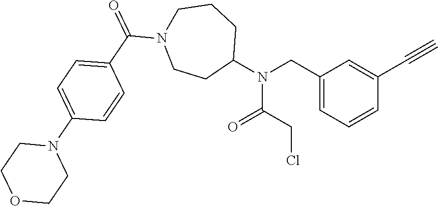 [SMILES: C#Cc1cccc(CN(C(=O)CCl)C2CCCN(C(=O)c3ccc(N4CCOCC4)cc3)CC2)c1] | 2-chloro-N-(3- ethynylbenzyl)-N-(1-(4- morpholinobenzoyl) azepan-4-yl)acetamide |

In one aspect, provided herein is an acceptable salt or solvate of a compound described in Table 7.

In some cases, the synthetic ligand is

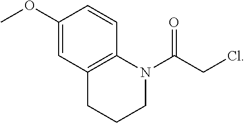 [SMILES: COc1ccc2c(c1)CCCN2C(=O)CCl]

In some cases, the synthetic ligand is

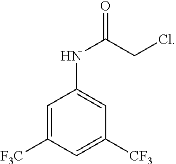 [SMILES: O=C(CCl)Nc1cc(C(F)(F)F)cc(C(F)(F)F)c1]

Any combination of the groups described above for the various variables is contemplated herein. Throughout the specification, groups and substituents thereof are chosen by one skilled in the field to provide stable moieties and compounds.

### Further Forms of Compounds

In one aspect, the compound of Formula (II), Formula (IIA), or Formula (IIB) possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

Compounds described herein may be formed as, and/or used as, acceptable salts. The type of acceptable salts, include, but are not limited to: (1) acid addition salts, formed by reacting the free base form of the compound with an acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure).

In one aspect, compounds are synthesized as described in the Examples section.

### NRF2-Regulated Proteins and Protein-Probe Adducts

In some embodiments, described herein are cysteine-containing proteins that are regulated by NRF2. In some instances, the cysteine-containing proteins are NRF2-regulated proteins illustrated in Tables 1A, 2, 3A, and/or 4. In some cases, the cysteine-containing proteins are NRF2-regulated proteins illustrated in Tables 1A. In some cases, the cysteine-containing proteins are NRF2-regulated proteins illustrated in Tables 2. In some cases, the cysteine-containing proteins are NRF2-regulated proteins illustrated in Table 3A. In some cases, the cysteine-containing proteins are NRF2-regulated proteins illustrated in Table 4.

In some instances, Tables 1A, 2, 3A, and 4 further illustrate one or more cysteine residues of a listed NRF2-regulated protein for interaction with a probe and/or a ligand described herein. In some cases, the cysteine residue number of a NRF2-regulated protein is in reference to the respective UNIPROT identifier.

In some instances, a cysteine residue illustrated in Tables 1A, 2, 3A, and/or 4 is located from 10 Å to 60 Å away from an active site residue of the respective NRF2-regulated protein. In some instances, the cysteine residue is located at least 10 Å, 12 Å, 15 Å, 20 Å, 25 Å, 30 Å, 35 Å, 40 Å, 45 Å, or 50 Å away from an active site residue of the respective NRF2-regulated protein. In some instances, the cysteine residue is located about 10 Å, 12 Å, 15 Å, 20 Å, 25 Å, 30 Å, 35 Å, 40 Å, 45 Å, or 50 Å away from an active site residue of the respective NRF2-regulated protein.

In some embodiments, described herein include a protein-probe adduct wherein the probe binds to a cysteine residue illustrated in Tables 1A, 2, 3A, and 4; wherein the probe has a structure represented by Formula (I):

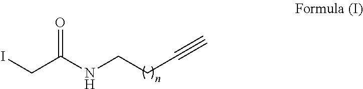

wherein,
n is 0-8.

In some instances, n is 1, 2, 3, 4, 5, 6, 7, or 8. In some instances, n is 1. In some instances, n is 2. In some instances, n is 3. In some instances, n is 4. In some instances, n is 5. In some instances, n is 6. In some instances, n is 7. In some instances, n is 8.

In some instances, the probe binds to a cysteine residue illustrated in Table 1A. In some instances, the probe binds to a cysteine residue illustrated in Table 2. In some instances, the probe binds to a cysteine residue illustrated in Table 3A. In some cases, the probe binds to a cysteine residue illustrated in Table 4.

In some embodiments, the protein is ubiquitin carboxyl-terminal hydrolase 7 (USP7). In some cases, the cysteine residue is C223, wherein the numbering of the amino acid position corresponds to the amino acid position with the UniProt Identifier Q93009. In some cases, the probe binds to C223 of USP7.

In some embodiments, the protein is B-cell lymphoma/leukemia 10 (BCL10). In some cases, the cysteine residue is C119 or C122, wherein the numberings of the amino acid positions correspond to the amino acid positions with the UniProt Identifier O95999. In some cases, the probe binds to C119 of BCL10. In other cases, the probe binds to C122 of BCL10.

In some embodiments, the protein is RAF proto-oncogene serine/threonine-protein kinase (RAF1). In some instances, the cysteine residue is C637, wherein the numbering of the amino acid position corresponds to the amino acid position with the UniProt Identifier P04049. In some cases, the probe binds to C637 of RAF1.

In some embodiments, the protein is nuclear receptor subfamily 2 group F member 6 (NR2F6). In some instances, the cysteine residue is C203 or C316, wherein the numberings of the amino acid positions correspond to the amino acid positions with the UniProt Identifier P10588. In some cases, the probe binds to C203 of NR2F6. In other cases, the probe binds to C316 of NR2F6.

In some embodiments, the protein is DNA-binding protein inhibitor ID-1 (ID1). In some instances, the cysteine residue is C17, wherein the numbering of the amino acid position corresponds to the amino acid position with the UniProt Identifier P41134. In some cases, the probe binds to C17 of ID1.

In some embodiments, the protein is Fragile X mental retardation syndrome-related protein 1 (FXR1). In some instances, the cysteine residue is C99, wherein the numbering of the amino acid position corresponds to the amino acid position with the UniProt Identifier P51114. In some cases, the probe binds to C99 or FXR1.

In some embodiments, the protein is Mitogen-activated protein kinase kinase kinase kinase 4 (MAP4K4). In some instances, the cysteine residue is C883, wherein the numbering of the amino acid position corresponds to the amino acid position with the UniProt Identifier O95819. In some cases, the probe binds to C883 of MAP4K4.

In some embodiments, the protein is Cathepsin B (CTSB). In some instances, the cysteine residue is C105 or C108, wherein the numberings of the amino acid positions correspond to the amino acid positions with the UniProt Identifier P07858. In some cases, the probe binds to C105 of CTSB. In other cases, the probe binds to C108 of CTSB.

In some embodiments, the protein is integrin beta-4 (ITGB4). In some instances, the cysteine residue is C245 or C288, wherein the numberings of the amino acid positions correspond to the amino acid positions with the UniProt Identifier P16144. In some cases, the probe binds to C245 of ITGB4. In other cases, the probe binds to C288 of ITGB4.

In some embodiments, the protein is TFIIH basal transcription factor complex helicase (ERCC2). In some instances, the cysteine residue is C663, wherein the numbering of the amino acid position corresponds to the amino acid position with the UniProt Identifier P18074. In some cases, the probe binds to C663 of ERCC2.

In some embodiments, the protein is nuclear receptor subfamily 4 group A member 1 (NR4A1). In some instances, the cysteine residue is C551, wherein the numbering of the amino acid position corresponds to the amino acid position with the UniProt Identifier P22736. In some cases, the probe binds to C551 of NR4A1.

In some embodiments, the protein is cytidine deaminase (CDA). In some instances, the cysteine residue is C8, wherein the numbering of the amino acid position corresponds to the amino acid position with the UniProt Identifier P32320. In some cases, the probe binds to C8 of CDA.

In some embodiments, the protein is sterol O-acyltransferase 1 (SOAT1). In some instances, the cysteine residue is C92, wherein the numbering of the amino acid position corresponds to the amino acid position with the UniProt Identifier P35610. In some cases, the probe binds to C92 of SOAT1.

In some embodiments, the protein is DNA mismatch repair protein Msh6 (MSH6). In some instances, the cysteine residue is C615, wherein the numbering of the amino acid position corresponds to the amino acid position with the UniProt Identifier P52701. In some cases, the probe binds to C615 of MSH6.

In some embodiments, the protein is telomeric repeat-binding factor 1 (TERF1). In some instances, the cysteine residue is C118, wherein the numbering of the amino acid position corresponds to the amino acid position with the UniProt Identifier P54274. In some cases, the probe binds to C118 of TERF1.

In some embodiments, the protein is NEDD8-conjugating enzyme Ubc12 (UBE2M). In some instances, the cysteine residue is C47, wherein the numbering of the amino acid position corresponds to the amino acid position with the UniProt Identifier P61081. In some cases, the probe binds to C47 of UBE2M.

In some embodiments, the protein is E3 ubiquitin-protein ligase TRIP12 (TRIP12). In some instances, the cysteine residue is C535, wherein the numbering of the amino acid position corresponds to the amino acid position with the UniProt Identifier Q14669. In some cases, the probe binds to C535 of TRIP12.

In some embodiments, the protein is ubiquitin carboxyl-terminal hydrolase 10 (USP10). In some instances, the cysteine residue is C94, wherein the numbering of the amino acid position corresponds to the amino acid position with the UniProt Identifier Q14694. In some cases, the probe binds to C94 of USP10.

In some embodiments, the protein is ubiquitin carboxyl-terminal hydrolase 30 (USP30). In some instances, the cysteine residue is C142, wherein the numbering of the amino acid position corresponds to the amino acid position with the UniProt Identifier Q70CQ3. In some cases, the probe binds to C142 of USP30.

In some embodiments, the protein is nucleus accumbens-associated protein 1 (NACC1). In some instances, the cysteine residue is C301, wherein the numbering of the amino acid position corresponds to the amino acid position with the UniProt Identifier Q96RE7. In some cases, the probe binds to C301 of NACC1.

In some embodiments, the protein is lymphoid-specific helicase (HELLS). In some instances, the cysteine residue is C277 or C836, wherein the numberings of the amino acid positions correspond to the amino acid positions with the UniProt Identifier Q9NRZ9. In some cases, the probe binds to C277 of HELLS. In other cases, the probe binds to C836 of HELLS.

In some embodiments, also described herein include a synthetic ligand that inhibits a covalent interaction between a protein and a probe, wherein in the absence of the synthetic ligand, the probe binds to a cysteine residue illustrated in Tables 1A, 2, 3A, and 4; and wherein the probe has a structure represented by Formula (I):

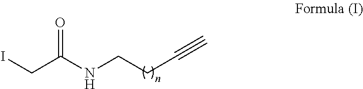

wherein,
n is 0-8.

In some instances, n is 1, 2, 3, 4, 5, 6, 7, or 8. In some instances, n is 1. In some instances, n is 2. In some instances, n is 3. In some instances, n is 4. In some instances, n is 5. In some instances, n is 6. In some instances, n is 7. In some instances, n is 8.

In some instances, the probe binds to a cysteine residue illustrated in Table 1A. In some instances, the probe binds to a cysteine residue illustrated in Table 2. In some instances, the probe binds to a cysteine residue illustrated in Table 3A. In some instances, the probe binds to a cysteine residue illustrated in Table 4.

In some instances, the protein is ubiquitin carboxyl-terminal hydrolase 7 (USP7) and the cysteine residue is C223, wherein the numbering of the amino acid position corresponds to the amino acid position with the UniProt Identifier Q93009. In some cases, the synthetic ligand inhibits a covalent interaction between C223 of USP7 and the probe.

In some instances, the protein is B-cell lymphoma/leukemia 10 (BCL10) and the cysteine residue is C119 or C122, wherein the numberings of the amino acid positions correspond to the amino acid positions with the UniProt Identifier O95999. In some cases, the synthetic ligand inhibits a covalent interaction between C119 or C122 of BCL10 and the probe.

In some instances, the protein is RAF proto-oncogene serine/threonine-protein kinase (RAF1) and the cysteine residue is C637, wherein the numbering of the amino acid position corresponds to the amino acid position with the UniProt Identifier P04049. In some cases, the synthetic ligand inhibits a covalent interaction between C637 of RAF 1 and the probe.

In some instances, the protein is nuclear receptor subfamily 2 group F member 6 (NR2F6) and the cysteine residue is C203 or C316, wherein the numberings of the amino acid positions correspond to the amino acid positions with the UniProt Identifier P10588. In some cases, the synthetic ligand inhibits a covalent interaction between C203 or C316 of NR2F6 and the probe.

In some instances, the protein is DNA-binding protein inhibitor ID-1 (ID1) and the cysteine residue is C17, wherein the numbering of the amino acid position corresponds to the amino acid position with the UniProt Identifier P41134. In some cases, the synthetic ligand inhibits a covalent interaction between C17 of ID1 and the probe.

In some instances, the protein is Fragile X mental retardation syndrome-related protein 1 (FXR1) and the cysteine residue is C99, wherein the numbering of the amino acid position corresponds to the amino acid position with the UniProt Identifier P51114. In some cases, the synthetic ligand inhibits a covalent interaction between C99 of FXR1 and the probe.

In some instances, the protein is Mitogen-activated protein kinase kinase kinase kinase 4 (MAP4K4) and the cysteine residue is C883, wherein the numbering of the amino acid position corresponds to the amino acid position with the UniProt Identifier O95819. In some cases, the synthetic ligand inhibits a covalent interaction between C883 of MAP4K4 and the probe.

In some instances, the protein is Cathepsin B (CTSB) and the cysteine residue is C105 or C108, wherein the numberings of the amino acid positions correspond to the amino acid positions with the UniProt Identifier P07858. In some cases, the synthetic ligand inhibits a covalent interaction between C108 of CTSB and the probe.

In some instances, the protein is integrin beta-4 (ITGB4) and the cysteine residue is C245 or C288, wherein the numberings of the amino acid positions correspond to the amino acid positions with the UniProt Identifier P16144. In some cases, the synthetic ligand inhibits a covalent interaction between C245 or C288 of ITGB4 and the probe.

In some instances, the protein is TFIIH basal transcription factor complex helicase (ERCC2) and the cysteine residue is C663, wherein the numbering of the amino acid position corresponds to the amino acid position with the UniProt Identifier P18074. In some cases, the synthetic ligand inhibits a covalent interaction between C663 of ERCC2 and the probe.

In some instances, the protein is nuclear receptor subfamily 4 group A member 1 (NR4A1) and the cysteine residue is C551, wherein the numbering of the amino acid position corresponds to the amino acid position with the UniProt Identifier P22736. In some cases, the synthetic ligand inhibits a covalent interaction between C551 of NR4A1 and the probe.

In some instances, the protein is cytidine deaminase (CDA) and the cysteine residue is C8, wherein the numbering of the amino acid position corresponds to the amino acid position with the UniProt Identifier P32320. In some cases, the synthetic ligand inhibits a covalent interaction between C8 of CDA and the probe.

In some instances, the protein is sterol O-acyltransferase 1 (SOAT1) and the cysteine residue is C92, wherein the numbering of the amino acid position corresponds to the amino acid position with the UniProt Identifier P35610. In some cases, the synthetic ligand inhibits a covalent interaction between C92 of SOAT1 and the probe.

In some instances, the protein is DNA mismatch repair protein Msh6 (MSH6) and the cysteine residue is C615, wherein the numbering of the amino acid position corresponds to the amino acid position with the UniProt Identifier P52701. In some cases, the synthetic ligand inhibits a covalent interaction between C615 of MSH6 and the probe.

In some instances, the protein is telomeric repeat-binding factor 1 (TERF1) and the cysteine residue is C118, wherein the numbering of the amino acid position corresponds to the amino acid position with the UniProt Identifier P54274. In some cases, the synthetic ligand inhibits a covalent interaction between C118 of TERF1 and the probe.

In some instances, the protein is NEDD8-conjugating enzyme Ubc12 (UBE2M) and the cysteine residue is C47, wherein the numbering of the amino acid position corresponds to the amino acid position with the UniProt Identifier P61081. In some cases, the synthetic ligand inhibits a covalent interaction between C47 of UBE2M and the probe.

In some instances, the protein is E3 ubiquitin-protein ligase TRIP12 (TRIP12) and the cysteine residue is C535, wherein the numbering of the amino acid position corresponds to the amino acid position with the UniProt Identifier Q14669. In some cases, the synthetic ligand inhibits a covalent interaction between C535 of TRIP12 and the probe.

In some instances, the protein is ubiquitin carboxyl-terminal hydrolase 10 (USP10) and the cysteine residue is C94, wherein the numbering of the amino acid position corresponds to the amino acid position with the UniProt Identifier Q14694. In some cases, the synthetic ligand inhibits a covalent interaction between C94 of USP10 and the probe.

In some instances, the protein is ubiquitin carboxyl-terminal hydrolase 30 (USP30) and the cysteine residue is C142, wherein the numbering of the amino acid position corresponds to the amino acid position with the UniProt Identifier Q70CQ3. In some cases, the synthetic ligand inhibits a covalent interaction between C142 of USP30 and the probe.

In some instances, the protein is nucleus accumbens-associated protein 1 (NACC1) and the cysteine residue is C301, wherein the numbering of the amino acid position corresponds to the amino acid position with the UniProt Identifier Q96RE7. In some cases, the synthetic ligand inhibits a covalent interaction between C301 of NACC1 and the probe.

In some instances, the protein is lymphoid-specific helicase (HELLS) and the cysteine residue is C277 or C836, wherein the numberings of the amino acid positions correspond to the amino acid positions with the UniProt Identifier Q9NRZ9. In some cases, the synthetic ligand inhibits a covalent interaction between C277 or C836 of HELLS and the probe.

In some cases, the synthetic ligand comprises a structure represented by Formula II:

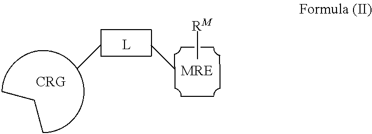

wherein,
CRG-L is optional, and when present is a covalent reactive group comprising a Michael acceptor moiety, a leaving group moiety, or a moiety capable of forming a covalent bond to the thiol group of a cysteine residue, and L is a linker;
MRE is a molecular recognition element that is capable of interacting with the protein; and
R^{M} is optional, and when present comprises a binding element that binds to a second protein or another compound.

In some cases, the Michael acceptor moiety comprises an alkene or an alkyne moiety.

In some instances, L is a cleavable linker. In other instances, L is a non-cleavable linker.

In some cases, MRE comprises a small molecule compound, a polynucleotide, a polypeptide or fragments thereof, or a peptidomimetic.

In some cases, the synthetic ligand has a structure represented by Formula (IIA) or Formula (IIB):

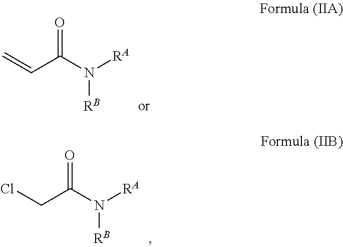

wherein,
each R^{A} and R^{B} is independently selected from the group consisting of H, D, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl; or
R^{A} and R^{B} together with the nitrogen to which they are attached form a substituted or unsubstituted 5, 6, 7 or 8-membered heterocyclic ring A, optionally having one additional heteroatom moiety independently selected from NR¹, O, or S; and
R¹ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl.

In some instances, R^{A} is substituted or unsubstituted aryl, substituted or unsubstituted C₁-C₃alkylene-aryl, substituted or unsubstituted heteroaryl, or substituted or unsubstituted C₁-C₃alkylene-heteroaryl.

In some instances, R^{B} is substituted or unsubstituted C₂-C₇heterocycloalkyl, substituted or unsubstituted aryl, or substituted or unsubstituted heteroaryl.

In some instances, R^{B} is substituted C₅-C₇heterocycloalkyl, substituted with —C(═O)R², wherein R² is substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl.

In some instances, R^{B} substituted or unsubstituted C₁-C₃alkylene-aryl.

In some instances, R^{A} is H or D.

In some instances, R^{B} is substituted aryl.

In some instances, R^{A} and R^{B} together with the nitrogen to which they are attached form a substituted 6 or 7-membered heterocyclic ring A.

In some instances, the heterocyclic ring A is substituted with —Y¹—R¹, wherein,
—Y¹— is selected from the group consisting of —O—, —S—, —S(═O)—, —S(═O)₂—, —S(═O)(═NR¹)—, —CH₂—, and —C(═O)—, and
R¹ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl.

In some cases, the synthetic ligand is: 2-chloro-1-(4-((6-methoxypyridin-3-yl)methyl)piperidin-1-yl)ethan-1-one; 2-chloro-1-(4-phenoxypiperidin-1-yl)ethan-1-one; 2-chloro-1-(4-phenoxyazepan-1-yl)ethan-1-one; methyl 4-acetamido-5-(4-(2-chloro-N-phenylacetamido)piperidin-1-yl)-5-oxopentanoate; N-(1-(3-acetamidobenzoyl)piperidin-4-yl)-2-chloro-N-phenylacetamide; 2-chloro-N-(1-(3-morpholinobenzoyl)piperidin-4-yl)-N-phenylacetamide; 2-chloro-N-phenyl-N-(1-(pyrimidine-4-carbonyl)piperidin-4-yl)acetamide; N-(1-benzoylazepan-4-yl)-2-chloro-N-phenylacetamide; 2-chloro-N-((1-(4-morpholinobenzoyl)piperidin-4-yl)methyl)-N-(pyrimidin-5-yl)acetamide; N-(1-(1H-pyrrolo[2,3-b]pyridine-2-carbonyl)piperidin-4-yl)-2-chloro-N-phenylacetamide; 3-((N-phenylacrylamido)methyl)benzoic acid; 3-acrylamido-N-phenyl-5-(trifluoromethyl)benzamide; N-(3-(piperidin-1-ylsulfonyl)-5-(trifluoromethyl)phenyl)acrylamide; 2-chloro-N-(3-(N-phenylsulfamoyl)-5-(trifluoromethyl)phenyl)acetamide; N-(1H-benzo[d]imidazol-5-yl)-N-benzyl-2-chloroacetamide; N-benzyl-2-chloro-N-(4-oxo-3,4-dihydroquinazolin-6-yl)acetamide; N-(3-(morpholine-4-carbonyl)benzyl)-N-phenylacrylamide; N-benzyl-4-((2-chloro-N-phenylacetamido)methyl)benzamide; 2-chloro-N-(3-fluorobenzyl)-N-(4-phenoxy-3-(trifluoromethyl)phenyl)acetamide; 2-chloro-N-(2,3-dichlorobenzyl)-N-(4-phenoxy-3-(trifluoromethyl)phenyl)acetamide; N-(2,3-dichlorobenzyl)-N-(4-phenoxy-3-(trifluoromethyl)phenyl)acrylamide; 2-chloro-N-(3-morpholinobenzyl)-N-(4-phenoxy-3-(trifluoromethyl)phenyl)acetamide; N-(3-(1H-1,2,4-triazol-1-yl)benzyl)-2-chloro-N-(4-phenoxy-3-(trifluoromethyl)phenyl)acetamide; 2-chloro-N-((3,4-dihydro-2H-benzo[b][1,4]dioxepin-7-yl)methyl)-N-(4-phenoxy-3-(trifluoromethyl)phenyl)acetamide; 5-(N-((6-chloropyridin-2-yl)methyl)acrylamido)-N-phenylpicolinamide; 2-chloro-N-(3-chloro-2-fluorobenzyl)-N-(6-chloropyridin-3-yl)acetamide; N-(4-(benzyloxy)-3-methoxybenzyl)-N-(5-(tert-butyl)-2-methoxyphenyl)-2-chloroacetamide; N-benzyl-2-chloro-N-(1-(2-methylbenzoyl)azepan-4-yl)acetamide; N-benzyl-2-chloro-N-(1-(4-morpholinobenzoyl)azepan-4-yl)acetamide; N-benzyl-2-chloro-N-(1-(4-phenoxybenzoyl)azepan-4-yl)acetamide; N-benzyl-2-chloro-N-(1-(1-phenylpiperidine-4-carbonyl)azepan-4-yl)acetamide; N-(1-(1H-benzo[d]imidazole-2-carbonyl)azepan-4-yl)-N-benzyl-2-chloroacetamide; N-(1-(1-naphthoyl)azepan-4-yl)-N-benzyl-2-chloroacetamide; N-(1-acetylazepan-4-yl)-N-benzyl-2-chloroacetamide; or 2-chloro-N-(3-ethynylbenzyl)-N-(1-(4-morpholinobenzoyl)azepan-4-yl)acetamide.

In some embodiments, the synthetic ligand further comprises a second moiety that interacts with a second protein. In some cases, the second protein is not a protein illustrated in Tables 1A, 2, 3A, and 4.

In some embodiments, additionally described herein include a protein binding domain wherein said protein binding domain comprises a cysteine residue illustrated in Tables 1A, 2, 3A, and 4, wherein said cysteine forms an adduct with a compound of Formula I,

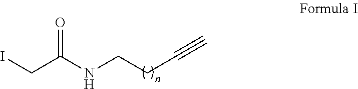

and wherein a compound of Formula IIA or Formula IIB interferes with the formation of the cysteine adduct by the compound of Formula I, wherein Formula (IIA) or Formula (IIB) have the structure:

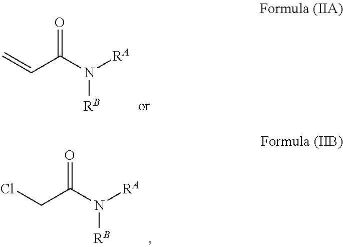

wherein,
each R^{A} and R^{B} is independently selected from the group consisting of H, D, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl; or
or R^{A} and R^{B} together with the nitrogen to which they are attached form a 5, 6, 7 or 8-membered heterocyclic ring A, optionally having one additional heteroatom moiety independently selected from NR¹, O, or S; wherein A is optionally substituted.

In some instances, n is 1, 2, 3, 4, 5, 6, 7, or 8. In some instances, n is 1. In some instances, n is 2. In some instances, n is 3. In some instances, n is 4. In some instances, n is 5. In some instances, n is 6. In some instances, n is 7. In some instances, n is 8.

In some instances, the cysteine residue is illustrated in Table 1A. In some instances, the cysteine residue is illustrated in Table 2. In some instances, the cysteine residue is illustrated in Table 3A. In some instances, the cysteine residue is illustrated in Table 4.

In some instances, the protein is ubiquitin carboxyl-terminal hydrolase 7 (USP7) and the cysteine residue is C223, wherein the numbering of the amino acid position corresponds to the amino acid position with the UniProt Identifier Q93009. In some cases, the protein binding domain comprises C223.

In some instances, the protein is B-cell lymphoma/leukemia 10 (BCL10) and the cysteine residue is C119 or C122, wherein the numberings of the amino acid positions correspond to the amino acid positions with the UniProt Identifier O95999. In some cases, the protein binding domain comprises C119 or C122.

In some instances, the protein is RAF proto-oncogene serine/threonine-protein kinase (RAF1) and the cysteine residue is C637, wherein the numbering of the amino acid position corresponds to the amino acid position with the UniProt Identifier P04049. In some cases, the protein binding domain comprises C637.

In some instances, the protein is nuclear receptor subfamily 2 group F member 6 (NR2F6) and the cysteine residue is C203 or C316, wherein the numberings of the amino acid positions correspond to the amino acid positions with the UniProt Identifier P10588. In some cases, the protein binding domain comprises C203 or C316.

In some instances, the protein is DNA-binding protein inhibitor ID-1 (ID1) and the cysteine residue is C17, wherein the numbering of the amino acid position corresponds to the amino acid position with the UniProt Identifier P41134. In some cases, the protein binding domain comprises C17.

In some instances, the protein is Fragile X mental retardation syndrome-related protein 1 (FXR1) and the cysteine residue is C99, wherein the numbering of the amino acid position corresponds to the amino acid position with the UniProt Identifier P51114. In some cases, the protein binding domain comprises C99.

In some instances, the protein is Mitogen-activated protein kinase kinase kinase kinase 4 (MAP4K4) and the cysteine residue is C883, wherein the numbering of the amino acid position corresponds to the amino acid position with the UniProt Identifier O95819. In some cases, the protein binding domain comprises C883.

In some instances, the protein is Cathepsin B (CTSB) and the cysteine residue is C105 or C108, wherein the numberings of the amino acid positions correspond to the amino acid positions with the UniProt Identifier P07858. In some cases, the protein binding domain comprises C105 or C108.

In some instances, the protein is integrin beta-4 (ITGB4) and the cysteine residue is C245 or C288, wherein the numberings of the amino acid positions correspond to the amino acid positions with the UniProt Identifier P16144. In some cases, the protein binding domain comprises C245 or C288.

In some instances, the protein is TFIIH basal transcription factor complex helicase (ERCC2) and the cysteine residue is C663, wherein the numbering of the amino acid position corresponds to the amino acid position with the UniProt Identifier P18074. In some cases, the protein binding domain comprises C663.

In some instances, the protein is nuclear receptor subfamily 4 group A member 1 (NR4A1) and the cysteine residue is C551, wherein the numbering of the amino acid position corresponds to the amino acid position with the UniProt Identifier P22736. In some cases, the protein binding domain comprises C551.

In some instances, the protein is cytidine deaminase (CDA) and the cysteine residue is C8, wherein the numbering of the amino acid position corresponds to the amino acid position with the UniProt Identifier P32320. In some cases, the protein binding domain comprises C8.

In some instances, the protein is sterol O-acyltransferase 1 (SOAT1) and the cysteine residue is C92, wherein the numbering of the amino acid position corresponds to the amino acid position with the UniProt Identifier P35610. In some cases, the protein binding domain comprises C92.

In some instances, the protein is DNA mismatch repair protein Msh6 (MSH6) and the cysteine residue is C615, wherein the numbering of the amino acid position corresponds to the amino acid position with the UniProt Identifier P52701. In some cases, the protein binding domain comprises C615.

In some instances, the protein is telomeric repeat-binding factor 1 (TERF1) and the cysteine residue is C118, wherein the numbering of the amino acid position corresponds to the amino acid position with the UniProt Identifier P54274. In some cases, the protein binding domain comprises C118.

In some instances, the protein is NEDD8-conjugating enzyme Ubc12 (UBE2M) and the cysteine residue is C47, wherein the numbering of the amino acid position corresponds to the amino acid position with the UniProt Identifier P61081. In some cases, the protein binding domain comprises C47.

In some instances, the protein is E3 ubiquitin-protein ligase TRIP12 (TRIP12) and the cysteine residue is C535, wherein the numbering of the amino acid position corresponds to the amino acid position with the UniProt Identifier Q14669. In some cases, the protein binding domain comprises C535.

In some instances, the protein is ubiquitin carboxyl-terminal hydrolase 10 (USP10) and the cysteine residue is C94, wherein the numbering of the amino acid position corresponds to the amino acid position with the UniProt Identifier Q14694. In some cases, the protein binding domain comprises C94.

In some instances, the protein is ubiquitin carboxyl-terminal hydrolase 30 (USP30) and the cysteine residue is C142, wherein the numbering of the amino acid position corresponds to the amino acid position with the UniProt Identifier Q70CQ3. In some cases, the protein binding domain comprises C142.

In some instances, the protein is nucleus accumbens-associated protein 1 (NACC1) and the cysteine residue is C301, wherein the numbering of the amino acid position corresponds to the amino acid position with the UniProt Identifier Q96RE7. In some cases, the protein binding domain comprises C301.

In some instances, the protein is lymphoid-specific helicase (HELLS) and the cysteine residue is C277 or C836, wherein the numberings of the amino acid positions correspond to the amino acid positions with the UniProt Identifier Q9NRZ9. In some cases, the protein binding domain comprises C277 or C836.

In some embodiments, further described herein is a method for identifying a synthetic ligand that interacts with a protein comprising a cysteine residue illustrated in Tables 1A, 2, 3A, and 4, comprising exposing, in a reaction vessel, the protein to the synthetic ligand and a probe that has a structure represented by Formula (I):

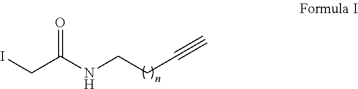

wherein,

n is 0-8; and

measuring the amount of the probe that has covalently bound to the cysteine residue relative to the amount of the probe that has covalently bound to the same cysteine residue in the absence of the synthetic ligand.

In some instances, the measuring includes one or more of the analysis methods described below.

In some instances, the cysteine residue is illustrated in Table 1A. In some instances, the cysteine residue is illustrated in Table 2. In some instances, the cysteine residue is illustrated in Table 3A. In some instances, the cysteine residue is illustrated in Table 4.

In some instances, the protein is ubiquitin carboxyl-terminal hydrolase 7 (USP7) and the cysteine residue is C223, wherein the numbering of the amino acid position corresponds to the amino acid position with the UniProt Identifier Q93009. In some cases, the synthetic ligand inhibits a covalent interaction between C223 of USP7 and the probe.

In some instances, the protein is B-cell lymphoma/leukemia 10 (BCL10) and the cysteine residue is C119 or C122, wherein the numberings of the amino acid positions correspond to the amino acid positions with the UniProt Identifier O95999. In some cases, the synthetic ligand inhibits a covalent interaction between C119 or C122 of BCL10 and the probe.

In some instances, the protein is RAF proto-oncogene serine/threonine-protein kinase (RAF1) and the cysteine residue is C637, wherein the numbering of the amino acid position corresponds to the amino acid position with the UniProt Identifier P04049. In some cases, the synthetic ligand inhibits a covalent interaction between C637 of RAF1 and the probe.

In some instances, the protein is nuclear receptor subfamily 2 group F member 6 (NR2F6) and the cysteine residue is C203 or C316, wherein the numberings of the amino acid positions correspond to the amino acid positions with the UniProt Identifier P10588. In some cases, the synthetic ligand inhibits a covalent interaction between C203 or C316 of NR2F6 and the probe.

In some instances, the protein is DNA-binding protein inhibitor ID-1 (ID1) and the cysteine residue is C17, wherein the numbering of the amino acid position corresponds to the amino acid position with the UniProt Identifier P41134. In some cases, the synthetic ligand inhibits a covalent interaction between C17 of ID1 and the probe.

In some instances, the protein is Fragile X mental retardation syndrome-related protein 1 (FXR1) and the cysteine residue is C99, wherein the numbering of the amino acid position corresponds to the amino acid position with the UniProt Identifier P51114. In some cases, the synthetic ligand inhibits a covalent interaction between C99 of FXR1 and the probe.

In some instances, the protein is Mitogen-activated protein kinase kinase kinase kinase 4 (MAP4K4) and the cysteine residue is C883, wherein the numbering of the amino acid position corresponds to the amino acid position with the UniProt Identifier O95819. In some cases, the synthetic ligand inhibits a covalent interaction between C883 of MAP4K4 and the probe.

In some instances, the protein is Cathepsin B (CTSB) and the cysteine residue is C105 or C108, wherein the numberings of the amino acid positions correspond to the amino acid positions with the UniProt Identifier P07858. In some cases, the synthetic ligand inhibits a covalent interaction between C108 of CTSB and the probe.

In some instances, the protein is integrin beta-4 (ITGB4) and the cysteine residue is C245 or C288, wherein the numberings of the amino acid positions correspond to the amino acid positions with the UniProt Identifier P16144. In some cases, the synthetic ligand inhibits a covalent interaction between C245 or C288 of ITGB4 and the probe.

In some instances, the protein is TFIIH basal transcription factor complex helicase (ERCC2) and the cysteine residue is C663, wherein the numbering of the amino acid position corresponds to the amino acid position with the UniProt Identifier P18074. In some cases, the synthetic ligand inhibits a covalent interaction between C663 of ERCC2 and the probe.

In some instances, the protein is nuclear receptor subfamily 4 group A member 1 (NR4A1) and the cysteine residue is C551, wherein the numbering of the amino acid position corresponds to the amino acid position with the UniProt Identifier P22736. In some cases, the synthetic ligand inhibits a covalent interaction between C551 of NR4A1 and the probe.

In some instances, the protein is cytidine deaminase (CDA) and the cysteine residue is C8, wherein the numbering of the amino acid position corresponds to the amino acid position with the UniProt Identifier P32320. In some cases, the synthetic ligand inhibits a covalent interaction between C8 of CDA and the probe.

In some instances, the protein is sterol O-acyltransferase 1 (SOAT1) and the cysteine residue is C92, wherein the numbering of the amino acid position corresponds to the amino acid position with the UniProt Identifier P35610. In some cases, the synthetic ligand inhibits a covalent interaction between C92 of SOAT1 and the probe.

In some instances, the protein is DNA mismatch repair protein Msh6 (MSH6) and the cysteine residue is C615, wherein the numbering of the amino acid position corresponds to the amino acid position with the UniProt Identifier P52701. In some cases, the synthetic ligand inhibits a covalent interaction between C615 of MSH6 and the probe.

In some instances, the protein is telomeric repeat-binding factor 1 (TERF1) and the cysteine residue is C118, wherein the numbering of the amino acid position corresponds to the amino acid position with the UniProt Identifier P54274. In some cases, the synthetic ligand inhibits a covalent interaction between C118 of TERF1 and the probe.

In some instances, the protein is NEDD8-conjugating enzyme Ubc12 (UBE2M) and the cysteine residue is C47, wherein the numbering of the amino acid position corresponds to the amino acid position with the UniProt Identifier P61081. In some cases, the synthetic ligand inhibits a covalent interaction between C47 of UBE2M and the probe.

In some instances, the protein is E3 ubiquitin-protein ligase TRIP12 (TRIP12) and the cysteine residue is C535, wherein the numbering of the amino acid position corresponds to the amino acid position with the UniProt Identifier Q14669. In some cases, the synthetic ligand inhibits a covalent interaction between C535 of TRIP12 and the probe.

In some instances, the protein is ubiquitin carboxyl-terminal hydrolase 10 (USP10) and the cysteine residue is C94, wherein the numbering of the amino acid position corresponds to the amino acid position with the UniProt Identifier Q14694. In some cases, the synthetic ligand inhibits a covalent interaction between C94 of USP10 and the probe.

In some instances, the protein is ubiquitin carboxyl-terminal hydrolase 30 (USP30) and the cysteine residue is C142, wherein the numbering of the amino acid position corresponds to the amino acid position with the UniProt Identifier Q70CQ3. In some cases, the synthetic ligand inhibits a covalent interaction between C142 of USP30 and the probe.

In some instances, the protein is nucleus accumbens-associated protein 1 (NACC1) and the cysteine residue is C301, wherein the numbering of the amino acid position corresponds to the amino acid position with the UniProt Identifier Q96RE7. In some cases, the synthetic ligand inhibits a covalent interaction between C301 of NACC1 and the probe.

In some instances, the protein is lymphoid-specific helicase (HELLS) and the cysteine residue is C277 or C836, wherein the numberings of the amino acid positions correspond to the amino acid positions with the UniProt Identifier Q9NRZ9. In some cases, the synthetic ligand inhibits a covalent interaction between C277 or C836 of HELLS and the probe.

### Cells, Analytical Techniques, and Instrumentation

In certain embodiments, described herein are methods for profiling one or more of NRF2-regulated proteins to determine a reactive or ligandable cysteine residue. In some instances, the methods comprise profiling the NRF2-regulated proteins in situ. In other instances, the methods comprise profiling the NRF2-regulated proteins in vitro. In some instances, the methods comprising profiling the NRF2-regulated proteins utilize a cell sample or a cell lysate sample. In some embodiments, the cell sample or cell lysate sample is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g. Danino rerio). In some cases, the worm cell is from a nematode (e.g. C. elegans). In some cases, the amphibian cell is from a frog. In some embodiments, the arthropod cell is from a tarantula or hermit crab.

In some embodiments, the cell sample or cell lysate sample is obtained from a mammalian cell. In some instances, the mammalian cell is an epithelial cell, connective tissue cell, hormone secreting cell, a nerve cell, a skeletal muscle cell, a blood cell, or an immune system cell.

Exemplary mammalian cells include, but are not limited to, 293A cell line, 293FT cell line, 293F cells, 293 H cells, HEK 293 cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, and PC12 cell line.

In some instances, the cell sample or cell lysate sample is obtained from cells of a tumor cell line. In some instances, the cell sample or cell lysate sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the cell sample or cell lysate sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of: acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the cell sample or cell lysate sample is obtained from a tumor cell line. Exemplary tumor cell line includes, but is not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS153, SU.86.86, SNU-182, SNU-423, SNU-449, SNU-475, SNU-387, Hs817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the cell sample or cell lysate sample is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the cell sample or cell lysate sample is a tissue sample, such as a sample obtained from a biopsy or a tumor tissue sample. In some embodiments, the cell sample or cell lysate sample is a blood serum sample. In some embodiments, the cell sample or cell lysate sample is a blood cell sample containing one or more peripheral blood mononuclear cells (PBMCs). In some embodiments, the cell sample or cell lysate sample contains one or more circulating tumor cells (CTCs). In some embodiments, the cell sample or cell lysate sample contains one or more disseminated tumor cells (DTC, e.g., in a bone marrow aspirate sample).

In some embodiments, the cell sample or cell lysate sample is obtained from the individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, will be examined under a microscope.

Sample Preparation and Analysis

In some embodiments, a sample solution comprises a cell sample, a cell lysate sample, or a sample comprising isolated proteins. In some instances, the sample solution comprises a solution such as a buffer (e.g. phosphate buffered saline) or a media. In some embodiments, the media is an isotopically labeled media. In some instances, the sample solution is a cell solution.

In some embodiments, the solution sample (e.g., cell sample, cell lysate sample, or comprising isolated proteins) is incubated with a compound of Formula (I) for analysis of protein-probe interactions. In some instances, the solution sample (e.g., cell sample, cell lysate sample, or comprising isolated proteins) is further incubated in the presence of an additional compound probe prior to addition of the compound of Formula (I). In other instances, the solution sample (e.g., cell sample, cell lysate sample, or comprising isolated proteins) is further incubated with a ligand, in which the ligand does not contain a photoreactive moiety and/or an alkyne group. In such instances, the solution sample is incubated with a probe and a ligand for competitive protein profiling analysis.

In some cases, the cell sample or the cell lysate sample is compared with a control. In some cases, a difference is observed between a set of probe protein interactions between the sample and the control. In some instances, the difference correlates to the interaction between the small molecule fragment and the proteins.

In some embodiments, one or more methods are utilized for labeling a solution sample (e.g. cell sample, cell lysate sample, or comprising isolated proteins) for analysis of probe protein interactions. In some instances, a method comprises labeling the sample (e.g. cell sample, cell lysate sample, or comprising isolated proteins) with an enriched media. In some cases, the sample (e.g. cell sample, cell lysate sample, or comprising isolated proteins) is labeled with isotope-labeled amino acids, such as ¹³C or ¹⁵N-labeled amino acids. In some cases, the labeled sample is further compared with a non-labeled sample to detect differences in probe protein interactions between the two samples. In some instances, this difference is a difference of a target protein and its interaction with a small molecule ligand in the labeled sample versus the non-labeled sample. In some instances, the difference is an increase, decrease or a lack of protein-probe interaction in the two samples. In some instances, the isotope-labeled method is termed SILAC, stable isotope labeling using amino acids in cell culture.

In some embodiments, a method comprises incubating a solution sample (e.g. cell sample, cell lysate sample, or comprising isolated proteins) with a labeling group (e.g., an isotopically labeled labeling group) to tag one or more proteins of interest for further analysis. In such cases, the labeling group comprises a biotin, a streptavidin, bead, resin, a solid support, or a combination thereof, and further comprises a linker that is optionally isotopically labeled. As described above, the linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with ¹³C and ¹⁵N atoms at one or more amino acid residue positions within the linker. In some cases, the biotin-linker moiety is a isotopically-labeled TEV-tag as described in Weerapana, et al., “Quantitative reactivity profiling predicts functional cysteines in proteomes,” Nature 468(7325): 790-795.

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of probe-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the probe-protein complex is further conjugated to a chromophore, such as a fluorophore. In some instances, the probe-protein complex is separated and visualized utilizing an electrophoresis system, such as through a gel electrophoresis, or a capillary electrophoresis. Exemplary gel electrophoresis includes agarose based gels, polyacrylamide based gels, or starch based gels. In some instances, the probe-protein is subjected to a native electrophoresis condition. In some instances, the probe-protein is subjected to a denaturing electrophoresis condition.

In some instances, the probe-protein after harvesting is further fragmentized to generate protein fragments. In some instances, fragmentation is generated through mechanical stress, pressure, or chemical means. In some instances, the protein from the probe-protein complexes is fragmented by a chemical means. In some embodiments, the chemical means is a protease. Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase Clp, Escherichia coli phage K1F endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase_M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electrospray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ⁸⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state ¹³Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the protein fragments are analyzed by method as described in Weerapana et al., “Quantitative reactivity profiling predicts functional cysteines in proteomes,” Nature, 468:790-795 (2010).

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

In some embodiments, a value is assigned to each of the protein from the probe-protein complex. In some embodiments, the value assigned to each of the protein from the probe-protein complex is obtained from the mass spectroscopy analysis. In some instances, the value is the area-under- the curve from a plot of signal intensity as a function of mass-to-charge ratio. In some instances, the value correlates with the reactivity of a Lys residue within a protein.

In some instances, a ratio between a first value obtained from a first protein sample and a second value obtained from a second protein sample is calculated. In some instances, the ratio is greater than 2.5, 3, 3.5, 4, 4.5, 5, 6, 7, 8, 9, 10, 11, 12, 13, 14, 15, 16, 17, 18, 19, or 20. In some cases, the ratio is at most 20.

In some instances, the ratio is calculated based on averaged values. In some instances, the averaged value is an average of at least two, three, or four values of the protein from each cell solution, or that the protein is observed at least two, three, or four times in each cell solution and a value is assigned to each observed time. In some instances, the ratio further has a standard deviation of less than 12, 10, or 8.

In some instances, a value is not an averaged value. In some instances, the ratio is calculated based on value of a protein observed only once in a cell population. In some instances, the ratio is assigned with a value of 20.

### Kits/Article of Manufacture

Disclosed herein, in certain embodiments, are kits and articles of manufacture for use with one or more methods described herein. In some embodiments, described herein is a kit for generating a protein comprising a photoreactive ligand. In some embodiments, such kit includes photoreactive small molecule ligands described herein, small molecule fragments or libraries and/or controls, and reagents suitable for carrying out one or more of the methods described herein. In some instances, the kit further comprises samples, such as a cell sample, and suitable solutions such as buffers or media. In some embodiments, the kit further comprises recombinant proteins for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include probes, test compounds, and one or more reagents for use in a method disclosed herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Heteroalkylene” refers to an alkyl radical as described above where one or more carbon atoms of the alkyl is replaced with a O, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized π-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

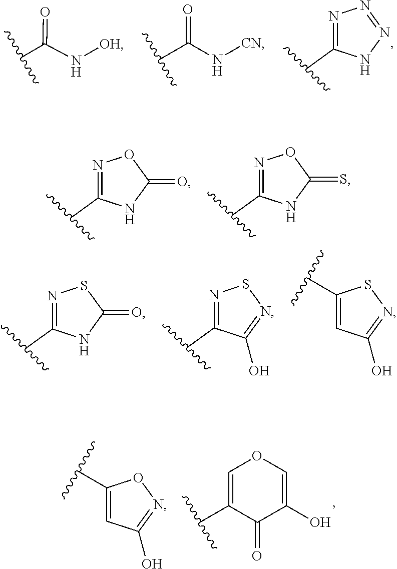

and the like.

“Cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cyclcoalkyl radicals include, for example, cyclopropyl, cyclobutyl, cyclopentyl, cyclohexyl, cycloheptyl, and cyclooctyl. In some embodiments, the monocyclic cyclcoalkyl is cyclopropyl, cyclobutyl, cyclopentyl or cyclohexyl. In some embodiments, the monocyclic cyclcoalkyl is cyclopentyl. Polycyclic radicals include, for example, adamantyl, norbornyl, decalinyl, and 3,4-dihydronaphthalen-1(2H)-one. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g. —NH₂, —NHR, —N(R)₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

The section headings used herein are for organizational purposes only and are not to be construed as limiting the subject matter described.

### EXAMPLES

These examples are provided for illustrative purposes only and not to limit the scope of the claims provided herein.

### Example 1

Table 1A and Table 1B illustrate proteins and cysteine site residues described herein.

TABLE 1A

| UNIPROT | RESIDUES | SYMBOL | DESCRIPTION |
|---|---|---|---|
| Q96RE7 | C301 | NACC1 | NACC1 Nucleus accumbens-associated protein 1 |
| Q14669 | C535 | TRIP12 | TRIP12 E3 ubiquitin-protein ligase TRIP12 |
| Q9NYG5 | C7 | ANAPC11 | ANAPC11 Anaphase-promoting complex subunit 11 |
| Q9UJX4 | C203 | ANAPC5 | ANAPC5 Anaphase-promoting complex subunit 5 |
| O14867 | C646 | BACH1 | BACH1 Transcription regulator protein BACH1 |
| Q9NV06 | C87 | DCAF13 | DCAF13 DDB1- and CUL4-associated factor 13 |
| Q96ME1 | C459, C468 | FBXL18 | FBXL18 F-box/LRR-repeat protein 18 |
| Q8N531 | C368 | FBXL6 | FBXL6 F-box/LRR-repeat protein 6 |
| Q9H2C0 | C248 | GAN | GAN Gigaxonin |
| O95714 | C1005 | HERC2 | HERC2 E3 ubiquitin-protein ligase HERC2 |
| Q14145 | C319 | KEAP1 | KEAP1 Kelch-like ECH-associated protein 1 |
| Q9NX47 | C188 | MARCH5 | MARCH5 E3 ubiquitin-protein ligase MARCH5 |
| O60291 | C428 | MGRN1 | MGRN1 E3 ubiquitin-protein ligase MGRN1 |
| Q96BF6 | C393 | NACC2 | NACC2 Nucleus accumbens-associated protein 2 |
| P49792 | C206 | RANBP2 | RANBP2 E3 SUMO-protein ligase RanBP2 |
| Q93009 | C223 | USP7 | USP7 Ubiquitin carboxyl-terminal hydrolase 7 |
| O95999 | C122, C119 | BCL10 | BCL10 B-cell lymphoma/leukemia 10 |
| P51114 | C99 | FXR1 | FXR1 Fragile X mental retardation syndrome-related protein |
| P41134 | C17 | ID1 | ID1 DNA-binding protein inhibitor ID-1 |
| P10588 | C203 | NR2F6 | NR2F6 Nuclear receptor subfamily 2 group F member 6 |
| P10588 | C316 | NR2F6 | NR2F6 Nuclear receptor subfamily 2 group F member 6 |
| P04049 | C637 | RAF1 | RAF1 RAF proto-oncogene serine/threonine-protein kinase |
| P32320 | C8 | CDA | CDA Cytidine deaminase |
| P07858 | C108, C105 | CTSB | CTSB Cathepsin B |
| P18074 | C663 | ERCC2 | ERCC2 TFIIH basal transcription factor complex helicase |
| Q9NRZ9 | C277 | HELLS | HELLS Lymphoid-specific helicase |
| Q9NRZ9 | C836 | HELLS | HELLS Lymphoid-specific helicase |
| P16144 | C245 | ITGB4 | ITGB4 Integrin beta-4 |
| P16144 | C288 | ITGB4 | ITGB4 Integrin beta-4 |
| O95819 | C883 | MAP4K4 | MAP4K4 Mitogen-activated protein kinase kinase kinase kin |
| P52701 | C615 | MSH6 | MSH6 DNA mismatch repair protein Msh6 |
| P22736 | C551 | NR4A1 | NR4A1 Nuclear receptor subfamily 4 group A member 1 |
| P35610 | C92 | SOAT1 | SOAT1 Sterol O-acyltransferase 1 |
| P54274 | C118 | TERF1 | TERF1 Telomeric repeat-binding factor 1 |
| P61081 | C47 | UBE2M | UBE2M NEDD8-conjugating enzyme Ubc12 |
| Q14694 | C94 | USP10 | USP10 Ubiquitin carboxyl-terminal hydrolase 10 |
| Q70CQ3 | C142 | USP30 | USP30 Ubiquitin carboxyl-terminal hydrolase 30 |
| Q9UHD8 | C375 | SEPT9 | SEPT9 Septin-9 |
| Q9UHD8 | C375, C375+ | SEPT9 | SEPT9 Septin-9 |
| Q9UHD8 | C531 | SEPT9 | SEPT9 Septin-9 |
| Q5JTZ9 | C609 | AARS2 | AARS2 Alanine-tRNA ligase, mitochondrial |
| O60706 | C709 | ABCC9 | ABCC9 ATP-binding cassette sub-family C member 9 |
| O60706 | C788 | ABCC9 | ABCC9 ATP-binding cassette sub-family C member 9 |
| Q8NE71 | C807 | ABCF1 | ABCF1 ATP-binding cassette sub-family F member 1 |
| Q9UG63 | C586 | ABCF2 | ABCF2 ATP-binding cassette sub-family F member 2 |
| Q9UG63 | C388 | ABCF2 | ABCF2 ATP-binding cassette sub-family F member 2 |
| Q8N2K0 | C15, C34 | ABHD12 | ABHD12 Monoacylglycerol lipase ABHD12 |
| Q9H845 | C507 | ACAD9 | ACAD9 Acyl-CoA dehydrogenase family member 9, |
| | | | mitochondria |
| Q9H568 | C197 | ACTL8 | ACTL8 Actin-like protein 8 |
| Q96D53 | C285, C285+ | ADCK4 | ADCK4 Uncharacterized aarF domain-containing protein kin |
| Q96D53 | C335 | ADCK4 | ADCK4 Uncharacterized aarF domain-containing protein kin |
| Q9BRR6 | C40 | ADPGK | ADPGK ADP-dependent glucokinase |
| Q8N556 | C251 | AFAP1 | AFAP1 Actin filament-associated protein 1 |
| Q96P47 | C848 | AGAP3 | AGAP3 Arf-GAP with GTPase, ANK repeat and PH |
| | | | domain-containing protein 3 |
| Q53EU6 | C306 | AGPAT9 | AGPAT9 Glycerol-3-phosphate acyltransferase 3 |
| Q8WYP5 | C693 | AHCTF1 | AHCTF1 Protein ELYS |
| P02765 | C132 | AHSG | AHSG Alpha-2-HS-glycoprotein |
| Q13155 | C306 | AIMP2 | AIMP2 Aminoacyl tRNA synthase complex-interacting |
| | | | multifunctional protein 2 |
| O00170 | C121 | AIP | AIP AH receptor-interacting protein |
| Q99996 | C3067 | AKAP9 | AKAP9 A-kinase anchor protein 9 |
| Q99996 | C3868 | AKAP9 | AKAP9 A-kinase anchor protein 9 |
| O60218 | C299 | AKR1B10 | AKR1B10 Aldo-keto reductase family 1 member B10 |
| Q04828 | C154 | AKR1C1 | AKR1C1 Aldo-keto reductase family 1 member C1 |
| P42330 | C154 | AKR1C3 | AKR1C3 Aldo-keto reductase family 1 member C3 |
| P17516 | C154 | AKR1C4 | AKR1C4 Aldo-keto reductase family 1 member C4 |
| P31749 | C310 | AKT1 | AKT1 RAC-alpha serine/threonine-protein kinase |
| P31751 | C311 | AKT2 | AKT2 RAC-beta serine/threonine-protein kinase |
| Q9Y243 | C307 | AKT3 | AKT3 RAC-gamma serine/threonine-protein kinase |
| P54886 | C612, C606 | ALDH18A1 | ALDH18A1 Delta-1-pyrroline-5-carboxylate synthase |
| P00352 | C303, C302 | ALDH1A1 | ALDH1A1 Retinal dehydrogenase 1 |
| P00352 | C303, C302 | ALDH1A1 | ALDH1A1 Retinal dehydrogenase 1 |
| P47895 | C314, C313 | ALDH1A3 | ALDH1A3 Aldehyde dehydrogenase family 1 member A3 |
| P47895 | C467 | ALDH1A3 | ALDH1A3 Aldehyde dehydrogenase family 1 member A3 |
| Q3SY69 | C445 | ALDH1L2 | ALDH1L2 Mitochondrial 10-formyltetrahydrofolate |
| | | | dehydrogen |
| Q3SY69 | C472 | ALDH1L2 | ALDH1L2 Mitochondrial 10-formyltetrahydrofolate |
| | | | dehydrogen |
| Q3SY69 | C608 | ALDH1L2 | ALDH1L2 Mitochondrial 10-formyltetrahydrofolate |
| | | | dehydrogenase |
| P51648 | C241, C237 | ALDH3A2 | ALDH3A2 Fatty aldehyde dehydrogenase |
| P51648 | C241, C237+, | ALDH3A2 | ALDH3A2 Fatty aldehyde dehydrogenase |
| | C249, C241+, | | |
| | C237 | | |
| P51648 | C249, C241+, | ALDH3A2 | ALDH3A2 Fatty aldehyde dehydrogenase |
| | C241 | | |
| P51648 | C241, C237+, | ALDH3A2 | ALDH3A2 Fatty aldehyde dehydrogenase |
| | C249, C241+, | | |
| | C237 | | |
| P51648 | C249, C241+, | ALDH3A2 | ALDH3A2 Fatty aldehyde dehydrogenase |
| | C241 | | |
| P51648 | C241, C237 | ALDH3A2 | ALDH3A2 Fatty aldehyde dehydrogenase |
| P60006 | C24 | ANAPC15 | ANAPC15 Anaphase-promoting complex subunit 15 |
| Q8IWZ3 | C181 | ANKHD1 | ANKHD1 Ankyrin repeat and KH domain-containing |
| | | | protein 1 |
| Q86XL3 | C674 | ANKLE2 | ANKLE2 Ankyrin repeat and LEM domain-containing |
| | | | protein 2 |
| O75179 | C210 | ANKRD17 | ANKRD17 Ankyrin repeat domain-containing protein 17 |
| Q9BTT0 | C87 | ANP32E | ANP32E Acidic leucine-rich nuclear phosphoprotein 32 |
| | | | family, member E |
| Q63HQ0 | C157 | AP1AR | AP1AR AP-1 complex-associated regulatory protein |
| P61966 | C47 | AP1S1 | AP1S1 AP-1 complex subunit sigma-1A |
| P56377 | C46 | AP1S2 | AP1S2 AP-1 complex subunit sigma-2 |
| Q9UPM8 | C1119 | AP4E1 | AP4E1 AP-4 complex subunit epsilon-1 |
| Q9UBZ4 | C27 | APEX2 | APEX2 DNA-(apurinic or apyrimidinic site) lyase 2 |
| Q6UXV4 | C74 | APOOL | APOOL Apolipoprotein O-like |
| O14497 | C336 | ARID1A | ARID1A AT-rich interactive domain-containing protein 1A |
| O14497 | C336, C336+ | ARID1A | ARID1A AT-rich interactive domain-containing protein 1A |
| P40616 | C80 | ARL1 | ARL1 ADP-ribosylation factor-like protein 1 |
| Q9NVP2 | C201, C189 | ASF1B | ASF1B Histone chaperone ASF1B |
| P00966 | C331 | ASS1 | ASS1 Argininosuccinate synthase |
| Q76L83 | C266 | ASXL2 | ASXL2 Putative Polycomb group protein ASXL2 |
| Q8NBU5 | C137 | ATAD1 | ATAD1 ATPase family AAA domain-containing protein 1 |
| Q8NBU5 | C359 | ATAD1 | ATAD1 ATPase family AAA domain-containing protein 1 |
| Q6PL18 | C635 | ATAD2 | ATAD2 ATPase family AAA domain-containing protein 2 |
| Q5T9A4 | C461+, C461 | ATAD3B | ATAD3B ATPase family AAA domain-containing protein |
| | | | 3B |
| Q7Z3C6 | C630 | ATG9A | ATG9A Autophagy-related protein 9A |
| Q7L8W6 | C88 | ATPBD4 | ATPBD4 ATP-binding domain-containing protein 4 |
| Q9UBB4 | C283 | ATXN10 | ATXN10 Ataxin-10 |
| O14965 | C33 | AURKA | AURKA Aurora kinase A |
| Q9UIG0 | C1045 | BAZ1B | BAZ1B Tyrosine-protein kinase BAZ1B |
| O75815 | C360 | BCAR3 | BCAR3 Breast cancer anti-estrogen resistance protein 3 |
| O75815 | C449 | BCAR3 | BCAR3 Breast cancer anti-estrogen resistance protein 3 |
| P20749 | C115 | BCL3 | BCL3 B-cell lymphoma 3 protein |
| Q02338 | C288 | BDH1 | BDH1 D-beta-hydroxybutyrate dehydrogenase, mitochondria |
| O14503 | C342 | BHLHE40 | BHLHE40 Class E basic helix-loop-helix protein 40 |
| P55957 | C15 | BID | BID BH3-interacting domain death agonist |
| Q96IK1 | C72 | BOD1 | BOD1 Biorientation of chromosomes in cell division protein |
| Q8NFC6 | C74 | BOD1L1 | BOD1L1 Biorientation of chromosomes in cell division |
| | | | protein |
| Q9Y3E2 | C20 | BOLA1 | BOLA1 BolA-like protein 1 |
| Q6PJG6 | C487 | BRAT1 | BRAT1 BRCA1-associated ATM activator 1 |
| Q6PJG6 | C539 | BRAT1 | BRAT1 BRCA1-associated ATM activator 1 |
| Q9NW68 | C49 | BSDC1 | BSDC1 BSD domain-containing protein 1 |
| O14981 | C939, C936 | BTAF1 | BTAF1 TATA-binding protein-associated factor 172 |
| Q9Y6E2 | C97+, C97 | BZW2 | BZW2 Basic leucine zipper and W2 domain-containing |
| | | | protein |
| Q14CZ0 | C79 | C16orf72 | C16orf72 UPF0472 protein C16orf72 |
| Q9HAS0 | C204 | C17orf75 | C17orf75 Protein Njmu-R1 |
| A6NDU8 | C244 | C5orf51 | C5orf51 UPF0600 protein C5orf51 |
| P20810 | C413 | CAST | CAST Calpastatin |
| Q96F63 | C78 | CCDC97 | CCDC97 Coiled-coil domain-containing protein 97 |
| O95273 | C300 | CCNDBP1 | CCNDBP1 Cyclin-D1-binding protein 1 |
| Q9UK58 | C87 | CCNL1 | CCNL1 Cyclin-L1 |
| Q8ND76 | C238 | CCNY | CCNY Cyclin-Y |
| Q8N7R7 | C258 | CCNYL1 | CCNYL1 Cyclin-Y-like protein 1 |
| Q9UK39 | C302 | CCRN4L | CCRN4L Nocturnin |
| P48643 | C429 | CCT5 | CCT5 T-complex protein 1 subunit epsilon |
| Q00587 | C161 | CDC42EP1 | CDC42EP1 Cdc42 effector protein 1 |
| Q9BXL8 | C130 | CDCA4 | CDCA4 Cell division cycle-associated protein 4 |
| O95674 | C286 | CDS2 | CDS2 Phosphatidate cytidylyltransferase 2 |
| Q9H3R5 | C35 | CENPH | CENPH Centromere protein H |
| Q53EZ4 | C159 | CEP55 | CEP55 Centrosomal protein of 55 kDa |
| Q53EZ4 | C236 | CEP55 | CEP55 Centrosomal protein of 55 kDa |
| Q76N32 | C695 | CEP68 | CEP68 Centrosomal protein of 68 kDa |
| Q9H078 | C572 | CLPB | CLPB Caseinolytic peptidase B protein homolog |
| P09497 | C199 | CLTB | CLTB Clathrin light chain B |
| Q969H4 | C42 | CNKSR1 | CNKSR1 Connector enhancer of kinase suppressor of ras 1 |
| Q99439 | C274, C290 | CNN2 | CNN2 Calponin-2 |
| Q15417 | C173+, C173 | CNN3 | CNN3 Calponin-3 |
| Q6PJW8 | C192 | CNST | CNST Consortin |
| Q9Y2Z9 | C178 | COQ6 | COQ6 Ubiquinone biosynthesis monooxygenase COQ6 |
| P31327 | C761, C761+ | CPS1 | CPS1 Carbamoyl-phosphate synthase |
| P50416 | C96 | CPT1A | CPT1A Carnitine O-palmitoyltransferase 1, liver isoform |
| P55060 | C939 | CSE1L | CSE1L Exportin-2 |
| O43310 | C501 | CTIF | CTIF CBP80/20-dependent translation initiation factor |
| O60716 | C692 | CTNND1 | CTNND1 Catenin delta-1 |
| P53634 | C258, C255, | CTSC | CTSC Dipeptidyl peptidase 1 |
| | C258+ | | |
| P53634 | C258+, C258, | CTSC | CTSC Dipeptidyl peptidase 1 |
| | C255, C255+ | | |
| P07339 | C329 | CTSD | CTSD Cathepsin D |
| Q9UBR2 | C132, C154, | CTSZ | CTSZ Cathepsin Z |
| | C126 | | |
| Q9UBR2 | C164 | CTSZ | CTSZ Cathepsin Z |
| Q9UBR2 | C170 | CTSZ | CTSZ Cathepsin Z |
| Q9UBR2 | C179 | CTSZ | CTSZ Cathepsin Z |
| Q9UBR2 | C214 | CTSZ | CTSZ Cathepsin Z |
| O43169 | C115 | CYB5B | CYB5B Cytochrome b5 type B |
| Q07973 | C113 | CYP24A1 | CYP24A1 1,25-dihydroxyvitamin D(3) 24-hydroxylase, |
| | | | mitocho |
| Q07973 | C303 | CYP24A1 | CYP24A1 1,25-dihydroxyvitamin D(3) 24-hydroxylase, |
| | | | mitocho |
| Q9HBI6 | C45 | CYP4F11 | CYP4F11 Cytochrome P450 4F11 |
| Q9HBI6 | C468+, C468 | CYP4F11 | CYP4F11 Cytochrome P450 4F11 |
| Q08477 | C468 | CYP4F3 | CYP4F3 Leukotriene-B(4) omega-hydroxylase 2 |
| Q9NPI6 | C39 | DCP1A | DCP1A mRNA-decapping enzyme 1A |
| Q13561 | C256, C240 | DCTN2 | DCTN2 Dynactin subunit 2 |
| Q7Z4W1 | C138 | DCXR | DCXR L-xylulose reductase |
| Q92499 | C406 | DDX1 | DDX1 ATP-dependent RNA helicase DDX1 |
| Q9NVP1 | C435, C435+ | DDX18 | DDX18 ATP-dependent RNA helicase DDX18 |
| Q9Y6V7 | C258 | DDX49 | DDX49 Probable ATP-dependent RNA helicase DDX49 |
| Q9Y2R4 | C536 | DDX52 | DDX52 Probable ATP-dependent RNA helicase DDX52 |
| Q9NY93 | C311, C298 | DDX56 | DDX56 Probable ATP-dependent RNA helicase DDX56 |
| Q15392 | C91 | DHCR24 | DHCR24 Delta(24)-sterol reductase |
| Q9BPW9 | C203 | DHRS9 | DHRS9 Dehydrogenase/reductase SDR family member 9 |
| Q14147 | C189 | DHX34 | DHX34 Probable ATP-dependent RNA helicase DHX34 |
| Q6P158 | C65 | DHX57 | DHX57 Putative ATP-dependent RNA helicase DHX57 |
| Q08211 | C1029 | DHX9 | DHX9 ATP-dependent RNA helicase A |
| Q08211 | C1029+, C1029 | DHX9 | DHX9 ATP-dependent RNA helicase A |
| Q9UNQ2 | C125 | DIMT1 | DIMT1 Probable dimethyladenosine transferase |
| Q8TDM6 | C1736 | DLG5 | DLG5 Disks large homolog 5 |
| Q8IXB1 | C703, C700 | DNAJC10 | DNAJC10 DnaJ homolog subfamily C member 10 |
| Q8IXB1 | C588 | DNAJC10 | DNAJC10 DnaJ homolog subfamily C member 10 |
| Q8IXB1 | C700 | DNAJC10 | DNAJC10 DnaJ homolog subfamily C member 10 |
| Q8NBA8 | C220 | DTWD2 | DTWD2 DTW domain-containing protein 2 |
| Q14204 | C978 | DYNC1H1 | DYNC1H1 Cytoplasmic dynein 1 heavy chain 1 |
| Q96F86 | C91 | EDC3 | EDC3 Enhancer of mRNA-decapping protein 3 |
| Q05639 | C370, C363 | EEF1A2 | EEF1A2 Elongation factor 1-alpha 2 |
| P26641 | C68 | EEF1G | EEF1G Elongation factor 1-gamma |
| Q12805 | C318, C320, | EFEMP1 | EFEMP1 EGF-containing fibulin-like extracellular matrix p |
| | C318+ | | |
| Q12805 | C332, C338 | EFEMP1 | EFEMP1 EGF-containing fibulin-like extracellular matrix p |
| Q12805 | C224 | EFEMP1 | EFEMP1 EGF-containing fibulin-like extracellular matrix p |
| Q12805 | C365 | EFEMP1 | EFEMP1 EGF-containing fibulin-like extracellular matrix p |
| Q7Z2Z2 | C124 | EFTUD1 | EFTUD1 Elongation factor Tu GTP-binding domain- |
| | | | containing |
| Q9BQ52 | C51 | ELAC2 | ELAC2 Zinc phosphodiesterase ELAC protein 2 |
| Q15723 | C470 | ELF2 | ELF2 ETS-related transcription factor Elf-2 |
| Q96N21 | C52 | ENTHD2 | ENTHD2 AP-4 complex accessory subunit tepsin |
| Q9H6S3 | C358 | EPS8L2 | EPS8L2 Epidermal growth factor receptor kinase substrate |
| O75477 | C310 | ERLIN1 | ERLIN1 Erlin-1 |
| O75477 | C310+, C310 | ERLIN1 | ERLIN1 Erlin-1 |
| Q96HE7 | C37, C35 | ERO1L | ERO1L ERO1-like protein alpha |
| Q96HE7 | C166 | ERO1L | ERO1L ERO1-like protein alpha |
| Q96HE7 | C241 | ERO1L | ERO1L ERO1-like protein alpha |
| Q96HE7 | C37 | ERO1L | ERO1L ERO1-like protein alpha |
| Q96HE7 | C99 | ERO1L | ERO1L ERO1-like protein alpha |
| Q9UJM3 | C146, C142 | ERRFI1 | ERRFI1 ERBB receptor feedback inhibitor 1 |
| Q9UJM3 | C113 | ERRFI1 | ERRFI1 ERBB receptor feedback inhibitor 1 |
| Q6NXG1 | C551 | ESRP1 | ESRP1 Epithelial splicing regulatory protein 1 |
| Q9H6T0 | C581 | ESRP2 | ESRP2 Epithelial splicing regulatory protein 2 |
| Q9BSJ8 | C604, C611 | ESYT1 | ESYT1 Extended synaptotagmin-1 |
| P38117 | C131 | ETFB | ETFB Electron transfer flavoprotein subunit beta |
| P38117 | C42 | ETFB | ETFB Electron transfer flavoprotein subunit beta |
| P38117 | C42+, C42 | ETFB | ETFB Electron transfer flavoprotein subunit beta |
| Q9NVH0 | C109 | EXD2 | EXD2 Exonuclease 3-5 domain-containing protein 2 |
| Q9NVH0 | C133 | EXD2 | EXD2 Exonuclease 3-5 domain-containing protein 2 |
| Q9NVH0 | C227 | EXD2 | EXD2 Exonuclease 3-5 domain-containing protein 2 |
| Q96KP1 | C541 | EXOC2 | EXOC2 Exocyst complex component 2 |
| Q5RKV6 | C117 | EXOSC6 | EXOSC6 Exosome complex component MTR3 |
| P00734 | C391 | F2 | F2 Prothrombin |
| Q6P2I3 | C215 | FAHD2B | FAHD2B Fumarylacetoacetate hydrolase domain- |
| | | | containing pr |
| Q5VSL9 | C769 | FAM40A | FAM40A Protein FAM40A |
| Q6ZRV2 | C550 | FAM83H | FAM83H Protein FAM83H |
| Q9NSD9 | C195 | FARSB | FARSB Phenylalanine--tRNA ligase beta subunit |
| Q9NYY8 | C283 | FASTKD2 | FASTKD2 FAST kinase domain-containing protein 2 |
| Q7L8L6 | C685, C689 | FASTKD5 | FASTKD5 FAST kinase domain-containing protein 5 |
| Q7L8L6 | C689+, C685, | FASTKD5 | FASTKD5 FAST kinase domain-containing protein 5 |
| | C689 | | |
| P37268 | C374 | FDFT1 | FDFT1 Squalene synthase |
| Q14192 | C51, C49 | FHL2 | FHL2 Four and a half LIM domains protein 2 |
| Q8N6M3 | C251 | FITM2 | FITM2 Fat storage-inducing transmembrane protein 2 |
| P21333 | C205, C210 | FLNA | FLNA Filamin-A |
| P21333 | C1260 | FLNA | FLNA Filamin-A |
| O75369 | C183, C178 | FLNB | FLNB Filamin-B |
| O75369 | C660 | FLNB | FLNB Filamin-B |
| P02751 | C2367, C2371 | FN1 | FN1 Fibronectin |
| P02751 | C76, C78 | FN1 | FN1 Fibronectin |
| P02751 | C2317 | FN1 | FN1 Fibronectin |
| Q12841 | C113 | FSTL1 | FSTL1 Follistatin-related protein 1 |
| Q9UI43 | C126 | FTSJ2 | FTSJ2 Putative ribosomal RNA methyltransferase 2 |
| Q8N0W3 | C582 | FUK | FUK L-fucose kinase |
| Q9BUM1 | C269 | G6PC3 | G6PC3 Glucose-6-phosphatase 3 |
| O14976 | C190 | GAK | GAK Cyclin-G-associated kinase |
| Q8WXI9 | C308 | GATAD2B | GATAD2B Transcriptional repressor p66-beta |
| Q8WXI9 | C308, C308+ | GATAD2B | GATAD2B Transcriptional repressor p66-beta |
| Q92538 | C158 | GBF1 | GBF1 Golgi-specific brefeldin A-resistance guanine nucl |
| Q96PP8 | C309 | GBP5 | GBP5 Guanylate-binding protein 5 |
| Q92947 | C115 | GCDH | GCDH Glutaryl-CoA dehydrogenase, mitochondrial |
| Q92616 | C1275 | GCN1L1 | GCN1L1 Translational activator GCN1 |
| Q92616 | C1362 | GCN1L1 | GCN1L1 Translational activator GCN1 |
| Q7L5L3 | C243, C245 | GDPD3 | GDPD3 Glycerophosphodiester phosphodiesterase domain- |
| | | | con |
| P57678 | C210 | GEMIN4 | GEMIN4 Gem-associated protein 4 |
| Q8TEQ6 | C1255 | GEMIN5 | GEMIN5 Gem-associated protein 5 |
| Q96RP9 | C146, C153 | GFM1 | GFM1 Elongation factor G, mitochondrial |
| P62873 | C294 | GNB1 | GNB1 Guanine nucleotide-binding protein G(I)/G(S)/G(T) |
| P62873 | C317 | GNB1 | GNB1 Guanine nucleotide-binding protein G(I)/G(S)/G(T) |
| P62879 | C294 | GNB2 | GNB2 Guanine nucleotide-binding protein G(I)/G(S)/G(T) |
| P62879 | C317 | GNB2 | GNB2 Guanine nucleotide-binding protein G(I)/G(S)/G(T) |
| P63244 | C182 | GNB2L1 | GNB2L1 Guanine nucleotide-binding protein subunit beta-2- |
| Q9BVP2 | C158 | GNL3 | GNL3 Guanine nucleotide-binding protein-like 3 |
| Q08379 | C356 | GOLGA2 | GOLGA2 Golgin subfamily A member 2 |
| P35052 | C401 | GPC1 | GPC1 Glypican-1 |
| Q3KR37 | C210 | GRAMD1B | GRAMD1B GRAM domain-containing protein 1B |
| Q12849 | C29 | GRSF1 | GRSF1 G-rich sequence factor 1 |
| Q12789 | C853 | GTF3C1 | GTF3C1 General transcription factor 30 polypeptide 1 |
| Q9Y5Q9 | C607 | GTF3C3 | GTF3C3 General transcription factor 30 polypeptide 3 |
| Q9NYZ3 | C198 | GTSE1 | GTSE1 G2 and S phase-expressed protein 1 |
| P84243 | C111 | H3F3B | H3F3B Histone H3.3 |
| P40939 | C470 | HADHA | HADHA Trifunctional enzyme subunit alpha, mitochondrial |
| P40939 | C550 | HADHA | HADHA Trifunctional enzyme subunit alpha, mitochondrial |
| P53701 | C46, C35 | HCCS | HCCS Cytochrome c-type heme lyase |
| P53701 | C66 | HCCS | HCCS Cytochrome c-type heme lyase |
| Q9H583 | C1899, C1895 | HEATR1 | HEATR1 HEAT repeat-containing protein 1 |
| Q9H583 | C1942 | HEATR1 | HEATR1 HEAT repeat-containing protein 1 |
| P68431 | C97, C111 | HIST1H3J | HIST1H3J Histone H3.1 |
| P68431 | C97, C111, | HIST1H3J | HIST1H3J Histone H3.1 |
| | C111+ | | |
| Q2TB90 | C517 | HKDC1 | HKDC1 Putative hexokinase HKDC1 |
| P01892 | C188 | HLA-A | HLA-A HLA class I histocompatibility antigen, A-2 alpha |
| P01889 | C188 | HLA-B | HLA-B HLA class I histocompatibility antigen, B-7 alpha |
| Q29960 | C188 | HLA-C | HLA-C HLA class I histocompatibility antigen, Cw-16 alph |
| F8VZB9 | C225 | HLA-C | HLA-C HLA class I histocompatibility antigen, Cw-14 alph |
| Q1KMD3 | C538 | HNRNPUL2 | HNRNPUL2 Heterogeneous nuclear ribonucleoprotein U- |
| | | | like pro |
| P84074 | C185 | HPCA | HPCA Neuron-specific calcium-binding protein hippocalcin |
| Q96IR7 | C168 | HPDL | HPDL 4-hydroxyphenylpyruvate dioxygenase-like protein |
| Q96IR7 | C82 | HPDL | HPDL 4-hydroxyphenylpyruvate dioxygenase-like protein |
| P15428 | C152 | HPGD | HPGD 15-hydroxyprostaglandin dehydrogenase |
| P15428 | C182 | HPGD | HPGD 15-hydroxyprostaglandin dehydrogenase |
| Q86YV9 | C695 | HPS6 | HPS6 Hermansky-Pudlak syndrome 6 protein |
| Q99714 | C58 | HSD17B10 | HSD17B10 3-hydroxyacyl-CoA dehydrogenase type-2 |
| Q6YN16 | C218+, C218 | HSDL2 | HSDL2 Hydroxysteroid dehydrogenase-like protein 2 |
| O43301 | C246 | HSPA12A | HSPA12A Heat shock 70 kDa protein 12A |
| O14558 | C46 | HSPB6 | HSPB6 Heat shock protein beta-6 |
| P10809 | C237 | HSPD1 | HSPD1 60 kDa heat shock protein, mitochondrial |
| A1L0T0 | C354 | ILVBL | ILVBL Acetolactate synthase-like protein |
| Q9NV31 | C107 | IMP3 | IMP3 U3 small nucleolar ribonucleoprotein protein IMP3 |
| P20839 | C327, C331 | IMPDH1 | IMPDH1 Inosine-5-monophosphate dehydrogenase 1 |
| Q27J81 | C284 | INF2 | INF2 Inverted formin-2 |
| Q27J81 | C898 | INF2 | INF2 Inverted formin-2 |
| Q8N201 | C1833 | INTS1 | INTS1 Integrator complex subunit 1 |
| Q96HW7 | C926 | INTS4 | INTS4 Integrator complex subunit 4 |
| Q8TEX9 | C350 | IPO4 | IPO4 Importin-4 |
| O00410 | C473 | IPO5 | IPO5 Importin-5 |
| P35568 | C436 | IRS1 | IRS1 Insulin receptor substrate 1 |
| P05556 | C301 | ITGB1 | ITGB1 Integrin beta-1 |
| Q14573 | C1558 | ITPR3 | ITPR3 Inositol 1,4,5-trisphosphate receptor type 3 |
| Q8IWB1 | C280+, C280, | ITPRIP | ITPRIP Inositol 1,4,5-trisphosphate receptor-interacting |
| | C288 | | |
| P14923 | C457 | JUP | JUP Junction plakoglobin |
| Q7LBC6 | C529 | KDM3B | KDM3B Lysine-specific demethylase 3B |
| Q15004 | C99 | KIAA0101 | KIAA0101 PCNA-associated factor |
| Q14807 | C72 | KIF22 | KIF22 Kinesin-like protein KIF22 |
| O95239 | C153 | KIF4A | KIF4A Chromosome-associated kinesin KIF4A |
| O95239 | C190 | KIF4A | KIF4A Chromosome-associated kinesin KIF4A |
| Q2VIQ3 | C153 | KIF4B | KIF4B Chromosome-associated kinesin KIF4B |
| Q2VIQ3 | C190 | KIF4B | KIF4B Chromosome-associated kinesin KIF4B |
| Q9BW19 | C663 | KIFC1 | KIFC1 Kinesin-like protein KIFC1 |
| P52294 | C210 | KPNA1 | KPNA1 Importin subunit alpha-1 |
| O60684 | C208 | KPNA6 | KPNA6 Importin subunit alpha-7 |
| Q14974 | C585 | KPNB1 | KPNB1 Importin subunit beta-1 |
| Q8N9T8 | C537 | KRI1 | KRI1 Protein KRI1 homolog |
| P13646 | C21 | KRT13 | KRT13 Keratin, type I cytoskeletal 13 |
| Q04695 | C40 | KRT17 | KRT17 Keratin, type I cytoskeletal 17 |
| Q04695 | C60 | KRT17 | KRT17 Keratin, type I cytoskeletal 17 |
| P19013 | C118 | KRT4 | KRT4 Keratin, type II cytoskeletal 4 |
| P02538 | C51 | KRT6A | KRT6A Keratin, type II cytoskeletal 6A |
| P02538 | C77 | KRT6A | KRT6A Keratin, type II cytoskeletal 6A |
| Q6KB66 | C244 | KRT80 | KRT80 Keratin, type II cytoskeletal 80 |
| Q6KB66 | C49 | KRT80 | KRT80 Keratin, type II cytoskeletal 80 |
| Q14533 | C427, C418 | KRT81 | KRT81 Keratin, type II cuticular Hb1 |
| Q14533 | C273 | KRT81 | KRT81 Keratin, type II cuticular Hb1 |
| O00515 | C428 | LAD1 | LAD1 Ladinin-1 |
| Q9Y4W2 | C469, C474 | LAS1L | LAS1L Ribosomal biogenesis protein LAS1L |
| Q9Y4W2 | C699, C706 | LAS1L | LAS1L Ribosomal biogenesis protein LAS1L |
| P80188 | C195 | LCN2 | LCN2 Neutrophil gelatinase-associated lipocalin |
| P18858 | C895 | LIG1 | LIG1 DNA ligase 1 |
| O14910 | C81 | LIN7A | LIN7A Protein lin-7 homolog A |
| Q7L5N7 | C223 | LPCAT2 | LPCAT2 Lysophosphatidylcholine acyltransferase 2 |
| Q96AG4 | C277 | LRRC59 | LRRC59 Leucine-rich repeat-containing protein 59 |
| P83369 | C52 | LSM11 | LSM11 U7 snRNA-associated Sm-like protein LSm11 |
| I3L420 | C80 | LSM14A | LSM14A Protein LSM14 homolog A |
| Q8ND56 | C85 | LSM14A | LSM14A Protein LSM14 homolog A |
| P43355 | C92 | MAGEA1 | MAGEA1 Melanoma-associated antigen 1 |
| O15479 | C301 | MAGEB2 | MAGEB2 Melanoma-associated antigen B2 |
| P52564 | C196, C196+ | MAP2K6 | MAP2K6 Dual specificity mitogen-activated protein kinase |
| P52564 | C196 | MAP2K6 | MAP2K6 Dual specificity mitogen-activated protein kinase |
| O43318 | C513 | MAP3K7 | MAP3K7 Mitogen-activated protein kinase kinase kinase 7 |
| Q3KQU3 | C361 | MAP7D1 | MAP7D1 MAP7 domain-containing protein 1 |
| Q3KQU3 | C373 | MAP7D1 | MAP7D1 MAP7 domain-containing protein 1 |
| Q969Z3 | C272 | MARC2 | MARC2 MOSC domain-containing protein 2, mitochondrial |
| Q9HCC0 | C267 | MCCC2 | MCCC2 Methylcrotonoyl-CoA carboxylase beta chain, |
| | | | mitoch |
| Q9HCC0 | C453 | MCCC2 | MCCC2 Methylcrotonoyl-CoA carboxylase beta chain, |
| | | | mitoch |
| O60318 | C1377 | MCM3AP | MCM3AP 80 kDa MCM3-associated protein |
| P33992 | C207 | MCM5 | MCM5 DNA replication licensing factor MCM5 |
| Q9NU22 | C1358 | MDN1 | MDN1 Midasin |
| Q9NU22 | C1394 | MDN1 | MDN1 Midasin |
| Q9NU22 | C333 | MDN1 | MDN1 Midasin |
| Q9NU22 | C3460 | MDN1 | MDN1 Midasin |
| Q9NU22 | C43 | MDN1 | MDN1 Midasin |
| Q9NU22 | C57 | MDN1 | MDN1 Midasin |
| Q9NU22 | C979 | MDN1 | MDN1 Midasin |
| A6NJ78 | C172 | METTL15 | METTL15 Probable methyltransferase-like protein 15 |
| Q6UX53 | C203, C202 | METTL7B | METTL7B Methyltransferase-like protein 7B |
| Q99685 | C208 | MGLL | MGLL Monoglyceride lipase |
| Q9NYL2 | C22 | MLTK | MLTK Mitogen-activated protein kinase kinase kinase MLT |
| Q9NYL2 | C571 | MLTK | MLTK Mitogen-activated protein kinase kinase kinase MLT |
| P29372 | C56 | MPG | MPG DNA-3-methyladenine glycosylase |
| Q7Z7H8 | C180 | MRPL10 | MRPL10 39S ribosomal protein L10, mitochondrial |
| Q9NX20 | C167 | MRPL16 | MRPL16 39S ribosomal protein L16, mitochondrial |
| Q9BZE1 | C203 | MRPL37 | MRPL37 39S ribosomal protein L37, mitochondrial |
| Q9NYK5 | C133 | MRPL39 | MRPL39 39S ribosomal protein L39, mitochondrial |
| O15235 | C93 | MRPS12 | MRPS12 28S ribosomal protein S12, mitochondrial |
| Q9Y399 | C250, C230, | MRPS2 | MRPS2 28S ribosomal protein S2, mitochondrial |
| | C227 | | |
| Q96EL2 | C103 | MRPS24 | MRPS24 28S ribosomal protein S24, mitochondrial |
| P82663 | C139, C141 | MRPS25 | MRPS25 28S ribosomal protein S25, mitochondrial |
| Q9NZJ7 | C385 | MTCH1 | MTCH1 Mitochondrial carrier homolog 1 |
| P03897 | C39 | MT-ND3 | MT-ND3 NADH-ubiquinone oxidoreductase chain 3 |
| P42345 | C423 | MTOR | MTOR Serine/threonine-protein kinase mTOR |
| P98088 | C4547, C4534 | MUC5AC | MUC5AC Mucin-5AC |
| P98088 | C1643 | MUC5AC | MUC5AC Mucin-5AC |
| P98088 | C2220 | MUC5AC | MUC5AC Mucin-5AC |
| P98088 | C2714 | MUC5AC | MUC5AC Mucin-5AC |
| P98088 | C4071 | MUC5AC | MUC5AC Mucin-5AC |
| P20591 | C42 | MX1 | MX1 Interferon-induced GTP-binding protein Mx1 |
| P35580 | C95 | MYH10 | MYH10 Myosin-10 |
| P35579 | C91 | MYH9 | MYH9 Myosin-9 |
| P35579 | C91, C91+ | MYH9 | MYH9 Myosin-9 |
| O14950 | C109 | MYL12B | MYL12B Myosin regulatory light chain 12B |
| Q96H55 | C755 | MYO19 | MYO19 Unconventional myosin-XIX |
| Q9NZM1 | C2013 | MYOF | MYOF Myoferlin |
| Q147X3 | C74 | NAA30 | NAA30 N-alpha-acetyltransferase 30 |
| P43490 | C287 | NAMPT | NAMPT Nicotinamide phosphoribosyltransferase |
| Q6XQN6 | C385 | NAPRT1 | NAPRT1 Nicotinate phosphoribosyltransferase |
| A2RRP1 | C1777, C1771 | NBAS | NBAS Neuroblastoma-amplified sequence |
| Q9HCD5 | C137 | NCOA5 | NCOA5 Nuclear receptor coactivator 5 |
| Q9UN36 | C321 | NDRG2 | NDRG2 Protein NDRG2 |
| O00483 | C44 | NDUFA4 | NDUFA4 NADH dehydrogenase |
| O75306 | C146 | NDUFS2 | NDUFS2 NADH dehydrogenase |
| O75251 | C183 | NDUFS7 | NDUFS7 NADH dehydrogenase |
| P25208 | C89, C85 | NFYB | NFYB Nuclear transcription factor Y subunit beta |
| Q6KC79 | C1754 | NIPBL | NIPBL Nipped-B-like protein |
| Q9BSC4 | C16 | NOL10 | NOL10 Nucleolar protein 10 |
| Q9BSC4 | C216 | NOL10 | NOL10 Nucleolar protein 10 |
| Q9H8H0 | C368 | NOL11 | NOL11 Nucleolar protein 11 |
| Q9H8H0 | C455 | NOL11 | NOL11 Nucleolar protein 11 |
| Q5C9Z4 | C661 | NOM1 | NOM1 Nucleolar MIF4G domain-containing protein 1 |
| O00567 | C112 | NOP56 | NOP56 Nucleolar protein 56 |
| O00567 | C384 | NOP56 | NOP56 Nucleolar protein 56 |
| Q8NDH3 | C81 | NPEPL1 | NPEPL1 Probable aminopeptidase NPEPL1 |
| P51843 | C200, C215 | NR0B1 | NR0B1 Nuclear receptor subfamily 0 group B member 1 |
| P51843 | C255 | NR0B1 | NR0B1 Nuclear receptor subfamily 0 group B member 1 |
| P51843 | C274 | NR0B1 | NR0B1 Nuclear receptor subfamily 0 group B member 1 |
| P51843 | C290 | NR0B1 | NR0B1 Nuclear receptor subfamily 0 group B member 1 |
| P51843 | C396 | NR0B1 | NR0B1 Nuclear receptor subfamily 0 group B member 1 |
| P24468 | C200 | NR2F2 | NR2F2 COUP transcription factor 2 |
| P46459 | C599 | NSF | NSF Vesicle-fusing ATPase |
| P78549 | C118 | NTHL1 | NTHL1 Endonuclease III-like protein 1 |
| Q9BSD7 | C184 | NTPCR | NTPCR Cancer-related nucleoside-triphosphatase |
| P30990 | C62 | NTS | NTS Neurotensin/neuromedin N |
| P53384 | C277 | NUBP1 | NUBP1 Cytosolic Fe-S cluster assembly factor NUBP1 |
| Q9Y5Y2 | C196, C199, | NUBP2 | NUBP2 Cytosolic Fe-S cluster assembly factor NUBP2 |
| | C202 | | |
| Q9Y5Y2 | C54 | NUBP2 | NUBP2 Cytosolic Fe-S cluster assembly factor NUBP2 |
| P53370 | C44 | NUDT6 | NUDT6 Nucleoside diphosphate-linked moiety X motif 6 |
| O75694 | C874, C863 | NUP155 | NUP155 Nuclear pore complex protein Nup155 |
| O75694 | C874 | NUP155 | NUP155 Nuclear pore complex protein Nup155 |
| Q92621 | C877 | NUP205 | NUP205 Nuclear pore complex protein Nup205 |
| O15381 | C431 | NVL | NVL Nuclear valosin-containing protein-like |
| Q6DKJ4 | C205 | NXN | NXN Nucleoredoxin |
| P00973 | C25 | OAS1 | OAS1 2-5-oligoadenylate synthase 1 |
| Q9H668 | C8 | OBFC1 | OBFC1 CST complex subunit STN1 |
| Q9NX40 | C38 | OCIAD1 | OCIAD1 OCIA domain-containing protein 1 |
| Q9Y5N6 | C88 | ORC6 | ORC6 Origin recognition complex subunit 6 |
| Q9H4L5 | C203 | OSBPL3 | OSBPL3 Oxysterol-binding protein-related protein 3 |
| O95747 | C191 | OXSR1 | OXSR1 Serine/threonine-protein kinase OSR1 |
| Q13153 | C411 | PAK1 | PAK1 Serine/threonine-protein kinase PAK 1 |
| Q13177 | C390 | PAK2 | PAK2 Serine/threonine-protein kinase PAK 2 |
| O75914 | C424 | PAK3 | PAK3 Serine/threonine-protein kinase PAK 3 |
| O95340 | C117 | PAPSS2 | PAPSS2 Bifunctional 3-phosphoadenosine 5-phosphosulfate |
| O95340 | C73 | PAPSS2 | PAPSS2 Bifunctional 3-phosphoadenosine 5-phosphosulfate |
| O95453 | C543 | PARN | PARN Poly(A)-specific ribonuclease PARN |
| Q15154 | C187 | PCM1 | PCM1 Pericentriolar material 1 protein |
| Q99447 | C30 | PCYT2 | PCYT2 Ethanolamine-phosphate cytidylyltransferase |
| Q8WUM4 | C40 | PDCD6IP | PDCD6IP Programmed cell death 6-interacting protein |
| Q29RF7 | C327 | PDS5A | PDS5A Sister chromatid cohesion protein PDS5 homolog A |
| Q8IZL8 | C191, C191+ | PELP1 | PELP1 Proline-, glutamic acid- and leucine-rich protein |
| O00541 | C153 | PES1 | PES1 Pescadillo homolog |
| O96011 | C153 | PEX11B | PEX11B Peroxisomal membrane protein 11B |
| Q92968 | C220 | PEX13 | PEX13 Peroxisomal membrane protein PEX13 |
| Q7Z412 | C173 | PEX26 | PEX26 Peroxisome assembly protein 26 |
| P56589 | C251 | PEX3 | PEX3 Peroxisomal biogenesis factor 3 |
| Q13608 | C564 | PEX6 | PEX6 Peroxisome assembly factor 2 |
| O15067 | C1285, C1287 | PFAS | PFAS Phosphoribosylformylglycinamidine synthase |
| P08237 | C170 | PFKM | PFKM 6-phosphofructokinase, muscle type |
| P08237 | C170+, C170 | PFKM | PFKM 6-phosphofructokinase, muscle type |
| P08237 | C709 | PFKM | PFKM 6-phosphofructokinase, muscle type |
| Q01813 | C360 | PFKP | PFKP 6-phosphofructokinase type C |
| P35232 | C69 | PHB | PHB Prohibitin |
| Q6IE81 | C546 | PHF17 | PHF17 Protein Jade-1 |
| Q8WWQ0 | C28 | PHIP | PHIP PH-interacting protein |
| O00443 | C514 | PIK3C2A | PIK3C2A Phosphatidylinositol 4-phosphate 3-kinase 02 |
| | | | domai |
| Q03405 | C198 | PLAUR | PLAUR Urokinase plasminogen activator surface receptor |
| Q6IQ23 | C542 | PLEKHA7 | PLEKHA7 Pleckstrin homology domain-containing family A |
| | | | mem |
| O60664 | C341 | PLIN3 | PLIN3 Perilipin-3 |
| O60664 | C60 | PLIN3 | PLIN3 Perilipin-3 |
| P53350 | C544 | PLK1 | PLK1 Serine/threonine-protein kinase PLK1 |
| Q04941 | C12, C16 | PLP2 | PLP2 Proteolipid protein 2 |
| Q04941 | C12 | PLP2 | PLP2 Proteolipid protein 2 |
| P13797 | C104 | PLS3 | PLS3 Plastin-3 |
| Q9NRX1 | C226 | PNO1 | PNO1 RNA-binding protein PNO1 |
| Q96AD5 | C61 | PNPLA2 | PNPLA2 Patatin-like phospholipase domain-containing prote |
| Q9NP87 | C119 | POLM | POLM DNA-directed DNA/RNA polymerase mu |
| O95602 | C613 | POLR1A | POLR1A DNA-directed RNA polymerase I subunit RPA1 |
| Q15165 | C42 | PON2 | PON2 Serum paraoxonase/arylesterase 2 |
| Q86W92 | C35 | PPFIBP1 | PPFIBP1 Liprin-beta-1 |
| P50336 | C167 | PPOX | PPOX Protoporphyrinogen oxidase |
| P50336 | C258 | PPOX | PPOX Protoporphyrinogen oxidase |
| O60831 | C28 | PRAF2 | PRAF2 PRA1 family protein 2 |
| O43663 | C531 | PRC1 | PRC1 Protein regulator of cytokinesis 1 |
| P30048 | C229 | PRDX3 | PRDX3 Thioredoxin-dependent peroxide reductase, |
| | | | mitochon |
| P30041 | C47 | PRDX6 | PRDX6 Peroxiredoxin-6 |
| Q9Y478 | C223 | PRKAB1 | PRKAB1 5-AMP-activated protein kinase subunit beta-1 |
| O75400 | C39 | PRPF40A | PRPF40A Pre-mRNA-processing factor 40 homolog A |
| O94906 | C807 | PRPF6 | PRPF6 Pre-mRNA-processing factor 6 |
| O94906 | C837 | PRPF6 | PRPF6 Pre-mRNA-processing factor 6 |
| Q9Y520 | C177 | PRRC2C | PRRC2C Protein PRRC2C |
| O14818 | C63 | PSMA7 | PSMA7 Proteasome subunit alpha type-7 |
| P62195 | C209 | PSMC5 | PSMC5 26S protease regulatory subunit 8 |
| Q96EY7 | C139 | PTCD3 | PTCD3 Pentatricopeptide repeat-containing protein 3, mit |
| Q14914 | C213 | PTGR1 | PTGR1 Prostaglandin reductase 1 |
| Q14914 | C239 | PTGR1 | PTGR1 Prostaglandin reductase 1 |
| Q15269 | C716 | PWP2 | PWP2 Periodic tryptophan protein 2 homolog |
| Q15269 | C86 | PWP2 | PWP2 Periodic tryptophan protein 2 homolog |
| P32322 | C262 | PYCR1 | PYCR1 Pyrroline-5-carboxylate reductase 1, mitochondrial |
| Q96C36 | C262 | PYCR2 | PYCR2 Pyrroline-5-carboxylate reductase 2 |
| Q96C36 | C95 | PYCR2 | PYCR2 Pyrroline-5-carboxylate reductase 2 |
| P47897 | C456 | QARS | QARS Glutamine-tRNA ligase |
| Q5XKP0 | C60 | QIL1 | QIL1 Protein QIL1 |
| Q9H0R6 | C512 | QRSL1 | QRSL1 Glutamyl-tRNA(Gln) amidotransferase subunit A, |
| | | | mit |
| Q6WKZ4 | C1007 | RAB11FIP1 | RAB11FIP1 Rab11 family-interacting protein 1 |
| Q6IQ22 | C68 | RAB12 | RAB12 Ras-related protein Rab-12 |
| P61106 | C40, C40+ | RAB14 | RAB14 Ras-related protein Rab-14 |
| Q9NX57 | C70 | RAB20 | RAB20 Ras-related protein Rab-20 |
| O14966 | C120 | RAB7L1 | RAB7L1 Ras-related protein Rab-7L1 |
| P53611 | C40 | RABGGTB | RABGGTB Geranylgeranyl transferase type-2 subunit beta |
| Q92878 | C157 | RAD50 | RAD50 DNA repair protein RAD50 |
| Q9Y3L5 | C140 | RAP2C | RAP2C Ras-related protein Rap-2c |
| O75884 | C127 | RBBP9 | RBBP9 Putative hydrolase RBBP9 |
| Q96T37 | C926 | RBM15 | RBM15 Putative RNA-binding protein 15 |
| Q8NDT2 | C859 | RBM15B | RBM15B Putative RNA-binding protein 15B |
| A0AV96 | C349 | RBM47 | RBM47 RNA-binding protein 47 |
| Q9Y256 | C314 | RCE1 | RCE1 CAAX prenyl protease 2 |
| Q8IZV5 | C288 | RDH10 | RDH10 Retinol dehydrogenase 10 |
| P35251 | C607 | RFC1 | RFC1 Replication factor C subunit 1 |
| A6NKT7 | C206 | RGPD3 | RGPD3 RanBP2-like and GRIP domain-containing protein 3 |
| Q9HBH0 | C162 | RHOF | RHOF Rho-related GTP-binding protein RhoF |
| Q8IXI2 | C175 | RHOT1 | RHOT1 Mitochondrial Rho GTPase 1 |
| Q6R327 | C1317 | RICTOR | RICTOR Rapamycin-insensitive companion of mTOR |
| Q5UIP0 | C312 | RIF1 | RIF1 Telomere-associated protein RIF1 |
| Q13671 | C223 | RIN1 | RIN1 Ras and Rab interactor 1 |
| Q6NUQ1 | C649 | RINT1 | RINT1 RAD50-interacting protein 1 |
| Q9BVS4 | C449 | RIOK2 | RIOK2 Serine/threonine-protein kinase RIO2 |
| O14730 | C22 | RIOK3 | RIOK3 Serine/threonine-protein kinase RIO3 |
| P27635 | C195 | RPL10 | RPL10 60S ribosomal protein L10 |
| P27635 | C49+, C49 | RPL10 | RPL10 60S ribosomal protein L10 |
| P62913 | C25, C21 | RPL11 | RPL11 60S ribosomal protein L11 |
| P62913 | C25, C21 | RPL11 | RPL11 60S ribosomal protein L11 |
| P50914 | C42 | RPL14 | RPL14 60S ribosomal protein L14 |
| P46776 | C70 | RPL27A | RPL27A 60S ribosomal protein L27a |
| P46779 | C13 | RPL28 | RPL28 60S ribosomal protein L28 |
| P39023 | C114 | RPL3 | RPL3 60S ribosomal protein L3 |
| Q969Q0 | C72, C77 | RPL36AL | RPL36AL 60S ribosomal protein L36a-like |
| P36578 | C208 | RPL4 | RPL4 60S ribosomal protein L4 |
| P36578 | C250 | RPL4 | RPL4 60S ribosomal protein L4 |
| P62424 | C174 | RPL7A | RPL7A 60S ribosomal protein L7a |
| Q6DKI1 | C184 | RPL7L1 | RPL7L1 60S ribosomal protein L7-like 1 |
| P05388 | C27 | RPLP0 | RPLP0 60S acidic ribosomal protein P0 |
| Q9BUL9 | C16 | RPP25 | RPP25 Ribonuclease P protein subunit p25 |
| Q9BUL9 | C16+, C16 | RPP25 | RPP25 Ribonuclease P protein subunit p25 |
| P62280 | C131 | RPS11 | RPS11 40S ribosomal protein S11 |
| P42677 | C40, C37 | RPS27 | RPS27 40S ribosomal protein S27 |
| P42677 | C37 | RPS27 | RPS27 40S ribosomal protein S27 |
| P42677 | C37, C37+ | RPS27 | RPS27 40S ribosomal protein S27 |
| Q71UM5 | C40, C37 | RPS27L | RPS27L 40S ribosomal protein S27-like |
| Q71UM5 | C37 | RPS27L | RPS27L 40S ribosomal protein S27-like |
| Q71UM5 | C37, C37+ | RPS27L | RPS27L 40S ribosomal protein S27-like |
| Q71UM5 | C77 | RPS27L | RPS27L 40S ribosomal protein S27-like |
| P61247 | C96+, C96 | RPS3A | RPS3A 40S ribosomal protein S3a |
| P22090 | C41 | RPS4Y1 | RPS4Y1 40S ribosomal protein S4, Y isoform 1 |
| Q8TD47 | C41 | RPS4Y2 | RPS4Y2 40S ribosomal protein S4, Y isoform 2 |
| P62753 | C100 | RPS6 | RPS6 40S ribosomal protein S6 |
| P56182 | C198 | RRP1 | RRP1 Ribosomal RNA processing protein 1 homolog A |
| P56182 | C62 | RRP1 | RRP1 Ribosomal RNA processing protein 1 homolog A |
| Q5JTH9 | C102 | RRP12 | RRP12 RRP12-like protein |
| Q5JTH9 | C317 | RRP12 | RRP12 RRP12-like protein |
| Q5JTH9 | C763 | RRP12 | RRP12 RRP12-like protein |
| Q16799 | C104, C113 | RTN1 | RTN1 Reticulon-1 |
| Q16799 | C678 | RTN1 | RTN1 Reticulon-1 |
| P28702 | C340 | RXRB | RXRB Retinoic acid receptor RXR-beta |
| P29034 | C94 | S100A2 | S100A2 Protein S100-A2 |
| Q9UPU9 | C20 | SAMD4A | SAMD4A Protein Smaug homolog 1 |
| Q5PRF9 | C20 | SAMD4B | SAMD4B Protein Smaug homolog 2 |
| Q9UHR5 | C172 | SAP30BP | SAP30BP SAP30-binding protein |
| Q9NVU7 | C206 | SDAD1 | SDAD1 Protein SDA1 homolog |
| Q9NVU7 | C405 | SDAD1 | SDAD1 Protein SDA1 homolog |
| P53992 | C1083 | SEC24C | SEC24C Protein transport protein Sec24C |
| P05120 | C79+, C79 | SERPINB2 | SERPINB2 Plasminogen activator inhibitor 2 |
| Q9BYW2 | C1281 | SETD2 | SETD2 Histone-lysine N-methyltransferase SETD2 |
| Q587I9 | C67 | SFT2D3 | SFT2D3 Vesicle transport protein SFT2C |
| Q15464 | C139, C141 | SHB | SHB SH2 domain-containing adapter protein B |
| P29353 | C248, C248+ | SHC1 | SHC1 SHC-transforming protein 1 |
| Q14493 | C72+, C72 | SLBP | SLBP Histone RNA hairpin-binding protein |
| Q9BXP2 | C911 | SLC12A9 | SLC12A9 Solute carrier family 12 member 9 |
| P43007 | C109+, C109 | SLC1A4 | SLC1A4 Neutral amino acid transporter A |
| O43772 | C283 | SLC25A20 | SLC25A20 Mitochondrial carnitine/acylcamitine carrier prot |
| Q9H936 | C271 | SLC25A22 | SLC25A22 Mitochondrial glutamate carrier 1 |
| P12235 | C257 | SLC25A4 | SLC25A4 ADP/ATP translocase 1 |
| P05141 | C257 | SLC25A5 | SLC25A5 ADP/ATP translocase 2 |
| P12236 | C257 | SLC25A6 | SLC25A6 ADP/ATP translocase 3 |
| Q6P1M0 | C560 | SLC27A4 | SLC27A4 Long-chain fatty acid transport protein 4 |
| Q9ULF5 | C364 | SLC39A10 | SLC39A10 Zinc transporter ZIP10 |
| Q15043 | C322 | SLC39A14 | SLC39A14 Zinc transporter ZIP14 |
| Q08AF3 | C875 | SLFN5 | SLFN5 Schlafen family member 5 |
| P51532 | C936 | SMARCA4 | SMARCA4 Transcription activator BRG1 |
| Q96GM5 | C460 | SMARCD1 | SMARCD1 SWI/SNF-related matrix-associated actin- |
| | | | dependent |
| Q14683 | C1115 | SMC1A | SMC1A Structural maintenance of chromosomes protein 1A |
| O95295 | C66 | SNAPIN | SNAPIN SNARE-associated protein Snapin |
| Q9Y5X2 | C455 | SNX8 | SNX8 Sorting nexin-8 |
| P08047 | C755 | SP1 | SP1 Transcription factor Sp1 |
| Q8NB90 | C459 | SPATA5 | SPATA5 Spermatogenesis-associated protein 5 |
| Q9BVQ7 | C309 | SPATA5L1 | SPATA5L1 Spermatogenesis-associated protein 5-like |
| | | | protein |
| Q9NUQ6 | C536, C533 | SPATS2L | SPATS2L SPATS2-like protein |
| O43278 | C331 | SPINT1 | SPINT1 Kunitz-type protease inhibitor 1 |
| P35270 | C159 | SPR | SPR Sepiapterin reductase |
| P11277 | C112 | SPTB | SPTB Spectrin beta chain, erythrocytic |
| Q01082 | C624, C619 | SPTBN1 | SPTBN1 Spectrin beta chain, non-erythrocytic 1 |
| O15020 | C115+, C115 | SPTBN2 | SPTBN2 Spectrin beta chain, non-erythrocytic 2 |
| Q9Y6N5 | C379 | SQRDL | SQRDL Sulfide: quinone oxidoreductase, mitochondrial |
| Q13501 | C290+, C289, | SQSTM1 | SQSTM1 Sequestosome-1 |
| | C290 | | |
| P12931 | C280+, C280 | SRC | SRC Proto-oncogene tyrosine-protein kinase Src |
| P12931 | C280 | SRC | SRC Proto-oncogene tyrosine-protein kinase Src |
| O75044 | C357 | SRGAP2 | SRGAP2 SLIT-ROBO Rho GTPase-activating protein 2 |
| P08240 | C621+, C621 | SRPR | SRPR Signal recognition particle receptor subunit alpha |
| Q9Y5M8 | C179 | SRPRB | SRPRB Signal recognition particle receptor subunit beta |
| Q9Y5M8 | C246 | SRPRB | SRPRB Signal recognition particle receptor subunit beta |
| Q08945 | C200 | SSRP1 | SSRP1 FACT complex subunit SSRP1 |
| Q9Y5Y6 | C801 | ST14 | ST14 Suppressor of tumorigenicity 14 protein |
| Q9Y5Y6 | C830 | ST14 | ST14 Suppressor of tumorigenicity 14 protein |
| Q8N1F8 | C1064 | STK11IP | STK11IP Serine/threonine-protein kinase 11-interacting pro |
| Q9UEW8 | C237 | STK39 | STK39 STE20/SPS1-related proline-alanine-rich protein ki |
| P53597 | C172, C181 | SUCLG1 | SUCLG1 Succinyl-CoA ligase |
| Q8IX01 | C540 | SUGP2 | SUGP2 SURP and G-patch domain-containing protein 2 |
| O94901 | C526 | SUN1 | SUN1 SUN domain-containing protein 1 |
| O94901 | C63 | SUN1 | SUN1 SUN domain-containing protein 1 |
| Q9Y5B9 | C574 | SUPT16H | SUPT16H FACT complex subunit SPT16 |
| Q8WXH0 | C39 | SYNE2 | SYNE2 Nesprin-2 |
| Q8WXH0 | C6161 | SYNE2 | SYNE2 Nesprin-2 |
| Q12962 | C174 | TAF10 | TAF10 Transcription initiation factor TFIID subunit 10 |
| Q15545 | C92 | TAF7 | TAF7 Transcription initiation factor TFIID subunit 7 |
| Q9BW92 | C322 | TARS2 | TARS2 Threonine-tRNA ligase, mitochondrial |
| Q8NHU6 | C1029 | TDRD7 | TDRD7 Tudor domain-containing protein 7 |
| Q15582 | C97 | TGFBI | TGFBI Transforming growth factor-beta-induced protein ig |
| Q8IXH7 | C195 | TH1L | TH1L Negative elongation factor C/D |
| Q07157 | C1727 | TJP1 | TJP1 Tight junction protein ZO-1 |
| Q96SK2 | C158 | TMEM209 | TMEM209 Transmembrane protein 209 |
| Q96SK2 | C301 | TMEM209 | TMEM209 Transmembrane protein 209 |
| Q9BTX1 | C468 | TMEM48 | TMEM48 Nucleoporin NDC1 |
| Q9BTX1 | C468+, C468 | TMEM48 | TMEM48 Nucleoporin NDC1 |
| Q96BY9 | C320 | TMEM66 | TMEM66 Store-operated calcium entry-associated regulatory |
| Q9NVH6 | C167 | TMLHE | TMLHE Trimethyllysine dioxygenase, mitochondrial |
| P42166 | C518 | TMPO | TMPO Lamina-associated polypeptide 2, isoform alpha |
| Q9C0C2 | C1175 | TNKS1BP1 | TNKS1BP1 182 kDa tankyrase-1-binding protein |
| Q8IZW8 | C427 | TNS4 | TNS4 Tensin-4 |
| O96008 | C86, C76, | TOMM40 | TOMM40 Mitochondrial import receptor subunit TOM40 |
| | C74 | | homolo |
| O96008 | C86, C76, | TOMM40 | TOMM40 Mitochondrial import receptor subunit TOM40 |
| | C74 | | homolo |
| O96008 | C86, C76, | TOMM40 | TOMM40 Mitochondrial import receptor subunit TOM40 |
| | C74 | | homolo |
| P11388 | C862 | TOP2A | TOP2A DNA topoisomerase 2-alpha |
| Q02880 | C426 | TOP2B | TOP2B DNA topoisomerase 2-beta |
| Q02880 | C883 | TOP2B | TOP2B DNA topoisomerase 2-beta |
| Q12888 | C1933 | TP53BP1 | TP53BP1 Tumor suppressor p53-binding protein 1 |
| O14773 | C365 | TPP1 | TPP1 Tripeptidyl-peptidase 1 |
| O14773 | C537, C522, | TPP1 | TPP1 Tripeptidyl-peptidase 1 |
| | C526 | | |
| Q9H4I3 | C366 | TRABD | TRABD TraB domain-containing protein |
| O75962 | C1713 | TRIO | TRIO Triple functional domain protein |
| Q15654 | C54, C47 | TRIP6 | TRIP6 Thyroid receptor-interacting protein 6 |
| Q15361 | C708 | TTF1 | TTF1 Transcription termination factor 1 |
| Q71U36 | C315, C316 | TUBA1A | TUBA1A Tubulin alpha-1A chain |
| Q71U36 | C316+, C315, | TUBA1A | TUBA1A Tubulin alpha-1A chain |
| | C316 | | |
| Q13748 | C20, C25, C4 | TUBA3D | TUBA3D Tubulin alpha-3C/D chain |
| Q13748 | C347 | TUBA3D | TUBA3D Tubulin alpha-3C/D chain |
| P68366 | C213, C200 | TUBA4A | TUBA4A Tubulin alpha-4A chain |
| P68366 | C129 | TUBA4A | TUBA4A Tubulin alpha-4A chain |
| P68366 | C376 | TUBA4A | TUBA4A Tubulin alpha-4A chain |
| P68366 | C376+, C376 | TUBA4A | TUBA4A Tubulin alpha-4A chain |
| Q9NY65 | C376 | TUBA8 | TUBA8 Tubulin alpha-8 chain |
| Q9NY65 | C376+, C376 | TUBA8 | TUBA8 Tubulin alpha-8 chain |
| A6NHL2 | C323, C322, | TUBAL3 | TUBAL3 Tubulin alpha chain-like 3 |
| | C322+, C323+ | | |
| P07437 | C201, C211 | TUBB | TUBB Tubulin beta chain |
| P07437 | C201, C211 | TUBB | TUBB Tubulin beta chain |
| Q9BVA1 | C201, C211 | TUBB2B | TUBB2B Tubulin beta-2B chain |
| Q9BVA1 | C201, C211 | TUBB2B | TUBB2B Tubulin beta-2B chain |
| P68371 | C201, C211 | TUBB4B | TUBB4B Tubulin beta-4B chain |
| P68371 | C201, C211 | TUBB4B | TUBB4B Tubulin beta-4B chain |
| Q9BUF5 | C201, C211 | TUBB6 | TUBB6 Tubulin beta-6 chain |
| Q9BUF5 | C201, C211 | TUBB6 | TUBB6 Tubulin beta-6 chain |
| Q2T9J0 | C284 | TYSND1 | TYSND1 Peroxisomal leader peptide-processing protease |
| Q9GZZ9 | C250 | UBA5 | UBA5 Ubiquitin-like modifier-activating enzyme 5 |
| Q9NPG3 | C420 | UBN1 | UBN1 Ubinuclein-1 |
| Q92575 | C144 | UBXN4 | UBXN4 UBX domain-containing protein 4 |
| Q9BZV1 | C125 | UBXN6 | UBXN6 UBX domain-containing protein 6 |
| Q9NYU1 | C1361 | UGGT2 | UGGT2 UDP-glucose: glycoprotein glucosyltransferase 2 |
| F8VZW7 | C77, C74 | Uncharacterized | Uncharacterized protein |
| H7BZ11 | C88, C83 | Uncharacterized | Uncharacterized protein |
| H7C455 | C156 | Uncharacterized | Uncharacterized protein |
| J3KR12 | C188 | Uncharacterized | Uncharacterized protein |
| H7C469 | C200 | Uncharacterized | Uncharacterized protein |
| H3BQZ7 | C538 | Uncharacterized | Uncharacterized protein |
| F5H5T6 | C83 | Uncharacterized | Uncharacterized protein |
| J3KR12 | C95 | Uncharacterized | Uncharacterized protein |
| H7BZ11 | C99 | Uncharacterized | Uncharacterized protein |
| P22695 | C192 | UQCRC2 | UQCRC2 Cytochrome b-c1 complex subunit 2, |
| | | | mitochondrial |
| Q9NVE5 | C50 | USP40 | USP40 Ubiquitin carboxyl-terminal hydrolase 40 |
| P46939 | C447 | UTRN | UTRN Utrophin |
| Q9BQE4 | C174 | VIMP | VIMP Selenoprotein S |
| A3KMH1 | C858 | VWA8 | VWA8 von Willebrand factor A domain-containing protein |
| Q9H3P2 | C141 | WHSC2 | WHSC2 Negative elongation factor A |
| Q9Y4P8 | C393 | WIPI2 | WIPI2 WD repeat domain phosphoinositide-interacting prot |
| Q9HD64 | C33 | XAGE1E | XAGE1E G antigen family D member 2 |
| Q9HD64 | C43 | XAGE1E | XAGE1E G antigen family D member 2 |
| Q9HAV4 | C1131 | XPO5 | XPO5 Exportin-5 |
| P07947 | C287 | YES1 | YES1 Tyrosine-protein kinase Yes |
| P49750 | C1772 | YLPM1 | YLPM1 YLP motif-containing protein 1 |
| Q9NPG8 | C337 | ZDHHC4 | ZDHHC4 Probable palmitoyltransferase ZDHHC4 |
| P17029 | C243 | ZKSCAN1 | ZKSCAN1 Zinc finger protein with KRAB and SCAN |
| | | | domains 1 |

TABLE 1B

| | | Liganded by | | Liganded by |
|---|---|---|---|---|
| UNIPROT | Compound 3 | Compound 3 | Compound 2 | Compound 2 |
| Q96RE7 | — | — | 13.585 | yes |
| Q14669 | 12.06 | yes | 2.2 | no |
| Q9NYG5 | 5.243333 | yes | 14 | no |
| Q9UJX4 | — | — | 8.186667 | yes |
| O14867 | 20 | yes | — | — |
| Q9NV06 | 7.315 | yes | 4.845 | no |
| Q96ME1 | — | — | 20 | yes |
| Q8N531 | 3.54 | no | 6.286667 | yes |
| Q9H2C0 | — | — | 6.935 | yes |
| O95714 | 20 | yes | — | — |
| Q14145 | 12.005 | yes | — | — |
| Q9NX47 | 20 | yes | 2.21 | no |
| O60291 | — | — | 8.625 | yes |
| Q96BF6 | 9.596667 | yes | 2.265 | no |
| P49792 | 6.155 | yes | — | — |
| Q93009 | 1.34 | no | 5.14 | yes |
| O95999 | 5.095 | yes | 8.59 | no |
| P51114 | 1.095 | no | 20 | yes |
| P41134 | 14.63667 | yes | 5.42 | yes |
| P10588 | — | — | 20 | yes |
| P10588 | — | — | 16.04 | yes |
| P04049 | 18.11 | yes | — | — |
| P32320 | 5.19 | no | 20 | yes |
| P07858 | 18.9 | yes | 1.31 | no |
| P18074 | 7.77 | yes | — | — |
| Q9NRZ9 | 20 | yes | — | — |
| Q9NRZ9 | 20 | yes | 4.63 | no |
| P16144 | 16.185 | yes | — | — |
| P16144 | — | — | 5.16 | yes |
| O95819 | 2.295 | no | 6.54 | yes |
| P52701 | 2.09 | no | 5.3 | yes |
| P22736 | — | — | 8.636667 | yes |
| P35610 | 20 | yes | 3.47 | no |
| P54274 | 11.525 | yes | — | — |
| P61081 | 3.12 | no | 5.155 | yes |
| Q14694 | 2.186667 | no | 5.22 | yes |
| Q70CQ3 | 20 | yes | — | — |
| Q9UHD8 | 20 | yes | 2.71 | no |
| Q9UHD8 | 13.57 | yes | 3.2425 | no |
| Q9UHD8 | 3.25 | no | 20 | yes |
| Q5JTZ9 | 2.245 | no | 5.82 | yes |
| O60706 | 19.89 | yes | — | — |
| O60706 | 11.915 | yes | 2.39 | no |
| Q8NE71 | 20 | yes | — | — |
| Q9UG63 | 6.395 | yes | 4.4 | no |
| Q9UG63 | 20 | yes | — | — |
| Q8N2K0 | 20 | yes | — | — |
| Q9H845 | 2.37 | no | 12.98 | yes |
| Q9H568 | — | — | 20 | yes |
| Q96D53 | 20 | yes | 20 | yes |
| Q96D53 | 20 | yes | 13.01 | yes |
| Q9BRR6 | 20 | yes | 1.55 | no |
| Q8N556 | 2.095 | no | 6.465 | yes |
| Q96P47 | 5.316667 | yes | — | — |
| Q53EU6 | 20 | yes | 20 | yes |
| Q8WYP5 | 9.523333 | yes | 2.673333 | no |
| P02765 | 6.996667 | yes | 3.67 | no |
| Q13155 | 3.643333 | no | 5.23 | yes |
| O00170 | 8.18 | yes | — | — |
| Q99996 | 14.825 | yes | 7.1 | yes |
| Q99996 | — | — | 20 | yes |
| O60218 | 12.18 | yes | — | — |
| Q04828 | 5.135 | yes | 4.21 | no |
| P42330 | 5.135 | yes | 4.21 | no |
| P17516 | 5.135 | yes | — | — |
| P31749 | 3.19 | no | 5.096667 | yes |
| P31751 | 3.19 | no | 5.096667 | yes |
| Q9Y243 | 3.19 | no | 5.096667 | yes |
| P54886 | 4.37 | no | 13.245 | yes |
| P00352 | 20 | yes | — | — |
| P00352 | 20 | yes | — | — |
| P47895 | 20 | yes | 20 | yes |
| P47895 | 20 | yes | — | — |
| Q3SY69 | 16.485 | yes | 8.89 | no |
| Q3SY69 | 7.955 | yes | 8.89 | no |
| Q3SY69 | 15 | yes | — | — |
| P51648 | 2.853333 | no | 20 | yes |
| P51648 | 4.52 | no | 20 | yes |
| P51648 | 2.95 | no | 20 | yes |
| P51648 | 4.52 | no | 20 | yes |
| P51648 | 2.95 | no | 20 | yes |
| P51648 | 2.853333 | no | 20 | yes |
| P60006 | — | — | 20 | yes |
| Q8IWZ3 | 20 | yes | — | — |
| Q86XL3 | — | — | 12.335 | yes |
| O75179 | 20 | yes | — | — |
| Q9BTT0 | 5.405 | yes | 3.61 | no |
| Q63HQ0 | 5.175 | yes | — | — |
| P61966 | — | — | 5.655 | yes |
| P56377 | — | — | 5.655 | yes |
| Q9UPM8 | 20 | yes | — | — |
| Q9UBZ4 | 6.46 | yes | — | — |
| Q6UXV4 | 20 | yes | 3.19 | no |
| O14497 | 3.16 | no | 12.355 | yes |
| O14497 | 1.854 | no | 6.095 | yes |
| P40616 | — | — | 6.49 | yes |
| Q9NVP2 | 3.005 | no | 5.23 | yes |
| P00966 | 6.665 | yes | 5.245 | yes |
| Q76L83 | 7.09 | yes | 3.49 | no |
| Q8NBU5 | 8.825 | yes | 5.1 | yes |
| Q8NBU5 | 6.745 | yes | 2.15 | no |
| Q6PL18 | — | — | 12.365 | yes |
| Q5T9A4 | 3.51 | no | 9.27 | yes |
| Q7Z3C6 | — | — | 13.175 | yes |
| Q7L8W6 | 11.82 | yes | 3.35 | no |
| Q9UBB4 | 20 | yes | 2.876667 | no |
| O14965 | 3.03 | no | 6.346667 | yes |
| Q9UIG0 | 20 | yes | — | — |
| O75815 | 20 | yes | 3.94 | no |
| O75815 | 4.19 | no | 6.51 | yes |
| P20749 | 17.72 | yes | 8.75 | no |
| Q02338 | — | — | 20 | yes |
| O14503 | — | — | 5.415 | yes |
| P55957 | — | — | 20 | yes |
| Q96IK1 | — | — | 6.01 | yes |
| Q8NFC6 | — | — | 6.01 | yes |
| Q9Y3E2 | 1.935 | no | 6.546667 | yes |
| Q6PJG6 | 8.08 | yes | 2.245 | no |
| Q6PJG6 | 7.386667 | yes | 1.255 | no |
| Q9NW68 | 20 | yes | — | — |
| O14981 | 2.47 | no | 6.07 | yes |
| Q9Y6E2 | — | — | 12.56 | yes |
| Q14CZ0 | 12.9 | yes | — | — |
| Q9HAS0 | 6.49 | no | 5.826667 | yes |
| A6NDU8 | 20 | yes | — | — |
| P20810 | 3.87 | no | 5.4 | yes |
| Q96F63 | — | — | 5.69 | yes |
| O95273 | 4.09 | no | 20 | yes |
| Q9UK58 | 10.795 | yes | 4.475 | no |
| Q8ND76 | — | — | 13.49 | yes |
| Q8N7R7 | 20 | yes | 13.49 | yes |
| Q9UK39 | 20 | yes | 20 | yes |
| P48643 | 7.65 | no | 8.645 | yes |
| Q00587 | — | — | 13.405 | yes |
| Q9BXL8 | 7.61 | yes | 3.23 | no |
| O95674 | 3.275 | no | 18.85333 | yes |
| Q9H3R5 | 5.53 | yes | 3.163333 | no |
| Q53EZ4 | 4.265 | no | 5.143333 | yes |
| Q53EZ4 | — | — | 5.855 | yes |
| Q76N32 | 13.895 | yes | — | — |
| Q9H078 | 3.49 | no | 5.825 | yes |
| P09497 | 6.413333 | yes | 4.69 | no |
| Q969H4 | 20 | yes | 3.28 | no |
| Q99439 | 2.665 | no | 5.27 | yes |
| Q15417 | 1.893333 | no | 7.2 | yes |
| Q6PJW8 | 12.74 | yes | 6.56 | yes |
| Q9Y2Z9 | 5.263333 | yes | 4.33 | no |
| P31327 | 6.376667 | yes | 4.113333 | no |
| P50416 | 20 | yes | — | — |
| P55060 | 1.69 | no | 6.195 | yes |
| O43310 | 6.285 | yes | — | — |
| O60716 | 5.983333 | yes | 3.73 | no |
| P53634 | 20 | yes | 1.398333 | no |
| P53634 | 20 | yes | 1.963333 | no |
| P07339 | — | — | 12.705 | yes |
| Q9UBR2 | 4.37 | no | 7.855 | yes |
| Q9UBR2 | 3.62 | no | 6.3 | yes |
| Q9UBR2 | 3.565 | no | 8.445 | yes |
| Q9UBR2 | 4.21 | no | 7.07 | yes |
| Q9UBR2 | 7.91 | yes | — | — |
| O43169 | 20 | yes | 20 | yes |
| Q07973 | 17.38 | yes | — | — |
| Q07973 | — | — | 5.195 | yes |
| Q9HBI6 | 20 | yes | 5.06 | no |
| Q9HBI6 | 13.105 | yes | 6.06 | yes |
| Q08477 | 13.105 | yes | — | — |
| Q9NPI6 | 8.89 | no | 5.22 | yes |
| Q13561 | 4.205 | no | 6.05 | yes |
| Q7Z4W1 | 1.913333 | no | 5.766667 | yes |
| Q92499 | 16.63 | yes | 2.415 | no |
| Q9NVP1 | 8.4475 | yes | 20 | yes |
| Q9Y6V7 | 2.515 | no | 20 | yes |
| Q9Y2R4 | 11.42667 | yes | 2.08 | no |
| Q9NY93 | 2.375 | no | 6.19 | yes |
| Q15392 | 16.06 | yes | 19.65 | yes |
| Q9BPW9 | — | — | 5.345 | yes |
| Q14147 | 20 | yes | — | — |
| Q6P158 | — | — | 5.125 | yes |
| Q08211 | 6.6 | no | 8.403333 | yes |
| Q08211 | 9.6775 | yes | 9.976667 | yes |
| Q9UNQ2 | 20 | yes | 5.47 | yes |
| Q8TDM6 | 18.63 | yes | 18.62 | no |
| Q8IXB1 | 20 | yes | 7.305 | yes |
| Q8IXB1 | 20 | yes | 10.36 | no |
| Q8IXB1 | 20 | yes | — | — |
| Q8NBA8 | 20 | yes | 20 | yes |
| Q14204 | — | — | 8.53 | yes |
| Q96F86 | — | — | 5.58 | yes |
| Q05639 | 8.55 | yes | 2.79 | no |
| P26641 | 16.95667 | yes | 8.79 | yes |
| Q12805 | 3.752 | no | 5.766667 | yes |
| Q12805 | 3.31 | no | 9.64 | yes |
| Q12805 | 3.195 | no | 6.75 | yes |
| Q12805 | — | — | 15.33333 | yes |
| Q7Z2Z2 | 20 | yes | — | — |
| Q9BQ52 | 2 | no | 6.546667 | yes |
| Q15723 | 2.87 | no | 6.63 | yes |
| Q96N21 | — | — | 20 | yes |
| Q9H6S3 | — | — | 20 | yes |
| O75477 | 20 | yes | 7.31 | no |
| O75477 | 6.203333 | yes | 9.825 | yes |
| Q96HE7 | 20 | yes | 20 | yes |
| Q96HE7 | 10.62 | yes | 6.48 | yes |
| Q96HE7 | 5.793333 | yes | 7.845 | yes |
| Q96HE7 | 20 | yes | — | — |
| Q96HE7 | — | — | 5.95 | yes |
| Q9UJM3 | 6.93 | no | 7.515 | yes |
| Q9UJM3 | 14.75667 | yes | 3.49 | no |
| Q6NXG1 | — | — | 7.326667 | yes |
| Q9H6T0 | 20 | yes | 17.715 | yes |
| Q9BSJ8 | 2.89 | no | 9.235 | yes |
| P38117 | 4.29 | no | 12.115 | yes |
| P38117 | 20 | yes | 1.35 | no |
| P38117 | 19.48667 | yes | 1.605 | no |
| Q9NVH0 | 6.8 | yes | 4.08 | no |
| Q9NVH0 | 6.443333 | yes | 2.33 | no |
| Q9NVH0 | 8.06 | no | 9.893333 | yes |
| Q96KP1 | 5.97 | no | 20 | yes |
| Q5RKV6 | 2.79 | no | 5.306667 | yes |
| P00734 | — | — | 14.525 | yes |
| Q6P2I3 | 12.845 | yes | 2.08 | no |
| Q5VSL9 | — | — | 20 | yes |
| Q6ZRV2 | 5.66 | no | 20 | yes |
| Q9NSD9 | 1.42 | no | 5.79 | yes |
| Q9NYY8 | 20 | yes | 2.145 | no |
| Q7L8L6 | 12.32 | yes | 4.23 | no |
| Q7L8L6 | 2.456667 | no | 11.732 | yes |
| P37268 | — | — | 5.315 | yes |
| Q14192 | 2.25 | no | 7.116667 | yes |
| Q8N6M3 | 20 | yes | 2.82 | no |
| P21333 | 6.65 | yes | 4.835 | no |
| P21333 | 2.02 | no | 6.833333 | yes |
| O75369 | 5.275 | yes | 5.03 | yes |
| O75369 | 8.96 | yes | 3.365 | no |
| P02751 | 7.255 | yes | 20 | yes |
| P02751 | — | — | 20 | yes |
| P02751 | 17.76 | yes | 20 | yes |
| Q12841 | 5 | no | 9.7 | yes |
| Q9UI43 | 14.34 | yes | 2.415 | no |
| Q8N0W3 | 2.24 | no | 20 | yes |
| Q9BUM1 | 20 | yes | — | — |
| O14976 | 20 | yes | 13.065 | yes |
| Q8WXI9 | 3.12 | no | 10.12 | yes |
| Q8WXI9 | 2.7225 | no | 6.716667 | yes |
| Q92538 | 2.693333 | no | 7.73 | yes |
| Q96PP8 | 20 | yes | 2.33 | no |
| Q92947 | 9.2 | yes | 1.54 | no |
| Q92616 | — | — | 12.18 | yes |
| Q92616 | 13.21 | yes | 1.51 | no |
| Q7L5L3 | 20 | yes | 20 | yes |
| P57678 | 9.49 | yes | 5.265 | yes |
| Q8TEQ6 | 17.185 | yes | 1.665 | no |
| Q96RP9 | 4.095 | no | 6.65 | yes |
| P62873 | 20 | yes | — | — |
| P62873 | 5.166667 | yes | 2.455 | no |
| P62879 | 13.41333 | yes | 3.63 | no |
| P62879 | 5.166667 | yes | 2.455 | no |
| P63244 | 10.905 | yes | 0.966667 | no |
| Q9BVP2 | 6.093333 | yes | 1.95 | no |
| Q08379 | 2.28 | no | 5.595 | yes |
| P35052 | — | — | 13.38333 | yes |
| Q3KR37 | 20 | yes | — | — |
| Q12849 | 20 | yes | — | — |
| Q12789 | 3.75 | no | 16.57333 | yes |
| Q9Y5Q9 | 14.39667 | yes | 8.09 | yes |
| Q9NYZ3 | 2.355 | no | 5.31 | yes |
| P84243 | 5.79 | yes | 3.996667 | no |
| P40939 | 18.85 | yes | 11.50667 | yes |
| P40939 | 9.243333 | yes | 5.39 | yes |
| P53701 | 3.58 | no | 6.19 | yes |
| P53701 | 12.335 | yes | 6.28 | yes |
| Q9H583 | 20 | yes | — | — |
| Q9H583 | — | — | 9.306667 | yes |
| P68431 | 5.56 | yes | 3.88 | no |
| P68431 | 7.155 | yes | 2.67 | no |
| Q2TB90 | 5.34 | yes | 1.715 | no |
| P01892 | — | — | 15.03333 | yes |
| P01889 | 20 | yes | 7.25 | yes |
| Q29960 | — | — | 15.03333 | yes |
| F8VZB9 | 20 | yes | — | — |
| Q1KMD3 | 14.30667 | yes | 4.893333 | no |
| P84074 | 19.54667 | yes | 8.465 | yes |
| Q96IR7 | 9.015 | yes | 5.05 | yes |
| Q96IR7 | 12.67 | yes | 1.65 | no |
| P15428 | 20 | yes | — | — |
| P15428 | 20 | yes | 20 | yes |
| Q86YV9 | 7.625 | yes | 1.555 | no |
| Q99714 | 3.86 | no | 5.526667 | yes |
| Q6YN16 | 13.755 | yes | 3.07 | no |
| O43301 | 5.88 | no | 5.47 | yes |
| O14558 | 3.885 | no | 6.1 | yes |
| P10809 | 4.28 | no | 5.665 | yes |
| A1L0T0 | 2.26 | no | 12.55 | yes |
| Q9NV31 | 3.32 | no | 17.03 | yes |
| P20839 | 15.02667 | yes | 20 | yes |
| Q27J81 | 13.695 | yes | 1.71 | no |
| Q27J81 | 20 | yes | 1.34 | no |
| Q8N201 | — | — | 9.043333 | yes |
| Q96HW7 | 5.46 | yes | 20 | yes |
| Q8TEX9 | 8.77 | no | 6.1 | yes |
| O00410 | — | — | 6.72 | yes |
| P35568 | 2.39 | no | 6.09 | yes |
| P05556 | 15.715 | yes | 3.74 | no |
| Q14573 | 2.54 | no | 5.42 | yes |
| Q8IWB1 | 10.51333 | yes | 3.51 | no |
| P14923 | 10.25 | yes | 3.33 | no |
| Q7LBC6 | 5.345 | yes | 5.92 | yes |
| Q15004 | 3.59 | no | 10.085 | yes |
| Q14807 | — | — | 13.11 | yes |
| O95239 | 20 | yes | 4.345 | no |
| O95239 | 7.59 | yes | 3.54 | no |
| Q2VIQ3 | 20 | yes | — | — |
| Q2VIQ3 | 7.59 | yes | 3.54 | no |
| Q9BW19 | 20 | yes | 3.73 | no |
| P52294 | — | — | 8.325 | yes |
| O60684 | — | — | 14.41 | yes |
| Q14974 | 15.66 | yes | 2.26 | no |
| Q8N9T8 | 2.08 | no | 11.34667 | yes |
| P13646 | 15.88 | yes | 19.81 | yes |
| Q04695 | 20 | yes | 13.84 | yes |
| Q04695 | 7.755 | yes | 5.485 | yes |
| P19013 | — | — | 9.7 | yes |
| P02538 | 12.215 | yes | 12.3 | yes |
| P02538 | 17.17 | yes | 12.92333 | yes |
| Q6KB66 | 20 | yes | 6.54 | no |
| Q6KB66 | 5.26 | yes | 12.715 | yes |
| Q14533 | 20 | yes | — | — |
| Q14533 | 11.87667 | yes | 2.71 | no |
| O00515 | 20 | yes | — | — |
| Q9Y4W2 | 20 | yes | — | — |
| Q9Y4W2 | 2.47 | no | 5.06 | yes |
| P80188 | 1.85 | no | 8.475 | yes |
| P18858 | 1.566667 | no | 5.27 | yes |
| O14910 | 16.73 | yes | — | — |
| Q7L5N7 | 5.42 | yes | 2.92 | no |
| Q96AG4 | 20 | yes | 11.39 | yes |
| P83369 | 14.01 | yes | — | — |
| I3L420 | 2.436667 | no | 5.39 | yes |
| Q8ND56 | 2.436667 | no | 5.39 | yes |
| P43355 | — | — | 20 | yes |
| O15479 | 3.72 | no | 8.943333 | yes |
| P52564 | 18.53333 | yes | 12.715 | yes |
| P52564 | — | — | 18.35 | yes |
| O43318 | 20 | yes | — | — |
| Q3KQU3 | 7.23 | yes | 4.935 | no |
| Q3KQU3 | 11.58333 | yes | 4.45 | no |
| Q969Z3 | 20 | yes | 20 | yes |
| Q9HCC0 | 1.01 | no | 5.783333 | yes |
| Q9HCC0 | 11.68667 | yes | 2.73 | no |
| O60318 | — | — | 12.385 | yes |
| P33992 | 20 | yes | 20 | yes |
| Q9NU22 | 20 | yes | — | — |
| Q9NU22 | 5.595 | yes | 2.196667 | no |
| Q9NU22 | 20 | yes | 8.745 | yes |
| Q9NU22 | 6.35 | yes | — | — |
| Q9NU22 | — | — | 20 | yes |
| Q9NU22 | — | — | 20 | yes |
| Q9NU22 | 20 | yes | 9.35 | no |
| A6NJ78 | 5.115 | yes | 6.77 | yes |
| Q6UX53 | 5.94 | yes | 0.965 | no |
| Q99685 | 4.305 | no | 13.87333 | yes |
| Q9NYL2 | 2.21 | no | 20 | yes |
| Q9NYL2 | 5.21 | yes | — | — |
| P29372 | — | — | 5.695 | yes |
| Q7Z7H8 | 20 | yes | 2.82 | no |
| Q9NX20 | 7.91 | yes | 3.13 | no |
| Q9BZE1 | 13.17 | yes | — | — |
| Q9NYK5 | 1.39 | no | 7.216667 | yes |
| O15235 | 6.01 | yes | 2.806667 | no |
| Q9Y399 | 6.415 | yes | — | — |
| Q96EL2 | 5.795 | yes | 3.46 | no |
| P82663 | 3.876667 | no | 5.61 | yes |
| Q9NZJ7 | — | — | 6.7 | yes |
| P03897 | 7.253333 | yes | 2.973333 | no |
| P42345 | 16.705 | yes | — | — |
| P98088 | — | — | 7.35 | yes |
| P98088 | — | — | 8.245 | yes |
| P98088 | — | — | 5.08 | yes |
| P98088 | 4.09 | no | 6.905 | yes |
| P98088 | — | — | 7.915 | yes |
| P20591 | 5.43 | yes | — | — |
| P35580 | — | no | 5.66 | yes |
| P35579 | — | — | 5.66 | yes |
| P35579 | 10.38 | yes | 3.36 | no |
| O14950 | 13.95667 | yes | 2.5 | no |
| Q96H55 | — | — | 5.55 | yes |
| Q9NZM1 | 20 | yes | 18.07 | no |
| Q147X3 | — | — | 6.113333 | yes |
| P43490 | 9.745 | yes | 3.26 | no |
| Q6XQN6 | 5.05 | yes | 2.32 | no |
| A2RRP1 | 2.515 | no | 13.655 | yes |
| Q9HCD5 | 1.88 | no | 5.34 | yes |
| Q9UN36 | 1.7 | no | 9.465 | yes |
| O00483 | 12.58 | yes | 2.19 | no |
| O75306 | 9.68 | yes | 3.836667 | no |
| O75251 | 20 | yes | 5.99 | yes |
| P25208 | — | — | 13.645 | yes |
| Q6KC79 | 20 | yes | — | — |
| Q9BSC4 | — | — | 7.826667 | yes |
| Q9BSC4 | — | — | 5.435 | yes |
| Q9H8H0 | 2.6 | no | 12.765 | yes |
| Q9H8H0 | 9.025 | yes | — | — |
| Q5C9Z4 | 20 | yes | 8.315 | yes |
| O00567 | 14.82333 | yes | 3.78 | no |
| O00567 | 20 | yes | 4.02 | no |
| Q8NDH3 | 2.1 | no | 15.38 | yes |
| P51843 | 10.62 | yes | — | — |
| P51843 | 15.795 | yes | 3.51 | no |
| P51843 | 18.19 | yes | 20 | yes |
| P51843 | 6.355 | yes | 2.875 | no |
| P51843 | 6.073333 | yes | 3.896667 | no |
| P24468 | 20 | yes | — | — |
| P46459 | 7.2 | yes | 2.475 | no |
| P78549 | 1.775 | no | 7.966667 | yes |
| Q9BSD7 | 6.003333 | yes | 1.81 | no |
| P30990 | — | — | 20 | yes |
| P53384 | 13.755 | yes | — | — |
| Q9Y5Y2 | 13.26 | yes | 5.48 | yes |
| Q9Y5Y2 | 5.196667 | yes | 1.715 | no |
| P53370 | 20 | yes | — | — |
| O75694 | 2.56 | no | 18.19333 | yes |
| O75694 | 2.04 | no | 20 | yes |
| Q92621 | 5.24 | yes | 2.95 | no |
| O15381 | 6.22 | yes | 2.805 | no |
| Q6DKJ4 | — | — | 15.525 | yes |
| P00973 | 1.583333 | no | 5.006667 | yes |
| Q9H668 | 1.295 | no | 8.325 | yes |
| Q9NX40 | 8.093333 | yes | 2.096667 | no |
| Q9Y5N6 | — | — | 6.64 | yes |
| Q9H4L5 | 3.595 | no | 6.226667 | yes |
| O95747 | 20 | yes | 4.66 | no |
| Q13153 | 20 | yes | 2.54 | no |
| Q13177 | 20 | yes | 2.54 | no |
| O75914 | 20 | yes | 2.54 | no |
| O95340 | 8.98 | yes | 2.15 | no |
| O95340 | 5.383333 | yes | 3.725 | no |
| O95453 | — | — | 20 | yes |
| Q15154 | 9.47 | yes | 3.14 | no |
| Q99447 | 7.12 | yes | 1.56 | no |
| Q8WUM4 | 3.27 | no | 11.295 | yes |
| Q29RF7 | 0.82 | no | 5.735 | yes |
| Q8IZL8 | 16.06 | yes | 8.13 | no |
| O00541 | 5.025 | yes | 15.65 | yes |
| O96011 | 4.53 | no | 17.275 | yes |
| Q92968 | 20 | yes | — | — |
| Q7Z412 | 16.745 | yes | 2.35 | no |
| P56589 | 20 | yes | 3.41 | no |
| Q13608 | 5.395 | yes | 3.52 | no |
| O15067 | 2.515 | no | 11.85667 | yes |
| P08237 | 2.01 | no | 8.613333 | yes |
| P08237 | 2.57 | no | 9.805 | yes |
| P08237 | — | — | 20 | yes |
| Q01813 | 5.565 | yes | 3.695 | no |
| P35232 | 3.39 | no | 5.545 | yes |
| Q6IE81 | — | — | 20 | yes |
| Q8WWQ0 | 6.815 | yes | 1.02 | no |
| O00443 | 15.945 | yes | — | — |
| Q03405 | — | — | 12.615 | yes |
| Q6IQ23 | 2.77 | no | 8.39 | yes |
| O60664 | 5.04 | yes | 2.005 | no |
| O60664 | 5.44 | yes | 1.54 | no |
| P53350 | 10.11667 | yes | 11.29 | yes |
| Q04941 | 8.945 | yes | 4.125 | no |
| Q04941 | 16.91 | yes | 6.99 | yes |
| P13797 | 1.79 | no | 8.24 | yes |
| Q9NRX1 | 6.746667 | yes | 4.906667 | no |
| Q96AD5 | 20 | yes | — | — |
| Q9NP87 | — | — | 6.95 | yes |
| O95602 | 9.645 | yes | 1.05 | no |
| Q15165 | 20 | yes | 5.2 | no |
| Q86W92 | 13.245 | yes | — | — |
| P50336 | — | — | 12.61 | yes |
| P50336 | 7.805 | yes | 8.3 | yes |
| O60831 | — | — | 6.365 | yes |
| O43663 | 1.655 | no | 5.393333 | yes |
| P30048 | 5.606667 | yes | 5.41 | yes |
| P30041 | 7.21 | yes | 10.51333 | yes |
| Q9Y478 | — | — | 12.485 | yes |
| O75400 | 3.056667 | no | 13.87 | yes |
| O94906 | 8.835 | yes | 4.255 | no |
| O94906 | 5.4 | yes | 3.483333 | no |
| Q9Y520 | 1.62 | no | 5.66 | yes |
| O14818 | 10.85333 | yes | 2.655 | no |
| P62195 | 6.26 | yes | 1.335 | no |
| Q96EY7 | — | — | 20 | yes |
| Q14914 | 3.71 | no | 5.365 | yes |
| Q14914 | 6.245 | yes | 1.69 | no |
| Q15269 | 4.245 | no | 5.46 | yes |
| Q15269 | 17.935 | yes | 3.61 | no |
| P32322 | 14.78333 | yes | 5.44 | no |
| Q96C36 | 14.78333 | yes | 5.44 | no |
| Q96C36 | 13.155 | yes | 1.995 | no |
| P47897 | 20 | yes | — | — |
| Q5XKP0 | 20 | yes | 2.1 | no |
| Q9H0R6 | 13.815 | yes | 20 | yes |
| Q6WKZ4 | 2.815 | no | 5.015 | yes |
| Q6IQ22 | — | — | 20 | yes |
| P61106 | 5.653333 | yes | 2.316667 | no |
| Q9NX57 | 6.805 | yes | — | — |
| O14966 | — | — | 17.89 | yes |
| P53611 | 3.22 | no | 5.105 | yes |
| Q92878 | 5.35 | yes | 2.566667 | no |
| Q9Y3L5 | 20 | yes | 9.735 | yes |
| O75884 | — | — | 5.405 | yes |
| Q96T37 | 17.69 | yes | 2.046667 | no |
| Q8NDT2 | 20 | yes | — | — |
| A0AV96 | 2.356667 | no | 5.43 | yes |
| Q9Y256 | 20 | yes | 20 | yes |
| Q8IZV5 | 20 | yes | 1.135 | no |
| P35251 | 20 | yes | — | — |
| A6NKT7 | 6.155 | yes | — | — |
| Q9HBH0 | 9.075 | yes | 1.235 | no |
| Q8IXI2 | 2.68 | no | 8.213333 | yes |
| Q6R327 | 6.673333 | yes | 4.96 | no |
| Q5UIP0 | 20 | yes | 3.68 | no |
| Q13671 | 20 | yes | — | — |
| Q6NUQ1 | 20 | yes | — | — |
| Q9BVS4 | — | — | 20 | yes |
| O14730 | 6.4 | yes | 20 | yes |
| P27635 | 5.34 | yes | 2.186667 | no |
| P27635 | 8.11 | yes | 2.532857 | no |
| P62913 | 3.116667 | no | 9.04 | yes |
| P62913 | 3.116667 | no | 9.04 | yes |
| P50914 | 5.38 | yes | 3.426667 | no |
| P46776 | 9.643333 | yes | 2.58 | no |
| P46779 | 5.136667 | yes | 2.46 | no |
| P39023 | 6.95 | yes | 14.33667 | yes |
| Q969Q0 | 5.036667 | yes | 2.356667 | no |
| P36578 | 12.5 | yes | 3.746667 | no |
| P36578 | 10.75333 | yes | 3.356667 | no |
| P62424 | 12.87667 | yes | 6.28 | no |
| Q6DKI1 | 7.38 | yes | 0.99 | no |
| P05388 | 7.093333 | yes | 7.41 | yes |
| Q9BUL9 | 2.476667 | no | 20 | yes |
| Q9BUL9 | 1.67 | no | 9.58 | yes |
| P62280 | 6.4 | yes | 1.69 | no |
| P42677 | 12.65333 | yes | — | — |
| P42677 | 16.76 | no | 9.805 | yes |
| P42677 | 20 | yes | 5.713333 | yes |
| Q71UM5 | 12.65333 | yes | — | — |
| Q71UM5 | 16.76 | no | 9.805 | yes |
| Q71UM5 | 20 | yes | 5.713333 | yes |
| Q71UM5 | 9.206667 | yes | 6.12 | no |
| P61247 | 5.28 | yes | 3.08 | no |
| P22090 | 9.32 | yes | 2.896667 | no |
| Q8TD47 | 9.32 | yes | 2.896667 | no |
| P62753 | 20 | yes | 2.943333 | no |
| P56182 | — | — | 6.925 | yes |
| P56182 | 18.565 | yes | 3.01 | no |
| Q5JTH9 | 20 | yes | 2.98 | no |
| Q5JTH9 | 9.07 | yes | 6.035 | yes |
| Q5JTH9 | 14.01 | yes | 7.15 | yes |
| Q16799 | 1.975 | no | 7.6 | yes |
| Q16799 | 20 | yes | — | — |
| P28702 | — | — | 9.585 | yes |
| P29034 | 6.525 | yes | 2.125 | no |
| Q9UPU9 | 7.405 | yes | — | — |
| Q5PRF9 | 7.405 | yes | — | — |
| Q9UHR5 | 5.975 | yes | 2.62 | no |
| Q9NVU7 | 5.425 | yes | 2.19 | no |
| Q9NVU7 | 20 | yes | 5.965 | yes |
| P53992 | — | — | 5.3 | yes |
| P05120 | 2.396667 | no | 8.8525 | yes |
| Q9BYW2 | 2.723333 | no | 6.125 | yes |
| Q587I9 | — | — | 7.625 | yes |
| Q15464 | 3.98 | no | 7.886667 | yes |
| P29353 | 1.85 | no | 10.335 | yes |
| Q14493 | 4.85 | no | 9.3325 | yes |
| Q9BXP2 | 20 | yes | — | — |
| P43007 | 20 | yes | 19.885 | yes |
| O43772 | 5.665 | yes | 2.115 | no |
| Q9H936 | — | — | 5.105 | yes |
| P12235 | 1.4 | no | 16.76 | yes |
| P05141 | 1.655 | no | 5.88 | yes |
| P12236 | 1.4 | no | 16.76 | yes |
| Q6P1M0 | 2.273333 | no | 8.24 | yes |
| Q9ULF5 | — | — | 14.025 | yes |
| Q15043 | 20 | yes | 20 | yes |
| Q08AF3 | 12.085 | yes | — | — |
| P51532 | 17.38333 | yes | 8.815 | yes |
| Q96GM5 | 4.02 | no | 6.95 | yes |
| Q14683 | 12.73 | yes | 3.92 | no |
| O95295 | 3.215 | no | 8.89 | yes |
| Q9Y5X2 | 20 | yes | — | — |
| P08047 | 4.775 | no | 7.703333 | yes |
| Q8NB90 | 20 | yes | — | — |
| Q9BVQ7 | 20 | yes | 8.76 | no |
| Q9NUQ6 | 5.05 | yes | 5.26 | yes |
| O43278 | — | — | 8.04 | yes |
| P35270 | 1.9 | no | 5.375 | yes |
| P11277 | 17.565 | yes | 9.79 | yes |
| Q01082 | 20 | yes | 16.49 | yes |
| O15020 | 17.565 | yes | 9.79 | yes |
| Q9Y6N5 | 15.29 | no | 14.265 | yes |
| Q13501 | 2.505 | no | 12.91333 | yes |
| P12931 | 3.311667 | no | 20 | yes |
| P12931 | 4.03 | no | 13.56667 | yes |
| O75044 | — | — | 5.34 | yes |
| P08240 | 20 | yes | 2.575 | no |
| Q9Y5M8 | 11.085 | yes | 2.73 | no |
| Q9Y5M8 | 13.595 | yes | 2.84 | no |
| Q08945 | 13.82 | yes | 11.04333 | yes |
| Q9Y5Y6 | — | — | 14.03 | yes |
| Q9Y5Y6 | 5.053333 | yes | 5.42 | no |
| Q8N1F8 | — | — | 5.74 | yes |
| Q9UEW8 | 20 | yes | 4.66 | no |
| P53597 | 6.38 | yes | 2.73 | no |
| Q8IX01 | 5.73 | yes | 3.635 | no |
| O94901 | 8.555 | yes | 0.84 | no |
| O94901 | 3.8 | no | 7.706667 | yes |
| Q9Y5B9 | 6.263333 | yes | 7.17 | no |
| Q8WXH0 | 4.375 | no | 16.235 | yes |
| Q8WXH0 | — | — | 6.495 | yes |
| Q12962 | 5.685 | yes | — | — |
| Q15545 | 20 | yes | — | — |
| Q9BW92 | 5.66 | yes | 4.875 | no |
| Q8NHU6 | 3.3 | no | 15.85333 | yes |
| Q15582 | — | — | 12.615 | yes |
| Q8IXH7 | 1.76 | no | 20 | yes |
| Q07157 | 2.62 | no | 8.59 | yes |
| Q96SK2 | 2.88 | no | 11.11 | yes |
| Q96SK2 | 2.92 | no | 7.055 | yes |
| Q9BTX1 | 20 | yes | 6.01 | yes |
| Q9BTX1 | 18.74667 | yes | 6.88 | yes |
| Q96BY9 | 4.805 | no | 7.155 | yes |
| Q9NVH6 | — | — | 13.815 | yes |
| P42166 | 8.52 | no | 6.213333 | yes |
| Q9C0C2 | 1.6 | no | 5.135 | yes |
| Q8IZW8 | 2.913333 | no | 5.565 | yes |
| O96008 | 2.865 | no | 6.03 | yes |
| O96008 | 2.865 | no | 6.03 | yes |
| O96008 | 2.865 | no | 6.03 | yes |
| P11388 | 20 | yes | 15.39 | no |
| Q02880 | 5.225 | yes | 3.765 | no |
| Q02880 | 17.22 | yes | 8.34 | yes |
| Q12888 | 1.973333 | no | 10.885 | yes |
| O14773 | 8.6 | yes | 2.51 | no |
| O14773 | 11.86333 | yes | 2.99 | no |
| Q9H4I3 | 11.29 | yes | 1.9 | no |
| O75962 | 20 | yes | 1.86 | no |
| Q15654 | 3.663333 | no | 6.823333 | yes |
| Q15361 | — | — | 6.57 | yes |
| Q71U36 | 5.156667 | yes | 2.333333 | no |
| Q71U36 | 6.163333 | yes | 2.146667 | no |
| Q13748 | 6.5 | yes | — | — |
| Q13748 | 5.293333 | yes | — | — |
| P68366 | 9.57 | yes | 3.75 | no |
| P68366 | 6.76 | yes | — | — |
| P68366 | 6.98 | yes | 4.29 | no |
| P68366 | 9.978 | yes | 3.958333 | no |
| Q9NY65 | 6.98 | yes | — | — |
| Q9NY65 | 7.691667 | yes | 5.003333 | yes |
| A6NHL2 | 5.156667 | yes | 2.28 | no |
| P07437 | 6.94 | yes | — | — |
| P07437 | 6.94 | yes | — | — |
| Q9BVA1 | 6.94 | yes | 2.56 | no |
| Q9BVA1 | 6.94 | yes | 2.56 | no |
| P68371 | 6.94 | yes | — | no |
| P68371 | 6.94 | yes | — | no |
| Q9BUF5 | 6.94 | yes | 2.56 | no |
| Q9BUF5 | 6.94 | yes | 2.56 | no |
| Q2T9J0 | 20 | yes | 19.22 | yes |
| Q9GZZ9 | 4.935 | no | 5.8 | yes |
| Q9NPG3 | 20 | yes | — | — |
| Q92575 | 1.61 | no | 13.81667 | yes |
| Q9BZV1 | 20 | yes | — | — |
| Q9NYU1 | 20 | yes | 20 | yes |
| F8VZW7 | 13.375 | yes | 2.55 | no |
| H7BZ11 | 5.036667 | yes | 2.356667 | no |
| H7C455 | — | — | 20 | yes |
| J3KR12 | 14.78333 | yes | 5.44 | no |
| H7C469 | — | — | 12.705 | yes |
| H3BQZ7 | 14.30667 | yes | 4.893333 | no |
| F5H5T6 | — | — | 20 | yes |
| J3KR12 | 13.155 | yes | 1.995 | no |
| H7BZ11 | 5.08 | yes | — | — |
| P22695 | 12.715 | yes | 1.99 | no |
| Q9NVE5 | — | — | 20 | yes |
| P46939 | 2.99 | no | 13.24 | yes |
| Q9BQE4 | — | — | 8.845 | yes |
| A3KMH1 | 8.855 | yes | 1.985 | no |
| Q9H3P2 | 20 | yes | — | — |
| Q9Y4P8 | 16.115 | yes | 13.12 | no |
| Q9HD64 | 6.135 | yes | — | — |
| Q9HD64 | 5.42 | yes | 2.663333 | no |
| Q9HAV4 | 2.383333 | no | 7.993333 | yes |
| P07947 | 2.66 | no | 19.3 | yes |
| P49750 | 2.29 | no | 20 | yes |
| Q9NPG8 | 7.84 | no | 20 | yes |
| P17029 | 1.75 | no | 12.05 | yes |

Table 2, Table 3 (e.g., Table 3A and Table 3B), and Table 4 illustrate additional exemplary lists of NRF2-regulated proteins and their respective cysteine sites of interaction.

Lengthy table referenced here
US20200278355A1-20200903-T00001
Please refer to the end of the specification for access instructions.

Lengthy table referenced here
US20200278355A1-20200903-T00002
Please refer to the end of the specification for access instructions.

Lengthy table referenced here
US20200278355A1-20200903-T00003
Please refer to the end of the specification for access instructions.

Lengthy table referenced here
US20200278355A1-20200903-T00004
Please refer to the end of the specification for access instructions.

### Example 2

Cell Lines

All cell lines were obtained from ATCC. All cells were maintained at 37° C. with 5% CO₂. HEK-293T cells were grown in DMEM (Corning) supplemented with 10% fetal bovine serum (FBS, Omega Scientific), penicillin (100 U/ml), streptomycin (100 μg/ml) and L-glutamine (2 mM). H2122, H460, A549, H1975, H358, H1792, and H2009 cells were grown in RPMI-1640 (Invitrogen) supplemented as above. H2009 cells were additionally supplemented with Insulin-Transferrin-Selenium (Invitrogen). For SILAC experiments, each cell line was passaged at least six times in SILAC RPMI (Thermo), which lack L-lysine and L-arginine, and supplemented with 10% (v/v) dialyzed FBS (Gemini), penicillin, streptomycin, L-glutamine (as above), and either [¹³C6, ¹⁵N₂]-L-lysine and [¹³C6, ¹⁵N₄]-L-arginine (100 mg/mL each) or L-lysine and L-arginine (100 mg/mL each). Heavy and light cells were maintained in parallel and cell aliquots were frozen after six passages in SILAC media and stored in liquid N₂ until needed. Whenever thawed, cells were passaged at least three times before being used in experiments.

cDNA Cloning and Mutagenesis

cDNAs encoding for NR0B1, SNW1, RBM45 were amplified from a cDNA pool generated from A549 cells and were subcloned into the FLAG-pRK5 or HA-pRK5 expression vectors. These cDNAs were also subcloned into the lentiviral expression vector FLAG-pLJM1 (Bar-Peled et al., Science 340, 1100-1106, 2013). The firefly luciferase gene was cloned into the lentiviral expression vector pLenti-pgk BLAST as described before (Goodwin et al., Mol. Cell 55, 436-450, 2014). Cysteine mutants were generated using QuikChange XLII site-directed mutagenesis (Agilent), using primers containing the desired mutations. All constructs were verified by DNA sequencing.

Mammalian Lentiviral shRNAs Expression

Lentiviral shRNAs targeting the messenger RNA for human NR0B1, SWN1, and AKR1B10 were cloned into pLKO.1 vector at the Age 1, EcoR1 sites.

shRNA-encoding plasmids were co-transfected with ΔVPR envelope and CMV VSV-G packaging plasmids into 2.5×10⁶ HEK-293T cells using the Xtremegene 9 transfection reagent (Sigma-Aldrich). Virus-containing supernatants were collected forty-eight hours after transfection and used to infect target cells in the presence of 10 μg/ml polybrene (Santa Cruz). Twenty-four hours post-infection, fresh media was added to the target cells which were allowed to recover for an additional twenty-four hours. Puromycin was then added to cells, which were analyzed immediately or on the 2nd or 3rd day after selection was added.

Generation of CRISPR-Mediated Knockout HEK-293T Cell Lines

sgRNAs targeting KEAP1 or NRF2 (described below) were designed, amplified, and cloned into transient pSpCas9-2A-Puro (Addgene, PX459). 1×10⁶ HEK-293T cells were transfected with the pSpCa9-2A-Puro plasmid containing sgRNAs targeting KEAP1 or NRF2. Following puromycin selection, clonal cells were isolated by flow cytometry and analyzed for the increased or decreased expression of NRF2 by immunoblot for KEAP1-null or NRF2-null cells, respectively.

Generation of CRISPR-Mediated Knockout H460 Cell Lines

NR0B1-null or CYP4F11-null H460 cells were generated using the protocol described in (Shalem et al., 2014). In brief, sgRNAs targeting NR0B1, CYP4F11 or AKR1B10 were designed, amplified, and cloned into transient Lenti-CRISPR v2 (Addgene). Mammalian lentiviral particles harboring sgRNA-encoding plasmids were generated as described above, with the exception that the viral supernatant was concentrated with LentiX (Clontech) prior to infection of H460 cells. Following 10 days of puromycin selection, clonal cells were isolated by flow cytometry and analyzed for decreased expression of NR0B1, CYP4F11 or AKR1B10 when compared to a parental population expressing a non-targeting sgRNA (CRISPR-CTRL).

Mammalian Lentiviral cDNA Expression

Mammalian lentiviral particles harboring cDNA-encoding plasmids were generated as described above, with the exception that the viral supernatant was concentrated with LentiX (Clontech) prior to infection of target cells. Cells were allowed to recover for 24 h followed by continuous selection with puromycin.

Identification of NR0B1 Interacting Proteins

Confluent 15 cm dishes of A549 stably or transiently expressing FLAG-NR0B1 or FLAG-METAP2, were rinsed with ice-cold PBS and were sonicated in the presence of Chaps IP buffer (0.3% Chaps, 40 mM Hepes pH 7.4, 50 mM KCl, 5 mM MgCl₂ and EDTA-free protease inhibitors (Sigma)). Following lysis, samples were clarified by centrifugation for 10 min at 16,000×g. FLAG-M2 beads (100 μL, 50:50 slurry) was added to the clarified supernatant and incubated for 3 h while rotating at 4° C. Beads were washed once with Chaps IP buffer and three times with Chaps IP buffer supplemented with 150 mM NaCl. Proteins were eluted with the FLAG peptide from the FLAG-M2 beads, run on a 4-20% Tris-glycine gel (Invitrogen) and stained with InstantBlue (Expedeon). Each lane was cut into 10 pieces and in-gel trypsin (Promega) digestion was performed. The resulting digests were analyzed by liquid chromatography tandem mass spectrometry (LC-MS/MS). MS2 spectra data were extracted from the raw file using RAW Convertor (version 1.000). MS2 spectra data were searched using the ProLuCID algorithm using a reverse concatenated, non-redundant variant of the Human UniProt database (release-2012_11). Cysteine residues were searched with a static modification for carboxyamidomethylation (+57.02146) and one differential modification for oxidized methionine (+15.9949). Spectral counts for proteins from FLAG-NR0B1 immunoprecipitates were compared to spectral counts for proteins from FLAG-METAP2 immunoprecipitates across 5-6 biological replicates. Interacting proteins were classified as those proteins whose corresponding peptides were enriched by greater that 20-fold in FLAG-NR0B1 immunoprecipitates compared to FLAG-METAP2 immunoprecipitates.

For identification of endogenous NR0B1 interacting proteins, A549, H2122 or H460 cell lysates were prepared as described above. The NR0B1 (Cell Signaling Technology), RagC (Cell Signaling Technology) or GAPDH (Santa Cruz) antibodies were added to each lysate and incubated with rotation at 4° C. for 1.5 h. Subsequently, protein G sepharose beads (50 μL, 50:50 slurry) were added to each sample and incubated for an additional 1.5 h. Beads were washed as described above and proteins were eluted with 8M urea at 30° C. for 1 h. Proteins were reduced by treatment with DTT (10 mM for 30 min at 65° C.) and cysteines were alkylated with iodoacetamide (20 mM for 30 min at 37° C.). Urea was diluted to 2M and proteins were digested with 2 μg of Trypsin (Promega). The resulting digests were analyzed by mass spectrometry as described below.

Co-Transfection Based Interaction Experiments

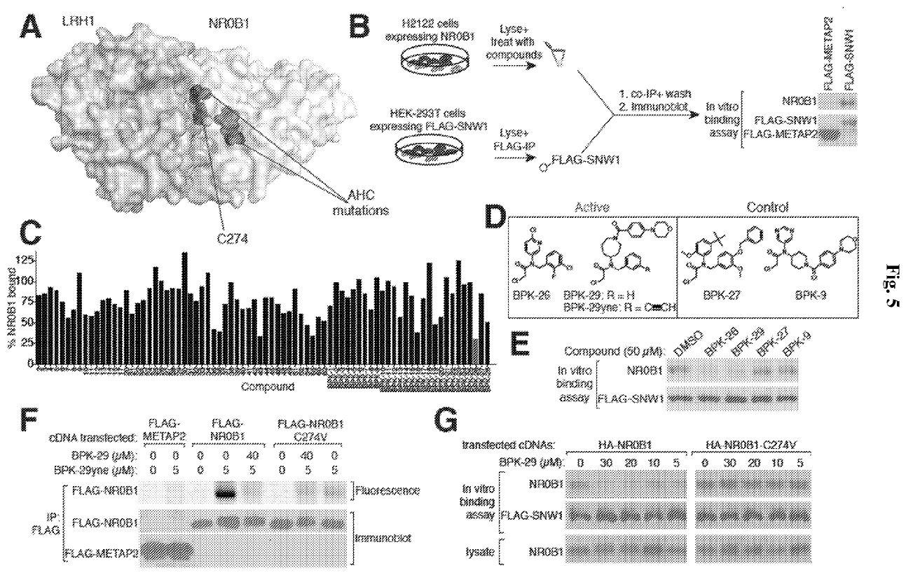

For transfection experiments, 4×10⁶ HEK-293T cells were plated in a 10 cm dish. The next day, cells were transfected with the pRK5-based cDNA expression plasmids indicated in the figures in the following amounts. Figure S4: 25 ng FLAG-RBM45, 100 ng FLAG-NR0B1, 200 ng HA-SNW1; FIG. 5 and FIG. 11: for in-vitro binding experiments: 5000 ng FLAG-SNW1; for in vitro binding experiments with transiently transfected NR0B1: 25 ng HA-NR0B1 or HA-NR0B1-C274V; for fluorescence experiments: 5000 ng Flag-NR0B1 or 5000 ng FLAG-NR0B1-C274V; FIG. 5S: for site of labeling experiments, 5000 ng FLAG-NR0B1. Following transfections, cells were grown for 48 h and processed as described below.

Compound Treatment for Assessment of Protein-Protein Interactions

Confluent 10 cm plates of indicated cell lines were rinsed once with warm PBS and incubated in serum/dye-free RPMI with indicated compounds or vehicle for 3 h at 37° C. Cells were washed once ice-cold PBS and snap frozen.

Cell Lysis and Immunoprecipitations

Cells were rinsed once with ice-cold PBS, and lysed by sonication in Triton IP buffer. Lysates were clarified by centrifugation at 16,000×g for 10 min. Samples were normalized to 1 mg ml⁻¹ and boiled following the addition of sample buffer. For FLAG- or HA-immunoprecipitations, FLAG or HA resins (30 μL, 50:50 slurry) were added to the pre-cleared lysates and incubated with rotation for 3 hours at 4° C. Following immunoprecipitation, the beads were washed once with IP buffer followed by 3 times with IP buffer containing 500 mM NaCl. Loading buffer (40 μL) was added to the immunoprecipitated proteins which were subsequently denatured by boiling. Proteins were resolved by SDS-PAGE, analyzed by immunoblotting and relative band intensities were quantified using ImageJ software.

In Vitro Binding Assay

H2122 clarified cell lysate (100 μL, 1 mg ml⁻¹) in IP-buffer were incubated with the indicated compounds or vehicle (DMSO) for 3 hours at 4° C. with rotation. Following treatment, 3 volumes of IP-buffer was added along with immobilized FLAG-SNW1 beads (30 μL, 50:50 slurry), which was incubated for an additional hour at 4° C. Beads were washed three times with IP-buffer supplemented with 500 mM NaCl. Immunoprecipitated proteins were resolved by SDS-PAGE and analyzed by immunoblotting. NR0B1 and HA-NR0B1 levels were determined by using the NR0B1 antibody (Cell Signaling). IC₅₀ curves were determined using Prism 6 (Graphpad) software, with maximum and minimum values set at 100% NR0B1 bound 0% NR0B1 bound respectively.

Immunofluorescence

Samples were prepared as follows. In brief, 1×10⁵ A549 cells stably expressing FLAG-RBM45 or FLAG-SNW1 were plated on poly-lysine coated glass coverslips in 12-well tissue culture plates. Forty-eight hours later, the culture media was removed and cells were fixed with 4% paraformaldehyde (Electron microscopy services). The slides were rinsed three times with PBS and cells were permeabilized with 0.05% Triton X-100 in PBS for 1 min. The slides were rinsed four times with PBS and incubated with primary antibodies in 5% normal donkey serum (Thermo) overnight at 4° C. After rinsing four times with PBS, the slides were incubated with secondary antibodies conjugated to the indicated fluorophores (Invitrogen) for 1 h at room temperature. Following an additional four washes with PBS, the slides were stained with Hoechst (Invitrogen) following the manufacturer's protocol. Slides were mounted on glass coverslips using Prolong Gold® Antifade reagent (Invitrogen) and imaged on Zeiss LSM 780 laser scanning confocal microscope. Images were processed using ImageJ software.

Measurement of Glycolytic Flux

Cells were plated on poly-L-lysine coated 96-well Seahorse plates (Seahorse Biosciences) after lentiviral infection with shNRF2 or shGFP and equilibrated for 1 h in DMEM (Sigma D6030) containing 2 mM glutamine in the absence of serum and glucose. Basal extracellular acidification rate (ECAR) was then analyzed in the Seahorse XFe96 flux analyzer (Seahorse Biosciences), followed by ECAR measurements after sequential injections of 10 mM glucose, 2 μM oligomycin and 100 mM 2-deoxyglucose (2-DG).

Measurement of Intracellular Glutathione Levels

H2122 or H1975 cells expressing shRNAs targeting a control or NRF2 were cultured in 6-well plates and total cellular glutathione content was determined using the Glutathione Assay Kit (Cayman Chemical) following the manufacturer's protocol. Absorbance from GSH reaction with DTNB was measured using a Biotek Synergy 2 microplate reader (Biotek).

Measurement of GAPDH Activity

2.5×10⁵ H2122 or H1975 cells expressing shRNAs targeting a control or NRF2 were cultured in 6-well plates and GAPDH activity was determined using Ambion KDalert GAPDH Assay Kit (Fisher) following the manufacture's protocol. This assay measures the conversion of NAD⁺ to NADH by GAPDH in the presence of glyceraldehyde-3-phosphate. The rate of NADH production correlated to an increase in fluorescence was measured by using a Biotek Synergy 2 microplate reader (Biotek).

Measurement of Cytosolic Hydrogen Peroxide Levels

Cytosolic hydrogen peroxide was measured using the Peroxyfluor-6 acetoxymethyl ester (PF6-AM) fluorescent probe as described in (Dickinson et al., Nat Chem Biol 7, 106-112, 2011). In brief, cells were washed twice with warm PBS and incubated with 250 nM of PF6-AM in serum-free RPMI for 20 min at 37° C. Cells were allowed to recover in complete RPMI for 1 h and were subsequently harvested and resuspended in sorting buffer (PBS+1% FBS). Flow cytometry acquisition was performed with BD FACSDiva™-driven BD™ LSR II flow cytometer (Becton, Dickinson and Company) which measured the increase in PF6-AM fluorescence. Data was analyzed with FlowJo software (Treestar Inc.)

Monolayer Proliferation Assay

Cells were cultured in 96-well plates at 3×10³ cells per well in 100 μl of RPMI. At the indicated time points 50 μl of Cell Titer Glo reagent (Promega) was added to each well and the luminescence read on a Biotek Synergy 2 microplate reader (Biotek).

qPCR Analysis

2.5×10⁵ cells/well of a 6-well plate were seeded the night before treatment. Cells were treated with the indicated concentrations of compound as denoted in the figure legends for 12 h. Total RNA was isolated using the RNeasy Kit (Qiagen) according to the manufacturer's protocol. cDNA amplification was preformed using iScript Reverse Transcription Supermix kit (Bio-Rad). qPCR primer sequences were obtained from PrimerBank and are listed below. qPCR analysis was performed on a ABI Real Time PCR System (Applied Biosystems) with the SYBR green Mastermix (Applied Biosystems). Relative gene expression was normalized to the 18S gene.

Gel-Based Competition of BPK-29Yne Labeling of NR0B1

4×10⁶ HEK-293T cells were seeded in poly-L-lysine coated 10 cm plates and transfected the next day with 5 μg of FLAG-NR0B1, FLAG-NR0B1-C274V, or FLAG-METAP2 cDNA in a pRK5-based expression vector. 48 h after transfection, cells were treated with indicated concentrations of BPK-29 or control compound BPK-27 for 3 h at 37° C. in DMEM containing 10% FBS and supplements as described in Cell Culture. BPK-29yne (5 μM) was then added and incubated for an additional 30 min at 37° C. FLAG immunoprecipitates were prepared as described above and following washes, the FLAG resin was resuspended in PBS (100 μL). To each sample, 12 μL of a freshly prepared “click” reagent mixture was added to conjugate the fluorophore to probe-labeled proteins. CuAAC reaction mixture consisted of TAMRA azide (1 μL of 12.5 mM stocks in DMSO, final concentration=125 μM), 1 mM tris(2-carboxyethyl)phosphine hydrochloride (TCEP; 2 μL of fresh 50× stock in water, final concentration=1 mM), ligand (6 μL 17× stock in DMSO:t-butanol 1:4, final concentration=100 μM) and 1 mM CuSO₄ (2 μL of 50× stock in water, final concentration=1 mM). Upon addition of the click mixture, each reaction was immediately mixed by vortexing and then allowed to react at ambient temperature for 1 h before quenching the reactions with 100 μL of loading buffer. Samples were boiled for 5 min and proteins were resolved by SDS-PAGE (10% acrylamide), and visualized by in-gel fluorescence on a Bio-Rad ChemiDoc MP flatbed fluorescence scanner. Samples were also analyzed by immunoblotting. Recombinantly expressed FLAG-tagged protein levels were determined with the FLAG antibody (Sigma). Gel fluorescence and imaging was processed using Image Lab (v 5.2.1) software.

Measurement of NR0B1 Degradation

7.5-8×10⁵ H460 cells were seeded the night before per well of a 6-well plate. Cells were treated with cycloheximide (100 μg/mL) for the indicated time points. Cells were rinsed in ice-cold PBS, scraped on ice and processed for immunoblot analysis as described above. Proteins were resolved by SDS-PAGE, analyzed by immunoblotting and NR0B1 band intensities were quantified using ImageJ software and compared to a loading control (Beta-actin or GAPDH).

RNA Sequencing

RNA was isolated by RNeasy Kit (Qiagen) and digested with DNase (Qiagen) from n=3 samples per condition (cells expressing shGFP, shNRF2_1, shNR0B1_1 or shSNW1_1 or treated with DMSO, 30 μM BPK-29 or 30 μM BPK-9). RNA integrity (RIN) numbers were determined using the Agilent TapeStation prior to library preparation. mRNA-seq libraries were prepared using the TruSeq RNA library preparation kit (version 2) according to the manufacturer's instructions (Illumina). Libraries were then quantified, pooled, and sequenced by single-end 50 base pairs using the Illumina HiSeq 2500 platform at the Salk Next-Generation Sequencing Core. Raw sequencing data were demultiplexed and converted into FASTQ files using CASAVA (version 1.8.2). Libraries were sequenced at an average depth of 15 million reads per sample.

The spliced read aligner STAR (Dobin et al., 2013) was used to align sequencing reads to the human hg19 genome. Gene-level read counts were obtained based on UCSC hg19 gene annotation. DESeq2 (Love et al., 2014) was used to calculate differential gene expression based on uniquely aligned reads, and p-values were adjusted for multiple hypothesis testing with the Benjamini-Hochberg method.

ChIP-seq Analysis

ChIP was conducted as previously described (Komashko et al., Genome Res 18, 521-532, 2008). H460 cells were fixed in 1% formaldehyde (Sigma) for 15 minutes at 25° C. After lysis, samples were sonicated using a biorupter sonicator (Diagenode) for 60 cycles (30 seconds per cycle/30 seconds cooling) at a high power level. Chromatin sheering was optimized to a size range of 200 to 600 bp. Chromatin (100 μg) was immunoprecipitated with the NR0B1 antibody (Cell Signaling Technology). For DNA sequencing, samples were prepared for library construction, flow cell preparation and sequencing were performed according to Illumina's protocols. Sequencing was accomplished on Illumina HiSeq 2500 using PE 2×125 bp reads with over 14 million clusters per sample.

Sequencing reads were aligned to the hg19 genome using bowtie2 (Langmead and Salzberg, Nat Methods 9, 357-359, 2012). Peak detection was carried out using HOMER, comparing the NR0B1 IP sample against a whole-cell extract (WCE) with default parameters for transcription factor-style analysis. This requires relevant peaks to be significantly enriched over WCE and the local region with an uncorrected Poisson distribution-based p-value threshold of 0.0001 and false discovery rate threshold of 0.001. These peaks were further restricted to a 2 kb window around annotated transcription start sites.

Correlation Analysis:

For shRNA gene expression analysis data, the correlation of gene expression levels between the shNR0B1-cells and shNRF2-cells and shNR0B1-cells and shSNW1-cells was calculated using Pearson's correlation coefficient, and a correlation analysis was performed to calculate the p-value.

Circos Plot

A graphical summary of NR0B1 genome-wide effects. The inner track shows the change in gene expression following NR0B1 knockdown (red indicates an increase, blue a decrease). The middle track shows the normalized peak height of the NR0B1 ChIP. Only genes with both significantly altered expression (adjusted p-value threshold of 0.01 and 1.5-fold expression threshold) and an NR0B1 peak near a TSS are shown.

A graphical summary of liganded cysteines in KEAP1-WT and KEAP1-mutant cell lines. The outer track denotes total liganded cysteines in a given cell line (cysteines were defined as liganded if they had an average R≥5 and were quantified in two or more replicates). Grey chords connect liganded cysteines that are found in two or more cell lines.

GSEA

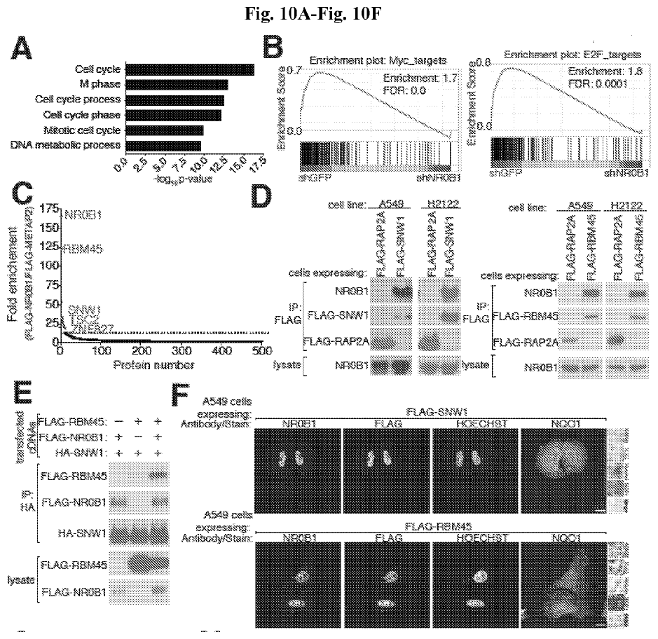

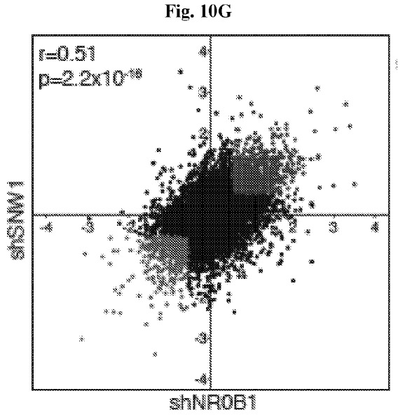

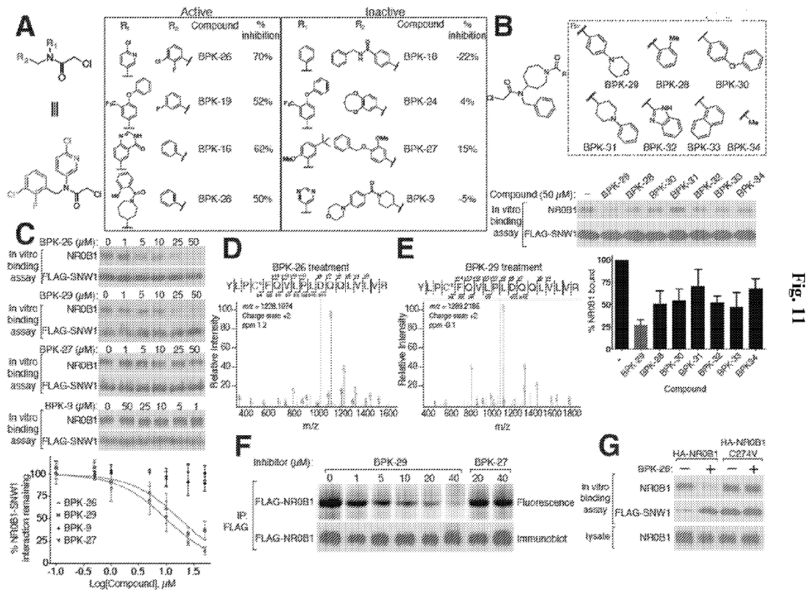

GSEA (Subramanian et al., PNAS 102, 15545-15550, 2005) was carried out using pre-ranked lists from FDR or fold change values, setting gene set permutations to 1000 and using either c1 collection in MSigDB version 4.0 (FIG. 10C).

Functional Gene Enrichment Analysis

Functional enrichment in gene sets was determined using the DAVID functional annotation tool (version 6.7) with “FAT” Gene Ontology terms (Huang da et al., Nat Protoc 4, 44-57, 2009).

isoTOP-ABPP Sample Preparation

Sample preparation and analysis were based on (Backus et al. Nature 534, 570-574, 2016) with modifications noted below.

For analysis of NR0B1 ligands or control compound reactivity, H460 cells or H460 cells expressing luciferase in a 10 cm plate were incubated with indicated compounds in serum/dye-free RPMI for 3 hours at 37° C. Cells were washed once ice-cold PBS and lysed in 1% Triton X-100 dissolved in PBS with protease inhibitors (Sigma) by sonication. Samples were clarified by centrifugation for 10 min at 16,000×g. Lysate was adjusted to 1.5 mg ml⁻¹ in 500 μL.

For analysis of cysteines regulated by NRF2, H2222 or H1975 cells expressing shGFP or shNRF2 were lysed and processed as described above. Lysate was adjusted to 1.5 mg ml⁻¹ in 500 μL.

For analysis of cysteines that change following induction of apoptosis, H2122 and H1975 cells were treated with DMSO or staurosporine (1 μM, 4 h) in full RPMI. H1975 cells were treated with DMSO or AZD9291 (1 μM, 24 h) in full RPMI. Cells were lysed as described above.

For analysis of ligandable cysteines in KEAP1-WT (H2122, H460 and A549) cells and KEAP1-mutant (H1975, H2009 (expressing the luciferase protein) and H358) cells, lysate was prepared as described in (Backus et al., 2016). Samples were treated with 500 μM of compound 2, 3 or vehicle for 1 h at room temperature.

isoTOP-ABPP IA-Alkyne Labeling and Click Chemistry

Samples were labeled for 1 h at ambient temperature with 100 μM iodoacetamide alkyne (1, IA-alkyne, 5 μL of 10 mM stock in DMSO). Samples were conjugated by copper-catalyzed azide-alkyne cycloaddition (CuAAC) to isotopically labeled, TEV-cleavable tags (TEV-tags). Heavy CuAAC reaction mixtures was added to the DMSO-treated or shGFP control samples and light CuAAC reaction mixture was added to compound-treated or shNRF2 samples. The CuAAC reaction mixture consisted of TEV tags (light or heavy, 10 μL of 5 mM stocks in DMSO, final concentration=100 μM), 1 mM tris(2-carboxyethyl)phosphine hydrochloride (TCEP; fresh 50× stock in water, final concentration=1 mM), ligand (17× stock in DMSO:t-butanol 1:4, final concentration=100 μM) and 1 mM CuSO₄ (50× stock in water, final concentration=1 mM). The samples were allowed to react for 1 h at which point the samples were centrifuged (16,000×g, 5 min, 4° C.). The resulting pellets were sonicated in ice-cold methanol (500 μL) and the resuspended light- and heavy-labeled samples were then combined pairwise and centrifuged (16,000×g, 5 min, 4° C.). The pellets were solubilized in PBS containing 1.2% SDS (1 mL) with sonication and heating (5 min, 95° C.) and any insoluble material was removed by an additional centrifugation step at ambient temperature (14,000×g, 1 min).

isoTOP-ABPP Streptavidin Enrichment

For each sample, 100 μL of streptavidin-agarose beads slurry (Fisher) was washed in 10 mL PBS and then resuspended in 6 mL PBS (final concentration 0.2% SDS in PBS). The SDS-solubilized proteins were added to the suspension of streptavidin-agarose beads and the bead mixture was rotated for 3 h at ambient temperature. After incubation, the beads were pelleted by centrifugation (1,400×g, 3 min) and were washed (2×10 mL PBS and 2×10 mL water).

isoTOP-ABPP Trypsin and TEV Digestion

The beads were transferred to eppendorftubes with 1 mL PBS, centrifuged (1,400×g, 3 min), and resuspended in PBS containing 6 M urea (500 μL). To this was added 10 mM DTT (25 μL of a 200 mM stock in water) and the beads were incubated at 65° C. for 15 mins. 20 mM iodoacetamide (25 μL of a 400 mM stock in water) was then added and allowed to react at 37° C. for 30 mins with shaking. The bead mixture was diluted with 900 μL PBS, pelleted by centrifugation (1,400×g, 3 min), and resuspended in PBS containing 2 M urea (200 μL). To this was added 1 mM CaCl₂ (2 μL of a 200 mM stock in water) and trypsin (2 μg, Promega, sequencing grade) and the digestion was allowed to proceed overnight at 37° C. with shaking. The beads were separated from the digest with Micro Bio-Spin columns (Bio-Rad) by centrifugation (1,000×g, 1 min), washed (2×1 mL PBS and 2×1 mL water) and then transferred to fresh eppendorf tubes with 1 mL water. The washed beads were washed once further in 140 μL TEV buffer (50 mM Tris, pH 8, 0.5 mM EDTA, 1 mM DTT) and then resuspended in 140 μL TEV buffer. 5 μL TEV protease (80 μM) was added and the reactions were rotated overnight at 29° C. The TEV digest was separated from the beads with Micro Bio-Spin columns by centrifugation (1,400×g, 3 min) and the beads were washed once with water (100 μL). The samples were then acidified to a final concentration of 5% (v/v) formic acid and stored at −80° C. prior to analysis.

isoTOP-ABPP Liquid-Chromatography-Mass-Spectrometry (LC-MS) Analysis

Samples processed for multidimensional liquid chromatography tandem mass spectrometry (MudPIT) were pressure loaded onto a 250 μm (inner diameter) fused silica capillary columns packed with C18 resin (Aqua 5 μm, Phenomenex). Samples were analyzed using an LTQVelos Orbitrap mass spectrometer (Thermo Scientific) coupled to an Agilent 1200-series quaternary pump. The peptides were eluted onto a biphasic column with a 5 μm tip (100 μm fused silica, packed with C18 (10 cm) and bulk strong cation exchange resin (3 cm, SCX, Phenomenex)) in a 5-step MudPIT experiment, using 0%, 30%, 60%, 90%, and 100% salt bumps of 500 mM aqueous ammonium acetate and using a gradient of 5-100% buffer B in buffer A (buffer A: 95% water, 5% acetonitrile, 0.1% formic acid; buffer B: 5% water, 95% acetonitrile, 0.1% formic acid) as has been described in (Weerapana et al., 2007). Data were collected in data-dependent acquisition mode with dynamic exclusion enabled (20 s, repeat of 2). One full MS (MS1) scan (400-1800 m/z) was followed by 30 MS2 scans (ITMS) of the nth most abundant ions.

isoTOP-ABPP Peptide and Protein Identification

The MS2 spectra data were extracted from the raw file using RAW Convertor (version 1.000). MS2 spectra data were searched using the ProLuCID algorithm (publicly available at http://fields.scripps.edu/downloads.php) using a reverse concatenated, non-redundant variant of the Human UniProt database (release-2012_11). Cysteine residues were searched with a static modification for carboxyamidomethylation (+57.02146) and up to two differential modification for either the light or heavy TEV tags or oxidized methionine (+464.28595, +470.29976, +15.9949 respectively).

MS2 spectra data were also searched using the ProLuCID algorithm using a custom database containing only selenocysteine proteins, which was generated from a reverse concatenated, nonredundant variant of the Human UniProt database (release-2012_11). In the database, selenocysteine residues (U) were replaced with cysteine (C) and were searched with a static modification for carboxyamidomethylation (+57.02146) and up to two differential modification for either the light or heavy TEV tags or oxidized methionine (+512.2304+ or +518.2442+15.9949). Peptides were required to have at least one tryptic terminus and to contain the TEV modification. ProLuCID data was filtered through DTASelect (version 2.0) to achieve a peptide false-positive rate below 1%.

isoTOP-ABPP R Value Calculation and Processing

The isoTOP-ABPP ratios (R values) of heavy/light for each unique peptide (DMSO/compound treated or shGFP/shNRF2) were quantified with in-house CIMAGE software (Weerapana et al., Nature 468, 790-795, 2010) using default parameters (3 MS1 acquisitions per peak and signal to noise threshold set to 2.5). Site-specific engagement of cysteine residues was assessed by blockade of IA-alkyne probe labelling. A maximal ratio of 20 was assigned for peptides that showed a ≥95% reduction in MS1 peak area from the experimental proteome (light TEV tag) when compared to the control proteome (DMSO, shGFP; heavy TEV tag). Ratios for unique peptide sequences entries were calculated for each experiment; overlapping peptides with the same modified cysteine (for example, different charge states, MudPIT chromatographic steps or tryptic termini) were grouped together and the median ratio is reported as the final ratio (R). Additionally, ratios for peptide sequences containing multiple cysteines were grouped together. Biological replicates of the same treatment and cell line were averaged if the standard deviation was below 60% of the mean; otherwise, for cysteines with at least one R value<4 per treatment, the lowest value of the ratio set was taken. For cysteines where all R values were ≥4, the average was reported. The peptide ratios reported by CIMAGE were further filtered to ensure the removal or correction of low-quality ratios in each individual data set. The quality filters applied were the following: removal of half tryptic peptides; removal of peptides which were detected only once across all data sets reported herein; removal of peptides with R=20 and only a single MS2 event triggered during the elution of the parent ion; manual annotation of all the peptides with ratios of 20, removing any peptides with low-quality elution profiles that remained after the previous curation steps.

For selenocysteines, the ratios of heavy/light for each unique peptide (DMSO/compound treated; isoTOP-ABPP ratios, R values) were quantified with in-house CIMAGE software using the default parameters described above, with the modification to allow the definition of selenocysteine (amino acid atom composition and atomic weights). Extracted ion chromatograms were manually inspected to ensure the removal of low quality ratios and false calls.

Cysteine residues were deemed to have significantly changed following NRF2 knockdown if they had R-values≥2.5. Changes in cysteine reactivity were considered reactivity based if a cysteine for a given protein had an R-value≥2.5 and all the remaining cysteines in that protein had R-values<1.5. If only one cysteine was identified per protein with an R value≥2.5, and if the corresponding change in the mRNA transcript was <1.5 (shGFP/shNRF2) then that change was also considered reactivity based. Changes in cysteine reactivity were considered expression based if a cysteine for a given protein had an R-value≥2.5 and all the remaining cysteines in that protein had R-values≥1.5. If only one cysteine was identified per protein with an R-value≥2.5, and if the corresponding change in the mRNA transcript was ≥1.5 (shGFP/shNRF2) then than change was also considered expression based. For datasets corresponding to changes in cysteine reactivity in H2122 cells expressing shNRF2 or shGFP at ‘Day 1/2’ two replicates were taken from the ‘Day 1’ time point and three replicates were taken from the ‘Day 2 time point’ (Tables 2 and 3). For datasets corresponding to changes in cysteine reactivity in H1975 cells expressing shNRF2 or shGFP at ‘Day 1/2’ two replicates were taken from the ‘Day 1’ time point and two replicates were taken from the ‘Day 2 time point’ (Tables 2 and 3). For datasets corresponding to changes in cysteine reactivity in H2122 cells expressing shNRF2 or shGFP at ‘Day1’ three replicates were used. Cysteine residues were designated as expression-based changes for this experiment if following NRF2 knockdown they had R-values≥2.5 and were considered unchanged if they had R-values<1.5 (Tables 2 and 3). Cysteines were considered significantly changed following staurosporine or AZD9291 treatment if they had R values≥2.5.

Cysteine residues were considered liganded in vitro by electrophilic fragments (compounds 2 or 3) if they had an average R-value≥5 and were quantified in at least 2 out of 3 replicates. Targets of NR0B1 ligands or control compounds were defined as those cysteine residues that had R-values≥3 in more than one biological replicate following ligand treatment in cells.

Protein Turnover

For analysis of protein turnover in H460 cells, confluent 10 cm plates were washed twice with warm PBS, then incubated in “heavy” RPMI for 3 h. Cells were washed once ice-cold PBS and lysed in 1% Triton 100-X dissolved in PBS with protease inhibitors (Sigma) by sonication. Lysate was adjusted to 1.5 mg ml⁻¹ in 2×500 μL. Samples were processed identically to other samples (lysates were adjusted to 1.5 mg ml⁻¹ in 2×500 μL), with the following modification: only isotopically light TEV tag was used. After the “click” reaction, both 2×500 μL were centrifuged (16,000×g, 5 min, 4° C.) and resuspended by sonication in ice-cold methanol (500 μL). Aliquots were then combined and resolubilized in PBS containing 1.2% SDS (1 mL) as detailed in isoTOP-ABPP IA-alkyne labeling and click chemistry. Samples were further processed and analyzed as detailed in: isoTOP-ABPP streptavidin enrichment, isoTOP-ABPP trypsin and TEV digestion, isoTOP-ABPP liquid-chromatography-mass-spectrometry (LC-MS) analysis, isoTOP-ABPP peptide and protein identification and isoTOP-ABPP R value calculation and processing with the following exceptions: Samples processed for protein turnover were searched with ProLuCID with mass shifts of SILAC labeled amino acids (+10.0083 R, +8.0142 K) in addition to carboxyamidomethylation modification (+57.02146) and two differential modification for either the light TEV tag or oxidize methionine (+464.28595, +15.9949 respectively). 1 peptide identification was required for each protein. ProLuCID data was filtered through DTASelect (version 2.0) to achieve a peptide false-positive rate below 1%. Ratios of light/heavy peaks were calculated using in-house CIMAGE software. Median SILAC ratios from one or more unique peptides were combined to generate R values. Proteins were required to be quantified in at least two biological replicates. The mean R values and standard deviation for multiple biological experiments were calculated from the average ratios from each replicate. Proteins were designated as rapid turnover if they had R-values≤8.

ABPP-SILAC Sample Preparation and LC-MS Analysis.

Isotopically labeled H460 cell lines were generated as described above. Light and heavy cells were treated with compounds (20 μM) or DMSO, respectively, for 3 h, followed by labeling with the BPK-29yne (5 μM) for 30 min. Cells were washed once ice-cold PBS and lysed in 1% Triton 100-X dissolved in PBS with protease inhibitors (Sigma) by sonication. Lysate was adjusted to 1.5 mg ml⁻¹ in 500 μL. Samples were conjugated by CuAAC to Biotin-PEG4-azide (5 μL of 10 mM stocks in DMSO, final concentration=100 μM). CuAAC “click” mix contained TCEP, TBTA ligand and CuSO4 as detailed for isoTOP-ABPP sample preparation. Samples were further processed as detailed in: isoTOP-ABPP streptavidin enrichment and isoTOP-ABPP trypsin TEV digestion with the following exception: after overnight incubation at 37° C. with trypsin, tryptic digests were separated from the beads with Micro Bio-Spin columns (Bio-Rad) by centrifugation (1,000×g, 1 min). Beads were rinsed once with water (200 μL) and combined with tryptic digests. The samples were then acidified to a final concentration of 5% (v/v) formic acid and stored at −80° C. prior to analysis. Samples were processed for multidimensional liquid chromatography tandem mass spectrometry (MudPIT) as described in isoTOP-ABPP liquid-chromatography-mass-spectrometry (LC-MS) with the exception that peptides were eluted using the 5-step MudPIT protocol with conditions: 0%, 25%, 50%, 80%, and 100% salt bumps of 500 mM aqueous ammonium acetate and using a gradient of 5-100% buffer B in buffer A (buffer A: 95% water, 5% acetonitrile, 0.1% formic acid; buffer B: 5% water, 95% acetonitrile, 0.1% formic acid).

ABPP-SILAC Peptide and Protein Identification and R Value Calculation and Processing

The MS2 spectra data were extracted and searched using RAW Convertor and ProLuCID algorithm as described in isoTOP-ABPP peptide and protein quantification. Briefly, cysteine residues were searched with a static modification for carboxyamidomethylation (+57.02146 C). Searches also included methionine oxidation as a differential modification (+15.9949 M) and mass shifts of SILAC labeled amino acids (+10.0083 R, +8.0142 K) and no enzyme specificity. Peptides were required to have at least one tryptic terminus and unlimited missed cleavage sites. 2 peptide identifications were required for each protein. ProLuCID data was filtered through DTASelect (version 2.0) to achieve a peptide false-positive rate below 1%. Ratios of heavy/light (DMSO/test compound) peaks were calculated using in-house CIMAGE software. Median SILAC ratios from two or more unique peptides were combined to generate R values. The mean R values and standard deviation for multiple biological experiments were calculated from the average ratios from each replicate. Targets of NR0B1 ligands or control compounds were defined as those proteins that had R-values≥2.5 in two or more biological replicates following ligand treatment in cells.

Site of Labeling

For site of labeling with BPK-29, 4×10⁶ HEK-293T cells were seeded in a 10 cm plate and transfected the next day with 5 μg of FLAG-NR0B1 cDNA in a pRK5-based expression vector. 48 hours after transfection, cells were treated with vehicle, BPK-29 (50 μM) in serum-free RPMI for 3 h at 37° C. FLAG immunoprecipitates were prepared as described above in Identification of NR0B1 interacting proteins. FLAG-NR0B1 was eluted from FLAG-M2 beads with 8M urea and subjected to proteolytic digestion, whereupon tryptic peptides harboring C274 were analyzed by LC-MS/MS. The resulting mass spectra were extracted using the ProLuCID algorithm designating a variable peptide modification (+252.986 and +386.1851 for BPK-26 and BPK-29, respectively) for all cysteine residues. For site of labeling with BPK-26, HEK-293T cell lysate transfected with FLAG-NR0B1 as described above was treated with vehicle or BPK-26 (100 μM) for 3 h at 4° C. FLAG immunoprecipitates were processed for proteomic analysis as described above.

Quantification and Statistical Analysis

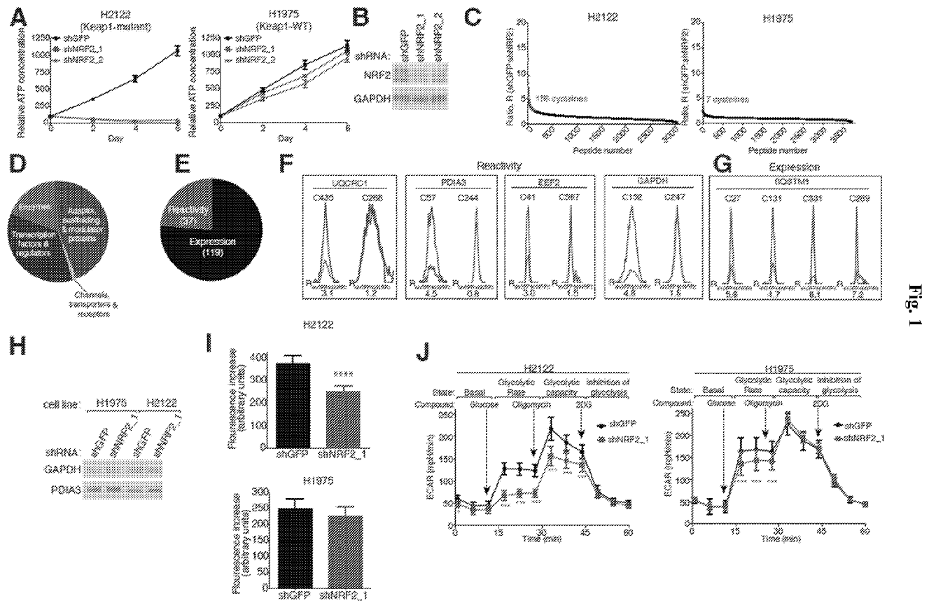

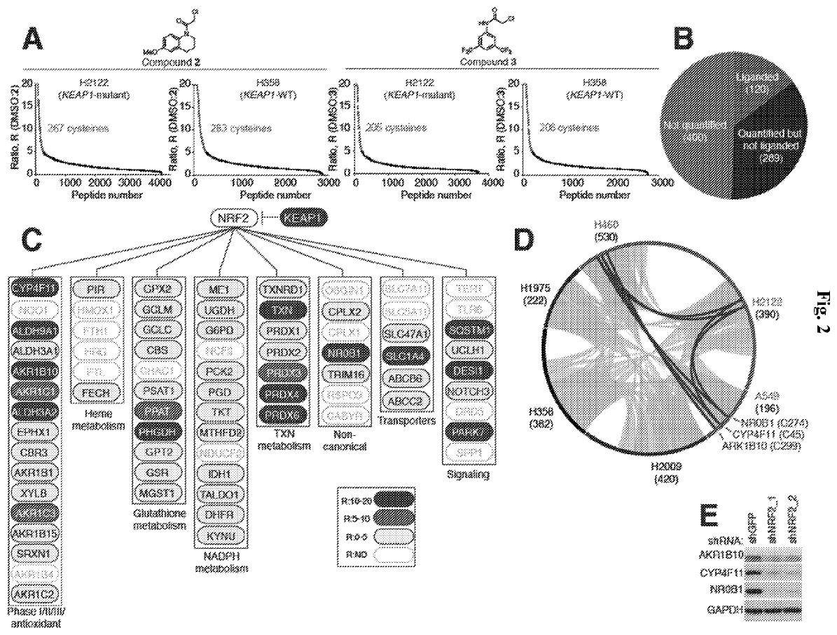

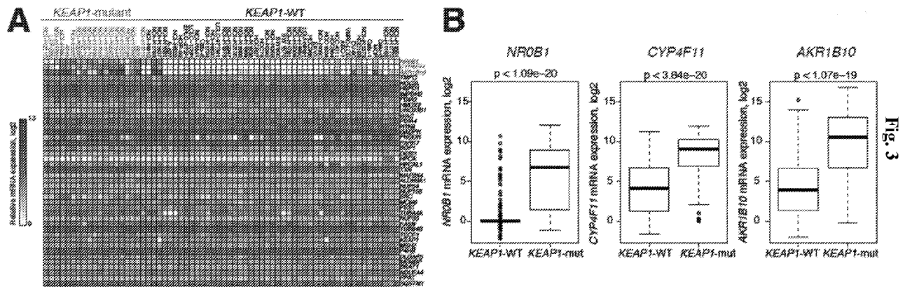

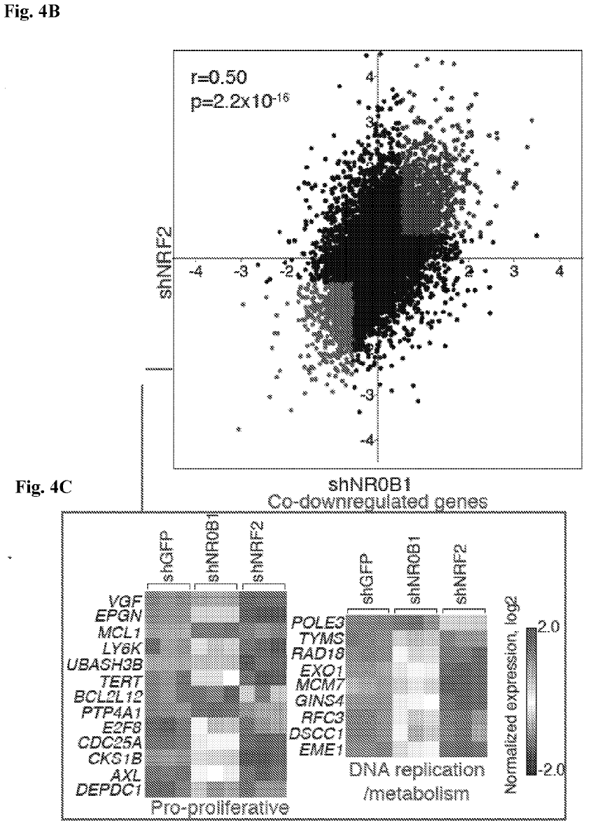

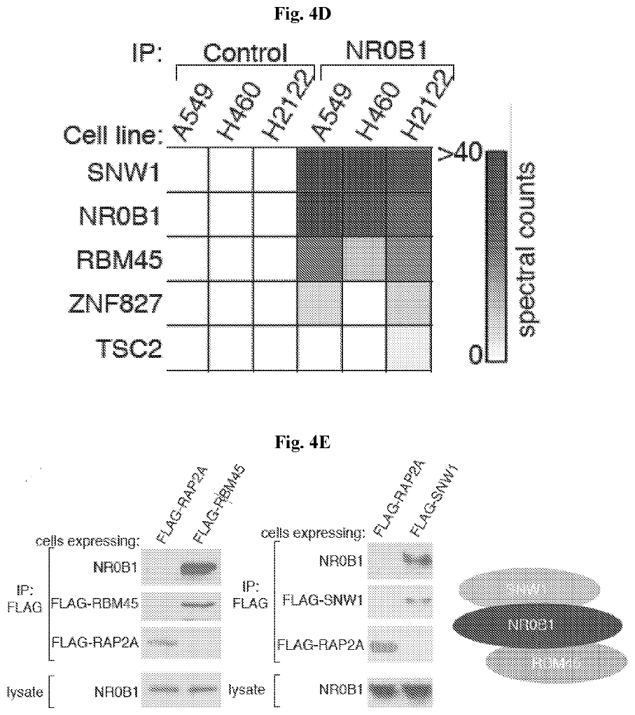

Statistical analysis was preformed using GraphPad Prism version 6 or 7 for Mac, GraphPad Software, La Jolla Calif. USA, or the R statistical programming language. Statistical values including the exact n and statistical significance are also reported in the Figures. Inhibition curves of the NR0B1-SNW1 interactions by NR0B1-ligand are fit as using log(inhibitor) vs % normalized remaining of NR0B1-SNW1 interaction and data points are plotted as the mean±SD (n=2-5 per group). NR0B1 half-life was calculated from a one-phase exponential decay curve plotted as mean±SD (4-10 per group). Statistical significance was defined as p<0.05 and determined by 2-tailed Student's t-test (FIG. 1I, FIG. 3B), two-way Anova with Bonferroni post-test analysis (FIG. 1J) or correlation analysis using Pearson product-moment correlation coefficient (FIG. 4B, FIG. 10G).

Mapping Cysteine Reactivity in KEAP1-WT and KEAP1-Mutant NSCLC Cells

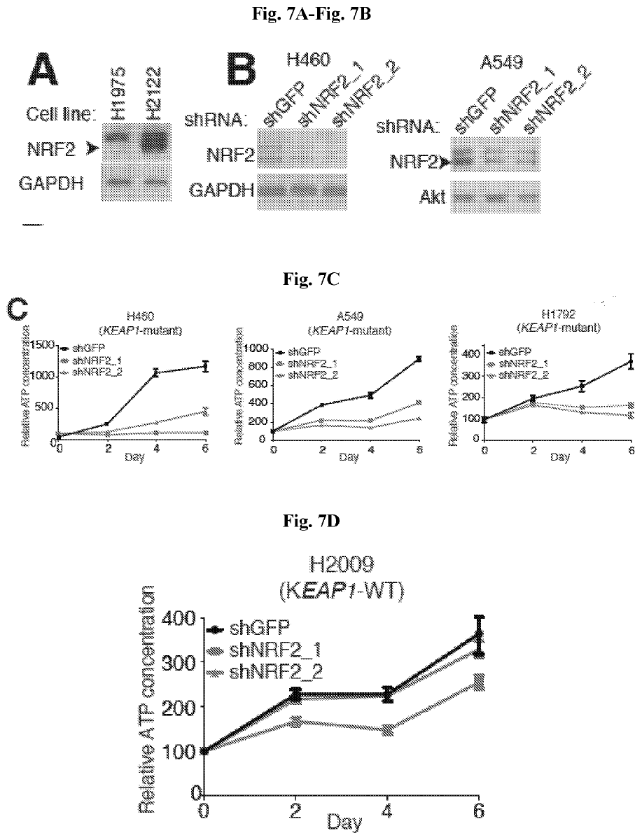

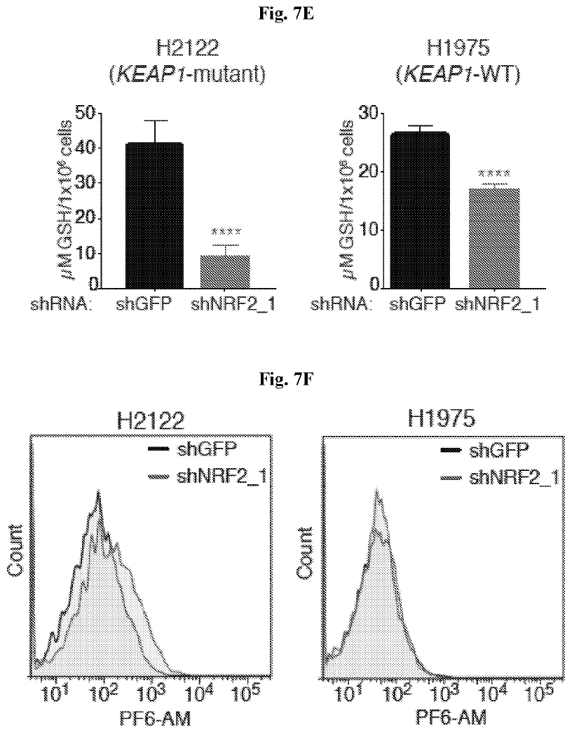

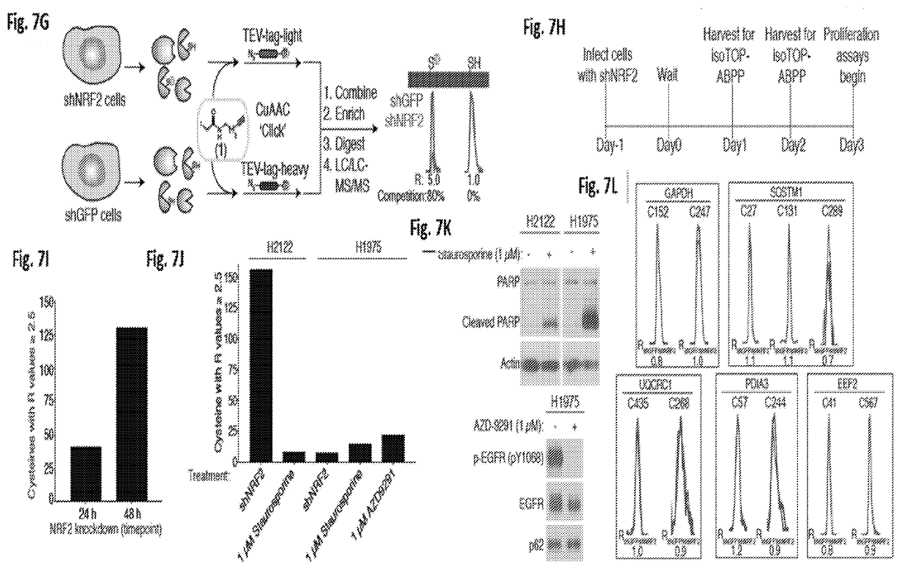

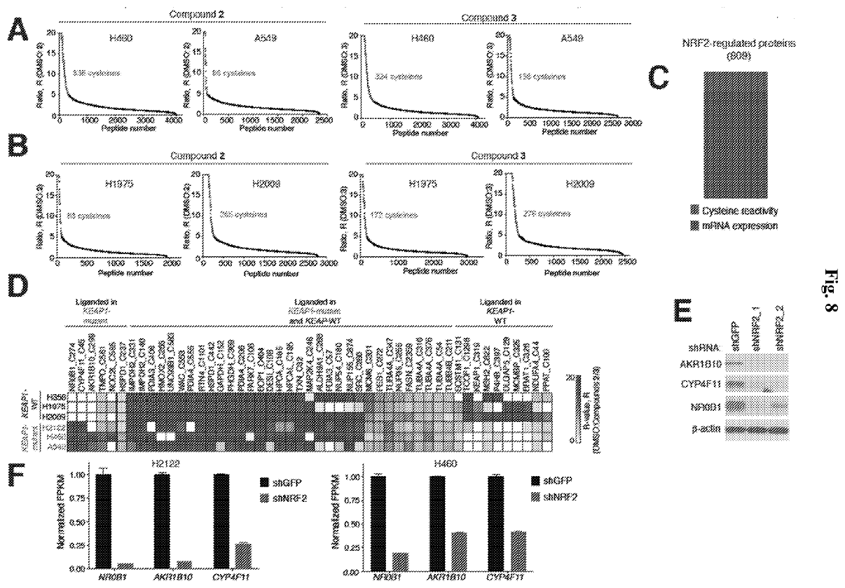

Several human NSCLC cell lines were identified that contain inactivating mutations in the gene encoding KEAP1 (H2122, H460, A549 and H1792), as well as additional NSCLC lines that were wild type (WT) for this gene (H1975 and H2009) (Tables 2 and 3). Small hairpin RNA (shRNA)-mediated knockdown of NRF2 in NSCLC cell lines with KEAP1 mutations, where NRF2 protein levels are stabilized (FIG. 7A), and impaired cell proliferation in conjunction with lowering NRF2 protein content (FIG. 1A, FIG. 1B, and FIGS. 7B-7C). In contrast, KEAP1-WT NSCLC lines were only marginally affected by NRF2-knockdown (FIG. 1A and FIG. 7D). Depletion of NRF2 in the KEAP1-mutant NSCLC line H2122 also led to a marked reduction in glutathione and a concomitant rise in cytosolic H₂O₂ compared to KEAP1-WT H1975 cells (FIGS. 7E-7F).

Cysteine reactivities in KEAP1-mutant (H2122) and KEAP1-WT (H1975) NSCLC lines were mapped following shRNA-mediated knockdown of NRF2 (shNRF2) using the isoTOP-ABPP platform, which employs a broadly reactive iodoacetamide alkyne (IA-alkyne, 1) probe for labeling, enriching, and quantifying cysteine residues in proteomes (FIG. 7G). Cells were evaluated at early (24, 48 h) time points following NRF2 knockdown (FIG. 7H) to minimize changes in cysteine reactivity that may have been indirectly caused by proliferation defects. NRF2-regulated cysteines were defined as those showing ≥2.5-fold changes in reactivity in shNRF2 cells compared to control shRNA (shGFP) cells (i.e., isoTOP-ABPP Ratio (R)≥2.5 for shGFP/shNRF2) and found that 156 cysteines of >3000 total quantified cysteines in H2122 cells satisfied this criterion (FIG. 1C and Tables 2 and 3). Approximately three times as many NRF2-regulated cysteines were observed on day 2 versus day 1 post-NRF2 knockdown in H2122 cells (FIG. 7I), which may reflect a proportional increase in changes caused by NRF2-regulated gene/protein expression (see below). In contrast, NRF2 depletion had minimal effects on cysteine reactivity in H1975 cells (FIG. 1C and Tables 2 and 3). It was also noted that several cysteines with prominent changes in shNRF2-H2122 cells were not detected in H1975 cells, likely reflecting that the proteins harboring these cysteines are themselves regulated by NRF2 (see below). It was further evaluated changes in cysteine reactivity in NSCLC cells caused by other anti-proliferative mechanisms—specifically treatment with the general kinase inhibitor staurosporine or the EGFR inhibitor AZD9291—neither of which caused substantive changes in cysteine reactivity in KEAP1-mutant or KEAP1-WT cells (FIGS. 7J-L and Tables 2 and 3). These results indicate that NRF2 disruption produces specific and widespread alterations in cysteine reactivity in KEAP1-mutant NSCLC cells.

NRF2-regulated cysteines were found in proteins from many different functional classes (FIG. 1D). In instances where all quantified cysteines for a given protein were altered in shNRF2-H2122 cells, it was concluded that the changes reflected an alteration in protein expression. In contrast, if only one of multiple cysteines for a given protein had a substantial reduction in IA-alkyne-reactivity (R≥2.5), with the other quantified cysteines remaining constant (R<1.5), it was noted that the change was reactivity-based. This analysis was supplemented by determining changes in gene expression in shNRF2-versus shGFP-H2122 cells by RNA sequencing (RNA-seq), which provided an expression estimate for proteins that contained only one quantified IA-alkyne-reactive cysteine. By combining the proteomic and gene expression analysis, it was determined that ˜80% of all changes in cysteine reactivity reflected alterations in protein abundance following NRF2-knockdown, with the remaining ˜20% identified as alterations in reactivity (FIG. 1E). Proteins harboring cysteines that underwent specific reactivity changes in shNRF2-H2122 cells were found in central pathways that include glycolysis (GAPDH), protein folding (PDIA3), protein translation (EEF2), and mitochondrial respiration (UQCRC1) (FIG. 1F). An example of a protein showing expression changes in shNRF2-H2122 cells was the canonical NRF2-regulated protein SQSTM1 (FIG. 1G). None of these cysteines were affected by NRF2 knockdown in H1975 cells (FIG. 7L).

A recent cysteine proteomics study performed in Kras-mutated mouse pancreatic cancer organoids deleted for NRF2 expression identified several redox-regulated cysteines (Chio et al., Cell 166, 963-976, 2016). It was noted, however, a minimal overall overlap (˜3%) in NRF2-regulated cysteines in the results compared to the study of Chio et al., which may reflect differences in the mode of NRF2 activation (KEAP1 mutations versus Kras/p53 mutations) tumor of origin (NSCLC versus pancreatic), species (human versus mouse), and/or method of assigning changes in cysteine reactivity (fold-change versus statistical).

The NRF2-regulated cysteines in PDIA3 (C57) and GAPDH (C152) are catalytic residues, designating them as candidate sites for NRF2 control over fundamental biochemical pathways in cancer cells. Another quantified cysteine outside of the GAPDH active site—C247 (FIG. 1F)—was unaltered in reactivity by NRF2 knockdown (FIG. 1F), and it was confirmed that GAPDH protein expression was unaffected in shNRF2 cells by immunoblotting (FIG. 1H). C152 in GAPDH is a redox-sensitive residue that is subject to S-sulphenylation and S-sulfhydration and in some instances is affected by pharmacologically induced forms of oxidative stress. Consistent with the conserved catalytic function performed by C152, shNRF2-H2122 cells, but not shNRF2-H1975 cells, showed decrease in GAPDH activity (FIG. 1I). NRF2 knockdown also produced reductions in basal glycolysis and maximal glycolytic rate that were more substantial in magnitude in H2122 cells compared to H1975 cells (FIG. 1J).

Mapping Cysteine Ligandability in KEAP1-WT and KEAP1-Mutant NSCLC Cells

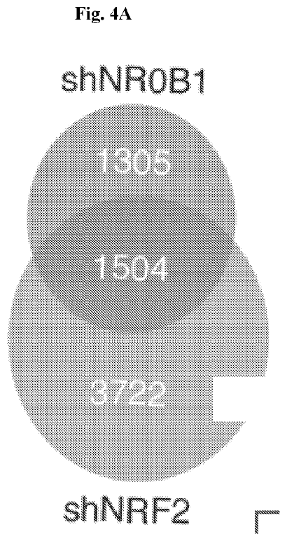

The ligandability of cysteines in NRF2-regulated proteins was investigated by performing competitive isoTOP-ABPP of proteomes from three KEAP1-mutant (H2122, H460 and A549) and three KEAP1-WT (H1975, H2009 and H358) NSCLC lines with two electrophilic fragments—2 and 3 (FIG. 2A)—that showed broad cysteine reactivity in previous studies (Backus et al., 2016). These compounds were referred to as ‘scout’ fragments capable of providing a global portrait of covalent small molecule-cysteine interactions in native biological systems.

From a total of ˜9700 cysteines quantified across the proteomes of six NSCLC lines, ˜1100 scout fragment-sensitive, or ‘liganded’, cysteines were identified (FIG. 2A and FIGS. 8A-8B). Next this ligandability map was overlayed with the fraction of proteins showing changes in cysteine reactivity and/or gene expression in shNRF2 cells (FIG. 8C), resulting in the identification of ˜120 NRF2-regulated proteins with liganded cysteines (FIG. 2B). These proteins populated diverse metabolic and signaling pathways known to be modulated by NRF2 (FIG. 2C), but most were observed in both KEAP1-mutant and KEAP1-WT cells (FIG. 2D and FIG. 8D), indicating that NRF2 influenced, but did not strictly control the expression of these proteins in NSCLCs. Opposing this general profile was a much more restricted subset of liganded proteins that were exclusive to KEAP1-mutant cells (FIG. 2D and FIG. 8D). These proteins included NR0B1 (liganded at C274), CYP4F11 (liganded at C45), and AKR1B10 (liganded at C299) (FIG. 2D and FIG. 8D), which was confirmed by RNA-seq and western blotting were all decreased following knockdown of NRF2 in KEAP1-mutant NSCLC cells (FIG. 2E and FIGS. 8E-8F).

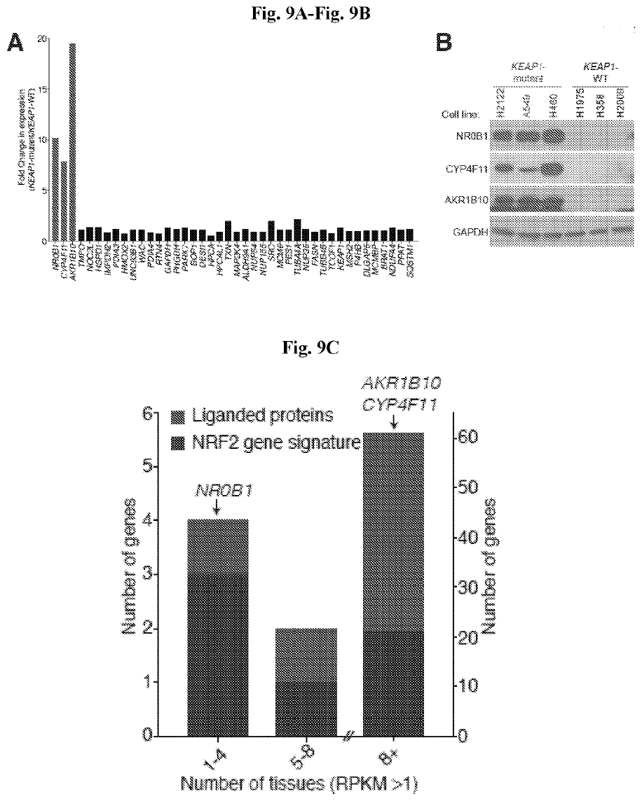

A broader survey of gene expression across >30 NSCLC lines confirmed the remarkably restricted expression of NR0B1, CYP4F11, and AKR1B10 to KEAP1-mutant cells (FIG. 3A and FIG. 9A). This expression profile was confirmed by western blotting (FIG. 9B) and was also observed in primary human lung adenocarcinoma (LUAD) tumors (FIG. 3B). NR0B1 and AKR1B10 have been shown to be important for the proliferation of certain cancers, including KEAP1-mutant NSCLC cells. The role of CYP4F11 in cancer cell growth has not been examined. Consistent with past work, it was found that shRNA knockdown of NR0B1 and AKR1B10 impaired the three-dimensional growth of H460 and H2122 cells. Similar effects were observed for CYP4F11. It was also found that CRISPR-mediated knockout of NR0B1 or CYP4F11 in H460 cells strongly reduced colony formation. Efforts to generate CRISPR knockout cells lacking AKR1B10 were unsuccessful.

NR0B1 Nucleates a Transcriptional Complex that Supports the NRF2 Gene Network

It was noted that most of these enzymes, as well as other NRF2-regulated genes and proteins, were expressed broadly across many human tissues. NR0B1, however, stood out as a striking contrast, being an atypical orphan nuclear receptor with very limited normal tissue expression. Structural studies have shown that NR0B1 possesses a very shallow pocket in place of the typical ligand-binding domain found in other nuclear receptors, indicating that NR0B1 may function as a “ligandless” adaptor or coregulatory protein. Consistent with this premise, NR0B1 acts as a transcriptional repressor of the nuclear receptors SF1 and LRH1 and supports development of Lydig and Serotoli cells in mice. Mutations in the NR0B1 gene lead to adrenal hypoplasia congenita (AHC) in human males. The biochemical and cellular functions of NR0B1 in human cancer and in particular, KEAP1-mutant cancer cells, however, remain poorly understood.

It was first assessed whether NR0B1 acts as a transcriptional regulator in KEAP1-mutant NSCLC cells. RNAseq analysis identified more than >2500 genes that were substantially altered (1.5-fold) in expression in shNR0B1 H460 cells, and ˜30% of these genes were located near transcriptional start sites (TSSs) bound by NR0B1 as determined by chromatin immunoprecipitation sequencing (ChIP-seq) (FIG. 4A). These results suggest that many of the NR0B1-regulated genes in NSCLC cells are in open chromatin under direct transcriptional control of NR0B1. Unbiased functional enrichment analysis (Huang da et al., 2009) revealed an overrepresentation of cell cycle-related and pro-proliferation functions in genes reduced in expression in shNR0B1 NSCLC cells (FIG. 10A) that included, for instance, strong E2F and Myc gene signatures (FIG. 10B). RNAseq analyses further revealed a substantial correlation in global gene expression changes induced by knockdown of NR0B1 or NRF2 in NSCLC cells (FIG. 4B), with >50% of the genes with substantially altered (>1.5 fold) expression in shNR0B1 cells showed a similar magnitude directional change in shNRF2 cells (FIG. 4B). Among the most co-downregulated genes were those involved in proliferation and DNA metabolism/replication (FIG. 4C), consistent with the enrichment of these terms in the NR0B1-regulated gene set (FIG. 10B).

Considering the established function of NR0B1 as a coregulatory protein that participates in nuclear receptor complexes, it was hypothesized that NR0B1 may interact with other proteins to regulate transcriptional pathways in KEAP1-mutant cancer cells. It was expressed a FLAG epitope-tagged form of NR0B1 in KEAP1-mutant NSCLC cells, immunoprecipitated NR0B1 from these cells, and identified associated proteins by mass spectrometry (MS)-based proteomics. Eleven proteins were substantially co-enriched (>20-fold) with NR0B1 compared to a control protein METAP2 (FIG. 10C). A subset of these proteins, including RBM45 and SNW1, were also confirmed by MS-based proteomics to interact with endogenous NR0B1 (FIG. 4D). Stably expressed FLAG-SNW1 and FLAG-RBM45, but not a control protein (FLAG-RAP2A), interacted with NR0B1 in multiple NSCLC cells (FIG. 4E and FIG. 10D), and both SNW1 and RBM45, like NR0B1, were localized to the nucleus of NSCLC cells (FIG. 10F). SNW1 did not directly interact with RBM45 in the absence of NR0B1 (FIG. 10E), indicating that NR0B1 bridges these two proteins to nucleate a multimeric protein complex (FIG. 4E). While very little is known about RBM45, SNW1 has been implicated as a transcriptional activator and found to interact with multiple nuclear receptors, including NR0B1, in large-scale yeast two-hybrid assays. Consistent with this role and with a coordinated function for SNW1 and NR0B1 in KEAP1-mutant cancer cells, RNAi-mediated knockdown of SNW1 produced a similar set of gene expression changes to those observed in shNR0B1 cells (FIG. 10G). SNW1 knockdown also blocked the anchorage independent growth of KEAP1-mutant NSCLC cells.

Covalent Small Molecules that Disrupt NR0B1 Protein Interactions

The liganded cysteine in NR0B1-C274—is located within a conserved “repression helix” that commonly possesses a LXXLL sequence in other nuclear receptors, but, in NR0B1, has been replaced by a PCFXXLP sequence, where the “C” is C274. Missense mutations within this general region of NR0B1 have been found to cause AHC (FIG. 5A), pointing to an important functional role for the repression helix. The hydrophobic residues in the repression helix of NR0B1, including C274, are solvent-exposed and appear to contribute to protein-protein interactions (FIG. 5A), suggesting that ligands targeting C274 might disrupt NR0B1 protein complexes.

Next, a chemical probe targeting C274 of NR0B1 was developed. Using an in vitro binding assay (FIG. 5B), an ˜80-member library of cysteine-reactive electrophilic compounds was screened at 50 μM for blockade of interactions between endogenous NR0B1 and recombinant FLAG-SNW1 in cell lysates (FIG. 5C). Among the hits (>50% blockade) were a series of N-disubstituted chloroacetamides (CAs), including BPK-26 (FIGS. 5D, 5E), that were selected for further investigation. The initial structure-activity relationship indicated more tolerance to substitution of the N-aryl compared to N-benzyl group of BPK-26, including a hit BPK-28 where the N-aryl group was replaced with an azepane group with only modest reductions in potency (FIG. 11A). Modifications to BPK-28, including installation of a morpholine group, generated compound BPK-29 (FIG. 5D) that recovered potency (FIG. 5E and FIG. 11B). Both BPK-26 and BPK-29 inhibited the NR0B1-SNW1 interaction with IC₅₀ values between 10-20 μM in vitro (FIG. 11C). The initial screen also identified structurally related, inactive control compounds—BPK-9 and BPK-27 (FIGS. 5C, 5D)—that did not inhibit the NR0B1-SNW1 interaction across a tested concentration range of 1-50 μM (FIG. 5E and FIG. 11C). Finally, it was confirmed by LC-MS/MS analysis that BPK-26 and BPK-29 covalently modified C274 of NR0B1 (FIGS. 11D, 11E).

An alkyne analogue of BKP-29 (BPK-29yne) was synthesized and found that this probe labeled WT-NR0B1, but not a C274V mutant (FIG. 5G), and this labeling was blocked by pre-treatment with BPK-29 in a concentration dependent manner (FIG. 5G and FIG. 11F). The C274V-NR0B1 mutant maintained binding to SNW1, but this protein-protein interaction was not sensitive to BPK-26 or BPK-29, supporting that these ligands disrupt the NR0B1 protein-protein interactions by covalently modifying C274 (FIG. 5G and FIG. 11G).

Cellular Studies with NR0B1 Ligands

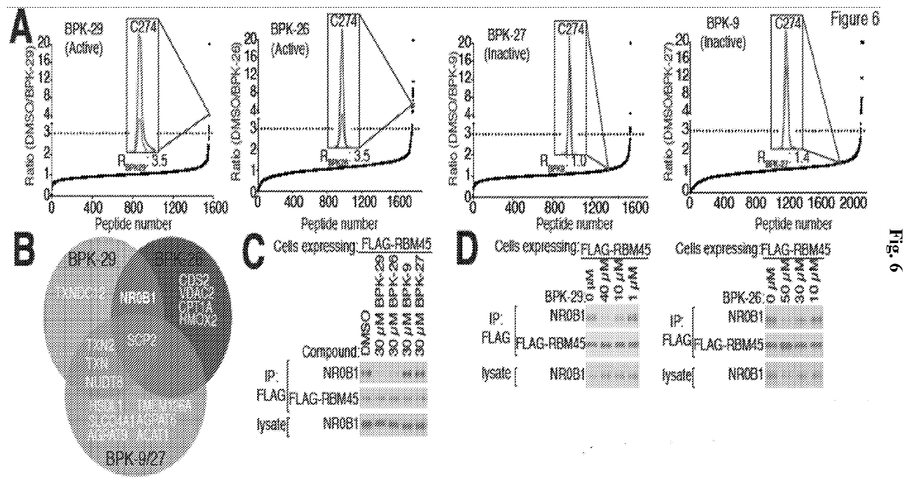

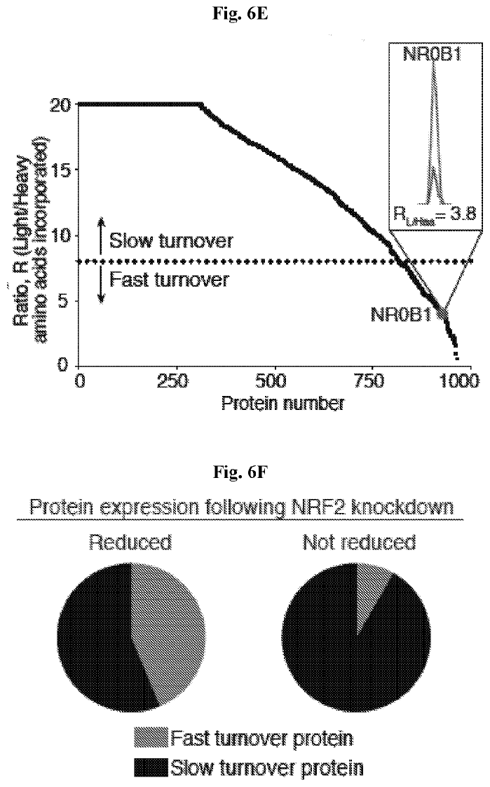

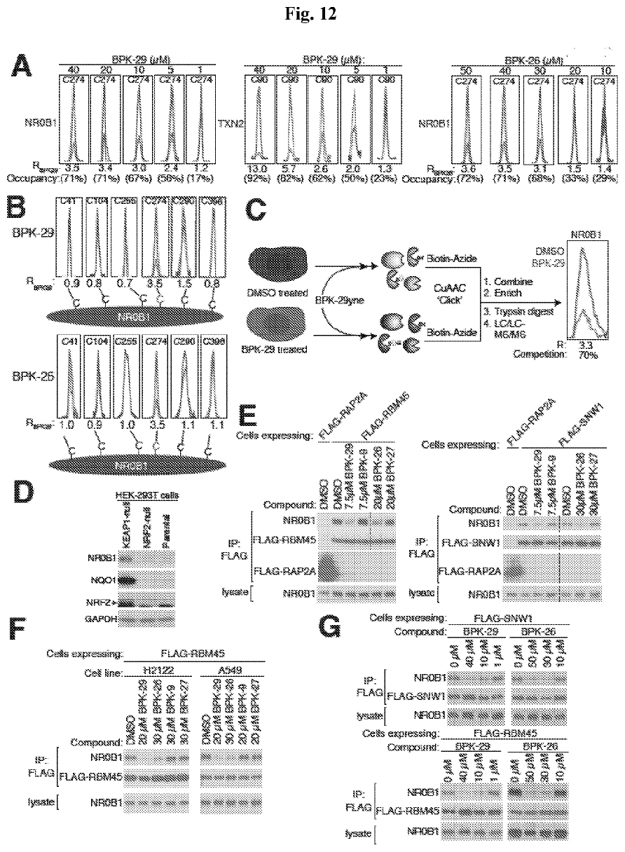

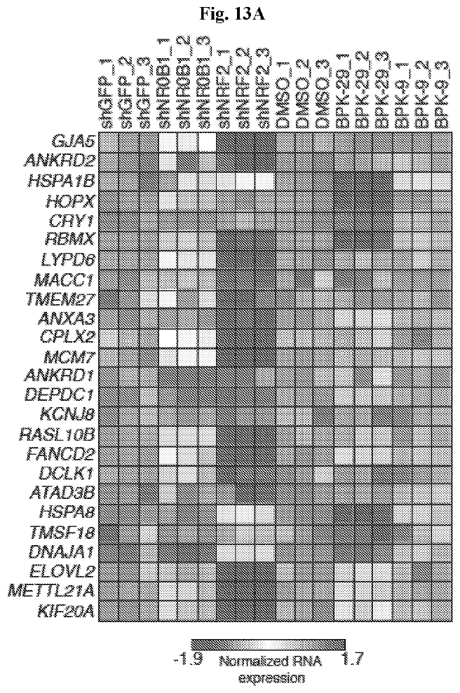

IsoTOP-ABPP confirmed the cellular engagement of C274 of NR0B1 by BPK-26 and BPK-29 in NSCLC cells (FIG. 6A and Table 5), with both compounds achieving ˜70% target occupancy when tested at 40 μM for 3 h (FIG. 6A and FIG. 12A). In contrast, the inactive control compounds BPK-9 and BPK-27 did not engage C274 (FIG. 6A and Table 5). Nine additional cysteines among the >1500 total cysteines quantified by isoTOP-ABPP cross-reacted with BPK-26 and/or BPK-29 in NSCLC cell proteomes (FIGS. 6A, 6B and Table 5), and most of these cysteines also reacted with the control compounds (FIG. 6B and Table 5). NR0B1 was the only target shared between BPK-26 and BPK-29 that did not cross-react with the control compounds (FIG. 6B and Table 5). C274 was also the only cysteine in NR0B1 engaged by BPK-26 and BPK-29 among several other quantified cysteines (FIG. 12B). BPK-29 displayed superior potency compared to BPK-26, achieving >50% engagement of C274 at 5 μM in NSCLC cells (FIG. 12A). The BPK-29yne probe was employed to further characterize the protein targets of BPK-29 in NSCLC cells following the chemical proteomic workflow outlined in FIG. 12C, which verified most of the targets mapped by isoTOP-ABPP and revealed another seven proteins engaged by BPK-29, all of which also cross-reacted with the control compounds (Table 5). Taken together, these data indicate that BPK-26 and BPK-29 substantially engage NR0B1 with good overall proteomic selectivity in KEAP1-mutant NSCLCs.

Next it was asked whether BPK-26 and BPK-29 inhibited NR0B1 protein interactions in cells using two complementary systems. First, KEAP1-null HEK293T cells were generated and found that these cells show elevated expression of NR0B1 (FIG. 12D). KEAP1-null HEK293T cells, or KEAP1-mutant NSCLC cells, were then engineered to stably express FLAG-tagged RMB45 or SNW1 and treated with BPK-26 and BPK-29 or inactive control compounds. In both cell systems, BPK-26 and BPK-29, but not control compounds, blocked the interactions of FLAG-tagged RMB45 or SNW1 with endogenous NR0B1 (FIG. 6C and FIG. 12E-F). BPK-29 blocked NR0B1-protein interactions with better potency than BPK-26 (FIG. 6D and FIG. 12G).

Based on its in situ activity (FIG. 6D and FIG. 12A, 12G) and selectivity (FIGS. 6A, 6B), BPK-29 was chosen for additional biological studies. Treatment of KEAP1-mutant NSCLC cells with BPK-29 (5 μM) blocked colony formation in soft agar. Control compounds BPK-9 and BPK-27 had much less of an effect. Exogenous expression of WT or a C274V mutant of NR0B1 albeit partially rescued the growth inhibition caused by BPK-29. In contrast, BPK-29 (5 μM), or NR0B1 knockdown, minimally affected the anchorage-independent growth of KEAP1-WT NSCLC cells.

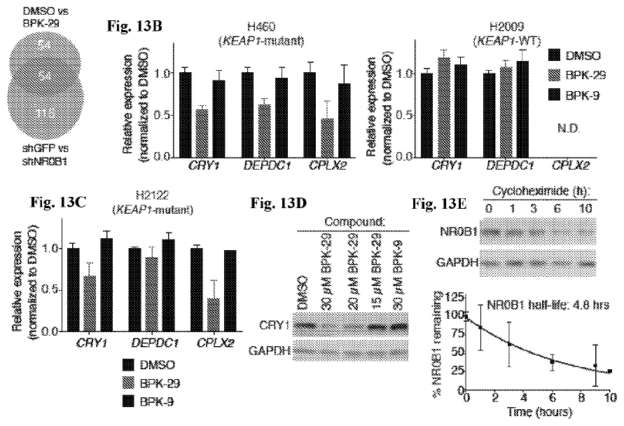

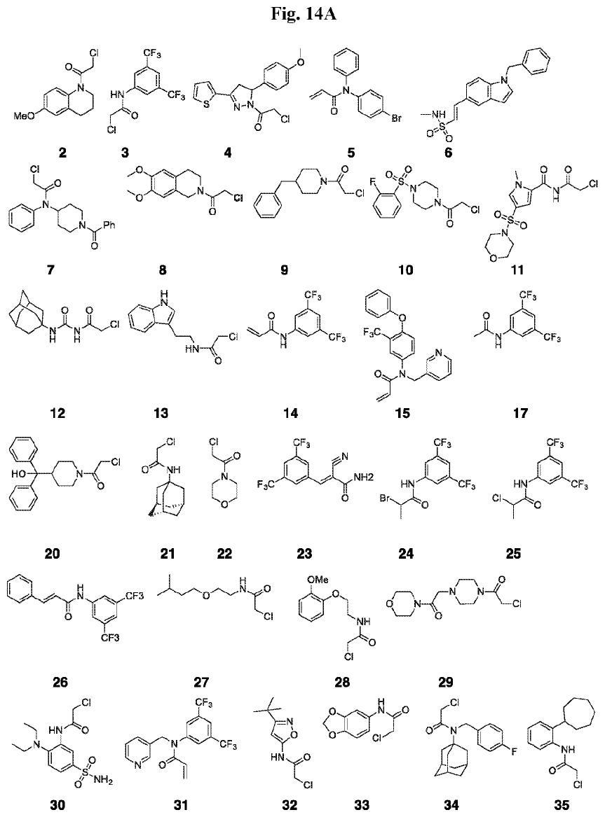

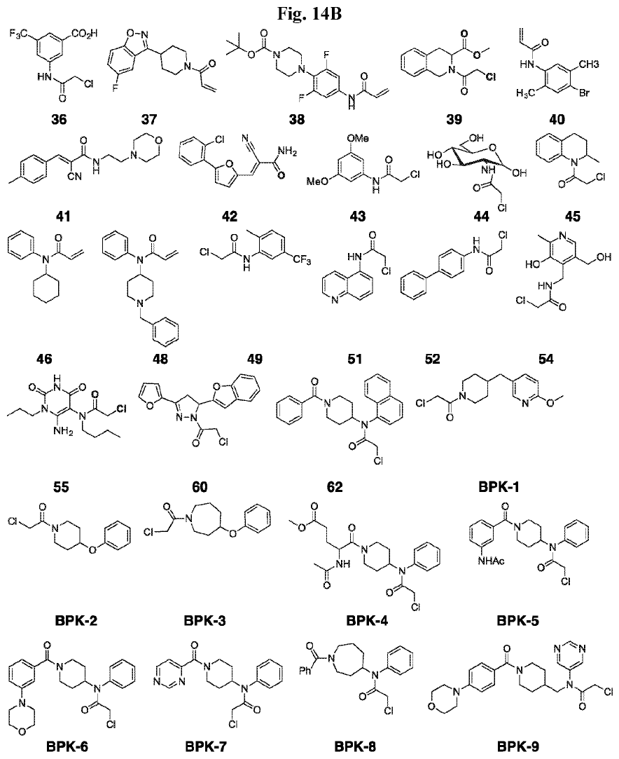

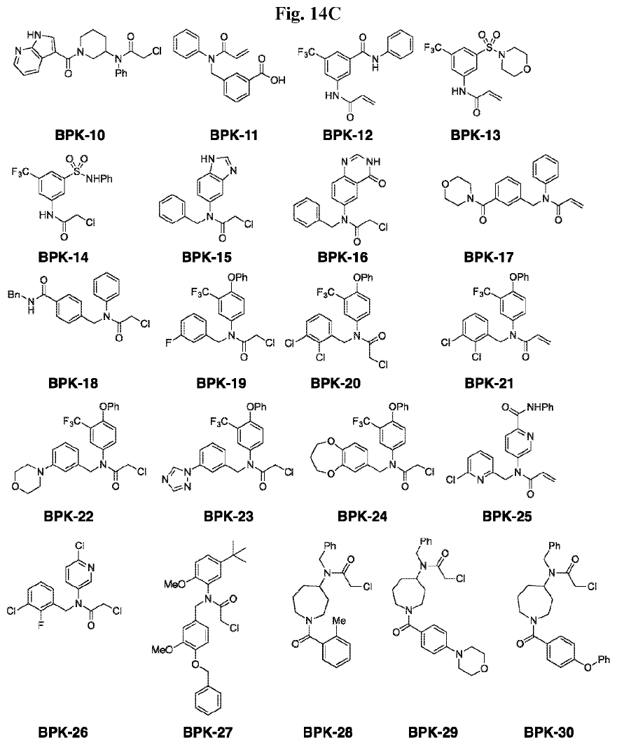

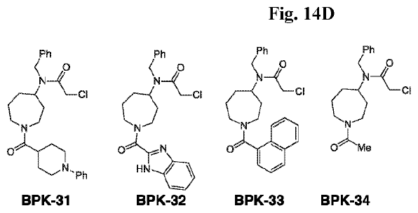

BPK-29 (30 μM, 12 h) also produced some of the gene expression changes caused by shRNA-mediated disruption of NR0B1 or NRF2 in KEAP1-mutant NSCLC cells (FIG. 13A), including reductions in CRY1, DEPDC1, and CPLX2 (FIG. 13B-C), which were not observed in KEAP1-WT NSCLC cells treated with BPK-29 (FIG. 13B). It was further confirmed that BPK-29-treated cells also showed a substantial reduction in CRY1 protein content (FIG. 13D). These gene and protein expression changes were not observed in KEAP1-mutant NSCLC cells treated with control compound BPK-9 (FIG. 13A-D).

In the course of studying the cellular activity of BPK-29, the concentration-dependent change in engagement of C274 of NR0B1 was less relative to other targets of the compound (FIG. 12A). Covalent ligands like BPK-29 engage proteins in a time-dependent manner, which led to speculate that differences in protein turnover rate in cells could affect the maximal absolute engagement of NR0B1 by BPK-29. Accordingly SILAC pulse-chase chemical proteomics experiments was performed in Keap1-mutant NSCLC cells, which revealed that NR0B1 was among a select subset of NRF2-regulated proteins that exhibit rapid turnover in NSCLC cells (FIG. 6G). These fast-turnover proteins generally corresponded to those that displayed early time point changes in protein abundance in our original isoTOP-ABPP analysis of shNRF2 cells (FIG. 6H). Similar results were obtained in KEAP1-mutant NSCLC cells treated with cycloheximide, which provided a half-life estimate for NR0B1 of ˜4.8 h (FIG. 13E). These findings demonstrate that NR0B1 is a short half-life protein in KEAP1-mutant NSCLC cells, possibly explaining its rapid decrease following NRF2 disruption and substantive, but incomplete engagement by BPK-29 in cells (FIG. 6A and FIG. 12A).

TABLE 5
Proteome-wide selectivity of NR0B1 ligand BPK-29

| | | BPK-29- | BPK-29- | | |
|---|---|---|---|---|---|
| | | competed | competed | Competed | Competed by |
| | | isoTOP-ABPP | BPK-29yne | residues | control ligands |
| UniProt ID | Protein | analysis^{#} | analysis* | (peptide) | BPK-9/27*^{,#} |
| P51843 | NR0B1 | Yes | Yes | C274 | No |
| Q8WV74 | NUDT8 | Yes | Yes | C207 | Yes |
| P22307 | SCP2 | Yes | Yes | C94 | Yes |
| P10599 | TXN | Yes | Yes | C35 | Yes |
| Q16881 | TXNRD1\hat{ } | Yes | Yes | U648 | Yes |
| O95881 | TXNDC12 | Yes | Yes | C66 | No |
| Q99757 | TXN2 | Yes | — | C90 | Yes |
| P00352 | ALDH1A1 | — | Yes | — | Yes |
| Q9BRX8 | FAM213A | — | Yes | — | Yes |
| Q9BVL4 | SELO\hat{ } | — | Yes | — | Yes |
| P78417 | GSTO1 | — | Yes | — | Yes |
| Q5TFE4 | NT5DC1 | — | Yes | — | Yes |
| Q9H7Z7 | PTGES2 | — | Yes | — | Yes |

\hat{ }Contains conserved functional (seleno)cysteine residue
*Competed defined as showing R value ≥ 2.5 at 20 μM of test compound
^{#}Competed defined as showing R value ≥ 3.0 at 40 μM of test compound
— BPK-29-competed protein or peptide not detected

### Example 3

### Synthetic Methodology

### Example S-1: Synthesis of 2-chloro-1-(4-((6-methoxypyridin-3-yl)methyl)piperidin-1-yl)ethan-1-one (BPK-1)

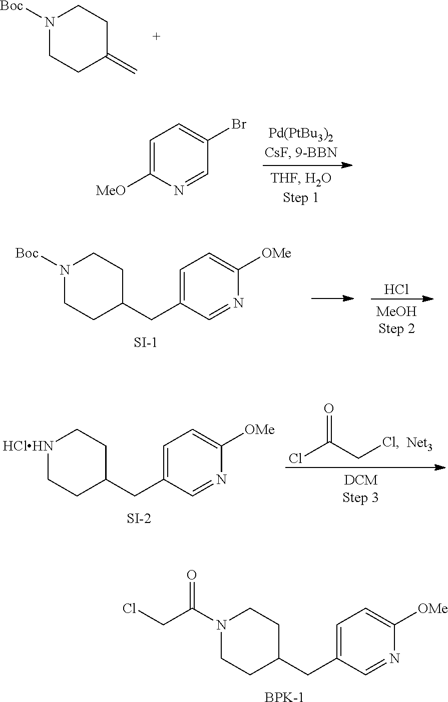

### Step 1.

Under an atmosphere of nitrogen, 9-BBN (0.5 M in THF, 5.1 mL, 2.53 mmol, 1.0 eq) was added to a solution tert-butyl 4-methylenepiperidine-1-carboxylate (500.0 mg, 2.53 mmol, 1.0 eq) in THF (12 mL) at 20° C. and the reaction was heated at reflux for 3 h. The mixture was then cooled down to 20° C., followed by the addition of CsF (769.0 mg, 5.06 mmol, 2.0 eq), 4-bromo-2-methoxy-pyridine (333.0 mg, 1.77 mmol, 0.7 eq), water (6 mL), and bis(tri-tert-butylphosphine)palladium(0) (38.8 mg, 0.076 mmol, 0.03 eq). The reaction was heated at reflux for 12 h and the progress was monitored by TLC (Petroleum ether: EtOAc=10: 1). Upon completion, the mixture was allowed to cool down and extracted with EtOAc (15 mL×3). The combined organic layers were washed with brine (50 mL), dried with anhydrous Na₂SO₄, filtered and concentrated in vacuo. The residue was purified by silica gel chromatography (Petroleum ether: EtOAc=50: 1 to 20: 1) to afford compound SI-1 (350.0 mg, 45%) as light-yellow oil, which was used in the next step without further purification. Step 2.

A mixture of compound SI-1 (250.0 mg, 0.82 mmol, 1.0 eq) in HCl/MeOH (4 M, 5 mL) was stirred at 15° C. for 2 h. Upon completion, the reaction was concentrated in vacuo to afford compound SI-2 (220.0 mg, HCl salt) as yellow oil, which was used in the next step without further purification. Step 3.

2-chloroacetyl chloride (57.0 μL, 0.72 mmol, 2.0 eq) was added to a solution of compound SI-2 (100.0 mg, 0.36 mmol, 1.0 eq, HCl salt) and NEt₃ (49.9 μL, 0.36 mmol, 1.0 eq) in DCM (5 mL) at 0° C. and the resulting mixture was stirred at 15° C. for 1 h. Upon completion, the reaction mixture was concentrated in vacuo and purified by prep. HPLC (TFA conditions) to afford the title compound (11.6 mg, 11%) as a light yellow solid. ¹H NMR (D₂O, 400 MHz) δ 8.32 (dd, J=9.1, 2.3 Hz, 1H), 8.09 (d, J=2.2 Hz, 1H), 7.44 (d, J=9.1 Hz, 1H), 4.38-4.21 (m, 3H), 4.16 (s, 3H), 3.93-3.84 (m, 1H), 3.18-3.09 (m, 1H), 2.77-2.64 (m, 3H), 2.01-1.86 (m, 1H), 1.78-1.66 (m, 2H), 1.29 (qd, J=12.6, 4.3 Hz, 1H), 1.17 (qd, J=12.7, 4.3 Hz, 1H). HRMS electrospray (m/z): [M+H]⁺ calcd for C₁₄H2₀C1N₂O₂: 283.1208, found: 283.1210.

### Example S-2: Synthesis of 2-chloro-1-(4-phenoxypiperidin-1-yl)ethan-1-one (BPK-2)

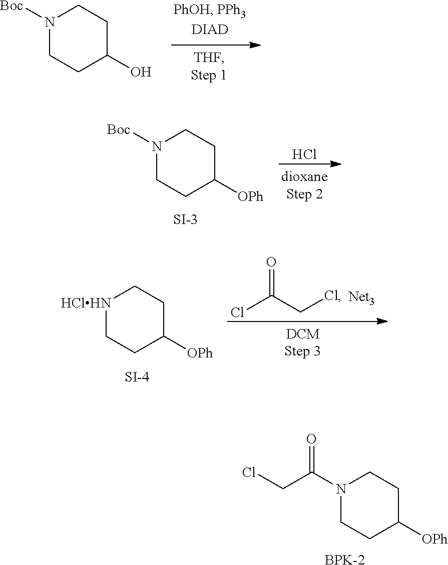

### Step 1.

DIAD (2.2 g, 10.9 mmol, 1.1 eq) was added to a solution of compound tert-butyl 4-hydroxypiperidine-1-carboxylate (2.0 g, 9.9 mmol, 1.0 eq), PPh₃ (2.9 g, 10.9 mmol, 1.1 eq.) and phenol (935.2 mg, 9.9 mmol, 1.0 eq) in THF (20 mL) at 0° C. The resulting mixture was stirred at 15° C. for 1 h, after which the solvent was removed under vacuum and the residue was purified by prep. HPLC (basic conditions) to afford tert-butyl 4-phenoxypiperidine-1-carboxylate (SI-3) as yellow oil.

### Step 2.

In a round-bottom flask HCl in dioxane (4 M, 3.6 mL, 4.0 eq) was added dropwise to a solution of compound SI-3 (1.0 g, 3.6 mmol, 1.0 eq) in dioxane (10 mL) at 0° C. The mixture was stirred at 15° C. for 1 h. Upon completion, the reaction mixture was concentrated under vacuum to afford compound SI-4 (500.0 mg) as an off-white solid, which was used in Step 3 without additional purification.

### Step 3.

Under an atmosphere of nitrogen, 2-chloroacetyl chloride (74 μL, 0.94 mmol, 2.0 eq) was added dropwise to a solution of compound SI-4 (100.0 mg, 0.47 mmol, 1.0 eq) and NEt₃ (261 μL, 1.87 mmol, 4.0 eq) in anhydrous DCM (1 mL) at 0° C. The mixture was stirred at 15° C. for 1 h. Upon completion, the reaction was quenched by the addition of water (50 mL) at 15° C., extracted with DCM (3×75 mL) and washed with brine (25 mL). The combined organic layers were dried over Na₂SO₄, filtered and concentrated under vacuum. The residue was purified by prep. HPLC (HCl conditions) to give compound the title compound as an off-white solid (49.5 mg, 42%). ¹H NMR (CDCl₃, 400 MHz) δ 7.33-7.27 (m, 2H), 6.97 (tt, J=7.4, 1.1 Hz, 1H), 6.94-6.90 (m, 2H), 4.63-4.56 (m, 1H), 4.10 (m, 2H), 3.86-3.63 (m, 3H), 3.50 (dt, J=13.8, 5.2 Hz, 1H), 2.05-1.83 (m, 4H). HRMS electrospray (m/z): [M+H]⁺ calcd for C₁₃H17ClNO₂: 254.0942, found: 254.0941.

### Example S-3: Synthesis of 2-chloro-1-(4-phenoxyazepan-1-yl)ethan-1-one (BPK-3)

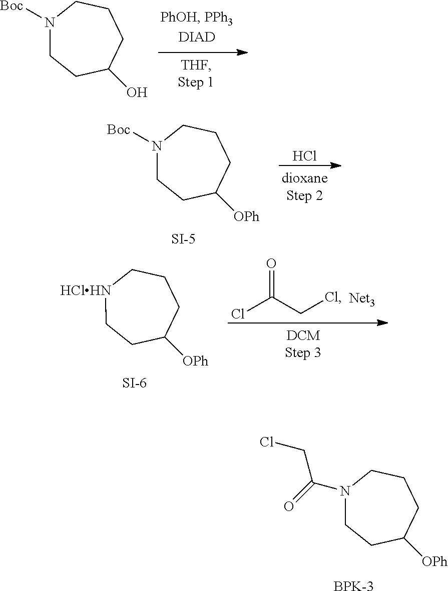

### Step 1.

DIAD (413.7 mg, 2.1 mmol, 1.1 eq) was added to a solution of tert-butyl 4-hydroxyazepane-1-carboxylate (400.4 mg, 1.9 mmol, 1.0 eq), PPh₃ (536.7 mg, 2.1 mmol, 1.1 eq) and phenol (175.0 mg, 1.9 mmol, 1.0 eq) in THF (4 mL) at 0° C. The resulting mixture was stirred at 15° C. for 16 h. Reaction progress was monitored by TLC (Petroleum ether: EtOAc=50: 1). Upon completion, the mixture was concentrated under vacuum and the residue was purified by silica gel chromatography to afford intermediate SI-5 as colorless oil (400.0 mg, 72%).

### Step 2.

In a round-bottom flask HCl in dioxane (4 M, 4.1 mL, 12.0 eq) was added dropwise to a solution of intermediate SI-5 (400.0 mg, 1.4 mmol, 1.0 eq) in dioxane (1 mL) at 0° C. The mixture was stirred at 15° C. for 1 h. Upon completion, the reaction mixture was concentrated under vacuum to afford compound SI-6 (300.0 mg, 94%) as a white solid, which was used in Step 3 without additional purification.

### Step 3.

Under an atmosphere of nitrogen, 2-chloroacetyl chloride (69.9 μL, 0.88 mmol, 2.0 eq) was added dropwise to a solution of amine SI-6 (100.0 mg, 0.44 mmol, 1.0 eq) and NEt₃ (245.0 μL, 1.76 mmol, 4.0 eq) in anhydrous DCM (1 mL) at 0° C. The mixture was stirred at 15° C. for 1 h. Upon completion, the reaction was quenched by the addition of water (50 mL) at 15° C., extracted with DCM (3×75 mL) and washed with brine (25 mL). The combined organic layers were dried over Na₂SO₄, filtered and concentrated under vacuum. The residue was purified by prep. HPLC (HCl conditions) to give compound the title compound as colorless oil (51.0 mg, 43%). ¹H NMR (CDCl₃, 400 MHz) δ 7.25-7.16 (m, 2H), 6.92-6.82 (m, 1H), 6.80 (d, J=8.1 Hz, 2H), 4.54-4.40 (m, 1H), 4.10-3.98 (m, 2H), 3.76-3.36 (m, 4H), 2.14-1.87 (m, 4H), 1.85-1.74 (m, 1H), 1.74-1.58 (m, 1H). HRMS electrospray (m/z): [M+H]⁺ calcd for C₁₄H1₉C1NO₂: 268.1099, found: 268.1100.

Compounds of Examples S-4-S-7 were synthesized from a common intermediate SI-8, which was obtained from compound SI-7 (Backus et al. 2016) as follows:

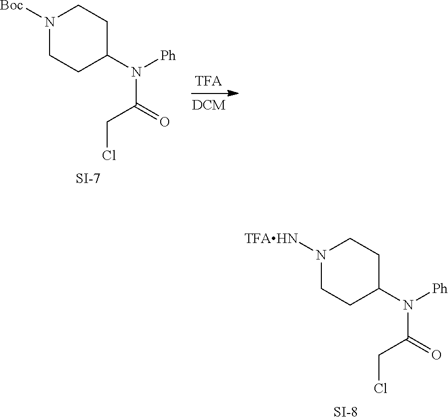

TFA (34.7 mL, 453.5 mmol, 10.0 eq) was added to a solution of compound SI-7 (16.0 g, 45.4 mmol, 1.0 eq) in DCM (20 mL) at 18° C. The resulting mixture was stirred at 18° C. for 3 h. Upon completion, the reaction mixture was concentrated in vacuo to give crude intermediate SI-8 (23.0 g) as yellow oil, which was used without further purification in the syntheses of Compounds of Examples S-4-S-7.

### Example S-4: Synthesis of methyl 4-acetamido-5-(4-(2-chloro-N-phenylacetamido)piperidin-1-yl)-5-oxopentanoate (BPK-4)

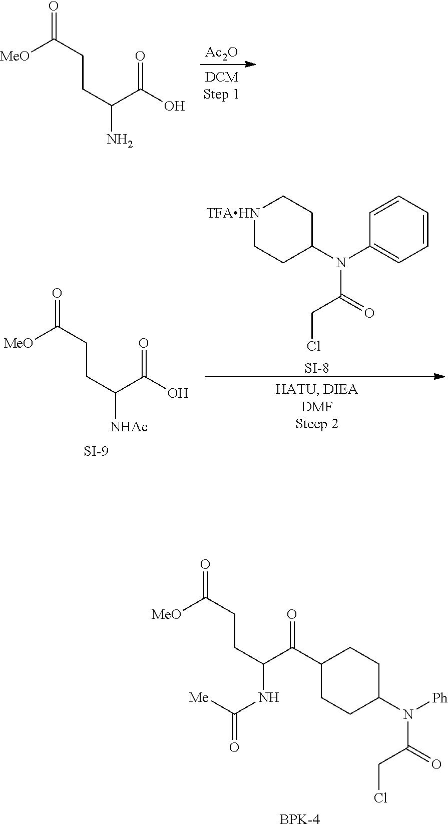

### Step 1.

Acetic anhydride (95.0 mg, 0.93 mmol, 1.5 eq) was added to a solution of 2-amino-5-methoxy-5-oxo-pentanoic acid (100.0 mg, 0.62 mmol, 1.0 eq) in DCM (2.0 mL) at room temperature and the resulting mixture was stirred at 30° C. for 16 h. Upon completion, the mixture was concentrated in vacuo to afford crude compound SI-9 (120.0 mg), which was used in the next step without additional purification.

### Step 2.

HATU (269.5 mg, 0.71 mmol, 1.2 eq) and DIEA (229.0 mg, 1.77 mmol, 3.0 eq) were added to a suspension of SI-9 (120.0 mg, 0.59 mmol, 1.0 eq) in DMF (2.0 mL). Intermediate SI-8 (238.3 mg, 0.68 mmol, 1.2 eq) was then added and the resulting mixture was stirred at 0° C. for 1 h. Upon completion, the reaction was acidified to pH 3 with HCl (0.5 M, 2 mL) and diluted with CH₃CN (1 mL). Purification by prep. HPLC (HCl conditions) afforded the title compound (16.0 mg, 6%) as a white solid. ¹H NMR (CDCl₃, 400 MHz) δ 7.50-7.41 (m, 3H), 7.18-7.06 (m, 2H), 6.51 (br, 1H), 4.99-4.73 (m, 2H), 4.62 (d, J=13.0 Hz, 1H), 4.26-4.10 (m, 1H), 3.70 (s, 2H), 3.67 (s, 2H), 3.64 (s, 1H), 3.25-3.11 (m, 1H), 2.76-2.61 (m, 1H), 2.45-2.20 (m, 3H), 2.08-1.85 (m, 6H), 1.42-1.16 (m, 2H). HRMS electrospray (m/z): [M+H]⁺ calcd for C₂₁H2₉C1N₃O₅: 438.1790, found: 438.1793.

### Example S-5: Synthesis of N-(1-(3-acetamidobenzoyl)piperidin-4-yl)-2-chloro-N-phenylacetamide (BPK-5)

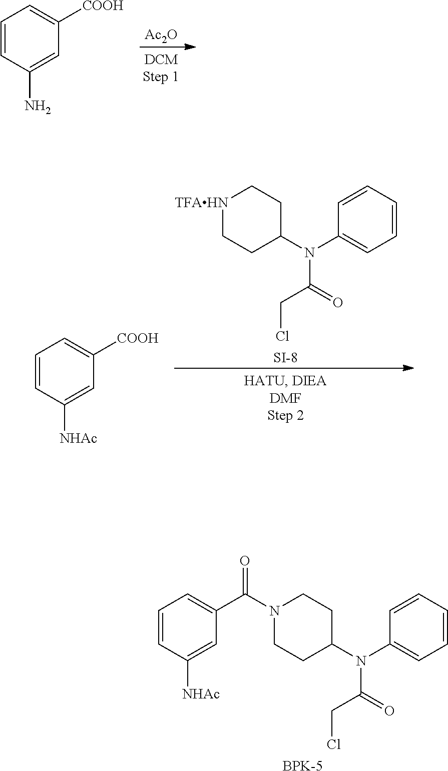

### Step 1.

Acetic anhydride (148.9 mg, 1.46 mmol, 2.0 eq) was added in one portion to a mixture of 3-aminobenzoic acid (100.0 mg, 0.73 mmol, 1.0 eq) in DCM (1 mL) at 15° C. The mixture was stirred at 15° C. for 16 h. Upon completion, the mixture was filtered and the filter cake was washed with DCM (3 mL), then dried in vacuo to afford 3-acetamidobenzoic acid (120.0 mg) as a white solid, which was used in the next step without further purification.

### Step 2.

To a suspension of 3-acetamidobenzoic acid (225.2 mg, 0.61 mmol, 1.1 eq, TFA) in DMF (2 mL) were added HATU (254.7 mg, 0.67 mmol, 1.2 eq) and DIEA (216.4 mg, 1.7 mmol, 3.0 eq) followed by Intermediate SI-8 (100.0 mg, 0.56 mmol, 1.0 eq). The resulting mixture was stirred at 0° C. for 2 h. Upon completion, the mixture was quenched with water (5 mL) and extracted with EtOAc (3×3 mL). The combined organic layers were washed with hydrochloric acid (3 mL, 0.5 M) and concentrated in vacuo. The residue was diluted with CH₃CN (5 mL) and purified by prep. HPLC (basic conditions) to afford the title compound (45.1 mg, 20%) as a white solid. ¹H NMR (CDCl₃, 400 MHz) δ 7.77 (s, 1H), 7.60-7.53 (m, 1H), 7.51-7.44 (m, 3H), 7.41-7.35 (m, 1H), 7.27 (t, J=7.7 Hz, 1H), 7.14 (br, 2H), 6.97 (d, J=7.7 Hz, 1H), 4.87-4.68 (m, 2H), 3.87-3.75 (m, 1H), 3.71 (s, 2H), 3.21-3.05 (m, 1H), 2.91-2.75 (m, 1H), 2.13 (s, 3H), 1.99-1.75 (m, 2H), 1.45-1.17 (m, 2H). HRMS electrospray (m/z): [M+H]⁺ calcd for C₂₂H₂₅C1N₃O₃: 414.1579, found: 414.1580.

### Example S-6: Synthesis of 2-chloro-N-(1-(3-morpholinobenzoyl)piperidin-4-yl)-N-phenylacetamide (BPK-6)

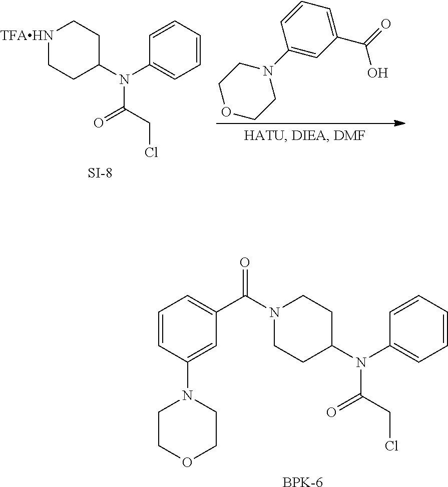

HATU (137.6 mg, 0.36 mmol, 1.5 eq) and DIEA (93.6 mg, 0.72 mmol, 3.0 eq) were added to a solution of intermediate SI-8 (100.0 mg, 0.27 mmol, 1.1 eq, TFA salt) in DMF (2 mL). 3-morpholinobenzoic acid (50.0 mg, 0.24 mmol, 1.0 eq) was then added and the resulting mixture was stirred at 15° C. for 16 h. Upon completion, the reaction mixture was diluted with CH₃CN (3 mL) and purified by prep. HPLC (HCl conditions) to afford the title compound (37.0 mg, 34%) as a white solid. ¹H NMR (CDCl₃, 400 MHz) δ 7.93-7.88 (m, 2H), 7.56 (t, J=7.7 Hz, 1H), 7.51-7.43 (m, 4H), 7.18 (s, 2H), 4.87-4.69 (m, 2H), 4.34 (s, 4H), 3.71 (s, 3H), 3.51 (s, 4H), 3.22 (br, 1H), 2.86 (br, 1H), 1.92 (br, 2H), 1.42 (br, 2H). HRMS electrospray (m/z): [M+H]⁺ calcd for C₂₄H2₉C1N₃O₃: 442.1892, found: 442.1892.

### Example S-7: Synthesis of 2-chloro-N-phenyl-N-(1-(pyrimidine-4-carbonyl)piperidin-4-yl)acetamide (BPK-7)

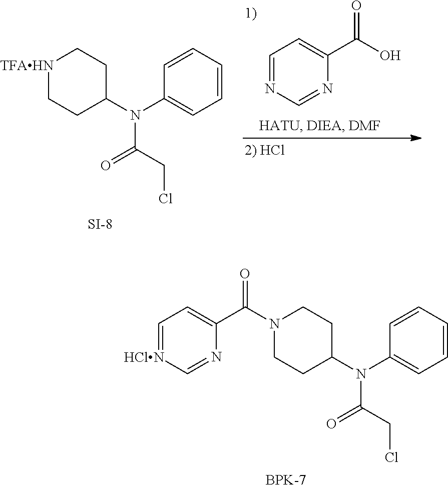

HATU (257.4 mg, 0.68 mmol, 1.2 eq) and DIEA (218.7 mg, 1.69 mmol, 3.0 eq) were added to a suspension of pyrimidine-4-carboxylic acid (70.0 mg, 0.56 mmol, 1.0 eq) in DMF (2 mL). Intermediate SI-8 (227.6 mg, 0.63 mmol, 1.1 eq, TFA salt) was then added and the resulting mixture was stirred at 0° C. for 2 h. Upon completion, the mixture was acidified to pH 3 with HCl (0.5 M, 2 mL), diluted with CH₃CN (1 mL) and purified by prep. HPLC (HCl conditions) to afford the title compound (74.9 mg, 34%, HCl salt) as a red solid. ¹H NMR (CDCl₃, 400 MHz) δ 9.31 (s, 1H), 9.00 (d, J=4.6 Hz, 1H), 7.77 (d, J=4.4 Hz, 1H), 7.51-7.43 (m, 3H), 7.15 (s, 2H), 4.92-4.82 (m, 1H), 4.75 (d, J=13.2 Hz, 1H), 3.93 (d, J=12.2 Hz, 1H), 3.71 (s, 2H), 3.23 (t, J=12.8 Hz, 1H), 2.91 (t, J=12.0 Hz, 1H), 1.95 (dd, J=37.9, 12.2 Hz, 2H), 1.50-1.36 (m, 2H). HRMS electrospray (m/z): [M+H]⁺ calcd for C₁₈H₂₀ClN₄O₂: 359.1269, found: 359.1272.

### Example S-8: Synthesis of N-(1-benzoylazepan-4-yl)-2-chloro-N-phenylacetamide (BPK-8)

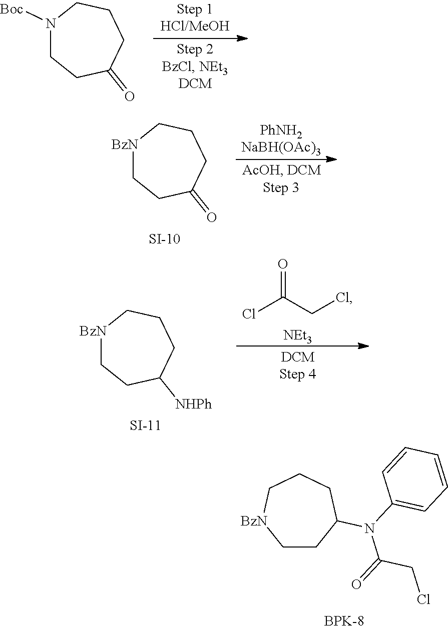

### Step 1.

A solution of tert-butyl 4-oxoazepane-1-carboxylate (1.00 g, 4.7 mmol, 1.0 eq) in HCl/MeOH (4 M, 10.0 mL, 8.5 eq) was stirred at 15° C. for 12 h. Upon completion, the reaction mixture was concentrated in vacuo to give crude azepan-4-one (750.0 mg, HCl salt) as a white solid, which was used in Step 2 without further purification.

### Step 2.

Benzoyl chloride (1.17 mL, 10.0 mmol, 2.0 eq) was added dropwise to a solution of azepan-4-one (0.75 g, 5.0 mmol, 1.0 eq, HCl salt) and NEt₃ (2.10 mL, 15.0 mmol, 3.0 eq) in DCM (50 mL) at 0° C. The resulting mixture was stirred at 15° C. for 3 h, quenched with water (10 mL) and extracted with DCM (3×15 mL). The combined organic layers were washed with brine (5 mL), dried with anhydrous Na₂SO₄, filtered and concentrated to afford crude compound SI-10 (0.50 g) as colorless oil, which was used in step 3 without additional purification.

### Step 3.

Under an atmosphere of nitrogen, AcOH (79.0 μL, 1.4 mmol, 1.0 eq) was added to a solution of compound SI-10 (300.0 mg, 1.4 mmol, 1.0 eq) and aniline (135.0 mg, 1.5 mmol, 1.05 eq) in anhydrous DCM (5 mL) at 15° C. The reaction was then stirred at 15° C. for 3 h. Subsequently, NaBH(OAc)₃ (585.3 mg, 2.8 mmol, 2.0 eq) was added and the reaction was stirred at 15° C. for an additional 12 h. After this time, LCMS showed that half of the starting material was consumed. The reaction was quenched by the addition of water (5 mL) and extracted with DCM (3×10 mL). The combined organic layers were washed with brine (5 mL), dried with anhydrous Na₂SO₄, filtered and concentrated. The residue was purified by prep. HPLC (basic conditions) to afford compound SI-11 (230.0 mg) as a yellow solid.

### Step 4.

Under an atmosphere of nitrogen, 2-chloroacetyl chloride (53 μL, 0.66 mmol, 2.0 eq) was added dropwise to a solution of compound SI-11 (150.0 mg, 0.51 mmol, 1.5 eq) and NEt₃ (92 μL, 0.66 mmol, 2.0 eq) in anhydrous DCM (3 mL) at 0° C. The mixture was stirred at 15° C. for 12 h. Upon completion, the reaction was concentrated in vacuo and the residue was purified by prep. HPLC (HCl conditions) to afford the title compound as an off-white solid (50.0 mg, 41%). The compound was analyzed and further used as the racemate (R:S=1:1). ¹H NMR (CDCl₃, 400 MHz) δ 7.51-7.42 (m, 6H), 7.39-7.31 (m, 6H), 7.26 (br, 4H), 7.22-7.07 (m, 4H), 4.66 (q, J=12.3 Hz, 2H), 4.17-4.06 (m, 1H), 3.84-3.74 (m, 1H), 3.70 (dd, J=9.3, 2.2 Hz, 4H), 3.57-3.18 (m, 6H), 2.15-1.33 (m, 12H). HRMS electrospray (m z): [M+H]⁺ calcd for C₂₁H2₄C1N₂O₂: 371.1521, found: 371.1519.

### Example S-9: Synthesis of 2-chloro-N-((1-(4-morpholinobenzoyl)piperidin-4-yl)methyl)-N-(pyrimidin-5-yl)acetamide (BPK-9)

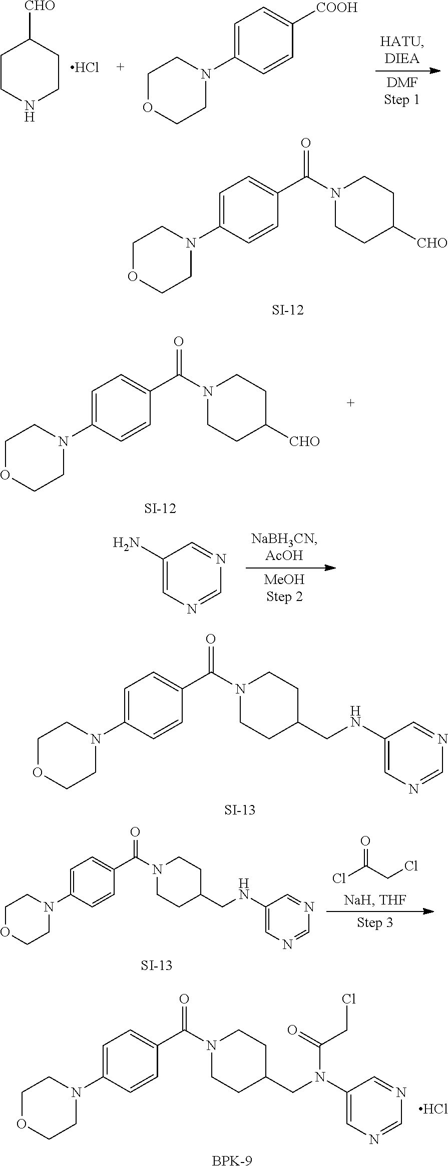

### Step 1.

HATU (6.10 g, 16.0 mmol, 1.2 eq) and DIEA (5.2 g, 40.1 mmol, 3.0 eq) were added to a solution of 4-morpholinobenzoic acid (3.05 g, 14.7 mmol, 1.1 eq) in DMF (30.0 mL). The resulting mixture was stirred at 20° C. for 1 h, after which piperidine-4-carbaldehyde (2.00 g, 13.4 mmol, 1.0 eq, HCl salt) was added to the mixture at 0° C. in several portions. The mixture was stirred at 20° C. for 16 h. Upon completion, the reaction was poured into water (300 mL) and extracted with DCM (3×100 mL). The combined organic layers were washed with brine (2×50 mL), dried over Na₂SO₄, filtered and concentrated in vacuo. Purification by prep. HPLC (TFA conditions) afforded compound SI-12 (1.15 g, 28%) as yellow oil.

### Step 2.

A solution of pyrimidin-5-amine (113.2 mg, 1.2 mmol, 1.2 eq), AcOH (68 μL, 1.2 mmol, 1.2 eq), and compound SI-12 (300.0 mg, 1.0 mmol, 1.0 eq) in anhydrous MeOH (3.0 mL) was stirred at 63° C. for 30 h. NaBH₃CN (187.0 mg, 3.0 mmol, 3.0 eq) was then added and the reaction mixture was stirred at 25° C. for additional 16 h. Upon completion, the reaction mixture was concentrated in vacuo, diluted with saturated aqueous NaHCO₃ (2 mL) and extracted with DCM (3×3 mL). The combined organic layers were dried over Na₂SO₄, filtered and concentrated. Purification by prep. HPLC (basic conditions) afforded compound SI-13 (185.0 mg, 48%) as colorless oil.

### Step 3.

NaH (21.0 mg, 0.5 mmol, 60% in oil, 5.0 eq) was added to a solution of compound SI-13 (40.0 mg, 0.1 mmol, 1.0 eq) in anhydrous THF (1.0 mL) at 0° C. and the resulting suspension was stirred at 25° C. for 30 min. The reaction mixture was then cooled to 0° C. and 2-chloroacetylchloride (17 μL, 0.21 mmol, 2.0 eq) was added dropwise. The reaction was stirred at 25° C. for additional 20 h and subsequently quenched by dropwise addition of HCl (3 M, 3 mL). The resulting mixture was then neutralized to pH 3-5 with saturated aqueous NaHCO₃ and extracted with DCM (3×2 mL). The combined organic layers were dried over Na₂SO₄, filtered and concentrated in vacuo. Purification by prep. HPLC (HCl conditions) afforded the title compound (23.0 mg, 44%, HCl salt) as a light yellow solid. ¹H NMR (DMSO-d₆, 400 MHz) δ 9.19 (s, 1H), 8.95 (s, 2H), 7.34 (d, J=8.7 Hz, 2H), 7.28 (d, J=8.5 Hz, 2H), 4.13 (s, 2H), 3.89-3.81 (m, 4H), 3.71-3.59 (m, 2H), 3.35-3.26 (m, 4H), 2.81 (s, 2H), 1.69 (d, J=17.3 Hz, 3H), 1.20-1.01 (m, 2H). Note: peak at 5.00 ppm (2H) overlaps with a broad signal of HCl. HRMS electrospray (m/z): [M+H]⁺ calcd for C₂₃H2₉C1N₅O₃: 458.1953, found: 458.1952.

### Example S-10: Synthesis of N-(1-(1H-pyrrolo[2,3-b]pyridine-2-carbonyl)piperidin-4-yl)-2-chloro-N-phenylacetamide (BPK-10)

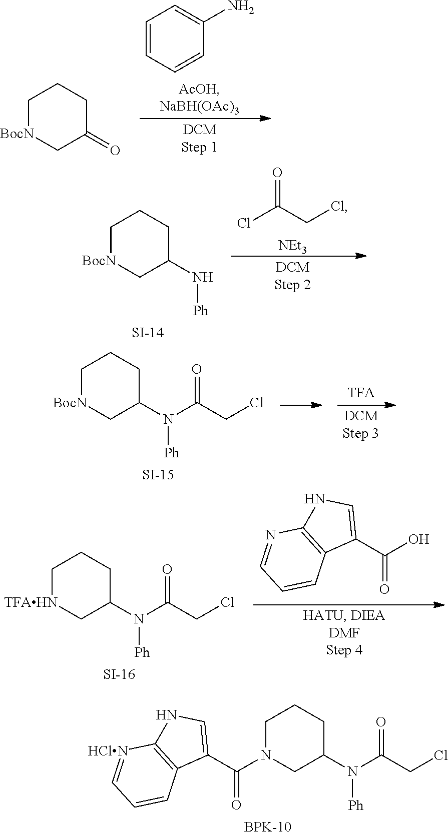

### Step 1.

Aniline (4.58 mL 50.2 mmol, 1.0 eq) and tert-butyl 3-oxopiperidine-1-carboxylate (10.0 g, 50.2 mmol, 1.0 eq) were added to a solution of AcOH (2.87 mL, 50.2 mmol, 1.0 eq) in anhydrous DCM (150 mL) and the mixture was stirred for 16 h. NaBH(OAc)₃ (21.3 g, 100 mmol, 2.0 eq) was then added and the reaction was stirred for an additional 3 h. Upon completion, the mixture was washed with saturated aqueous NaHCO₃ (50 mL) and brine (50 mL), dried with anhydrous Na₂SO₄, filtered and concentrated in vacuo to afford the intermediate SI-14 (15.0 g) as yellow oil, which was used in the next step without further purification.

### Step 2.

2-chloroacetyl chloride (8.63 mL, 109.0 mmol, 2.0 eq) was added dropwise to a solution of intermediate SI-14 (15.0 g, 54.3 mmol, 1.0 eq) and NEt₃ (30.0 mL, 217.0 mmol, 4.0 eq) in DCM (1 mL) at 0° C. The mixture was warmed to ambient temperature and stirred for 2 h. Upon completion, the reaction was quenched with water (15 mL) and extracted with DCM (3×5 mL). The combined organic layers were washed with brine (3×5 mL), dried over Na₂SO₄, filtered and concentrated under reduced pressure to give intermediate SI-15 (13.0 g) as yellow oil, which was used directly in the next step.

### Step 3.

TFA (1.51 mL, 20.4 mmol, 3.0 eq) was added dropwise to a solution of intermediate SI-15 (2.40 g, 6.8 mmol, 1.0 eq) in DCM (2 mL) at 0° C. The mixture was then warmed to ambient temperature and stirred for 2 h. Upon completion, the reaction was quenched with water (2 mL) and extracted with DCM (3×2 mL). The combined organic layers were washed with brine (3×2 mL), dried over Na₂SO₄, filtered and concentrated under reduced pressure to afford intermediate SI-16 (1.30 g) as yellow oil, which was used in the next step without additional purification.

### Step 4.

A solution of HATU (281.4 mg, 0.74 mmol, 1.2 eq) and DIEA (323.0 μL, 1.9 mmol, 3.0 eq) in DMF (2 mL) was added to a solution of 1H-pyrrolo[2,3-b]pyridine-3-carboxylic acid (100.0 mg, 0.62 mmol, 1.0 eq) in DMF and the resulting mixture was stirred for 30 min. Intermediate SI-16 (187.0 mg, 0.74 mmol, 1.2 eq) was then added and the mixture was stirred at 0° C. for another 1.5 h. Upon completion, the reaction was quenched with water (1 mL) and extracted with DCM (3×1 mL). The combined organic layers were dried over Na₂SO₄, filtered and concentrated under reduced pressure. The resulting residue was re-dissolved in CH₃CN (1 mL) and water (0.5 mL) and purified by prep. HPLC (HCl conditions) to afford the title compound (70.0 mg, 25%, HCl salt) as yellow oil. ¹H NMR (DMSO-d₆, 400 MHz) δ 13.15 (s, 1H), 8.51-8.42 (m, 2H), 8.11 (s, 1H), 7.51-7.41 (m, 4H), 7.35 (d, J=5.9 Hz, 2H), 4.60-4.43 (m, 2H), 4.18 (s, 1H), 3.83 (s, 2H), 2.82-2.56 (m, 2H), 1.87 (d, J=10.7 Hz, 1H), 1.67 (d, J=12.6 Hz, 1H), 1.60-1.46 (m, 1H), 1.16-1.02 (m, 1H). HRMS electrospray (m/z): [M+H]⁺ calcd for C₂₁H2₂C1N₄O₂: 397.1426, found: 397.1425.

### Example S-11: Synthesis of 3-((N-phenylacrylamido)methyl)benzoic acid (BPK-11)

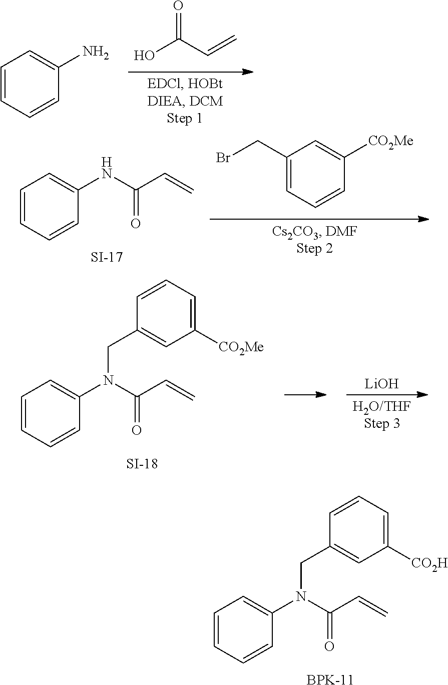

### Step 1.

A solution of acrylic acid (1.10 mL, 16.11 mmol, 1.5 eq), 1-ethyl-3-(3-dimethylaminopropyl)carbodiimide (3.09 g, 16.11 mmol, 1.5 eq), DIEA (5.6 mL, 32.22 mmol, 3.0 eq), and 1-hydroxybenzotriazole (1.45 g, 10.74 mmol, 1.0 eq) in DCM (20 mL) was stirred at 20° C. for 1 h, after which aniline (1.00 g, 10.74 mmol, 1.0 eq) was added dropwise at 0° C. The reaction was stirred at 20° C. for 11 hours and the reaction progress was monitored by TLC (Petroleum ether:EtOAc=1:3). Upon completion, the mixture was diluted with water (20 mL) and extracted with dichloromethane (20 mL×2). The combined organic layers were washed with brine (50 mL), dried with anhydrous Na₂SO₄, filtered and concentrated in vacuo. The residue was purified by silica gel chromatography (Petroleum ether:EtOAc=10:1) to afford compound SI-17 (300.0 mg, 7%) as an off-white solid.

### Step 2.

A mixture of compound SI-17 (150.0 mg, 1.02 mmol, 1.0 eq), methyl 3-(bromomethyl)benzoate (233.0 mg, 1.02 mmol, 1.0 eq) and cesium carbonate (665.0 mg, 2.04 mmol, 2.0 eq) in DMF (3 mL) was stirred at 20° C. for 12 hours. Upon completion, the reaction was quenched with water (15 mL) and extracted with EtOAc (10 mL×2). The combined organic layers were washed with water (15 mL×3) and brine (15 mL), dried over anhydrous Na₂SO₄, filtered and concentrated in vacuo to afford compound SI-18 (120 mg) as yellow oil.

### Step 3.

A solution of lithium hydroxide monohydrate (28.4 mg, 0.68 mmol, 2.0 eq) in water (3 mL) was added dropwise to a solution of compound SI-18 (100.0 mg, 0.34 mmol, 1.0 eq) in THF (3 mL) at 20° C. and the mixture was stirred at 20° C. for 12 hours. Upon completion, the mixture was concentrated in vacuo and the crude product was purified by prep. HPLC (HCl conditions) to afford the target product the title compound (28.0 mg, 29%) as an off-white solid. ¹H NMR (CDCl₃, 400 MHz) δ 7.99 (d, J=7.7 Hz, 1H), 7.92 (s, 1H), 7.54 (d, J=7.6 Hz, 1H), 7.43-7.29 (m, 4H), 7.02 (d, J=7.1 Hz, 2H), 6.47 (d, J=16.7 Hz, 1H), 6.05 (dd, J=16.8, 10.3 Hz, 1H), 5.58 (d, J=10.4 Hz, 1H), 5.05 (s, 2H). HRMS electrospray (m/z): [M+H]⁺ calcd for C₂₃H2₆C1N₄O₂: 282.1125, found: 282.1124.

### Example S-12: Synthesis of 3-acrylamido-N-phenyl-5-(trifluoromethyl)benzamide (BPK-12)

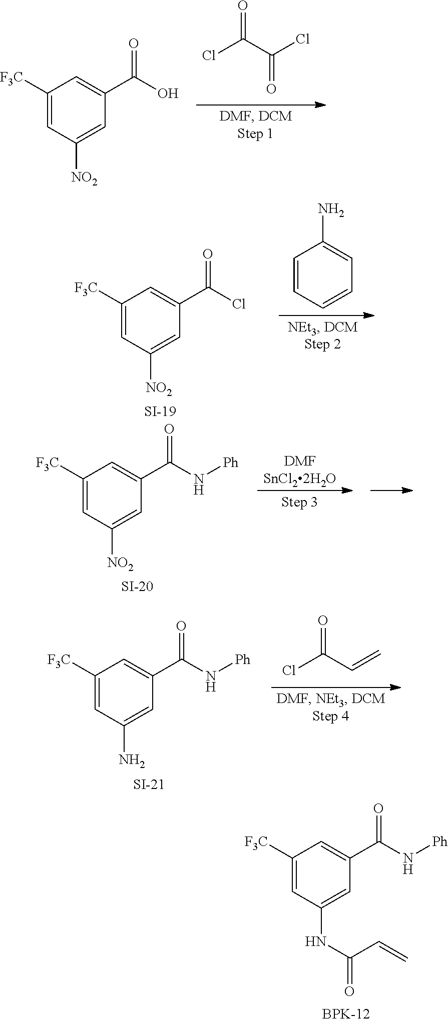

### Step1.

Oxalyl dichloride (140.0 mg, 1.1 mmol, 1.3 eq) and DMF (50 μL) were added to a solution of 3-nitro-5-(trifluoromethyl)benzoic acid (200.0 mg, 0.85 mmol, 1.0 eq) in DCM (2.0 mL). The mixture was stirred at 40° C. for 3 h. The reaction was then concentrated in vacuo to afford compound SI-19 (250.0 mg) as light yellow oil, which was used in the next step without additional purification.

### Step2.

NEt₃ (71.8 mg, 0.71 mmol, 3.0 eq) and aniline (22.0 mg, 0.24 mmol, 1.0 eq) were added to a solution of SI-19 (60.0 mg, 0.24 mmol, 1.0 eq) in DCM (1.0 mL) and the resulting mixture was stirred at 15° C. for 18 h. Upon completion, the reaction was concentrated in vacuo to afford compound SI-20 (80.0 mg) as a light yellow solid, which was used in the next step without additional purification.

### Step 3.

SnCl₂.2H₂O (215.3 mg, 0.95 mmol, 4.0 eq) and DMF (174 μg, 2.4 μmol, 0.01 eq) were added to a solution of compound SI-20 (74.0 mg, 0.24 mmol, 1.0 eq) in EtOH (1.0 mL) and the resulting mixture was stirred at 80° C. for 2 h. Upon completion, the reaction was quenched with aqueous NaHCO₃ (2 mL), stirred for 5 min and extracted with DCM (3×2 mL). The combined organic layers were dried with Na₂SO₄, filtered and concentrated in vacuo to afford SI-21 (90.0 mg) as light yellow oil, which was used in the next step without additional purification.

### Step 4.

Acryloyl chloride (23.6 mg, 0.26 mmol, 0.8 eq) and DMF (0.2 mg, 3.1 μmol, 0.01 eq) were added to a solution of compound SI-21 (90.0 mg, 0.32 mmol, 1.0 eq) in DCM (1.0 mL) and the resulting mixture was stirred at 15° C. for 18 h. Upon completion, the mixture was concentrated in vacuo and the resulting residue was purified by prep. HPLC (FA conditions) to afford the title compound (20.0 mg, 18%) as a white solid. ¹H NMR (DMSO-d₆, 400 MHz) δ 10.77-10.72 (m, 1H), 10.50 (s, 1H), 8.42 (s, 1H), 8.37 (s, 1H), 8.03 (s, 1H), 7.76 (d, J=7.9 Hz, 2H), 7.38 (t, J=7.9 Hz, 2H), 7.14 (t, J=7.4 Hz, 1H), 6.46 (dd, J=17.0, 9.9 Hz, 1H), 6.34 (dd, J=17.0, 2.0 Hz, 1H), 5.86 (dd, J=9.9, 1.9 Hz, 1H). HRMS electrospray (m/z): [M+H]+ calcd for C₁₇H1₄F3N₂O₂: 335.1002, found: 335.1002

### Example S-13: Synthesis of N-(3-(piperidin-1-ylsulfonyl)-5-(trifluoromethyl)phenyl)acrylamide (BPK-13)

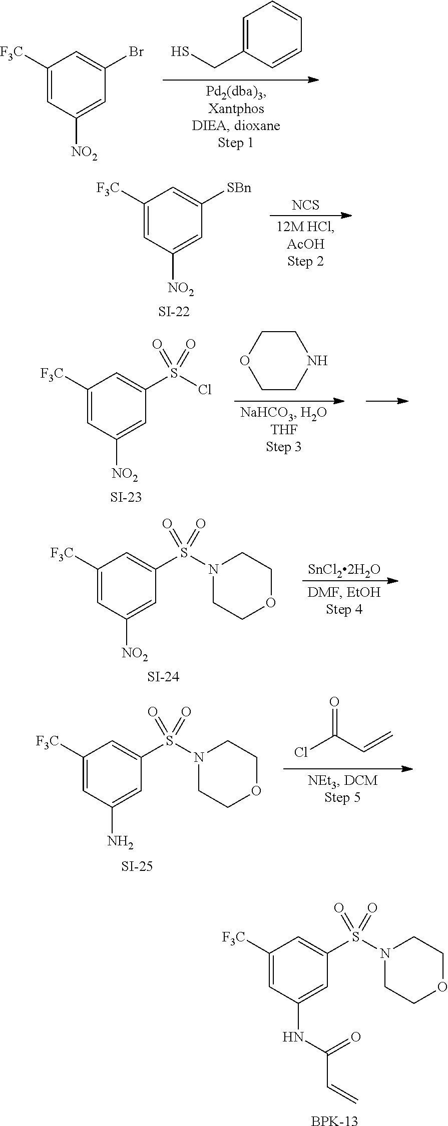

### Step 1.

Under an atmosphere of nitrogen, a two-neck round-bottom flask was charged with 1-bromo-3-nitro-5-(trifluoromethyl)benzene (11.50 g, 42.6 mmol, 1.0 eq), Pd₂(dba)₃ (1.17 g, 1.3 mmol, 0.03 eq), Xantphos (1.23 g, 2.1 mmol, 0.05 eq), DIEA (14.9 mL, 85.2 mmol, 2.0 eq), and 1,4-dioxane (90 mL). The flask was fitted with a reflux condenser and stirred at 80° C. for 10 min, after which benzylthiol (5.5 mL, 46.9 mmol, 1.1 eq) was added. The mixture was stirred at 80° C. for an additional 20 min and monitored by TLC (Petroleum ether: EtOAc=20: 1). Upon completion, the reaction was quenched with aqueous NaHCO₃ (100 mL) and extracted with ethyl acetate (3×100 mL). The combined organic layers were washed with brine (50 mL), dried with anhydrous Na₂SO₄, filtered and concentrated in vacuo. The resulting residue was passed through a short silica gel plug (Petroleum ether) to afford crude SI-22 (15.0 g) as a yellow liquid, which was used in the next step without additional purification.

### Step 2.

NCS (17.05 g, 127.7 mmol, 4.0 eq) was added to a solution of compound SI-22 (10.0 g, 31.9 mmol, 1.0 eq) in HCl (12 M, 12.5 mL, 4.7 eq) and AcOH (60 mL) at 0° C. The mixture was stirred at 25° C. for 16 h and monitored by TLC (Petroleum ether: EtOAc=20: 1). Upon completion, the reaction was poured into ice water (500 mL) and extracted with ethyl acetate (3×50 mL). The combined organic layers were washed with brine (500 mL), dried with anhydrous Na₂SO₄, filtered and concentrated in vacuo to afford crude compound SI-23 (13.0 g), which was used without additional purification for the synthesis of compounds of Examples S-13 and S-14.

### Step 3.

A solution of intermediate SI-23 (180.0 mg, 0.62 mmol, 1.0 eq) in THF (1 mL) was added to a solution of NaHCO₃ (313.3 mg, 3.7 mmol, 6.0 eq) and morpholine (54.7 μL, 0.62 mmol, 1.0 eq) in water (10 mL) at 0° C. The resulting mixture was stirred at 25° C. for 16 h and monitored by TLC (Petroleum ether: EtOAc=1: 1). Upon completion, the reaction was quenched with water (5 mL) and extracted with ethyl acetate (3×5 mL). The combined organic layers were washed with brine (5 mL), dried with anhydrous Na₂SO₄, filtered and concentrated in vacuo. The resulting residue was purified by silica gel chromatography (Petroleum ether: EtOAc=5: 1) to give compound SI-24 (200.0 mg, 95%) as a white solid.

### Step 4.

SnCl₂.2H₂O (400.0 mg, 1.77 mmol, 3.1 eq) was added to a mixture of intermediate SI-24 (190.0 mg, 0.56 mmol, 1.0 eq) and DMF (2.2 μL, 27.9 μmol, 0.05 eq) in EtOH (2.0 mL). The mixture was stirred at 78° C. for 16 h. Upon completion, the reaction was quenched by adjusting the pH to pH 9 with saturated aqueous NaHCO₃ (10 mL) and the resulting mixture was extracted with ethyl acetate (3×5 mL). The combined organic layers were washed with brine (5 mL), dried with anhydrous Na₂SO₄, filtered and concentrated in vacuo to afford crude SI-25 (150.0 mg) as a yellow solid, which was used in the next step without further purification.

### Step 5.

Acryloyl chloride (18.9 μL, 0.23 mmol, 1.0 eq) was added to a solution of compound SI-25 (70.0 mg, 0.23 mmol, 1.0 eq) and NEt₃ (62.5 μL, 0.45 mmol, 2.0 eq) in anhydrous DCM (1 mL) at 0° C. and the mixture was stirred at 25° C. for 3 h. Upon completion, the reaction was concentrated in vacuo, the resulting residue was re-dissolved in CH₃CN (2 mL) and water (3 mL) and purified by prep. HPLC (FA conditions) to give the title compound (26.0 mg, 32%) as a white solid. ¹H NMR (DMSO-d₆, 400 MHz) δ 10.91 (s, 1H), 8.42-8.40 (m, 1H), 8.34 (t, J=1.8 Hz, 1H), 7.65-7.62 (m, 1H), 6.48-6.31 (m, 2H), 5.89 (dd, J=9.5, 2.4 Hz, 1H), 3.67-3.62 (m, 4H), 2.97-2.92 (m, 4H). HRMS electrospray (m/z): [M+H]⁺ calcd for C₁₄H1₆F3N₂O₄S: 365.0777, found: 365.0776.

### Example S-14: Synthesis of 2-chloro-N-(3-(N-phenylsulfamoyl)-5-(trifluoromethyl)phenyl)acetamide (BPK-14)

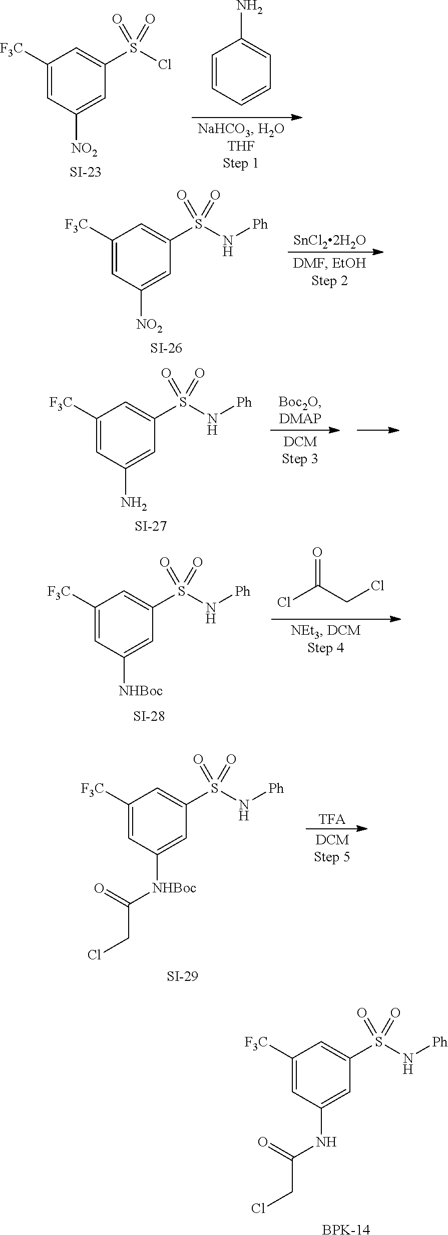

Intermediate SI-23 was synthesized according to the procedure described above.

### Step 1.

A solution of intermediate SI-23 (1.30 g, 4.49 mmol, 1.0 eq) in THF (7 mL) was added to a solution of NaHCO₃ (2.26 g, 26.9 mmol, 6.0 eq) and aniline (410.0 μL, 4.49 mmol, 1.0 eq) in water (70 mL) at 0° C. The resulting mixture was stirred at 25° C. for 2 h and monitored by TLC (Petroleum ether: EtOAc=10: 1). Upon completion, the reaction was quenched with water (5 mL) and extracted with ethyl acetate (3×5 mL). The combined organic layers were washed with brine (5 mL), dried with anhydrous Na₂SO₄, filtered and concentrated in vacuo. The resulting residue was purified by silica gel chromatography (Petroleum ether: EtOAc=100: 1, then 10: 1) to give compound SI-24 (450 mg, 29%) as a white solid.

### Step 2.

SnCl₂.2H₂O (929.6 mg, 4.12 mmol, 3.2 eq) was added to a solution of intermediate SI-24 (450.0 mg, 1.30 mmol, 1.0 eq) and DMF (5.1 μL, 65 μmol, 0.05 eq) in EtOH (5.0 mL). The mixture was stirred at 78° C. for 4 h. Upon completion, the reaction was quenched by adjusting the pH to pH 9 with saturated aqueous NaHCO₃ (10 mL) and the resulting mixture was extracted with ethyl acetate (3×5 mL). The combined organic layers were washed with brine (5 mL), dried with anhydrous Na₂SO₄, filtered and concentrated in vacuo to afford crude SI-25 (200.0 mg) as a yellow oil, which was used in the next step without further purification.

### Step 3.

DMAP (50.2 mg, 0.41 mmol, 1.0 eq) was added to a mixture of intermediate SI-27 (130.0 mg, 0.41 mmol, 1.0 eq), tert-butoxycarbonyl tert-butyl carbonate (94.4 μL, 0.41 mmol, 1.0 eq), and NEt₃ (170.9 μL, 1.23 mmol, 3.0 eq) in DCM (3 mL) at 25° C. The mixture was stirred at 25° C. for 2 h. Upon completion, the reaction was concentrated in vacuo and the residue was re-dissolved in CH₃CN (3 mL). The target product was purified by prep. HPLC (basic conditions) to afford SI-28 as a yellow solid.

### Step 4.

2-chloroacetyl chloride (15.3 μL, 0.19 mmol, 2.0 eq) was added to a solution of SI-28 (40.0 mg, 96 μmol, 1.0 eq) and NEt₃ (40.0 μL, 0.29 mmol, 3.0 eq) in DCM (1 mL) at 0° C. and the mixture was stirred at 25° C. for 1 h. Upon completion, the reaction was quenched with water (1 mL) and extracted with ethyl acetate (3×2 mL). The combined organic layers were washed with brine (2 mL), dried over anhydrous Na₂SO₄, filtered and concentrated in vacuo to afford SI-29 (40.0 mg) as yellow oil, which was used in the next step without further purification.

### Step 5.

TFA (200 μL, 2.70 mmol, 33.3 eq) was added to a solution of intermediate SI-29 (40.0 mg, 81 μmol, 1.0 eq) in DCM (2 mL) and the mixture was stirred at 25° C. for 1 h. Upon completion, the reaction was diluted with CH₃CN (3 mL) and purified by prep. HPLC (FA conditions) to afford the title compound (20.0 mg, 63%) as a yellow solid. ¹H NMR (DMSO-d₆, 400 MHz) δ 10.95 (s, 1H), 8.29 (m, 1H), 8.16 (m, 1H), 7.67 (s, 1H), 7.26-7.20 (m, 2H), 7.08-7.01 (m, 3H), 4.30 (s, 2H). HRMS electrospray (m/z): [M+H]⁺ calcd for C₁₅H1₃C1F₃N₂O₃S: 393.0282, found: 393.0281.

### Example S-15: Synthesis of N-(1H-benzo[d]imidazol-5-yl)-N-benzyl-2-chloroacetamide (BPK-15)

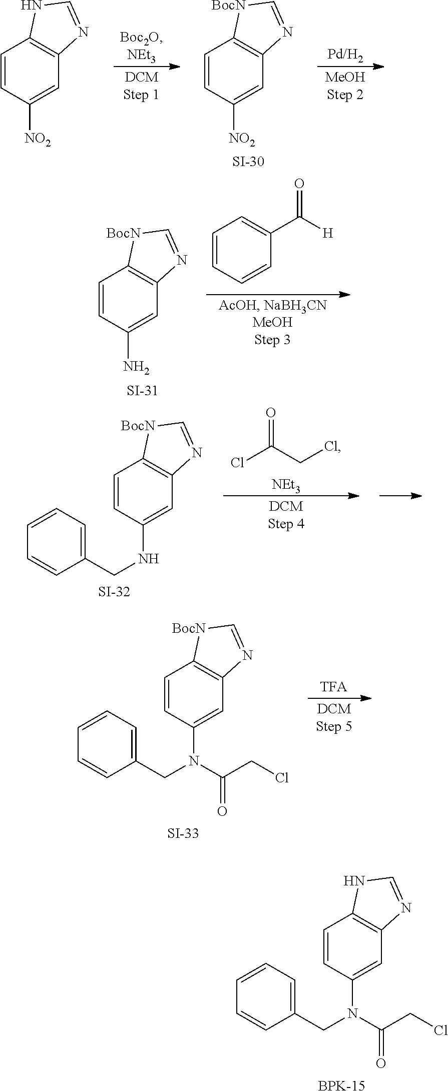

### Step 1.

Boc₂O (2.82 mL, 12.7 mmol, 2.0 eq) was added to a mixture of 6-nitro-1H-benzimidazole (1.00 g, 6.13 mmol, 1.0 eq) and NEt₃ (1.70 mL, 12.3 mmol, 2.0 eq) in DCM (10.0 mL). The mixture was stirred at 25° C. for 2 h and the reaction progress was monitored by TLC (DCM: MeOH=50: 1) and LCMS. Upon completion, the reaction mixture was concentrated in vacuo and purified by silica gel chromatography (Petroleum ether: EtOAc=50: 1, then 10: 1) to afford compound SI-30 (1.60 g, 99%) as a white solid.

### Step 2.

Under an atmosphere of nitrogen, Pd/C (200.0 mg, 10%) was added to a solution of intermediate SI-30 (1.60 g, 6.08 mmol, 1.0 eq) in MeOH (50 mL). The mixture was degassed under vacuum and purged with H₂ several times. The mixture was stirred under H₂ (50 psi) at 25° C. for 16 h. Upon completion, the reaction mixture was filtered and concentrated to give SI-31 (1.40 g) as colorless oil which was used in step 3 without further purification.

### Step 3.

Benzaldehyde (191 μL, 1.89 mmol, 1.1 eq) was added to a solution of compound SI-31 (400.0 mg, 1.71 mmol, 1.0 eq) in anhydrous MeOH (2 mL) and the reaction was stirred at 25° C. for 2 h. Subsequently, NaBH₃CN (215.5 mg, 3.43 mmol, 2.0 eq) was added at 0° C. and the mixture was stirred at 25° C. for an additional 14 h. Upon completion, the reaction was quenched by the addition of saturated aqueous NaHCO₃ (10 mL) and extracted with ethyl acetate (3×10 mL). The combined organic layers were washed with brine (5 mL), dried over anhydrous Na₂SO₄, filtered and concentrated in vacuo. The solution was then purified by prep. HPLC (basic conditions) to afford intermediate SI-32 (300.0 mg, 54%) as colorless oil.

### Step 4.

2-chloroacetyl chloride (74 μL, 0.93 mmol, 2.0 eq) was added dropwise to a solution of compound SI-32 (150.0 mg, 0.46 mmol, 1.0 eq) and NEt₃ (257 μL, 1.86 mmol, 4.0 eq) in anhydrous DCM (2 mL) at 0° C. and the mixture was stirred at 25° C. for 2 h. Upon completion, the reaction was quenched by the addition of saturated aqueous NaHCO₃ (2 mL) and then extracted with DCM (5 mL). The organic layer was dried over anhydrous Na₂SO₄, filtered and concentrated in vacuo to afford compound SI-33 (180.0 mg) as yellow oil, which was used in the next step without further purification.

### Step 5.

TFA (800 μL, 10.8 mmol, 24 eq) was added dropwise to a solution of compound SI-33 (180.0 mg, 0.45 mmol, 1.0 eq) in DCM (4 mL) and the mixture was stirred at 25° C. for 16 h. Upon completion, the reaction was concentrated in vacuo and the residue was re-dissolved in CH₃CN (2 mL). The target product was purified by prep. HPLC (basic conditions) to afford the title compound (25.0 mg, 19%) as a white solid. ¹H NMR (CDCl₃, 400 MHz) δ 8.12 (s, 1H), 7.62 (d, J=8.5 Hz, 1H), 7.34 (s, 1H), 7.25-7.16 (m, 5H), 6.94 (dd, J=8.5, 2.0 Hz, 1H), 4.96 (s, 2H), 3.89 (s, 2H). HRMS electrospray (m/z): [M+H]⁺ calcd for C₁₆H1₅C1N₃O: 300.0898, found: 300.0896.

### Example S-16: Synthesis of N-benzyl-2-chloro-N-(4-oxo-3,4-dihydroquinazolin-6-yl)acetamide (BPK-16)

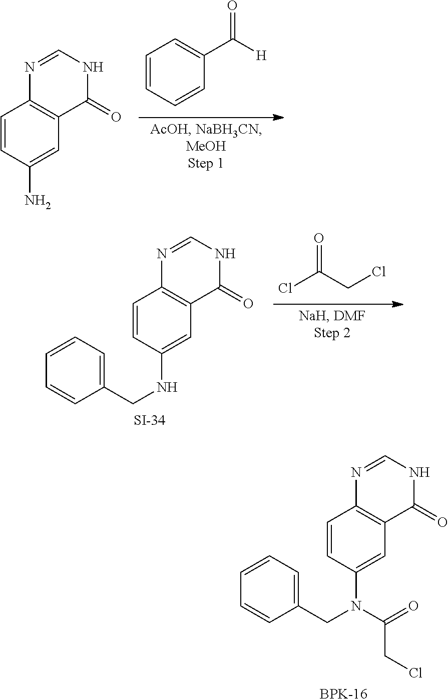

### Step 1.

NaBH₃CN (117.0 mg, 1.86 mmol, 2.0 eq) was added to a solution of AcOH (53.3 μL, 0.93 mmol, 1.0 eq), benzaldehyde (108.7 mg, 1.02 mmol, 1.1 eq), and 6-aminoquinazolin-4(3H)-one (150.0 mg, 0.93 mmol, 1.0 eq) in anhydrous MeOH (1 mL) and the resulting mixture was stirred at 15° C. for 16 h. Upon completion, the reaction was quenched with saturated aqueous NaHCO₃ (10 mL) and extracted with EtOAc (3×10 mL). The combined organic layers were washed with brine (5 mL), dried over anhydrous Na₂SO₄, filtered and concentrated in vacuo to afford compound SI-34 (200.0 mg) as a white solid, which was used in the next step without additional purification.

### Step 2.

NaH (101.9 mg, 2.55 mmol, 60% in oil, 4.0 eq) was added to a solution of compound SI-34 (160.0 mg, 0.64 mmol, 1.0 eq) in anhydrous DMF (1 mL) at 0° C. and the reaction was stirred at 0° C. for 30 min. 2-chloroacetyl chloride (101 μL, 1.27 mmol, 2.0 eq) was then added dropwise and the mixture was stirred at 0° C. for another 30 min. Upon completion, the reaction was concentrated in vacuo, the remaining residue was re-dissolved in CH₃CN (2 mL) and water (1 mL) and purified by prep. HPLC (HCl conditions) to afford compound the title compound (10.0 mg, 5%) as a yellow solid. ¹H NMR (DMSO-d₆, 400 MHz) δ 8.50-8.37 (m, 1H), 7.96-7.91 (m, 1H), 7.78-7.68 (m, 2H), 7.33-7.13 (m, 5H), 5.00-4.87 (m, 2H), 4.20-4.03 (m, 2H). HRMS electrospray (m z): [M+H]+ calcd for C₁₇H1₅C1N₃O₂: 328.0847, found: 328.0849.

### Example S-17: Synthesis of N-(3-(morpholine-4-carbonyl)benzyl)-N-phenylacrylamide (BPK-17)

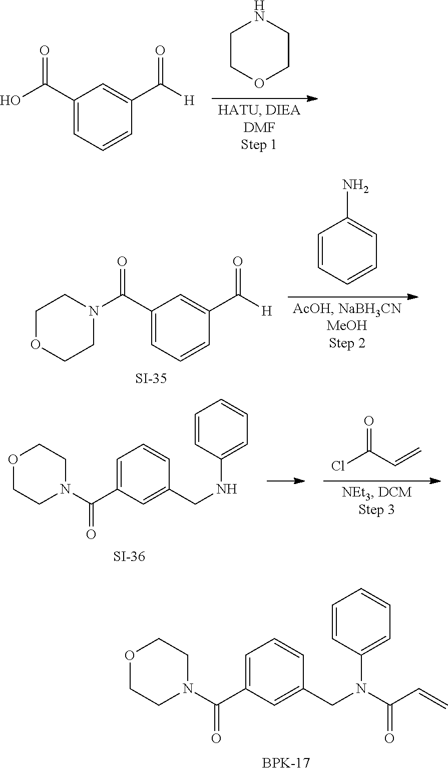

### Step 1.

A solution of DIEA (5.8 mL, 33.3 mmol, 5.0 eq), HATU (3.80 g, 10 mmol, 1.5 eq) and 3-formylbenzoic acid (1.0 g, 6.7 mmol, 1.0 eq) in DMF (10 mL) was stirred at 25° C. for 30 min. Morpholine (586 μL, 6.7 mmol, 1.0 eq) was then added and the reaction mixture was stirred for another 1.5 h. Upon completion, the reaction was quenched with water (20 mL) and extracted with DCM (3×10 mL). The combined organic layers were washed with brine (3×10 mL), dried over Na₂SO₄, filtered and concentrated under reduced pressure to give product compound SI-35 (1.20 g) as yellow oil.

### Step 2.

Compound SI-36 was synthesized following the procedure detailed for compound SI-34. In particular, AcOH (0.98 mL, 17.1 mmol, 5.5 eq) was added to a solution of compound SI-35 (750 mg, 3.1 mmol, 1.0 eq) and aniline (312.3 μL, 3.42 mmol, 1.1 eq) in DCM (5 mL) at 25° C. After stirring for 30 min, NaBH₃CN (430 mg, 6.8 mmol, 2.2 eq) was added to the mixture at 0° C. The mixture was then stirred at 25° C. for another 1.5 h. Upon completion, the reaction was quenched with water (10 mL) and extracted with DCM (3×5 mL). The combined organic layers were washed with brine (3×5 mL), dried over Na₂SO₄, filtered and concentrated in vacuo to afford compound SI-36 (880.0 mg) as yellow oil, which was used into the next step without further purification.

### Step 3.

Acryloyl chloride (181 μL, 2.22 mmol, 2.0 eq) was added dropwise to a solution of compound SI-36 (330.0 mg, 1.11 mmol, 1.0 eq) and NEt₃ (769 μL, 5.55 mmol, 5.0 eq) in DCM (1 mL) at 0° C. and the resulting mixture was stirred at 25° C. for 2 h. Upon completion, the reaction was quenched with water (3 mL) and extracted with DCM (3×1 mL). The combined organic layers were washed with brine (3×2 mL), dried over Na₂SO₄, filtered and concentrated under reduced pressure. The resulting residue was re-dissolved in CH₃CN and water, and purified by prep. HPLC (TFA conditions) to give the title compound (92.0 mg, 20%) as yellow oil. ¹H NMR (DMSO-d₆, 400 MHz) δ 7.38-7.32 (m, 3H), 7.29 (t, J=8.1 Hz, 2H), 7.23 (d, J=7.4 Hz, 1H), 7.12-7.06 (m, 3H), 6.23 (dd, J=16.8, 2.2 Hz, 1H), 6.05-5.92 (m, 1H), 5.61 (dd, J=10.1, 2.2 Hz, 1H), 4.97 (s, 2H), 3.67-3.38 (m, 6H), 3.13 (s, 2H). HRMS electrospray (m/z): [M+H]⁺ calcd for C₂₁H2₃N₂O₃: 351.1703, found: 351.1703.

### Example S-18: Synthesis of N-benzyl-4-((2-chloro-N-phenylacetamido)methyl)benzamide (BPK-18)

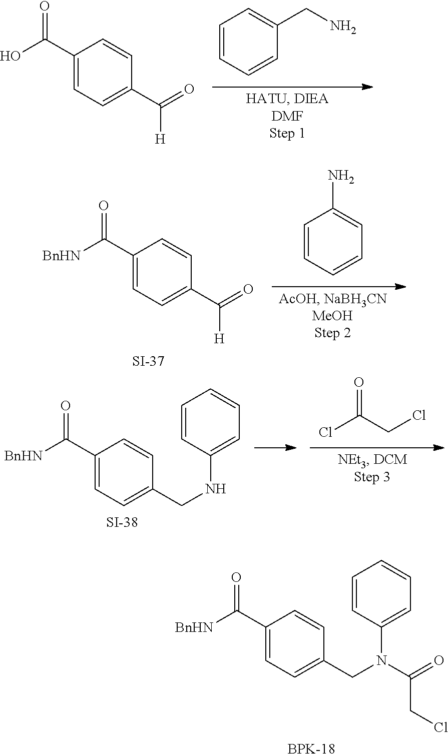

### Step 1.

HATU (3.80 g, 10.0 mmol, 1.5 eq) and benzylamine (728 μL, 6.7 mmol, 1.0 eq) were added to a solution of DIEA (5.81 mL, 33.3 mmol, 5.0 eq) in DMF (10 mL) and the mixture was stirred at 25° C. for 30 min. 4-formylbenzoic acid (1.00 g, 6.7 mmol, 1.0 eq) was then added to the reaction and the resulting mixture was stirred for another 1.5 h. Upon completion, the reaction was quenched with water (20 mL) and extracted with DCM (3×10 mL). The combined organic layers were washed with brine (3×10 mL), dried over Na₂SO₄ filtered and concentrated under reduced pressure to afford compound SI-37 (800 mg) as yellow oil, which was used in the next step without additional purification.

### Step 2.

AcOH (895 μL, 15.7 mmol, 5.1 eq) and aniline (286 μL, 3.1 mmol, 1.0 eq) were added to a solution of compound SI-37 (750 mg, 3.1 mmol, 1.0 eq) in DCM (5 mL) at 25° C. After stirring for 0.5 h, NaBH₃CN (393 mg, 6.2 mmol, 2.0 eq) was added to the mixture at 0° C. The mixture was then stirred at 25° C. for another 1.5 h. Upon completion, the reaction was quenched with water (10 mL) and extracted with DCM (3×5 mL). The combined organic layers were washed with brine (3×5 mL), dried over Na₂SO₄, filtered and concentrated in vacuo to afford compound SI-38 (600 mg) as yellow oil, which was used in the next step without further purification.

### Step 3.

2-chloroacetyl chloride (105 μL, 1.33 mmol, 2.0 eq) was added dropwise to a solution of compound SI-38 (210 mg, 0.66 mmol, 1.0 eq) and NEt₃ (460 μL, 3.32 mmol, 5.0 eq) in DCM (1.0 mL) at 0° C. and the resulting mixture was stirred at 25° C. for 2 h. Upon completion, the reaction was quenched with water (3 mL) and extracted with DCM (3×1 mL). The combined organic layers were washed with brine (3×2 mL), dried over Na₂SO₄, filtered and concentrated under reduced pressure. The resulting residue was re-dissolved in CH₃CN and water, and purified by prep. HPLC (HCl conditions) to give compound the title compound (27.0 mg, 10%) as yellow oil. ¹H NMR (DMSO-d₆, 400 MHz) δ 7.77 (d, J=8.3 Hz, 2H), 7.43-7.14 (m, 14H), 4.92 (s, 2H), 4.43 (s, 2H), 4.04 (s, 2H). HRMS electrospray (m/z): [M+H]⁺ calcd for C₂₃H2₂C1N₂O₂: 393.1364, found: 393.1365.

### Example S-19: Synthesis of 2-chloro-N-(3-fluorobenzyl)-N-(4-phenoxy-3-(trifluoromethyl)phenyl)acetamide (BPK-19)

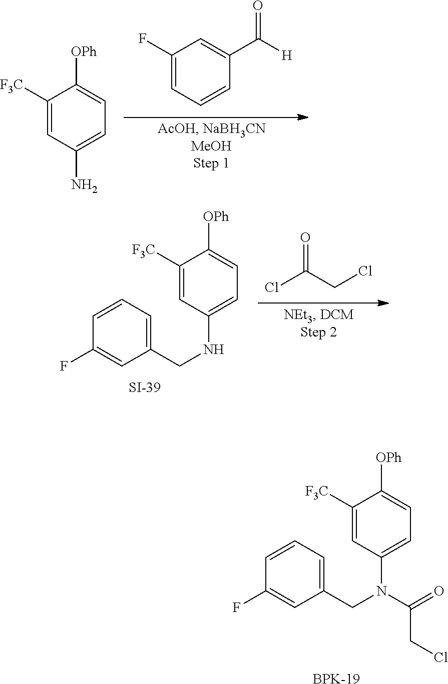

### Step 1.

A mixture of 4-phenoxy-3-(trifluoromethyl)aniline (200.0 mg, 0.79 mmol, 1.0 eq), AcOH (54.2 μL, 0.95 mmol, 1.2 eq) and 3-fluorobenzaldehyde (91.4 μL, 0.86 mmol, 1.1 eq) in anhydrous MeOH (3 mL) was stirred at 63° C. for 16 h. NaBH₃CN (148.9 mg, 2.37 mmol, 3.0 eq) was then added at 0° C. and the mixture was stirred at 25° C. for additional 4 h with the reaction progress monitored by TLC (Petroleum ether: EtOAc=10: 1). Upon completion, the mixture was concentrated in vacuo, the resulting residue was re-dissolved in saturated aqueous NaHCO₃ (2 mL) and extracted with DCM (3×3 mL). The combined organic layers were dried over Na₂SO₄, filtered and concentrated in vacuo to give compound SI-39 (240.0 mg) as yellow oil, which was used in the next step without further purification.

### Step 2.

2-chloroacetyl chloride (61.6 μL, 0.78 mmol, 2.0 eq) was added dropwise to a solution of compound SI-39 (140.0 mg, 0.39 mmol, 1.0 eq) and NEt₃ (269 μL, 1.94 mmol, 5.0 eq) in anhydrous DCM (1.5 mL) at 0° C. and the resulting mixture was stirred at 25° C. for 2 h. Upon completion, the mixture was concentrated in vacuo and the remaining residue was re-dissolved in aqueous NaHCO₃ (2 mL) and extracted with DCM (3×3 mL). The combined organic layers were dried over Na₂SO₄, filtered and concentrated in vacuo. Purification by prep. HPLC (HCl conditions) afforded compound the title compound (30.0 mg, 18%) as colorless oil. ¹H NMR (CDCl₃, 400 MHz) δ 7.44 (t, J=7.9 Hz, 2H), 7.40 (d, J=2.2 Hz, 1H), 7.33-7.23 (m, 2H), 7.12-7.07 (m, 3H), 7.04-6.95 (m, 3H), 6.86 (d, J=8.8 Hz, 1H), 4.89 (s, 2H), 3.89 (s, 2H). HRMS electrospray (m/z): [M+H]+ calcd for C₂₂H₁₇ClF₄NO₂: 438.0878, found: 438.0877.

### General Procedure for the synthesis of compounds Examples S-20-S-24

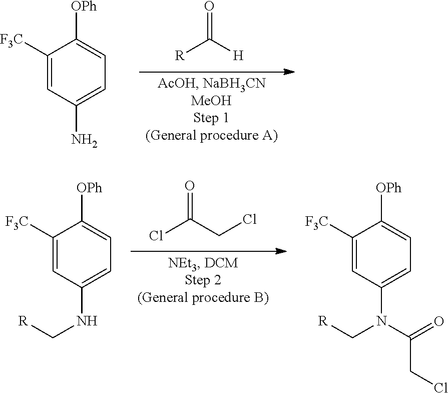

### General Procedure A.

A mixture of aldehyde (1.0 eq), AcOH (1.2 eq) and 4-phenoxy-3-(trifluoromethyl)aniline (1.0 eq) in anhydrous MeOH was stirred at 25° C. for 1 h. NaBH₃CN (3.0 eq) was added at 0° C. and the reaction mixture was stirred at 25° C. for 2h. Upon completion, the mixture was concentrated in vacuo, the remaining residue was re-dissolved in saturated aqueous NaHCO₃ (2 mL) and extracted with DCM (3×3 mL). The combined organic layers were dried over Na₂SO₄, filtered and concentrated in vacuo to afford the corresponding intermediate, which was used in the next step without further purification.

### General Procedure B.

2-chloroacetylchloride (2.0 eq) was added dropwise to a solution of intermediate from procedure A (1.0 eq) and NEt₃ (5.0 eq) in anhydrous DCM at 0° C. and the mixture was stirred at 25° C. for 2 h. Upon completion, the reaction mixture was concentrated in vacuo, the remaining residue was re-dissolved in saturated aqueous NaHCO₃ and extracted with DCM. The combined organic layers were then dried over Na₂SO₄, filtered, concentrated in vacuo and purified by prep. HPLC to give the desired compound.

### Example S-20: Synthesis of 2-chloro-N-(2,3-dichlorobenzyl)-N-(4-phenoxy-3-(trifluoromethyl)phenyl)acetamide (BPK-20)

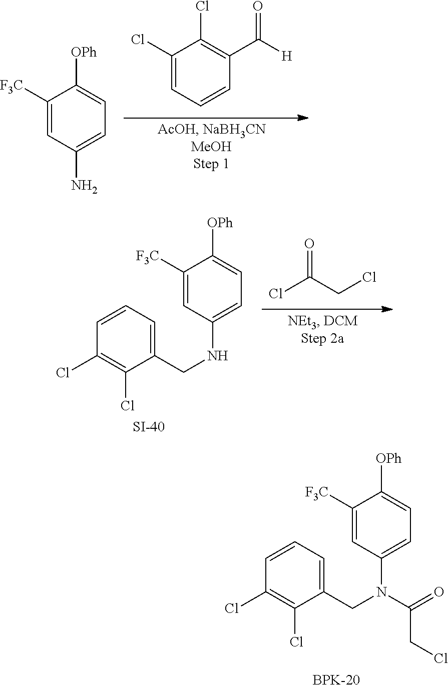

### Step 1.

Compound SI-40 was synthesized according to general procedure A from 2,3-dichlorobenzaldehyde (206.5 g, 1.18 mol), AcOH (81 mL, 1.42 mol), 4-phenoxy-3-(trifluoromethyl)aniline (300.0 g, 1.18 mol, 1.0 eq), and NaBH₃CN (222.5 g, 3.54 mol). Aqueous work up afforded SI-40 (450.0 g) as yellow oil, which was used in the next step without further purification.

### Step 2a.

Compound BPK-20 was synthesized according to general procedure B from SI-40 (125.0 mg, 0.30 mmol), Et₃N (210 μL, 1.52 mmol), and 2-chloroacetyl chloride (48.2 μL, 0.61 mmol). Aqueous extraction, followed by purification by prep. HPLC (HCl conditions) afforded the title compound (63.1 mg, 42%) as light yellow oil. ¹H NMR (CDCl₃, 400 MHz) δ 7.42-7.37 (m, 4H), 7.30 (d, J=7.8, 1H), 7.25-7.16 (m, 2H), 7.13 (dd, J=8.8, 2.7 Hz, 1H), 7.07-7.02 (m, 2H), 6.83 (d, J=8.8 Hz, 1H), 5.08 (s, 2H), 3.89 (s, 2H). HRMS electrospray (m/z): [M+H]+ calcd for C₂₂H1₆C1₃F3NO₂: 488.0193, found: 488.0192.

### Example S-21: Synthesis of N-(2,3-dichlorobenzyl)-N-(4-phenoxy-3-(trifluoromethyl)phenyl)acrylamide (BPK-21)

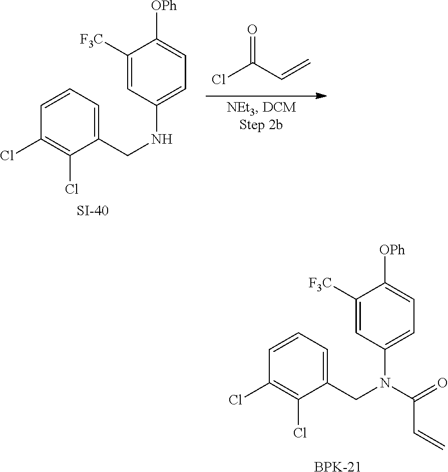

### Step 2b.

NEt₃ (210 μL, 1.52 mmol, 5.0 eq) and acryloyl chloride (49.5 μL, 0.61 mmol, 2.0 eq) were added to a solution of compound SI-40 (125.0 mg, 0.30 mmol, 1.0 eq) in anhydrous DCM (1.5 mL) at 0° C. and the mixture was stirred at 25° C. for 2 h. Upon completion, the mixture was concentrated in vacuo, the remaining residue was re-dissolved in saturated aqueous NaHCO₃ (2 mL) and extracted with DCM (3×3 mL). The combined organic layers were dried over Na₂SO₄, filtered, concentrated in vacuo and purified by prep. HPLC (basic conditions) to give the title compound (82.0 mg, 57%) as light yellow oil. ¹H NMR (CDCl₃, 400 MHz) δ 7.42-7.36 (m, 4H), 7.30 (dd, J=7.8, 1.6 Hz, 1H), 7.23-7.16 (m, 2H), 7.11-7.07 (m, 1H), 7.06-7.02 (m, 2H), 6.83 (d, J=8.8 Hz, 1H), 6.48 (dd, J=16.7, 1.8 Hz, 1H), 6.09 (dd, J=16.7, 10.3 Hz, 1H), 5.67 (dd, J=10.3, 1.8 Hz, 1H), 5.13 (s, 2H). HRMS electrospray (m z): [M+H]⁺ calcd for C₂₃H₁₇C1₂F3NO₂: 466.0583, found: 466.0582.

### Example S-22: Synthesis of 2-chloro-N-(3-morpholinobenzyl)-N-(4-phenoxy-3-(trifluoromethyl)phenyl)acetamide (BPK-22)

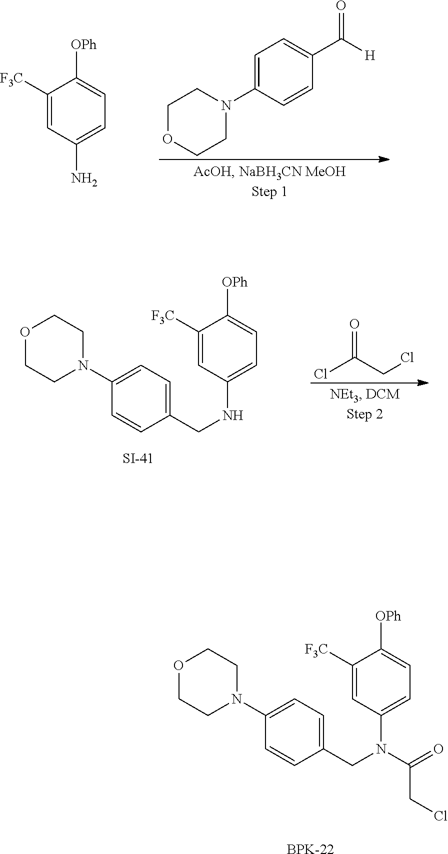

### Step 1

Compound SI-41 was synthesized according to general procedure A from 3-morpholinobenzaldehyde (225.7 mg, 1.18 mmol), AcOH (81.0 μL, 1.42 mmol), 4-phenoxy-3-(trifluoromethyl)aniline (300.0 mg, 1.18 mmol), and NaBH₃CN (222.5 mg, 3.54 mmol). Aqueous work up afforded Compound SI-41 (480.0 mg) as yellow oil, which was used in the next step without further purification.

### Step 2.

Compound BPK-22 was synthesized according to general procedure K from Compound SI-41 (125.0 mg, 0.29 mmol), Et₃N (202 μL, 1.46 mmol), and 2-chloroacetyl chloride (46.4 μL, 0.58 mmol). Aqueous work up, followed by purification by prep. HPLC (HCl conditions) afforded the title compound (104.9 mg, 65%) as light yellow oil. ¹H NMR (CDCl₃, 400 MHz) δ 7.41 (t, J=7.8 Hz, 2H), 7.34 (d, J=2.6 Hz, 1H), 7.23 (t, J=7.5 Hz, 1H), 7.18 (t, J=7.8 Hz, 1H), 7.08-7.03 (m, 3H), 6.84-6.79 (m, 2H), 6.77 (s, 1H), 6.64 (d, J=7.5 Hz, 1H), 4.82 (s, 2H), 3.87-3.80 (m, 6H), 3.13-3.07 (m, 4H). HRMS electrospray (m/z): [M+H]⁺ calcd for C₂₆H2₅C1F₃N₂O₃: 505.1500, found: 505.1500.

### Example S-23: Synthesis of N-(3-(1H-1,2,4-triazol-1-yl)benzyl)-2-chloro-N-(4-phenoxy-3-(trifluoromethyl)phenyl)acetamide (BPK-23)

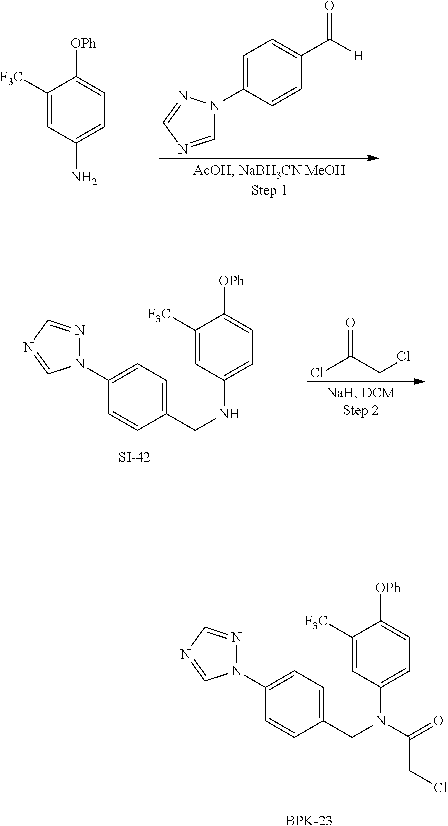

### Step 1.

Compound SI-42 was synthesized according to general procedure A from 4-(1H-1,2,4-triazol-1-yl)benzaldehyde (171.0 mg, 0.99 mmol), AcOH (67.8 μL, 1.18 mmol), 4-phenoxy-3-(trifluoromethyl)aniline (250.0 mg, 0.99 mmol), and NaBH₃CN (186.1 mg, 2.96 mmol). Aqueous work up afforded compound SI-42 (240.0 mg) as yellow oil, which was used in the next step without further purification.

### Step 2.

2-chloroacetyl chloride (15.5 μL, 0.19 mmol, 1.0 eq) was added to a solution of compound SI-42 (80.0 mg, 0.19 mmol, 1.0 eq) and NaH (9.4 mg, 0.39 mmol, 2.0 eq) at 0° C. and the reaction was stirred at 25° C. for 2h. Upon completion, the reaction mixture was concentrated in vacuo. The resulting residue was diluted with CH₃CN (2 mL) and water (1 mL) and purified by prep. HPLC (HCl conditions) to afford the title compound (10.0 mg, 10%) as yellow oil. ¹H NMR (CDCl₃, 400 MHz) δ 8.78 (s, 1H), 8.02 (s, 1H), 7.54-7.47 (m, 2H), 7.32 (t, J=7.4 Hz, 1H), 7.30-7.21 (m, 3H), 7.15-7.05 (m, 3H), 6.99 (d, J=7.9 Hz, 1H), 6.92 (d, J=7.9 Hz, 2H), 6.69 (d, J=7.9 Hz, 1H), 4.81 (s, 2H), 3.75 (s, 2H). HRMS electrospray (m/z): [M+H]⁺ calcd for C₂₄H1₉C1F₃N₄O₂: 487.1143, found: 487.1143.

### Example S-24: Synthesis of 2-chloro-N-((3,4-dihydro-2H-benzo[b][1,4]dioxepin-7-yl)methyl)-N-(4-phenoxy-3-(trifluoromethyl)phenyl)acetamide (BPK-24)

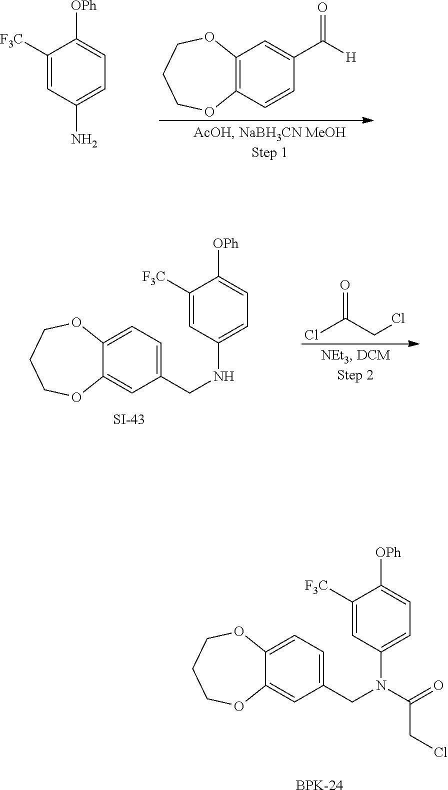

### Step 1.

Compound SI-43 was synthesized according to general procedure A from 3,4-dihydro-2H-benzo[b][1,4]dioxepine-7-carbaldehyde (175.9 mg, 0.99 mmol), AcOH (67.8 μL, 1.18 mmol), 4-phenoxy-3-(trifluoromethyl)aniline (250.0 mg, 0.99 mmol), and NaBH₃CN (186.1 mg, 2.96 mmol). Aqueous work up afforded compound SI-43 (400.0 mg) as yellow oil, which was used in the next step without further purification.

### Step 2.

Compound BPK-24 was synthesized according to general procedure B from compound SI-43 (200.0 mg, 0.48 mmol, 1.0 eq), Et₃N (333.7 μL, 2.41 mmol, 5.0 eq), and 2-chloroacetyl chloride (76.6 μL, 0.96 mmol, 2.0 eq). Aqueous work up, followed by prep. HPLC (HCl conditions) afforded the title compound (105.0 mg, 44%) as light yellow oil. ¹H NMR (CDCl₃, 400 MHz) δ 7.38 (t, J=6.9 Hz, 2H), 7.27 (s, 1H), 7.19 (t, J=7.4 Hz, 1H), 7.03 (d, J=7.9 Hz, 3H), 6.89-6.67 (m, 4H), 4.73 (s, 2H), 4.19-4.08 (m, 4H), 3.80 (s, 2H), 2.13 (s, 2H). HRMS electrospray (m/z): [M+H]+ calcd for C₂₅H2₂C1F₃NO₄: 492.1184, found: 492.1182.

### Example S-25: Synthesis of 5-(N-((6-chloropyridin-2-yl)methyl)acrylamido)-N-phenylpicolinamide (BPK-25)

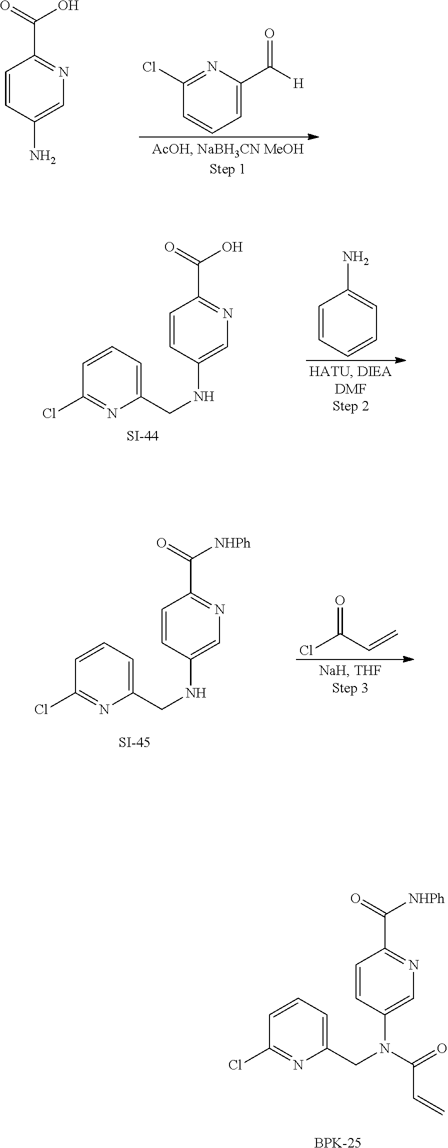

### Step 1.

NaBH₃CN (408.4 mg, 6.50 mmol, 2.0 eq) was added to a solution of AcOH (185.85 μL, 3.25 mmol, 1.0 eq), 5-aminopicolinic acid (448.9 mg, 3.25 mmol, 1.0 eq) and 6-chloropyridine-2-carbaldehyde (460.0 mg, 3.25 mmol, 1.0 eq) in anhydrous MeOH (5.0 mL). The reaction was stirred at 25° C. for 16 h. Upon completion, the reaction was concentrated in vacuo to afford compound SI-44 (1.00 g) as a yellow solid.

### Step 2.

DIEA (3.97 mL, 22.8 mmol, 3.0 eq) was added to a solution of aniline (1.39 mL, 15.2 mmol, 2.0 eq), HATU (3.46 g, 9.10 mmol, 1.2 eq), and compound SI-44 (2.00 g, 7.58 mmol, 1.0 eq) in DMF (15 mL) and the resulting mixture was stirred at 25° C. for 16 h. Upon completion, the reaction was quenched with water (20 mL) and extracted with ethyl acetate (3×10 mL). The combined organic layers were washed with brine (10 mL), dried with anhydrous Na₂SO₄, filtered and concentrated in vacuo. The resulting residue was purified by silica gel chromatography (Petroleum ether: EtOAc=10: 1, then 0: 1) to afford compound SI-45 (1.00 g) as yellow oil.

### Step 3.

NaH (63.8 mg, 1.59 mmol, 60% in oil, 3.0 eq) was added to a solution of SI-45 (300.0 mg, 0.53 mmol, 1.0 eq, 60% pure) in anhydrous THF (2 mL) at 0° C. and the reaction was stirred at 0° C. for 2 h. Acryloyl chloride (86.6 μL, 1.06 mmol, 2.0 eq) was added at 0° C. and the reaction mixture was stirred at 25° C. for 14 h. Upon completion, the mixture was concentrated in vacuo, the resulting residue was re-dissolved in CH₃CN (3 mL) and saturated aqueous NaHCO₃ (1 mL) and purified by prep. HPLC (basic conditions) to afford the title compound (14.0 mg, 7% yield) as yellow oil. ¹H NMR (DMSO-d₆, 400 MHz) δ 10.63 (s, 1H), 8.69 (d, J=2.4 Hz, 1H), 8.20 (d, J=8.4 Hz, 1H), 8.06 (dd, J=8.4, 2.5 Hz, 1H), 7.90-7.80 (m, 3H), 7.44-7.32 (m, 4H), 7.12 (t, J=7.4 Hz, 1H), 6.30-6.24 (m, 2H), 5.76-5.71 (m, 1H), 5.13 (s, 2H). HRMS electrospray (m/z): [M+H]⁺ calcd for C₂₁H1₈C1N₄O₂: 393.1113, found: 393.1114.

### Example S-26: Synthesis of 2-chloro-N-(3-chloro-2-fluorobenzyl)-N-(6-chloropyridin-3-yl)acetamide (BPK-26)

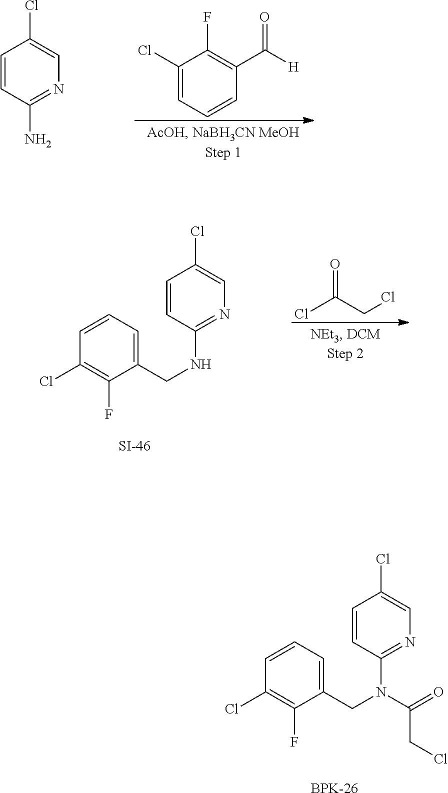

### Step 1.

NaBH₃CN (118.9 mg, 1.89 mmol, 2.0 eq) was added to a solution of AcOH (54.1 μL, 0.95 mmol, 1.0 eq), 5-chloropyridin-2-amine (121.6 mg, 0.95 mmol, 1.0 eq), and 3-chloro-2-fluorobenzaldehyde (150.0 mg, 0.95 mmol, 1.0 eq) in anhydrous MeOH (2 mL) and the reaction was stirred at 25° C. for 2 h. Upon completion, the reaction was quenched with saturated aqueous NaHCO₃ (10 mL) and extracted with ethyl acetate (3×10 mL). The combined organic layers were washed with brine (5 mL), dried over anhydrous Na₂SO₄, filtered and concentrated in vacuo to afford compound SI-46 (250.0 mg) as yellow solid, which was used in the next step without additional purification.

### Step 2.

2-chloroacetyl chloride (82.1 μL, 1.03 mmol, 2.0 eq) was added to a solution of NEt₃ (358 μL, 2.58 mmol, 5.0 eq) and compound SI-46 (140.0 mg, 0.52 mmol, 1.0 eq) in anhydrous DCM (2 mL) at 0° C. and the reaction was stirred at 25° C. for 2 h. Upon completion, the reaction mixture was concentrated in vacuo. The resulting residue was re-dissolved in CH₃CN (2 mL) and water (1 mL) and purified by prep. HPLC (HCl condition) to afford compound the title compound (28.0 mg, 14%) as colorless oil. ¹H NMR (DMSO-d₆, 400 MHz) δ 8.38 (d, J=2.7 Hz, 1H), 7.87 (d, J=8.6 Hz, 1H), 7.59 (d, J=8.5 Hz, 1H), 7.54-7.45 (m, 1H), 7.35-7.28 (m, 1H), 7.20-7.15 (m, 1H), 4.98 (s, 2H), 4.17 (s, 2H). HRMS electrospray (m/z): [M+H]+ calcd for C₁₄H₁₁Cl₃FN₂O: 346.9915, found: 346.9916.

### Example S-27: Synthesis of N-(4-(benzyloxy)-3-methoxybenzyl)-N-(5-(tert-butyl)-2-methoxyphenyl)-2-chloroacetamide (BPK-27)

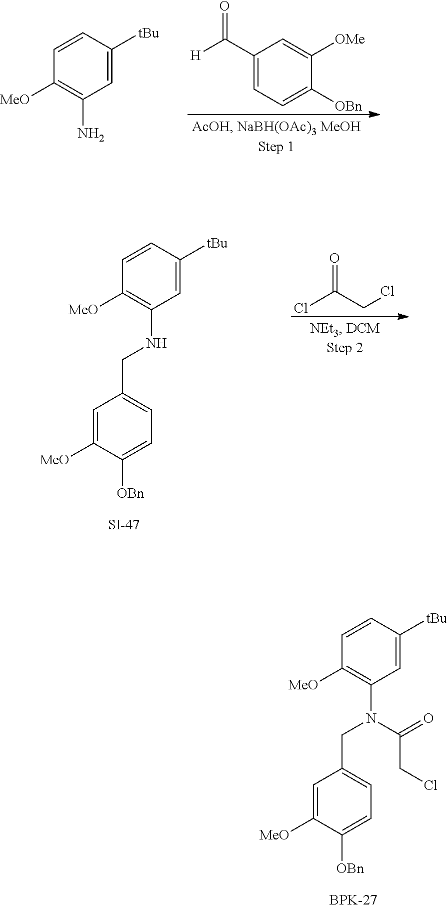

### Step 1.

AcOH (15.0 μL, 0.27 mmol, 1.2 eq) and NaBH(OAc)₃ (52.8 mg, 0.25 mmol, 1.1 eq) were added to a solution of 5-(tert-butyl)-2-methoxyaniline (44.3 mg, 0.25 mmol, 1.1 eq) and 4-(benzyloxy)-3-methoxybenzaldehyde (53.6 mg, 0.22 mmol, 1.0 eq) in dicholoroethane (1.5 mL) and the mixture was stirred at 25° C. for 16 h. Upon completion, the reaction was concentrated under a stream of nitrogen and the resulting residue was re-dissolved in saturated aqueous NaHCO₃ solution (2 mL) and extracted with ethyl acetate (3×2 mL). The combined organic layers were washed with brine (3 mL), dried over anhydrous Mg₂SO₄, filtered and concentrated under a stream of nitrogen. The resulting residue was re-dissolved in DCM and purified by silica gel chromatography (15-25% EtOAc/hexanes) to afford SI-47 (59.7 mg, 67%).

### Step 2.

2-chloroacetyl chloride (35.2 μL 0.44 mmol, 3.0 eq) was added dropwise to a solution of SI-47 (59.7 mg, 0.15 mmol, 1.0 eq) and pyridine (55.5 μL, 0.77 mmol, 5.2 eq) at 0° C. and the resulting mixture was stirred at 25° C. for 16 h. Upon completion, the reaction mixture was concentrated under a stream of nitrogen. The residue was re-dissolved in saturated aqueous NaHCO₃ solution (2 mL) and diethyl ether (2 mL), stirred for 20 min, and further extracted with diethyl ether (2×2 mL). The combined organic layers were dried over anhydrous MgSO₄, filtered and concentrated under a stream of nitrogen. The resulting residue was re-dissolved in DCM and purified by silica gel chromatography (15-35% EtOAc/hexanes) to afford the title compound (42.6 mg, 60%) as light yellow oil. ¹H NMR (CDCl₃, 400 MHz) δ 7.40 (d, J=7.4 Hz, 2H), 7.34 (t, J=7.6 Hz, 2H), 7.30-7.26 (m, 2H), 6.83 (d, J=8.6 Hz, 1H), 6.77 (d, J=1.4 Hz, 1H), 6.73 (d, J=2.4 Hz, 1H), 6.71 (d, J=8.2 Hz, 1H), 6.56-6.53 (m, 1H), 5.25 (d, J=13.9 Hz, 1H), 5.11 (s, 2H), 4.19 (d, J=13.9 Hz, 1H), 3.82 (d, J=5.1 Hz, 2H), 3.80 (s, 3H), 3.70 (s, 3H), 1.14 (s, 9H). HRMS electrospray (m/z): [M+H]+ calcd for C₂₈H3₃C1NO₄: 482.2093, found: 482.2094.

### Synthesis of Intermediate SI-50 as a Common Precursor for Compounds of Examples S-28-S-34

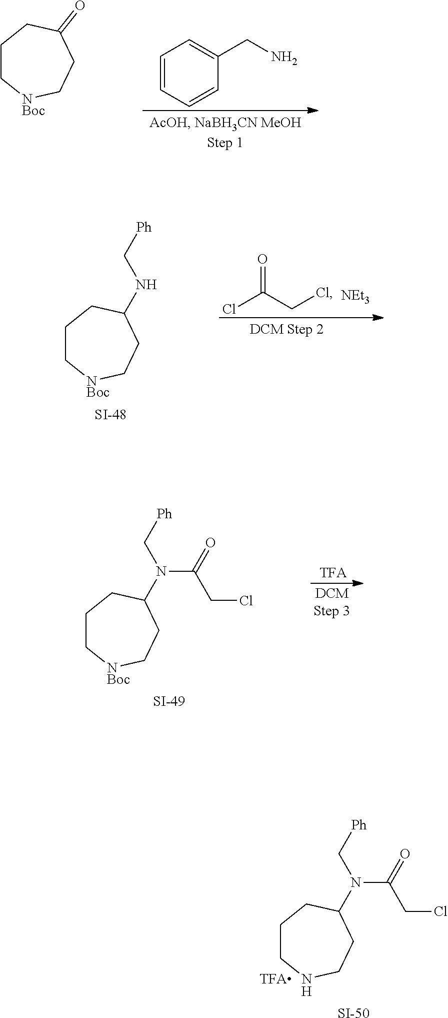

### Step 1.

AcOH (53.6 μL, 0.94 mmol, 2.0 eq) was added to a solution of tert-butyl 4-oxoazepane-1-carboxylate (100.0 mg, 0.47 mmol, 1.0 eq) and BnNH₂ (61.5 μL, 0.56 mmol, 1.2 eq) in MeOH (5 mL) at 25° C. The reaction was stirred for 30 min, after which NaBH₃CN (44.2 mg, 0.70 mmol, 1.5 eq) was added at 0° C. and the mixture was stirred at 25° C. for additional 1.5 h. Upon completion, the reaction was quenched by the addition of water (10 mL) and extracted with DCM (3×5 mL). The combined organic layers were dried over Na₂SO₄ and concentrated to give crude compound SI-48 (120.0 mg) as yellow oil, which was used in step 2 without further purification.

### Step 2.

Under an atmosphere of nitrogen, 2-chloroacetyl chloride (1.55 mL, 19.7 mmol, 1.2 eq) was added dropwise to a solution of compound SI-48 (5.0 g, 16.4 mmol, 1.0 eq) and NEt₃ (5.0 g, 49.3 mmol, 3.0 eq) in anhydrous DCM (2 mL) at 0° C. The resulting mixture was stirred at 15° C. for 2 h. Upon completion, the reaction was quenched by the addition of water (10 mL) at 15° C. and extracted with DCM (3×5 mL). The combined organic layers were dried over Na₂SO₄, filtered and concentrated to give compound SI-49 as yellow oil (4.5 g), which was used in the next step without additional purification.

### Step 3.

TFA (1.17 mL, 15.75 mmol, 5.0 eq) was added to a solution of compound SI-49 (1.20 g, 3.15 mmol, 1.0 eq) in DCM (10 mL) and the mixture was stirred at 25° C. for 1.5 h. Upon completion, the reaction was quenched by the addition of water (20 mL) and extracted with DCM (3×10 mL). The combined organic layers were dried over Na₂SO₄, filtered and concentrated to give compound SI-50 (800.0 mg) as yellow oil, which was used as an intermediate in the synthesis of compounds E94 in the next step without additional purification.

### Example S-28: Synthesis of N-benzyl-2-chloro-N-(1-(2-methylbenzoyl)azepan-4-yl)acetamide (BPK-28)

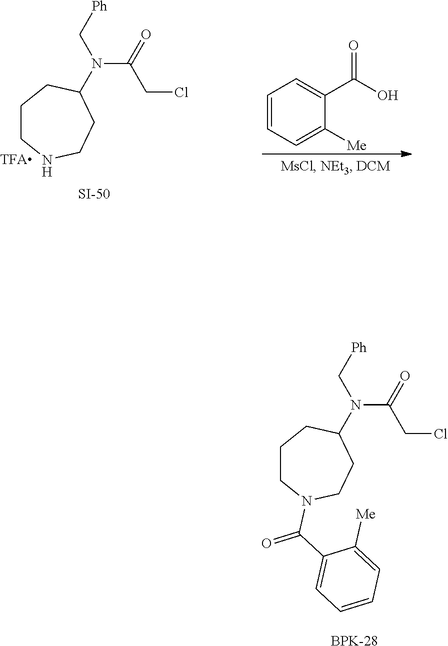

A solution of compound SI-50 (150.0 mg, 0.53 mmol, 1.0 eq), NEt₃ (370 μL, 2.67 mmol, 5.0 eq), and 2-methylbenzoic acid (82 μL, 0.64 mmol, 1.2 eq) in DCM (0.5 mL) was stirred at 0° C. for 30 min. MsCl (82.7 μL, 1.07 mmol, 2.0 eq) was then added and the mixture was stirred at 25° C. for additional 1.5 h. Upon completion, the reaction was quenched with water (5 mL) and extracted with DCM (3×5 mL). The combined organic layers were dried over Na₂SO₄, filtered and concentrated. The residue was purified by prep. HPLC (FA conditions) to give the title compound (58.0 mg, 27%) as a white solid. ¹H NMR (CDCl₃, 400 MHz) δ 7.44-6.97 (m, 9H), 4.77-4.41 (m, 2H), 4.40-3.76 (m, 4H), 3.44-2.94 (m, 3H), 2.34-2.21 (m, 3H), 2.16-1.89 (m, 2H), 1.87-1.48 (m, 4H). HRMS electrospray (m z): [M+H]⁺ calcd for C₂₃H2₈C1N₂O₂: 399.1834, found: 399.1835.

### Example S-29: Synthesis of N-benzyl-2-chloro-N-(1-(4-morpholinobenzoyl)azepan-4-yl)acetamide (BPK-29)

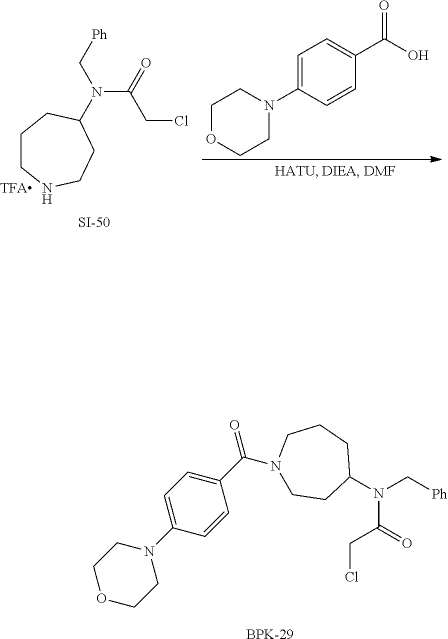

HATU (196.5 mg, 0.52 mmol, 1.2 eq) and DIEA (166.9 mg, 1.29 mmol, 3.0 eq) were added to a suspension of 4-morpholinobenzoic acid (98.2 mg, 0.47 mmol, 1.1 eq) in DMF (2.0 mL), followed by intermediate SI-50 (170.0 mg, 0.43 mmol, 1.0 eq, TFA salt). The reaction mixture was stirred at 0° C. for 1 h. Upon completion, the reaction was poured onto ice-water (3 mL) and extracted with ethyl acetate (3×3 mL). The combined organic layers were washed with brine (3 mL), dried over Na₂SO₄, filtered and concentrated. The residue was purified by prep. HPLC (HCl conditions) to afford the title compound (44.5 mg, 19%) as a white solid. ¹H NMR (CDCl₃, 400 MHz) δ 7.87 (br, 2H), 7.58-7.25 (m, 5H), 7.24-7.13 (m, 2H), 4.68-4.42 (m, 2H), 4.41-4.09 (m, 5H), 4.02-3.76 (m, 3H), 3.53 (br, 4H), 3.46-3.08 (m, 3H), 2.16-1.47 (m, 6H). HRMS electrospray (m/z): [M+H]⁺ calcd for C₂₆H₃₃ClN₃O₃: 470.2205, found: 470.2202.

### Example S-30: Synthesis of N-benzyl-2-chloro-N-(1-(4-phenoxybenzoyl)azepan-4-yl)acetamide (BPK-30)

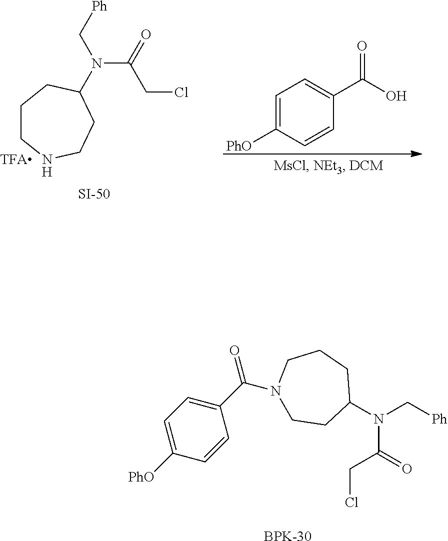

A solution of intermediate SI-50 (150.0 mg, 0.53 mmol, 1.0 eq), NEt₃ (370 μL, 2.67 mmol, 5.0 eq), and MsCl (82.7 μL, 1.1 mmol, 2.1 eq) in DCM (0.5 mL) was stirred at 0° C. for 30 min. 4-phenoxybenzoic acid (137.3 mg, 0.64 mmol, 1.2 eq) was then added and the mixture was stirred at 25° C. for another 1.5 h. Upon completion, the reaction was quenched with water (5 mL) and extracted with DCM (3×5 mL). The combined organic layers were dried over Na₂SO₄, filtered and concentrated. The residue was purified by prep. HPLC (FA conditions) to give the title compound (23.0 mg, 9%) as a white solid. ¹H NMR (CDCl₃, 400 MHz) δ 7.58-7.10 (m, 10H), 7.10-6.83 (m, 4H), 4.76-3.71 (m, 6H), 3.67-3.20 (m, 3H), 2.12-1.54 (m, 6H). HRMS electrospray (m/z): [M+H]⁺ calcd for C₂₈H₃₀ClN₂O₃: 477.1939, found: 477.1940.

### Example S-31: Synthesis of N-benzyl-2-chloro-N-(1-(1-phenylpiperidine-4-carbonyl)azepan-4-yl)acetamide (BPK-31)

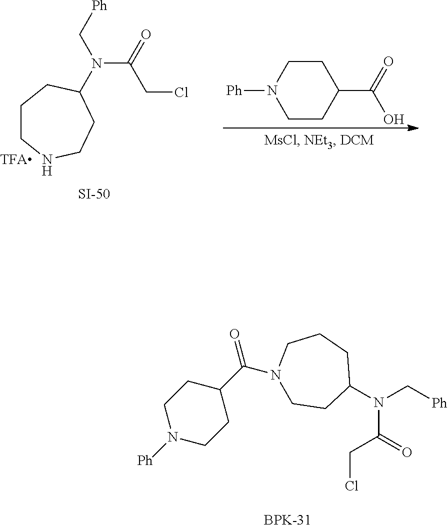

MsCl (74.2 μL, 0.96 mmol, 2.0 eq) was added to a solution of 1-phenylpiperidine-4-carboxylic acid (100.0 mg, 0.49 mmol, 1.0 eq) and intermediate SI-50 (164.2 mg, 0.58 mmol, 1.2 eq) in CH₃CN (2.0 mL) at 0° C. Subsequently, 3-methylpyridine (141.8 μL, 1.46 mmol, 3.0 eq) was added and the reaction mixture was stirred at 25° C. for 16 h. Upon completion, the reaction was quenched with water (2 mL) and concentrated. The residue was purified by prep. HPLC (HCl conditions) to give the title compound (8.0 mg, 4%) as a white solid. ¹H NMR (Methanol-d₄, 400 MHz) δ 7.69-7.50 (m, 5H), 7.43-7.18 (m, 5H), 4.74-4.53 (m, 2H), 4.50-4.34 (m, 1H), 4.17 (d, J=8.9 Hz, 1H), 4.00 (s, 1H), 3.85-3.35 (m, 8H), 3.25-3.03 (m, 1H), 2.31-1.53 (m, 10H). HRMS electrospray (m/z): [M+H]⁺ calcd for C₂₇H3₅C1N₃O₂: 468.2412, found: 468.2411.

### Example S-32: Synthesis of N-(1-(1H-benzo[d]imidazole-2-carbonyl)azepan-4-yl)-N-benzyl-2-chloroacetamide (BPK-32)

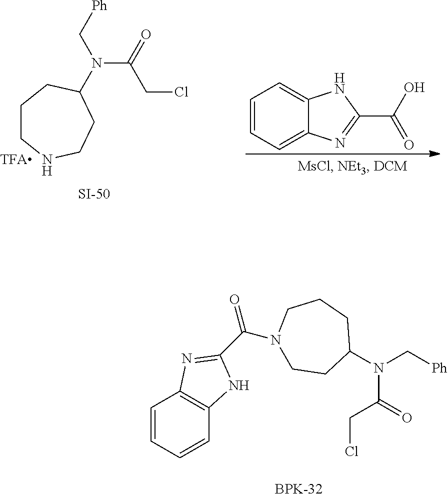

A solution of 1H-benzimidazole-2-carboxylic acid (104.0 mg, 0.64 mmol, 1.2 eq), NEt₃ (370 μL, 2.67 mmol, 5.0 eq), and MsCl (82.7 μL, 1.1 mmol, 2.1 eq) in DCM (0.5 mL) was stirred at 0° C. for 30 min. Intermediate SI-50 (150.0 mg, 0.53 mmol, 1.0 eq) was then added and the mixture was stirred at 25° C. for another 1.5 h. Upon completion, the reaction was quenched with water (5 mL) and extracted with DCM (3×5 mL). The combined organic layers were dried over Na₂SO₄, filtered and concentrated. The residue was purified by prep. HPLC (HCl conditions) to give the title compound (31.0 mg, 13%) as a white solid. ¹H NMR (CDCl₃, 400 MHz) δ 7.75-7.64 (m, 2H), 7.40-7.14 (m, 7H), 4.89-4.44 (m, 3H), 4.44-4.13 (m, 2H), 4.09-3.90 (m, 2H), 3.90-3.27 (m, 2H), 2.21-1.70 (m, 6H). HRMS electrospray (m/z): [M+H]⁺ calcd for C₂₃H2₆C1N₄O₂: 425.1739, found: 425.1736.

### Example S-33: Synthesis of N-(1-(1-naphthoyl)azepan-4-yl)-N-benzyl-2-chloroacetamide (BPK-33)

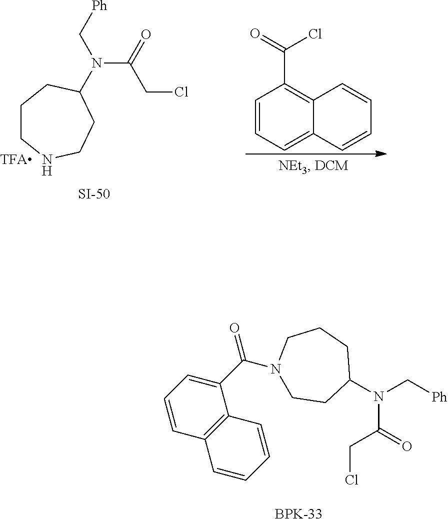

A solution of intermediate SI-50 (50.0 mg, 0.18 mmol, 1.0 eq), NEt₃ (74.1 μL, 0.53 mmol, 3.0 eq), and naphthalene-1-carbonylchloride (26.7 μL, 0.18 mmol, 1.0 eq) in DCM (1.0 mL) was stirred at 25° C. for 2 h. Upon completion, the reaction was quenched with water (5 mL) and extracted with DCM (3×5 mL). The combined organic layers were dried over Na₂SO₄, filtered and concentrated. The residue was purified by prep. HPLC (basic conditions) to give the title compound (9.0 mg, 11%) as a white solid. ¹H NMR (DMSO-d₆, 400 MHz) δ 8.03-7.91 (m, 2H), 7.79-7.08 (m, 10H), 4.73-4.16 (m, 4H), 4.14-3.78 (m, 2H), 3.26-2.80 (m, 3H), 2.12-1.87 (m, 2H), 1.88-1.63 (m, 2H), 1.62-1.42 (m, 2H). HRMS electrospray (m/z): [M+H]⁺ calcd for C₂₆H2₈C1N₂O₂: 435.1834, found: 435.1836.

### Example S-34: Synthesis of N-(1-acetylazepan-4-yl)-N-benzyl-2-chloroacetamide (BPK-34)

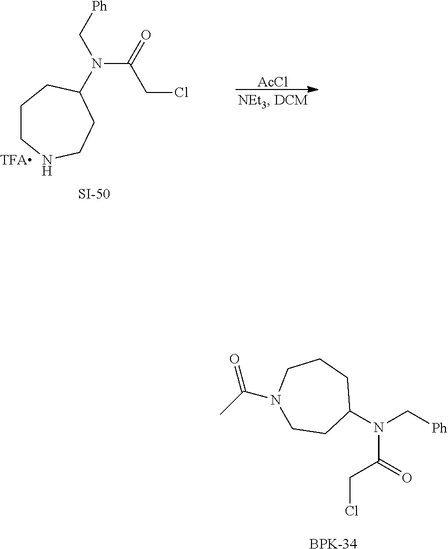

A solution of acetyl chloride (38.1 μL, 0.53 mmol, 1.5 eq), SI-50 (100.0 mg, 0.36 mmol, 1.0 eq), and NEt₃ (148.1 μL 1.07 mmol, 3.0 eq) in DCM (2.0 mL) was stirred at 25° C. for 2 h. Upon completion, the reaction was quenched with water (10 mL) and extracted with DCM (3×5 mL). The combined organic layers were dried over Na₂SO₄, filtered and concentrated. The residue was purified by prep. HPLC (basic conditions) to afford the title compound (7.0 mg, 6%) as colorless oil. ¹H NMR (DMSO-d₆, 400 MHz) δ 7.38 (t, J=7.7 Hz, 1H), 7.32-7.22 (m, 2H), 7.23-7.14 (m, 2H), 4.63-4.41 (m, 3H), 4.25-3.55 (m, 2H), 3.54-3.36 (m, 2H), 3.33-3.02 (m, 2H), 2.01-1.90 (m, 3H), 1.86-1.52 (m, 6H). HRMS electrospray (m z): [M+H]⁺ calcd for C₁₇H2₄C1N₂O₂: 323.1521, found: 323.1523.

### Example S-35: Synthesis of 2-chloro-N-(3-ethynylbenzyl)-N-(1-(4-morpholinobenzoyl)azepan-4-yl)acetamide (BPK-29-yne)

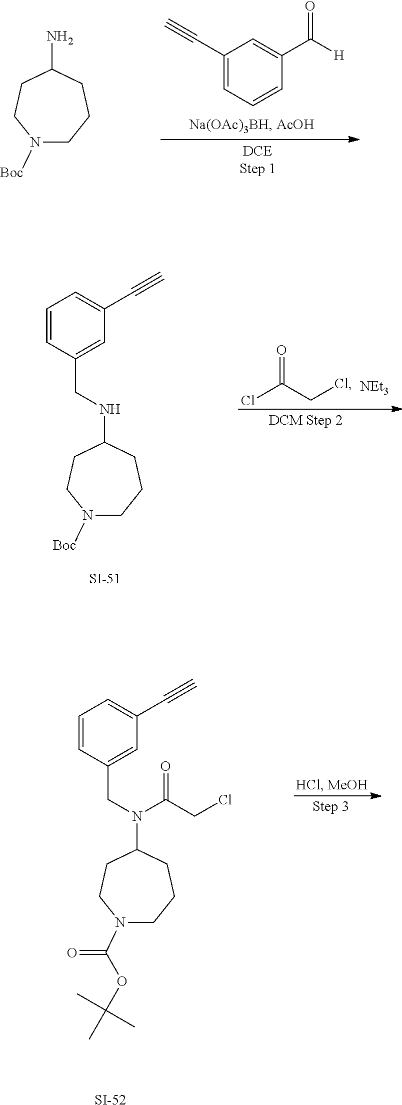

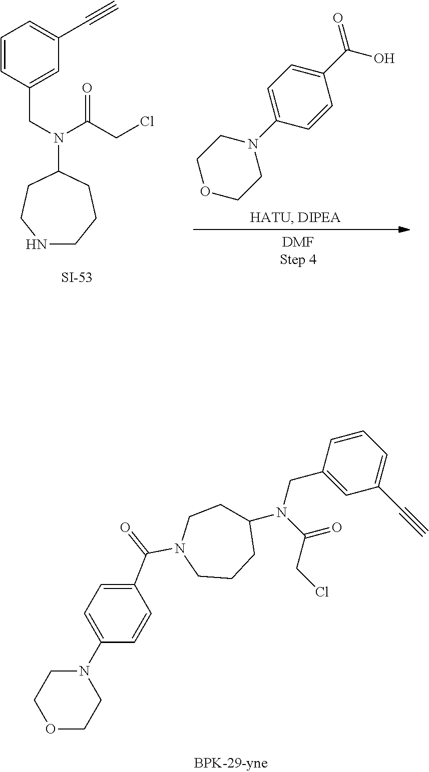

### Step 1.

AcOH (229 μL, 4 mmol, 2.0 eq) was added to a solution of tert-butyl 4-aminoazepane-1-carboxylate (428.6 mg, 2 mmol, 1.0 eq) and 3-ethynylbenzaldehyde (260.4 mg, 2.0 mmol, 1.0 eq) in MeOH (40 mL) at 25° C. The reaction was stirred for 30 min, cooled down to 0° C. after which NaBH₃CN (188.5 mg, 3.0 mmol, 1.5 eq) was added and the mixture was stirred at 25° C. for additional 1.5 h. Upon completion, the reaction was quenched by the addition of water (50 mL) and extracted with DCM (3×50 mL). The combined organic layers were dried over Na₂SO₄, filtered and concentrated to give crude compound SI-51 (654.1 mg) as pale yellow oil, which was used in step 2 without further purification.

### Step 2.

2-chloroacetyl chloride (200 μL, 2.5 mmol, 1.25 eq) was added dropwise to a solution of SI-51 (654.1 mg, 2 mmol, 1.0 eq) and NEt₃ (693.5 μL, 5 mmol, 2.5 eq) in anhydrous DCM (10 mL) at 0° C. The resulting mixture was stirred at room temperature for 1 h. Upon completion, the reaction was quenched by the addition of water (50 mL) and extracted with DCM (3×50 mL). The combined organic layers were dried over Na₂SO₄, filtered and concentrated to give compound SI-52 as pale yellow oil (875.8 mg, crude), which was used in the next step without additional purification.

### Step 3.

Methanolic HCl (7.8 mL, 6.2 mmol, 3.1 eq, 1.25 M) was added to a solution of compound SI-52 (857.8 mg, crude from 2 mmol scale reaction, 1.0 eq) and the mixture was stirred at 25° C. overnight. Upon completion, methanol was removed and the title compound was passed through a silica gel plug (0-10% MeOH/CH₂Cl₂) to afford SI-53 (504.4 mg) as an off-white solid, which was used in the next step without additional purification.

### Step 4.

HATU (66.1 mg, 0.18 mmol, 1.25 eq) and DIEA (24.4 μL, 0.14 mmol, 1.0 eq) were added to a suspension of 4-morpholinobenzoic acid (29.0 mg, 0.14 mmol, 1.0 eq) in DMF (1.0 mL) and the reaction was stirred for 5 min at ambient temperature. A solution of SI-53 (50.0 mg, 0.15 mmol, 1.1 eq) and DIEA (48.4 μL, 0.28 mmol, 2.0 eq) was then added dropwise and the reaction mixture was stirred for an additional 1 h. Upon completion, the reaction was quenched by the addition of water (5 mL) and extracted with ethyl acetate (3×5 mL). The combined organic layers were washed with brine (3 mL), dried over Na₂SO₄, filtered and concentrated. The residue was purified by prep. TLC (EtOAc), followed by trituration with cold Et₂O to afford the title compound (21.6 mg, 31%) as a white solid. ¹H NMR (D₂O, 400 MHz) δ 7.47-7.14 (m, 6H), 6.97 (br, 2H), 4.74-4.32 (m, 3H), 4.17 (s, 1H), 4.13-3.91 (m, 1H), 3.91-3.72 (m, 5H), 3.74-3.33 (m, 4H), 3.21 (br, 4H), 2.18-1.65 (m, 6H). HRMS electrospray (m/z): [M+H]⁺ calcd for C₂₈H3₂C1N₃O₃: 494.2204, found: 494.2211.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

LENGTHY TABLES
The patent application contains a lengthy table section. A copy of the table is available in electronic form from the USPTO web site (http://seqdata.uspto.gov/?pageRequest=docDetail&DocID=US20200278355A1). An electronic copy of the table will also be available from the USPTO upon request and payment of the fee set forth in 37 CFR 1.19(b)(3).

What is claimed is:

1. A protein-probe adduct wherein the probe binds to a cysteine residue illustrated in Tables 1A, 2, 3A, and 4; wherein the probe has a structure represented by Formula (I): 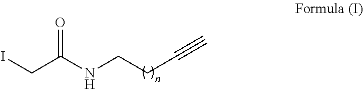
wherein,
n is 0-8.

2. The protein-probe adduct of claim 1, wherein the protein is ubiquitin carboxyl-terminal hydrolase 7 (USP7) and the cysteine residue is C223, wherein the numbering of the amino acid position corresponds to the amino acid position with the UniProt Identifier Q93009.

3. The protein-probe adduct of claim 1, wherein the protein is B-cell lymphoma/leukemia 10 (BCL10) and the cysteine residue is C119 or C122, wherein the numberings of the amino acid positions correspond to the amino acid positions with the UniProt Identifier O95999.

4. The protein-probe adduct of claim 1, wherein the protein is RAF proto-oncogene serine/threonine-protein kinase (RAF1) and the cysteine residue is C637, wherein the numbering of the amino acid position corresponds to the amino acid position with the UniProt Identifier P04049.

5. The protein-probe adduct of claim 1, wherein the protein is nuclear receptor subfamily 2 group F member 6 (NR2F6) and the cysteine residue is C203 or C316, wherein the numberings of the amino acid positions correspond to the amino acid positions with the UniProt Identifier P10588.

6. The protein-probe adduct of claim 1, wherein the protein is DNA-binding protein inhibitor ID-1 (ID1) and the cysteine residue is C17, wherein the numbering of the amino acid position corresponds to the amino acid position with the UniProt Identifier P41134.

7. The protein-probe adduct of claim 1, wherein the protein is Fragile X mental retardation syndrome-related protein 1 (FXR1) and the cysteine residue is C99, wherein the numbering of the amino acid position corresponds to the amino acid position with the UniProt Identifier P51114.

8. The protein-probe adduct of claim 1, wherein the protein is Mitogen-activated protein kinase kinase kinase kinase 4 (MAP4K4) and the cysteine residue is C883, wherein the numbering of the amino acid position corresponds to the amino acid position with the UniProt Identifier O95819.

9. The protein-probe adduct of claim 1, wherein the protein is Cathepsin B (CTSB) and the cysteine residue is C105 or C108, wherein the numberings of the amino acid positions correspond to the amino acid positions with the UniProt Identifier P07858.

10. The protein-probe adduct of claim 1, wherein the protein is integrin beta-4 (ITGB4) and the cysteine residue is C245 or C288, wherein the numberings of the amino acid positions correspond to the amino acid positions with the UniProt Identifier P16144.

11. The protein-probe adduct of claim 1, wherein the protein is TFIIH basal transcription factor complex helicase (ERCC2) and the cysteine residue is C663, wherein the numbering of the amino acid position corresponds to the amino acid position with the UniProt Identifier P18074.

12. The protein-probe adduct of claim 1, wherein the protein is nuclear receptor subfamily 4 group A member 1 (NR4A1) and the cysteine residue is C551, wherein the numbering of the amino acid position corresponds to the amino acid position with the UniProt Identifier P22736.

13. The protein-probe adduct of claim 1, wherein the protein is cytidine deaminase (CDA) and the cysteine residue is C8, wherein the numbering of the amino acid position corresponds to the amino acid position with the UniProt Identifier P32320.

14. The protein-probe adduct of claim 1, wherein the protein is sterol O-acyltransferase 1 (SOAT1) and the cysteine residue is C92, wherein the numbering of the amino acid position corresponds to the amino acid position with the UniProt Identifier P35610.

15. The protein-probe adduct of claim 1, wherein the protein is DNA mismatch repair protein Msh6 (MSH6) and the cysteine residue is C615, wherein the numbering of the amino acid position corresponds to the amino acid position with the UniProt Identifier P52701.

16. The protein-probe adduct of claim 1, wherein the protein is telomeric repeat-binding factor 1 (TERF1) and the cysteine residue is C118, wherein the numbering of the amino acid position corresponds to the amino acid position with the UniProt Identifier P54274.

17. The protein-probe adduct of claim 1, wherein the protein is NEDD8-conjugating enzyme Ubc12 (UBE2M) and the cysteine residue is C47, wherein the numbering of the amino acid position corresponds to the amino acid position with the UniProt Identifier P61081.

18. The protein-probe adduct of claim 1, wherein the protein is E3 ubiquitin-protein ligase TRIP12 (TRIP12) and the cysteine residue is C535, wherein the numbering of the amino acid position corresponds to the amino acid position with the UniProt Identifier Q14669.

19. The protein-probe adduct of claim 1, wherein the protein is ubiquitin carboxyl-terminal hydrolase 10 (USP10) and the cysteine residue is C94, wherein the numbering of the amino acid position corresponds to the amino acid position with the UniProt Identifier Q14694.

20. The protein-probe adduct of claim 1, wherein the protein is ubiquitin carboxyl-terminal hydrolase 30 (USP30) and the cysteine residue is C142, wherein the numbering of the amino acid position corresponds to the amino acid position with the UniProt Identifier Q70CQ3.

21. The protein-probe adduct of claim 1, wherein the protein is nucleus accumbens-associated protein 1 (NACC1) and the cysteine residue is C301, wherein the numbering of the amino acid position corresponds to the amino acid position with the UniProt Identifier Q96RE7.

22. The protein-probe adduct of claim 1, wherein the protein is lymphoid-specific helicase (HELLS) and the cysteine residue is C277 or C836, wherein the numberings of the amino acid positions correspond to the amino acid positions with the UniProt Identifier Q9NRZ9.

23. The protein-probe adduct of claim 1, wherein n is 3.

24. A synthetic ligand that inhibits a covalent interaction between a protein and a probe, wherein in the absence of the synthetic ligand, the probe binds to a cysteine residue illustrated in Tables 1A, 2, 3A, and 4; and wherein the probe has a structure represented by Formula (I): 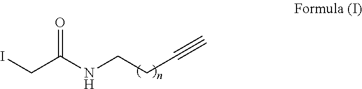
wherein
n is 0-8.
